(19) United States
(12) Patent Application Publication
(10) Pub. No.: US 2024/0391862 A1
(43) Pub. Date: Nov. 28, 2024

(54) (1,4,5-TRISUBSTITUTED-1H-PYRAZOL-3-YL)OXY-2-ALKOXY ALKYL ACIDS AND THEIR DERIVATIVES, THEIR SALTS AND THEIR USE AS HERBICIDAL AGENTS

(71) Applicant: Bayer AKtiengesellschaft, Leverkusen (DE)

(72) Inventors: Estella BUSCATO, Leverkusen (DE); Hendrik HELMKE, Leverkusen (DE); Harald JAKOBI, Leverkusen (DE); Thomas MÜLLER, Leverkusen (DE); Birgit BOLLENBACH-WAHL, Leverkusen (DE); Jan DITTGEN, Leverkusen (DE); Elmar GATZWEILER, Leverkusen (DE); Guido BOJACK, Leverkusen (DE)

(21) Appl. No.: 18/570,398

(22) PCT Filed: Jun. 23, 2022
(86) PCT No.: PCT/EP2022/067124

§ 371 Date: Dec. 14, 2023

(30) Foreign Application Priority Data
Jun. 25, 2021 (EP) 21181914.9

(51) Int. Cl. C07C 59/40; A01N 37/06; A01N 43/40; A01N 43/60; C07D 213/61; C07D 213/74; C07D 213/77; C07D 401/04; C07D 401/12; C07D 401/14

(52) U.S. Cl. CPC C07C 59/40; A01N 37/06; A01N 43/40; A01N 43/60; C07D 213/61; C07D 213/74; C07D 213/77; C07D 401/04; C07D 401/12; C07D 401/14

(57) ABSTRACT

The present invention relates to novel, herbicidally active (1,4,5-trisubstituted 1H-pyrazol-3-yl)oxy-2-alkoxyalkyl acids and their derivatives of the general formula (I) and their agrochemically compatible salts, N-oxides, hydrates, and hydrates of the salts and N-oxides, to processes for preparation thereof and to the use thereof for control of broadleaved weeds and weed grasses in crops of useful plants, and for general control of broadleaved weeds and weed grasses in areas of the environment where plant growth is troublesome.

The derivatives of the (1,4,5-trisubstituted 1H-pyrazol-3-yl)oxy-2-alkoxyalkyl acids include in particular their esters and/or amides.

The present invention relates to novel, herbicidally active (1,4,5-trisubstituted 1H-pyrazol-3-yl)oxy-2-alkoxyalkyl acids and their derivatives of the general formula (I) and their agrochemically compatible salts, N-oxides, hydrates, and hydrates of the salts and N-oxides, to processes for preparation thereof and to the use thereof for control of broadleaved weeds and weed grasses in crops of useful plants, and for general control of broadleaved weeds and weed grasses in areas of the environment where plant growth is troublesome.

The derivatives of the (1,4,5-trisubstituted 1H-pyrazol-3-yl)oxy-2-alkoxyalkyl acids include in particular their esters, salts and/or amides.

The prior art discloses biological effects of substituted 1,5-diphenylpyrazolyl-3-oxoacetic acids and processes for preparing these compounds. DE 2828529 A1 describes the preparation and the lipid-lowering action of 1,5-diphenylpyrazolyl-3-oxoacetic acids.

CN 101284815 discloses 1,5-diphenylpyrazolyl-3-oxoacetic acids as bactericidally active agrochemicals. Journal of Heterocyclic Chemistry (2012), 49(6), 1370-1375 describes further syntheses and the fungicidal action of 1,5-diphenylpyrazolyl-3-oxoacetic acids.

WO 2008/083233 A2 describes 1,5-diphenylpyrazolyl-3-oxyalkyl acids substituted in the 4 position of the pyrazole and derivatives thereof as substances that are suitable for breaking up cell aggregates. Ethyl [(4-chloro-1,5-diphenyl-1H-pyrazol-3-yl)oxy]acetate is specifically disclosed.

WO2020/245044 A1 describes 1-phenyl-5-azinylpyrazolyl-3-oxyalkyl acids having substitution in the 1 position of the pyrazole, and derivatives thereof, as substances having herbicidal action.

The inventive (1,4,5-trisubstituted 1H-pyrazol-3-yl)oxy-2-alkoxyalkyl acids and derivatives thereof differ from the already known 1,5-diphenylpyrazolyl-3-oxoacetic acids by the specific R² radical=methoxy, ethoxy.

In addition, the synthesis of some 4-chloro-1,5-diphenylpyrazolyl-3-oxyacetic acids and ethyl esters thereof is described in European Journal of Organic Chemistry (2011), 2011 (27), 5323-5330.

It is an object of the present invention to provide novel pyrazole derivatives, namely (1,4,5-trisubstituted 1H-pyrazol-3-yl)oxy-2-alkoxyalkyl acids and derivatives thereof, which can be used as herbicides or plant growth regulators, having good herbicidal action and a broad spectrum of efficacy against harmful plants and/or having high selectivity in crops of useful plants.

The object is achieved by (1,4,5-trisubstituted 1H-pyrazol-3-yl)oxy-2-alkoxyalkyl acids wherein the substituent R²=methoxy or ethoxy and which feature very good herbicidal action and additionally also have very good selectivities.

Surprisingly, these compounds are highly effective against a broad range of economically important weed grasses and broadleaved weeds. At the same time, the compounds exhibit good crop plant compatibility. Therefore, given good efficacy against harmful plants, they can be used selectively in crop plants.

The present invention therefore provides (1,4,5-trisubstituted 1H-pyrazol-3-yl)oxy-2-alkoxyalkyl acids, and derivatives thereof, of general formula (I)

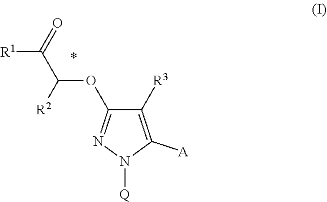

and their agrochemically acceptable salts, N-oxides, hydrates, and hydrates of the salts and N-oxides, where
A is selected from the group consisting of A1, A2 and A3

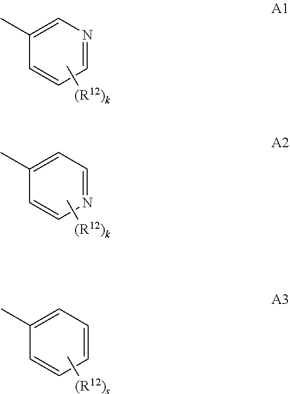

Q is selected from the group consisting of Q1-Q16:

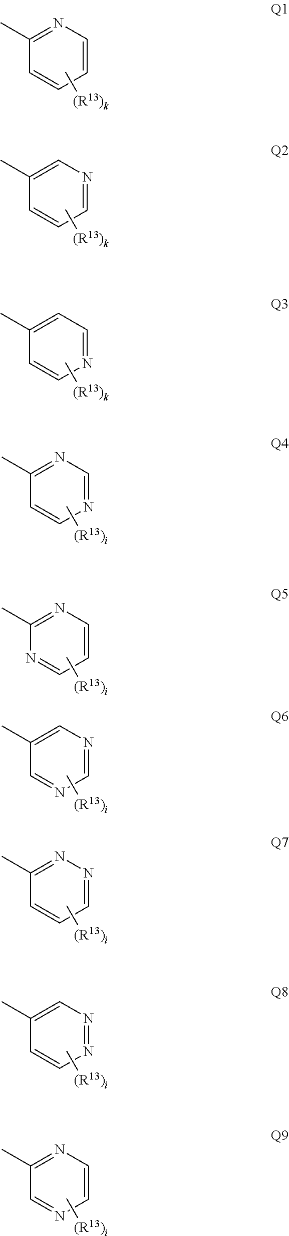

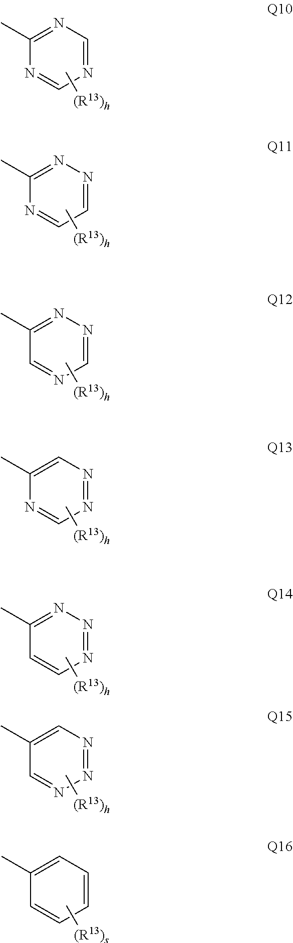

R¹ is OR^{1a}, NR⁹R¹⁰;
R^{1a} is hydrogen or
   is (C₁-C₆)-alkyl, (C₃-C₆)-cycloalkyl, which is unsubstituted or in each case independently substituted by “m” radicals selected from the group consisting of COOR⁵, halogen, (C₁-C₆)-alkyl, (C₁-C₆)-haloalkyl, (C₃-C₆)-cycloalkyl, (C₁-C₆)-alkoxy, cyano and nitro or
   is (C₂-C₄)-alkenyl, (C₂-C₄)-alkynyl or
   is (C₁-C₆)-alkyl-SO—(C₁-C₆)-alkyl-, (C₁-C₆)-alkyl-SO₂—(C₁-C₆)-alkyl- or
   is heterocyclyl, heteroaryl, aryl or
   is heterocyclyl-(C₁-C₄)-alkyl-, heteroaryl-(C₁-C₄)-alkyl-, aryl-(C₁-C₄)-alkyl-, which is unsubstituted or in each case independently substituted by “m” radicals selected from the group consisting of halogen, (C₁-C₆)-alkyl, (C₁-C₆)-haloalkyl;
R⁹ is hydrogen, (C₁-C₁₂)-alkyl;
R¹⁰ is hydrogen, aryl, heteroaryl, heterocyclyl, (C₁-C₁₂)-alkyl, (C₃-C₈)-cycloalkyl, (C₃-C₈)-cycloalkyl-(C₁-C₇)-alkyl-, (C₂-C₁₂)-alkenyl, (C₈-C₇)-cycloalkenyl, (C₂-C₁₂)-alkynyl, S(O)ₙR⁵, cyano, OR⁵, SO₂NR⁶R⁷, CO₂R⁸, COR^{B}, where the abovementioned alkyl, cycloalkyl, alkenyl, cycloalkenyl and alkynyl radicals are unsubstituted or each independently substituted by “m” radicals selected from the group consisting of optionally mono- or polysubstituted aryl, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or
R⁹ and R¹⁰ together with the nitrogen atom to which they are bonded form a saturated or partly or fully unsaturated five-, six- or seven-membered ring which is optionally substituted by “m” radicals from the group consisting of halogen, (C₁-C₆)-alkyl, (C₁-C₆)-haloalkyl, OR⁵, S(O)ₙR⁵, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl, aryl;
R⁶ is hydrogen, (C₁-C₆)-alkyl, (C₃-C₆)-cycloalkyl, (C₁-C₆)-haloalkyl, aryl;
R⁷ is hydrogen, (C₁-C₆)-alkyl, (C₃-C₆)-cycloalkyl, (C₃-C₄)-alkenyl, (C₃-C₄)-alkynyl;
R⁸ is hydrogen, (C₁-C₆)-alkyl, (C₃-C₆)-cycloalkyl, (C₃-C₄)-alkenyl, (C₁-C₆)-alkyl-COO(C₁-C₂)-alkyl or (C₃-C₄)-alkynyl;
R² is methoxy, ethoxy;
R³ is halogen, cyano, isocyano, nitro, (C₁-C₆)-alkyl, (C₃-C₆)-cycloalkyl, (C₁-C₆)-haloalkyl, (C₃-C₆)-halocycloalkyl, (C₁-C₆)-alkylcarbonyl-, (C₁-C₆)-haloalkylcarbonyl-, (C₁-C₆)-alkyloxycarbonyl-, (C₂-C₃)-alkenyl, (C₂-C₃)-haloalkenyl, (C₂-C₃)-alkynyl, (C₂-C₃)-haloalkynyl, (C₁-C₆)-alkyl-S(O)ₙ and (C₁-C₆)-haloalkyl-S(O)ₙ, CHO and NH₂;
R¹² is halogen, cyano, nitro, (C₁-C₆)-alkyl, (C₁-C₆)-haloalkyl;
R¹³ is halogen, cyano, nitro, (C₁-C₆)-alkyl, (C₁-C₆)-haloalkyl, (C₁-C₆)-alkylcarbonyl, (C₁-C₆)-haloalkylcarbonyl, (C₁-C₆)-alkoxycarbonyl, (C₁-C₆)-alkoxy, (C₁-C₆)-haloalkoxy, (C₁-C₆)-alkylS(O)ₙ, (C₂-C₃)-alkenyl, (C₂-C₃)-haloalkenyl, (C₂-C₃)-alkynyl, (C₂-C₃)-haloalkynyl;
h is 0, 1 or 2;
i is 0, 1, 2 or 3;
k is 0, 1, 2, 3 or 4;
m is 0, 1 or 2;
n is 0, 1 or 2;
o is 0, 1 or 2;
p is 0 or 1;
q is 0 or 1;
r is 3, 4, 5 or 6;
s is 0, 1, 2, 3, 4 or 5.

There follows a description of preferred, particularly preferred and very particularly preferred definitions of each of the individual substituents.

This results in various embodiments for the compound of the general formula (I).

Preference is given to compounds of the general formula (I) in which
A is A1-1, A1-2, A1-3, A1-4, A2-1, A3-1, A3-2, A3-3, A3-4 and A3-5

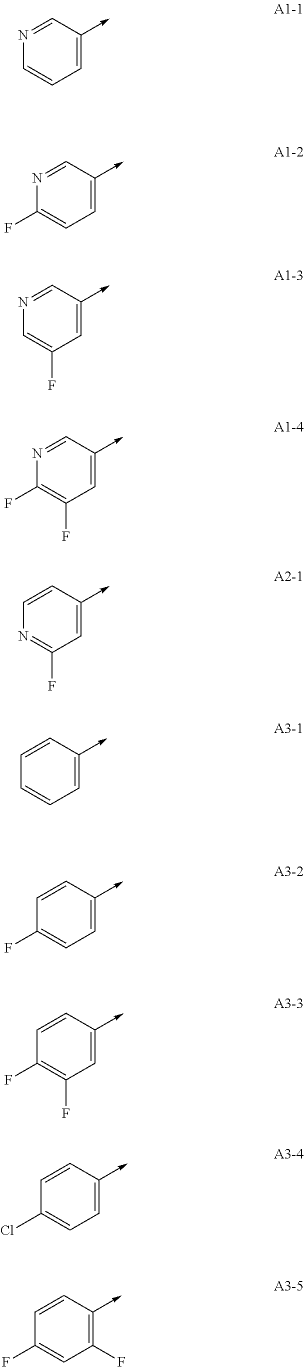

Q is selected from the group consisting of Q, Q2, Q9 and Q16

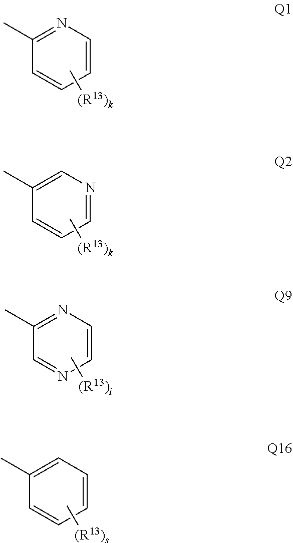

R¹ is OR^{1a}, NR⁹R¹⁰,
R^{1a} is hydrogen or
   is (C₁-C₆)-alkyl, (C₃-C₆)-cycloalkyl, which is unsubstituted or in each case independently substituted by “m” radicals selected from the group consisting of COOR⁵, halogen, (C₁-C₆)-alkyl, (C₁-C₆)-haloalkyl, (C₃-C₆)-cycloalkyl, (C₁-C₆)-alkoxy, cyano and nitro or
   is (C₂-C₄)-alkenyl, (C₂-C₄)-alkynyl or
   is (C₁-C₆)-alkyl-SO—(C₁-C₆)-alkyl-, (C₁-C₆)-alkyl-SO₂—(C₁-C₆)-alkyl-, aryl-(C₁-C₄)-alkyl-, which is unsubstituted or in each case independently substituted by “m” radicals selected from the group consisting of halogen, (C₁-C₆)-alkyl, (C₁-C₆)-haloalkyl;
R⁹ is hydrogen, (C₁-C₆)-alkyl;
R¹⁰ is hydrogen, phenyl, (C₁-C₆)-alkyl, (C₃-C₆)-cycloalkyl, (C₃-C₆)-cycloalkyl-(C₁-C₄)-alkyl-, (C₂-C₄)-alkenyl, (C₅-C₇)-cycloalkenyl, (C₂-C₄)-alkynyl, S(O)ₙR⁵, cyano, OR⁵, SO₂NR⁶R⁷, CO₂R⁸, COR⁸, where the abovementioned alkyl, cycloalkyl, alkenyl, cycloalkenyl and alkynyl radicals are unsubstituted or each independently substituted by “m” radicals selected from the group consisting of optionally mono- or polysubstituted phenyl, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸; or
R⁹ and R¹⁰ together with the nitrogen atom to which they are bonded form a saturated or partly or fully unsaturated five-, six- or seven-membered ring which is optionally substituted by “m” radicals from the group consisting of halogen, (C₁-C₆)-alkyl, (C₁-C₆)-haloalkyl, OR⁵, S(O)ₙR⁵, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl or phenyl;
R⁶ is hydrogen, (C₁-C₆)-alkyl, (C₃-C₆)-cycloalkyl, (C₁-C₆)-haloalkyl or phenyl;
R⁷ is hydrogen, (C₁-C₆)-alkyl, (C₃-C₆)-cycloalkyl, (C₃-C₄)-alkenyl or (C₃-C₄)-alkynyl;
R⁸ is hydrogen, (C₁-C₆)-alkyl, (C₃-C₆)-cycloalkyl, (C₃-C₄)-alkenyl or (C₃-C₄)-alkynyl;
R² is methoxy, ethoxy;
R³ is halogen, cyano, isocyano, nitro, (C₁-C₄)-alkyl, (C₃-C₆)-cycloalkyl, (C₁-C₆)-haloalkyl, (C₃-C₆)-halocycloalkyl, (C₂-C₃)-alkenyl, (C₂-C₃)-haloalkenyl, (C₂-C₃)-alkynyl, (C₂-C₃)-haloalkynyl;
R¹³ is halogen, cyano, nitro, (C₁-C₆)-alkyl, (C₁-C₆)-haloalkyl, (C₁-C₆)-alkoxy, (C₁-C₆)-haloalkoxy, (C₁-C₆)-alkylS(O)ₙ, (C₂-C₃)-alkenyl, (C₂-C₃)-haloalkenyl, (C₂-C₃)-alkynyl, (C₂-C₃)-haloalkynyl;
i is 0, 1 or 2;
k is 0, 1, 2, 3 or 4;
m is 0, 1, 2;
n is 0, 1, 2;
o is 0, 1, 2;
p is 0 or 1;
q is 0 or 1;
r is 3, 4, 5 or 6;
s is 0, 1, 2, 4, 5.

Particular preference is given to compounds of the general formula (I) in which
A is A1-1, A1-2, A1-3, A1-4, A2-1, A3-1, A3-2, A3-3, A3-4 and A3-5

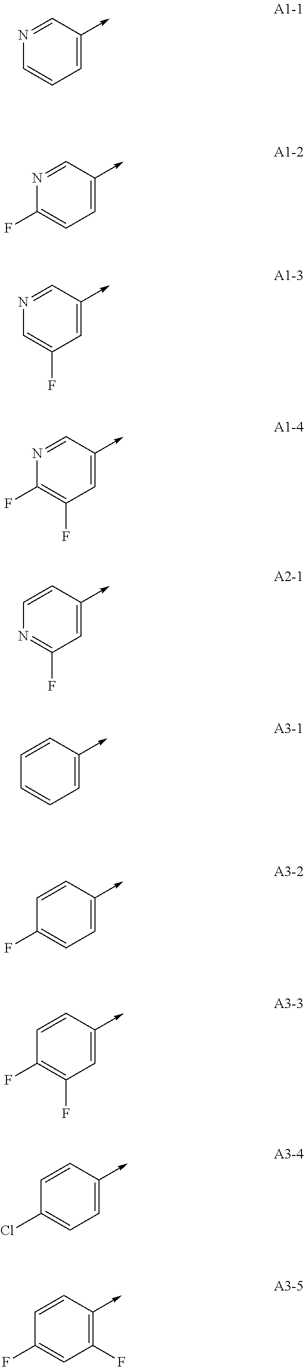

Q is selected from the group consisting of Q1, Q2, Q9 and Q16

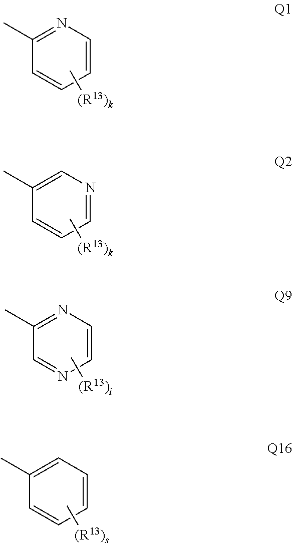

R¹ is OR^{1a}, NR⁹R¹⁰;
R^{1a} is hydrogen or
   is (C₁-C₆)-alkyl, (C₃-C₆)-cycloalkyl, which is unsubstituted or in each case independently substituted by “m” radicals selected from the group consisting of COOR⁵, halogen, (C₁-C₄)-alkyl, (C₁-C₄)-haloalkyl or
   is aryl-(C₁-C₄)-alkyl- which is unsubstituted or in each case independently substituted by “m” radicals selected from the group consisting of halogen, (C₁-C₄)-alkyl, (C₁-C₄)-haloalkyl;
R⁹ is hydrogen;
R¹⁰ is (C₁-C₄)-alkyl, S(O)ₙR⁵, SO₂NR⁶R⁷, CO₂R⁸, where the abovementioned radicals are unsubstituted or each independently substituted by “m” radicals selected from the group consisting of phenyl, S(O)ₙR⁵, SO₂NR⁶R⁷, CO₂R⁸, NR⁶CO₂R⁸;
R⁵ is ethyl, methyl, CF₃ or CH₂CF₃;
R⁶ is hydrogen;
R⁷ is hydrogen, methyl or ethyl;
R⁸ is methyl or ethyl;
R² is methoxy, ethoxy;
R³ is halogen, cyano, nitro, (C₁-C₄)-alkyl, (C₃-C₆)-cycloalkyl, (C₁-C₄)-haloalkyl, (C₃-C₆)-halocycloalkyl;
R¹³ is fluorine, chlorine, bromine, cyano, methyl, ethyl, methoxy, ethoxy, CF₃, OCF₃;
i is 0, 1 or 2;
k is 0, 1 or 2;
m is 0, 1 or 2;
n is 0, 1 or 2;
s is 0, 1 or 2.

Very particular preference is given to compounds of the general formula (I) in which A is selected from the group consisting of
A is A1-1, A1-2, A1-3, A1-4, A2-1, A3-1, A3-2, A3-3, A3-4 and A3-5

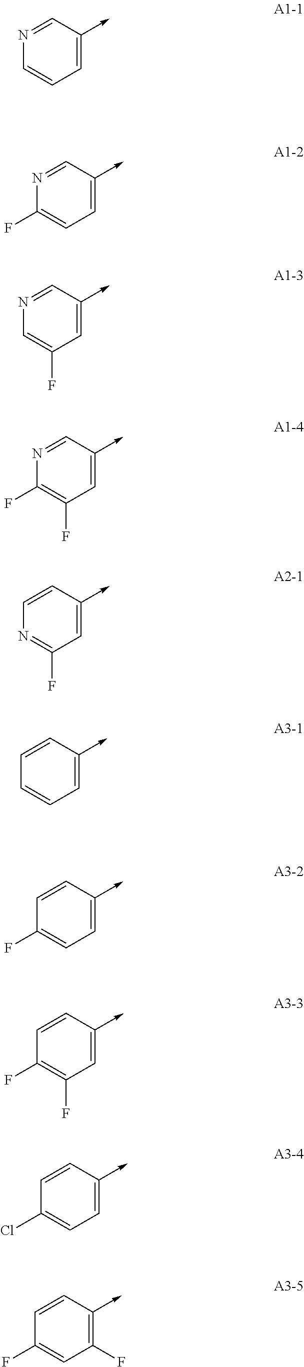

Q is selected from the group consisting of Q1, Q9 and Q16

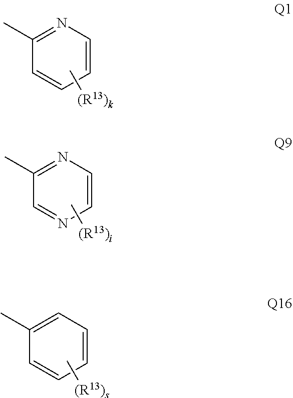

R¹ is OR^{1a},
R^{1a} is hydrogen, ethyl, methyl, —CH₂CH(CH₃)COOmethyl, —CH₂CH₂COOmethyl;
R² is ethoxy, methoxy;
R³ is chlorine, bromine, iodine, cyano, cyclopropyl, CF₂CF₃, CHF₂ or CF₃;
R¹³ is fluorine, chlorine, methyl, MeS(O), MeS or CF₃;
i is 0, 1 or 2;
k is 0, 1 or 2;
s is 0, 1 or 2.

The present invention further provides compounds of the formula (Is)

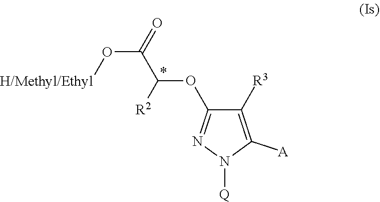

where the above-described definitions are applicable, including all preferred, particularly preferred and very particularly preferred definitions.

The present invention further provides compounds of the formula (It)

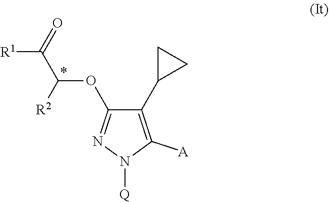

where the above-described definitions are applicable, including all preferred, particularly preferred and very particularly preferred definitions.

The present invention further provides compounds of the formula (Iu)

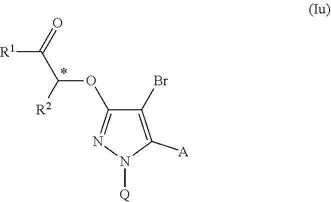

where the above-described definitions are applicable, including all preferred, particularly preferred and very particularly preferred definitions.

The present invention further provides compounds of the formula (Iv)

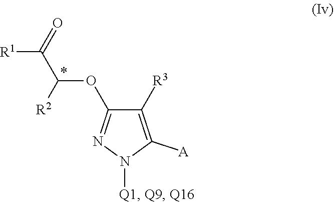

where the above-described definitions are applicable, including all preferred, particularly preferred and very particularly preferred definitions.

The present invention further provides compounds of the formula (Iw)

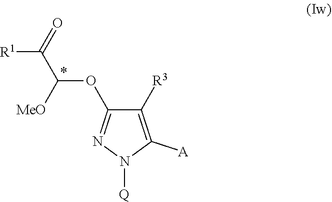

where the above-described definitions are applicable, including all preferred, particularly preferred and very particularly preferred definitions.

The present invention further provides compounds of the formula (Ix)

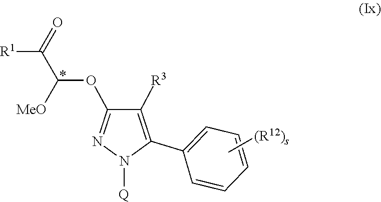

where the above-described definitions are applicable, including all preferred, particularly preferred and very particularly preferred definitions.

The present invention further provides compounds of the formula (Iy)

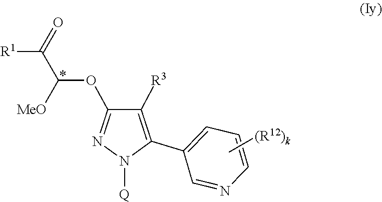

where the above-described definitions are applicable, including all preferred, particularly preferred and very particularly preferred definitions.

The present invention further provides compounds of the formula (Iz)

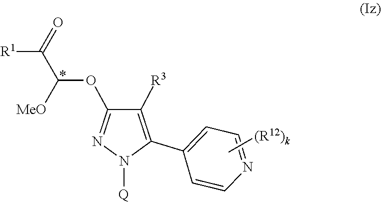

where the above-described definitions are applicable, including all preferred, particularly preferred and very particularly preferred definitions.

The present invention further provides compounds of the formula (V)

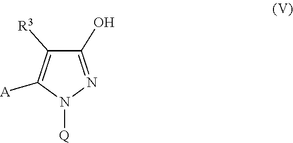

where the above-described definitions are applicable, including all preferred, particularly preferred and very particularly preferred definitions.

In all the formulae specified hereinafter, the substituents and symbols have the same meaning as described in formula (I), unless defined differently.

Not encompassed are combinations which contravene the laws of nature and which the person skilled in the art would therefore rule out on the basis of their knowledge.

Alkyl denotes saturated straight-chain or branched hydrocarbyl radicals having the number of carbon atoms specified in each case, e.g. C₁-C₁₂-alkyl, preferably C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Halogen-substituted alkyl denotes straight-chain or branched alkyl groups where some or all of the hydrogen atoms in these groups may be replaced by halogen atoms, e.g. C₁-C₆-haloalkyl, preferably C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

Alkenyl denotes unsaturated straight-chain or branched hydrocarbyl radicals having the number of carbon atoms stated in each case and one double bond in any position, for example C₂-C₈-alkenyl, preferably C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl denotes straight-chain or branched hydrocarbyl radicals having the number of carbon atoms specified in each case and one triple bond in any position, e.g. C₂-C₁₂-alkynyl, preferably C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Cycloalkyl means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene.

In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl(norbornyl), adamantan-1-yl and adamantan-2-yl.

In the case of substituted cycloalkyl, spirocyclic aliphatic systems are also included, for example spiro[2.2]pent-1-yl, spiro[2.3]hex-1-yl and spiro[2.3]hex-4-yl, 3-spiro[2.3]hex-5-yl.

Cycloalkenyl denotes a carbocyclic, nonaromatic, partially unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

Alkoxy means saturated straight-chain or branched alkoxy radicals having the number of carbon atoms specified in each case, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Halogen-substituted alkoxy denotes straight-chain or branched alkoxy radicals having the number of carbon atoms specified in each case, where some or all of the hydrogen atoms in these groups may be replaced by halogen atoms as specified above, e.g. C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-1,2-difluoroethoxy, 2,2-dichloro-2-fluoroethoxy, 2,2,2-trichloroethoxy, pentafluoroethoxy and 1,1,1-trifluoroprop-2-oxy.

Aryl means a phenyl which is optionally substituted by 0-5 radicals from the group consisting of fluorine, chlorine, bromine, iodine, cyano, hydroxy, (C₁-C₃)-alkyl, (C₁-C₃)-alkoxy, (C₃-C₄)-cycloalkyl, (C₂-C₃)-alkenyl or (C₂-C₃)-alkynyl.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, for example 1-oxa-5-azaspiro[2.3]hexyl. Unless defined differently, the heterocyclic ring preferably contains 3 to 9 ring atoms, especially 3 to 6 ring atoms, and one or more, preferably 1 to 4, especially 1, 2 or 3, heteroatoms in the heterocyclic ring, preferably from the group of N, O and S, but no two oxygen atoms should be directly adjacent, for example with one heteroatom from the group of N, O and S: 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or 3- or 4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partly or fully hydrogenated heterocyclic radical having two heteroatoms from the group of N, O and S, for example 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyridazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl. Structural examples of heterocycles which are optionally substituted further are also listed below:

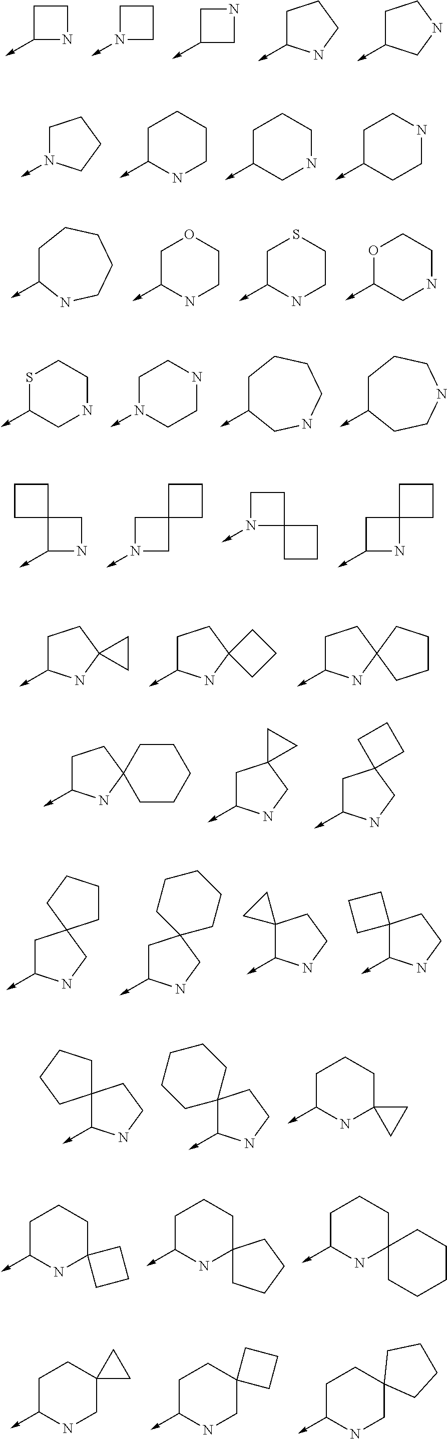

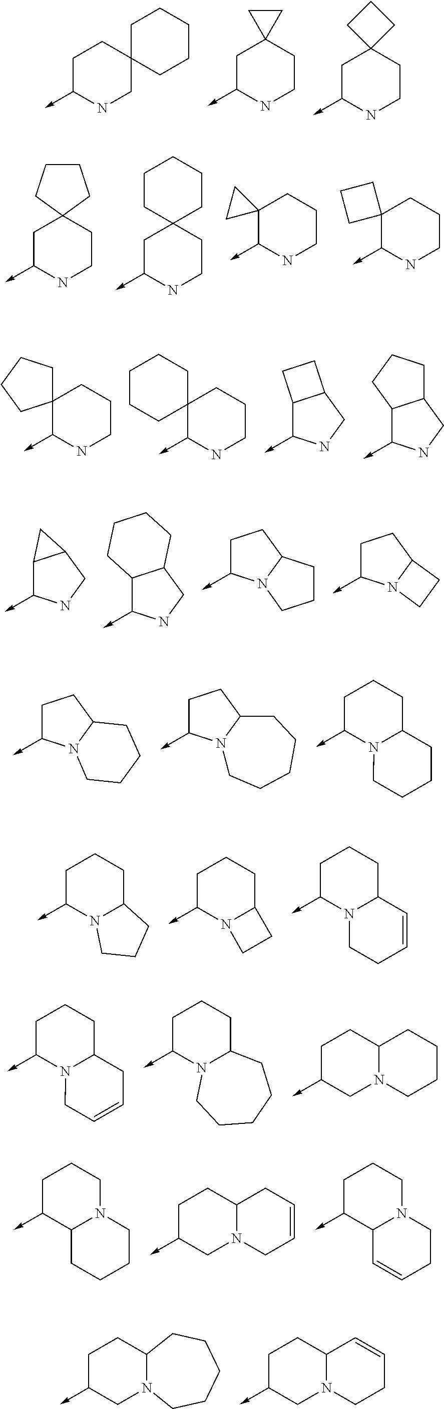

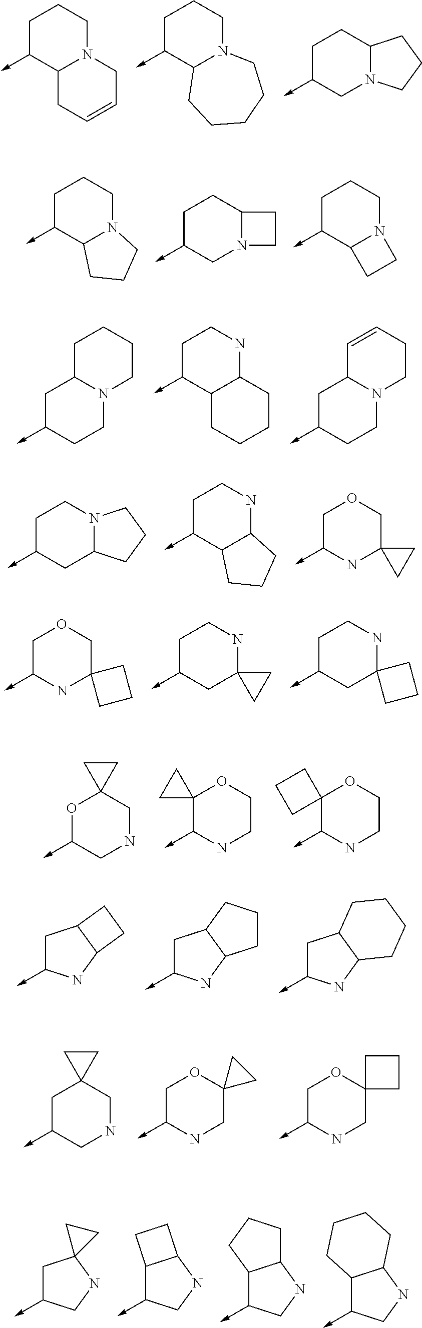

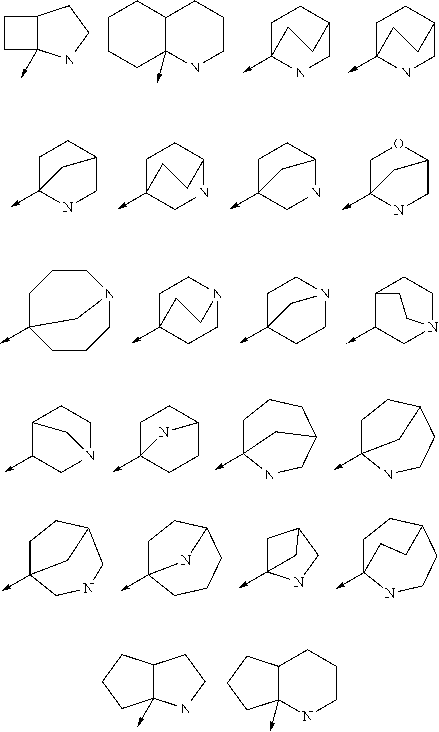

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the abovementioned substituents, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent —N(O)—, —S(O)— (also SO for short) and —S(O)₂— (also SO₂ for short) groups in the heterocyclic ring. In the case of —N(O)— and —S(O)— groups, both enantiomers in each case are included.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” means fluorine, chlorine, bromine or iodine. If the term is used for a radical, “halogen” means a fluorine, chlorine, bromine or iodine atom.

According to the nature of the substituents defined above, the compounds of the formula (I) have acidic properties and are able to form salts, and if appropriate also internal salts or adducts, with inorganic or organic bases or with metal ions. If the compounds of the formula (I) bear hydroxyl, carboxyl or other groups which induce acidic properties, these compounds can be reacted with bases to give salts. Suitable bases are, for example, hydroxides, carbonates, hydrogencarbonates of the alkali metals and alkaline earth metals, especially those of sodium, potassium, magnesium and calcium, and also ammonia, primary, secondary and tertiary amines having (C₁-C₄)-alkyl groups, mono-, di- and trialkanolamines of (C₁-C₄)-alkanols, choline and chlorocholine, and organic amines, such as trialkylamines, morpholine, piperidine or pyridine. These salts are compounds in which the acidic hydrogen is replaced by an agriculturally suitable cation, for example metal salts, especially alkali metal salts or alkaline earth metal salts, especially sodium and potassium salts, or else ammonium salts, salts with organic amines or quaternary ammonium salts, for example with cations of the formula [NRR′R″R′″]⁺ in which R to R′″ are each independently an organic radical, especially alkyl, aryl, aralkyl or alkylaryl. Also useful are alkylsulfonium and alkylsulfoxonium salts, such as (C₁-C₄)-trialkylsulfonium and (C₁-C₄)-trialkylsulfoxonium salts.

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. These salts then contain the conjugate base of the acid as anion.

Suitable substituents present in deprotonated form, for example sulfonic acids or carboxylic acids, are capable of forming inner salts with groups, such as amino groups, which can be protonated for their part.

If a group is polysubstituted by radicals, this means that this group is substituted by one or more identical or different radicals from those mentioned.

In all the formulae specified hereinafter, the substituents and symbols have the same meaning as described in formula (I), unless defined differently. Arrows in a chemical formula denote the points at which it is joined to the rest of the molecule.

There follows a description of preferred, particularly preferred and very particularly preferred definitions of each of the individual substituents. The other substituents of the general formula (I) which are not specified hereinafter have the definition given above.

The present compounds of the general formula (I) have, at the second carbon of the alkyl acid structure, a chiral carbon atom which, in the structure shown below, is indicated by the marker (*):

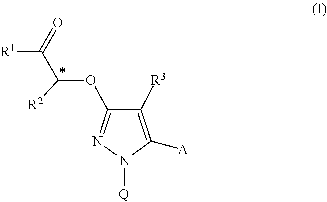

According to the rules of Cahn, Ingold and Prelog (CIP rules), this carbon atom can have either an (R) configuration or an (S) configuration.

The present invention encompasses compounds of the general formula (I) both with (S) and with (R) configuration, meaning that the present invention encompasses the compounds of the general formula (I) in which the carbon atom in question has
(1) an (R) configuration; or
(2) an (S) configuration.

In addition, the scope of the present invention also encompasses
(3) any mixtures of compounds of the general formula (I) having an (R) configuration (compounds of the general formula (I-(R)) with compounds of the general formula (I) having an (S) configuration (compounds of the general formula (I-S)), the present invention also encompassing a racemic mixture of the compounds of the general formula (I) having (R) and (S) configuration.

In addition, depending on the respective radicals chosen, further stereoelements may be present in the compounds of the general formula (I) according to the invention.

TABLE A

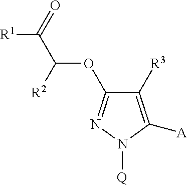

| Example | | | | | |
|---|---|---|---|---|---|
| number | Q | R³ | A | R² | R¹ |
| I-01 | 2-fluorophenyl | Br | 6-fluoropyridin-3-yl | ethoxy | ethoxy |
| I-02 | 3-fluoropyridin-2-yl | Br | 4-chlorophenyl | ethoxy | ethoxy |
| I-03 | 3-fluoropyridin-2-yl | Br | 4-chlorophenyl | methoxy | methoxy |
| I-04 | 2-fluorophenyl | Br | 6-fluoropyridin-3-yl | methoxy | methoxy |
| I-05 | 3-fluoropyridin-2-yl | Br | 6-fluoropyridin-3-yl | methoxy | methoxy |
| I-06 | 3-fluoropyridin-2-yl | Br | phenyl | methoxy | methoxy |
| I-07 | 3-fluoropyridin-2-yl | Br | 2,4-difluorophenyl | methoxy | methoxy |
| I-08 | 3-fluoropyridin-2-yl | Br | 2,4-difluorophenyl | ethoxy | ethoxy |
| I-09 | 4-chloro-2- | Br | 2,4-difluorophenyl | ethoxy | ethoxy |
| | fluorophenyl | | | | |
| I-10 | 2-fluorophenyl | Br | 6-fluoropyridin-3-yl | methoxy | rac-(2R)-3-methoxy-2- |
| | | | | | methyl-3-oxopropoxy |
| I-11 | 4-chloro-2- | Br | 2,4-difluorophenyl | methoxy | methoxy |
| | fluorophenyl | | | | |
| I-12 | 2-fluorophenyl | Br | 6-fluoropyridin-3-yl | methoxy | OH |
| I-13 | 2-fluorophenyl | Br | 6-fluoropyridin-3-yl | methoxy | 3-methoxy-3- |
| | | | | | oxopropoxy |
| I-14 | 3-fluoropyridin-2-yl | Br | 6-fluoropyridin-3-yl | methoxy | ethoxy |
| I-15 | 6-fluoropyridin-3-yl | Br | 6-fluoropyridin-3-yl | methoxy | methoxy |
| I-16 | 3-chloro-2- | Cl | 5-fluoropyridin-3-yl | methoxy | methoxy |
| | fluorophenyl | | | | |
| I-17 | 3-chloro-2- | Br | 5-fluoropyridin-3-yl | methoxy | methoxy |
| | fluorophenyl | | | | |
| I-18 | 3-chloro-2- | Br | 5-fluoropyridin-3-yl | methoxy | methoxy |
| | fluorophenyl | | | | |
| I-19 | 3-chloro-2- | I | 5-fluoropyridin-3-yl | methoxy | methoxy |
| | fluorophenyl | | | | |
| I-20 | 2-fluorophenyl | Br | 6-fluoropyridin-3-yl | methoxy | 3-methoxy-3- |
| | | | | | oxopropoxy |
| I-21 | 2-fluorophenyl | Br | 6-fluoropyridin-3-yl | methoxy | 3-methoxy-3- |
| | | | | | oxopropoxy |
| I-22 | 2-fluorophenyl | Br | 6-fluoropyridin-3-yl | methoxy | ethoxy |
| I-23 | 2-fluorophenyl | Br | 6-fluoropyridin-3-yl | methoxy | ethoxy |
| I-24 | 2-fluorophenyl | Br | 6-fluoropyridin-3-yl | methoxy | OH |
| I-25 | 2-fluorophenyl | Br | 6-fluoropyridin-3-yl | methoxy | OH |
| I-26 | phenyl | Cl | 6-fluoropyridin-3-yl | methoxy | methoxy |
| I-27 | 2-fluoro-3- | Cl | 3,4-difluorophenyl | methoxy | methoxy |
| | methylphenyl | | | | |
| I-28 | 2,5-difluorophenyl | I | 5-fluoropyridin-3-yl | methoxy | methoxy |
| I-29 | 2,5-difluorophenyl | Br | 5-fluoropyridin-3-yl | methoxy | methoxy |
| I-30 | phenyl | Br | 6-fluoropyridin-3-yl | methoxy | methoxy |
| I-31 | 2,5-difluorophenyl | cyclopropyl | 5-fluoropyridin-3-yl | methoxy | methoxy |
| I-32 | 2,5-difluorophenyl | Br | 5-fluoropyridin-3-yl | methoxy | OH |
| I-33 | 2,5-difluorophenyl | Br | 5-fluoropyridin-3-yl | methoxy | ethoxy |
| I-34 | 2,5-difluorophenyl | cyclopropyl | 5-fluoropyridin-3-yl | methoxy | OH |
| I-35 | pyrazin-2-yl | Cl | 6-fluoropyridin-3-yl | methoxy | methoxy |
| I-36 | pyrazin-2-yl | Br | 6-fluoropyridin-3-yl | methoxy | methoxy |
| I-37 | 2,5-difluorophenyl | cyclopropyl | 5-fluoropyridin-3-yl | methoxy | ethoxy |
| I-38 | 3-fluoropyridin-2-yl | Cl | 6-fluoropyridin-3-yl | methoxy | methoxy |
| I-39 | 3-fluoropyridin-2-yl | I | 6-fluoropyridin-3-yl | methoxy | methoxy |
| I-40 | 3-fluoropyridin-2-yl | cyclopropyl | 6-fluoropyridin-3-yl | methoxy | methoxy |
| I-41 | 3-fluoropyridin-2-yl | trifluoromethyl | 6-fluoropyridin-3-yl | methoxy | methoxy |
| I-42 | pyrazin-2-yl | Cl | 6-fluoropyridin-3-yl | methoxy | OH |
| I-43 | pyrazin-2-yl | Br | 6-fluoropyridin-3-yl | methoxy | OH |
| I-44 | 3-fluoropyridin-2-yl | Cl | 6-fluoropyridin-3-yl | methoxy | OH |
| I-45 | 3-fluoropyridin-2-yl | Br | 6-fluoropyridin-3-yl | methoxy | OH |
| I-46 | pyrazin-2-yl | Cl | 6-fluoropyridin-3-yl | methoxy | ethoxy |
| I-47 | pyrazin-2-yl | Br | 6-fluoropyridin-3-yl | methoxy | ethoxy |
| I-48 | 3-fluoropyridin-2-yl | Cl | 6-fluoropyridin-3-yl | methoxy | ethoxy |
| I-49 | 2-fluorophenyl | I | 6-fluoropyridin-3-yl | methoxy | methoxy |
| I-50 | 2-fluorophenyl | Cl | 6-fluoropyridin-3-yl | ethoxy | ethoxy |
| I-51 | 2-fluorophenyl | Br | 6-fluoropyridin-3-yl | ethoxy | OH |
| I-52 | 2-fluorophenyl | Cl | 6-fluoropyridin-3-yl | ethoxy | OH |
| I-53 | 2-fluorophenyl | Cl | 6-fluoropyridin-3-yl | ethoxy | 3-methoxy-3- |
| | | | | | oxopropoxy |
| I-54 | 2-fluorophenyl | Cl | 6-fluoropyridin-3-yl | ethoxy | rac-(2R)-3-methoxy-2- |
| | | | | | methyl-3-oxopropoxy |
| I-55 | 3-fluoropyridin-2-yl | trifluoromethyl | 6-fluoropyridin-3-yl | methoxy | ethoxy |
| I-56 | 3-fluoropyridin-2-yl | cyclopropyl | 6-fluoropyridin-3-yl | methoxy | OH |
| I-57 | 3-fluoropyridin-2-yl | trifluoromethyl | 6-fluoropyridin-3-yl | methoxy | OH |
| I-58 | 3-fluoropyridin-2-yl | cyclopropyl | 6-fluoropyridin-3-yl | methoxy | ethoxy |
| I-59 | 2-fluorophenyl | Br | 6-fluoropyridin-3-yl | ethoxy | 3-methoxy-3- |
| (rac) | | | | | oxopropoxy |
| I-60 | 2-fluorophenyl | 1,1,2,2,2- | 6-fluoropyridin-3-yl | methoxy | methoxy |
| | | pentafluoro- | | | |
| | | ethyl | | | |
| I-61 | 2-fluorophenyl | cyclopropyl | 6-fluoropyridin-3-yl | methoxy | 3-methoxy-3- |
| | | | | | oxopropoxy |
| I-62 | 2-fluorophenyl | trifluoromethyl | 6-fluoropyridin-3-yl | methoxy | 3-methoxy-3- |
| | | | | | oxopropoxy |
| I-63 | 2-fluorophenyl | Br | 6-fluoropyridin-3-yl | ethoxy | 3-methoxy-3- |
| (enantio- | | | | | oxopropoxy |
| mer 1) | | | | | |
| I-64 | 2-fluorophenyl | Br | 6-fluoropyridin-3-yl | ethoxy | 3-methoxy-3- |
| (enantio- | | | | | oxopropoxy |
| mer 2) | | | | | |
| I-65 | 2-fluorophenyl | Br | 6-fluoropyridin-3-yl | ethoxy | OH |
| I-66 | 2-fluorophenyl | Br | 6-fluoropyridin-3-yl | ethoxy | ethoxy |
| (enantio- | | | | | |
| mer 1) | | | | | |
| I-67 | 2-fluorophenyl | Br | 6-fluoropyridin-3-yl | ethoxy | ethoxy |
| (enantio- | | | | | |
| mer 2) | | | | | |
| I-67 | 2-fluorophenyl | Br | 6-fluoropyridin-3-yl | ethoxy | ethoxy |
| I-68 | 3-fluoropyridin-2-yl | cyclopropyl | 5-fluoropyridin-3-yl | methoxy | OH |
| I-69 | 2-fluorophenyl | cyclopropyl | 6-fluoropyridin-3-yl | methoxy | OH |
| I-70 | 2-fluorophenyl | trifluoromethyl | 6-fluoropyridin-3-yl | methoxy | methoxy |
| I-71 | 2-fluorophenyl | cyclopropyl | 6-fluoropyridin-3-yl | methoxy | methoxy |
| I-72 | 3-fluoropyridin-2-yl | I | 5-fluoropyridin-3-yl | methoxy | methoxy |
| I-73 | 3-fluoropyridin-2-yl | cyclopropyl | 5-fluoropyridin-3-yl | methoxy | methoxy |
| I-74 | 2-fluorophenyl | cyclopropyl | 6-fluoropyridin-3-yl | methoxy | 3-methoxy-3- |
| | | | | | oxopropoxy |
| I-75 | 2-fluorophenyl | cyclopropyl | 6-fluoropyridin-3-yl | methoxy | 3-methoxy-3- |
| | | | | | oxopropoxy |
| I-76 | 3-fluoropyridin-2-yl | cyclopropyl | 5-fluoropyridin-3-yl | methoxy | 3-methoxy-3- |
| | | | | | oxopropoxy |
| I-77 | 3-fluoropyridin-2-yl | cyclopropyl | 5-fluoropyridin-3-yl | methoxy | 3-methoxy-3- |
| | | | | | oxopropoxy |
| I-78 | 2,5-difluorophenyl | cyclopropyl | 5-fluoropyridin-3-yl | methoxy | 3-methoxy-3- |
| | | | | | oxopropoxy |
| I-79 | 2,5-difluorophenyl | cyclopropyl | 5-fluoropyridin-3-yl | methoxy | 3-methoxy-3- |
| | | | | | oxopropoxy |
| I-80 | 3-fluoropyridin-2-yl | cyclopropyl | 6-fluoropyridin-3-yl | methoxy | 3-methoxy-3- |
| | | | | | oxopropoxy |
| I-81 | 3-fluoropyridin-2-yl | cyclopropyl | 6-fluoropyridin-3-yl | methoxy | 3-methoxy-3- |
| | | | | | oxopropoxy |
| I-82 | 3-fluoropyridin-2-yl | cyclopropyl | 5-fluoropyridin-3-yl | methoxy | 3-methoxy-3- |
| | | | | | oxopropoxy |
| I-83 | 2,5-difluorophenyl | cyclopropyl | 5-fluoropyridin-3-yl | methoxy | 3-methoxy-3- |
| | | | | | oxopropoxy |
| I-84 | 3-fluoropyridin-2-yl | cyclopropyl | 5-fluoropyridin-3-yl | methoxy | OH |
| I-85 | 3-fluoropyridin-2-yl | cyclopropyl | 5-fluoropyridin-3-yl | methoxy | OH |
| I-86 | 3-fluoropyridin-2-yl | cyclopropyl | 5-fluoropyridin-3-yl | methoxy | methoxy |
| I-87 | 3-fluoropyridin-2-yl | cyclopropyl | 5-fluoropyridin-3-yl | methoxy | methoxy |
| I-88 | 3-fluoropyridin-2-yl | cyclopropyl | 6-fluoropyridin-3-yl | methoxy | 3-methoxy-3- |
| | | | | | oxopropoxy |
| I-89 | 3-fluoropyridin-2-yl | cyclopropyl | 6-fluoropyridin-3-yl | methoxy | OH |
| I-90 | 3-fluoropyridin-2-yl | cyclopropyl | 6-fluoropyridin-3-yl | methoxy | OH |
| I-91 | 3-fluoropyridin-2-yl | Br | 5-fluoropyridin-3-yl | methoxy | methoxy |
| I-92 | 2-fluoro-3- | Br | 2-fluoropyridin-4-yl | methoxy | OH |
| | methylphenyl | | | | |
| I-93 | 2-fluoro-3- | cyclopropyl | 6-fluoropyridin-3-yl | methoxy | methoxy |
| | methylphenyl | | | | |
| I-94 | 2-fluoro-3- | Br | 6-fluoropyridin-3-yl | methoxy | ethoxy |
| | methylphenyl | | | | |
| I-95 | 2-fluoro-3- | Br | 6-fluoropyridin-3-yl | methoxy | 3-methoxy-3- |
| | methylphenyl | | | | oxopropoxy |
| I-96 | 2-fluoro-3- | Br | 2-fluoropyridin-4-yl | methoxy | ethoxy |
| | methylphenyl | | | | |
| I-97 | 2-fluoro-3- | Br | 2-fluoropyridin-4-yl | methoxy | 3-methoxy-3- |
| | methylphenyl | | | | oxopropoxy |
| I-98 | 2-fluoro-3- | Br | 5-fluoropyridin-3-yl | methoxy | methoxy |
| | methylphenyl | | | | |
| I-99 | 2-fluoro-3- | Cl | 6-fluoropyridin-3-yl | methoxy | methoxy |
| | methylphenyl | | | | |
| I-100 | 2-fluoro-3- | Cl | 5-fluoropyridin-3-yl | methoxy | methoxy |
| | methylphenyl | | | | |
| I-101 | 3-methylsulfanyl- | I | 6-fluoropyridin-3-yl | methoxy | methoxy |
| | pyridin-2-yl | | | | |
| I-102 | 3- | Cl | 6-fluoropyridin-3-yl | methoxy | methoxy |
| | (chloromethylthio)- | | | | |
| | pyridin-2-yl | | | | |
| I-103 | 3-methylsulfinyl- | Cl | 6-fluoropyridin-3-yl | methoxy | methoxy |
| | pyridin-2-yl | | | | |
| I-104 | 3-methylsulfinyl- | I | 6-fluoropyridin-3-yl | methoxy | methoxy |
| | pyridin-2-yl | | | | |
| I-105 | pyridin-2-yl | Cl | 4-fluorophenyl | methoxy | methoxy |
| I-106 | 2-fluorophenyl | cyclopropyl | 6-fluoropyridin-3-yl | methoxy | ethoxy |
| I-106 | 2-fluorophenyl | cyclopropyl | 6-fluoropyridin-3-yl | methoxy | ethoxy |
| I-107 | 3-fluoropyridin-2-yl | cyclopropyl | 6-fluoropyridin-3-yl | methoxy | methoxy |
| I-108 | 3-fluoropyridin-2-yl | cyclopropyl | 6-fluoropyridin-3-yl | methoxy | methoxy |
| I-109 | 2,5-difluorophenyl | cyclopropyl | 5-fluoropyridin-3-yl | methoxy | OH |
| I-110 | 2,5-difluorophenyl | cyclopropyl | 5-fluoropyridin-3-yl | methoxy | OH |
| I-111 | 2,5-difluorophenyl | cyclopropyl | 5-fluoropyridin-3-yl | methoxy | methoxy |
| I-112 | 2,5-difluorophenyl | cyclopropyl | 5-fluoropyridin-3-yl | methoxy | methoxy |
| I-113 | 3-fluoropyridin-2-yl | cyclopropyl | 5-fluoropyridin-3-yl | methoxy | ethoxy |
| I-114 | 2-fluoro-3- | Br | 6-fluoropyridin-3-yl | methoxy | methoxy |
| | methylphenyl | | | | |
| I-115 | 2-fluoro-3- | Br | 2-fluoropyridin-4-yl | methoxy | methoxy |
| | methylphenyl | | | | |
| I-116 | 2-fluoro-3- | cyclopropyl | 6-fluoropyridin-3-yl | methoxy | ethoxy |
| | methylphenyl | | | | |
| I-117 | 2-fluoro-3- | cyclopropyl | 6-fluoropyridin-3-yl | methoxy | 3-methoxy-3- |
| | methylphenyl | | | | oxopropoxy |
| I-118 | 3-chloro-2- | Br | 5-fluoropyridin-3-yl | methoxy | ethoxy |
| | fluorophenyl | | | | |
| I-119 | 3-chloro-2- | Br | 5-fluoropyridin-3-yl | methoxy | 3-methoxy-3- |
| | fluorophenyl | | | | oxopropoxy |
| I-120 | 2-fluorophenyl | difluoromethyl | 6-fluoropyridin-3-yl | methoxy | methoxy |
| I-121 | 2-fluorophenyl | difluoromethyl | 6-fluoropyridin-3-yl | methoxy | 3-methoxy-3- |
| | | | | | oxopropoxy |
| I-122 | pyridin-2-yl | Br | 4-fluorophenyl | methoxy | methoxy |

A further aspect of the invention relates to the preparation of the inventive compounds of the general formula (I). The compounds of the invention can be prepared in various ways.

The compounds of the general formula (Ib) according to the invention are synthesized, as shown in Scheme 1, via an amide coupling of an acid of the general formula (Ia) with an amine of the general formula (II) in the presence of an amide coupling reagent such as, for example, T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-supported reagents, for example polymer-supported dicyclohexylcarbodiimide, are also suitable for this coupling reaction. The reaction takes place preferably within the temperature range between 0° C. and 80° C., in a suitable solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene. For T3P peptide coupling conditions see Organic Process Research & Development 2009, 13, 900-906.

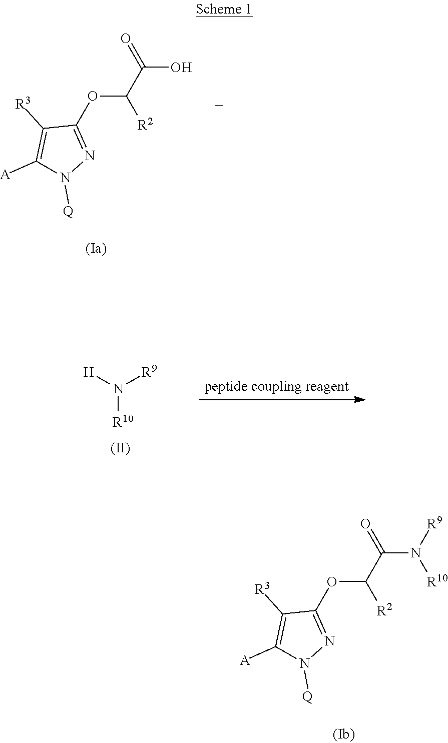

The acid of the general formula (Ia) is synthesized analogously to methods known to the person skilled in the art by ester hydrolysis of the compound of the general formula (Ic). (Scheme 2). The hydrolysis can be carried out in the presence of a base or a Lewis acid. The base may be a hydroxide salt of an alkali metal (for example lithium, sodium or potassium), and the hydrolysis reaction preferably takes place within the temperature range between room temperature and 120° C.

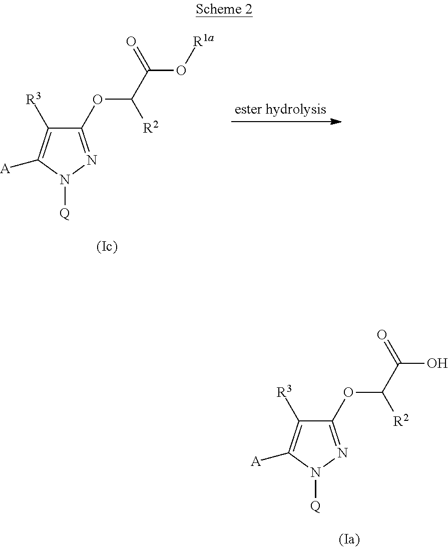

The compound of the general formula (Ic) is synthesized by alkylation of hydroxypyrazole of the general formula (III) with an alpha-halocarboxylic ester of the general formula (IV) in the presence of a base, by or analogously to methods known to the person skilled in the art (see Scheme 3). The base used may be a carbonate salt of an alkali metal. The base is preferably a carbonate salt of an alkali metal selected from the group consisting of lithium, sodium, potassium and caesium, and the reaction preferably takes place within a temperature range between room temperature and 150° C. in an appropriate solvent, for example dichloromethane, acetonitrile, N,N-dimethylformamide or ethyl acetate. See, by way of example, J. Med. Chem. 2011, 54(16), 5820-5835 and WO2010/010154. The “X” radical is, for example, chlorine, bromine or iodine.

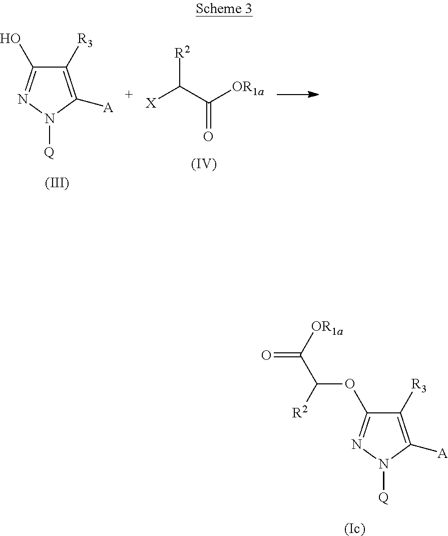

Scheme 4 describes the synthesis of the compound of the general formula (VII, R³=Cl, Br, I) by reaction of a 3-hydroxypyrazole of the general formula (V) with an electrophilic halogenating reagent of the general formula (VI), for example N-chlorosuccinimide (VI, X=Cl), N-bromosuccinimide (VI, X=Br) or N-iodosuccinimide (VI, X=I). In an analogous manner, it is also possible to use other electrophilic reagents, for example electrophilic nitrating reagents such as nitrating acid, nitronium tetrafluoroborate or ammonium nitrate/trifluoroacetic acid (when R³=nitro) or electrophilic fluorinating reagents, such as DAST, Selectfluor or N-fluorobenzenesulfonimide (when R³=F). The reaction preferably takes place within a temperature range between 0° C. and 120° C. in an appropriate solvent, for example N,N-dimethylformamide, 1,2-dichloroethane or acetonitrile.

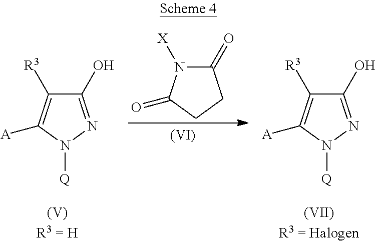

Scheme 5 describes the synthesis of the halogenated pyrazole of the general formula (Ie) by reaction of a 4H-pyrazole of the general formula (Id) with a halosuccinimide of the general formula (VI) in an appropriate solvent, for example N,N-dimethylformamide.

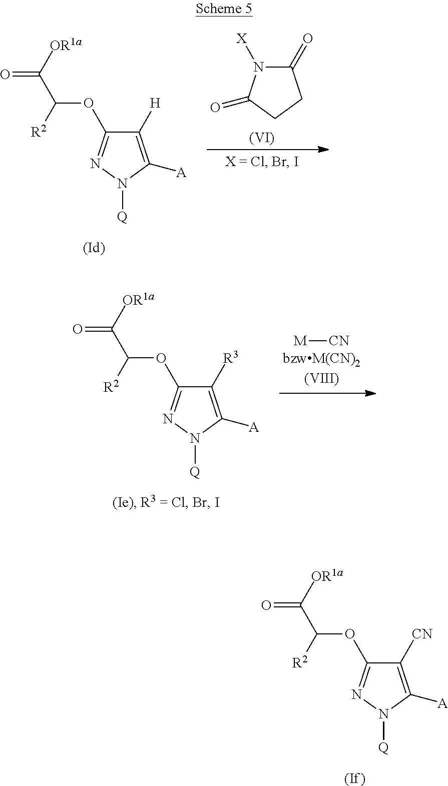

A 4-cyanopyrazole of the general formula (If) can be prepared, for example, by reaction of a compound of the formula (Ie) in a suitable solvent with a metal cyanide M-CN or M(CN)₂ (VIII) with addition of an appropriate amount of a transition metal catalyst, especially palladium catalysts such as palladium(0)tetrakis(triphenylphosphine) or palladium diacetate or bis(triphenylphosphine)palladium(II) dichloride, preferably at elevated temperature in an organic solvent, for example 1,2-dimethoxyethane or N,N-dimethylformamide (Scheme 5). Alternatively, nickel catalysts such as nickel(II) acetylacetonate or bis(triphenylphosphine)nickel(II) chloride preferably find use at elevated temperature in an organic solvent, for example 1,2-dimethoxyethane or N,N-dimethylformamide. The “M” radical in the metal cyanide M-CN or M(CN)₂ (VIII) represents, for example, zinc, lithium, potassium or sodium. Cross-coupling methods that are suitable in general are those described in R. D. Larsen, Organometallics in Process Chemistry 2004 Springer Verlag, in I. Tsuji, Palladium Reagents and Catalysts 2004 Wiley, and in M. Beller, C. Bolm, Transition Metals for Organic Synthesis 2004 VCH-Wiley. Further suitable synthesis methods are described in Chem. Rev. 2006, 106, 2651; Platinum Metals Review, 2009, 53, 183; Platinum Metals Review 2008, 52, 172 and Acc. Chem. Res. 2008, 41, 1486.

The 3-hydroxypyrazoles (V) can be prepared analogously to methods known from the literature, as described, for example, in Adv. Synth. Catal. 2014, 356, 3135-3147, in a two-stage synthesis method from substituted 3-azinylpropionic acid derivatives and phenyl hydrazines (XI) (Scheme 6). The compounds of the general formula (XII) are synthesized here via an amide coupling of an acid of the general formula (X) with an arylhydrazine or hetarylhydrazine of the general formula (XI) in the presence of an amide coupling reagent such as, for example, T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide (see Chemistry of Peptide Synthesis, Ed. N. Leo Benoiton, Taylor & Francis, 2006, ISBN-10: 1-57444-454-9). Polymer-bound reagents, for example polymer-bound dicyclohexylcarbodiimide, are also suitable for this coupling reaction. The reaction takes place preferably within the temperature range between 0° C. and 80° C., in an appropriate solvent, for example dichloromethane, tetrahydrofuran, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 6). For T3P peptide coupling conditions see Organic Process Research & Development 2009, 13, 900-906.

This is followed by the cyclization of the hydrazide (XII) in the presence of a copper halide, for example copper(I) iodide, copper(I) bromide, or of an acid such as methanesulfonic acid. The reaction preferably takes place in the temperature range between 0° C. and 120° C., in an appropriate solvent, for example 1,2-dichloroethane, acetonitrile, N,N-dimethylformamide, n-propanol or ethyl acetate.

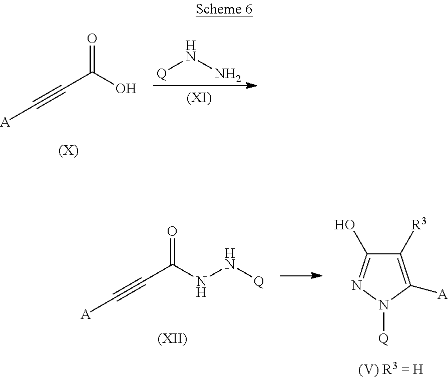

Alternatively, the hydroxypyrazoles of the general formula (V; R³=H) are synthesized from substituted azinylacrylic acid derivatives (XIII) and phenyl hydrazines (XI), as described, for example, in J. Heterocyclic Chem., 49, 130 (2012), as in Scheme 7.

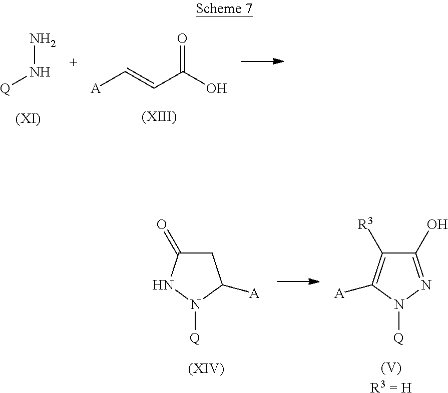

The compounds of the general formula (XIV) can be synthesized here via an amide coupling of a substituted propynoic acid of the general formula (XIII) with an arylhydrazine or hetarylhydrazine of the general formula (XI) in the presence of an amide coupling reagent such as, for example, T3P, dicyclohexylcarbodiimide, N-(3-dimethylaminopropyl)-N′-ethylcarbodiimide, N,N′-carbonyldiimidazole, 2-chloro-1,3-dimethylimidazolium chloride or 2-chloro-1-methylpyridinium iodide. The reaction takes place preferably within the temperature range between 0° C. and 80° C., in an appropriate solvent, for example dichloromethane, acetonitrile, N,N-dimethylformamide or ethyl acetate, and in the presence of a base, for example triethylamine, N,N-diisopropylethylamine or 1,8-diazabicyclo[5.4.0]undec-7-ene (see Scheme 7). The 3-hydroxypyrazoles of the general formula (V) are synthesized by reaction in the second reaction step of the compounds of the general formula (XIV) in the presence of an iron halide such as iron(III) chloride. The reaction preferably takes place in the temperature range between 0° C. and 120° C. in an appropriate solvent such as 1,2-dichloroethane, acetonitrile, N,N-dimethylformamide or ethyl acetate.

N-Arylpyrazoles of the general formula (XVII) can be prepared by N-arylation of a protected 3-hydroxypyrazole of the general formula (XV) with an aryl halide of the general formula (XVI) in the presence of a copper halide, for example copper(I) iodide. The reaction takes place preferably within the temperature range between 0° C. and 120° C., in an appropriate solvent, for example acetonitrile or N,N-dimethylformamide, and in the presence of a base, for example triethylamine, caesium carbonate (see Scheme 8). The protected 3-hydroxypyrazoles of the general formula (XV) that serve as starting material can be prepared to methods analogously known to the person skilled in the art (Chem. Med. Chem. 2015, 10, 1184-1199).

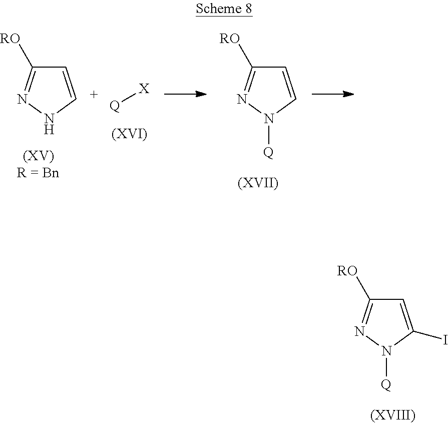

The 5-iodopyrazoles of the general formula (XVIII) are subsequently synthesized by reaction of the N-arylpyrazoles of the general formula (XVII) in the presence of a base, for example lithium diisopropylamide, and iodine. The reaction preferably takes place in the temperature range between −78° C. and −60° C., in an appropriate solvent, for example diethyl ether or tetrahydrofuran (see Scheme 8).

A bisarylpyrazole of the formula (XIX) can be prepared, for example, by reaction of an iodopyrazole of the formula (XVIII) in a suitable solvent with a reagent M-A with addition of an appropriate amount of a transition metal catalyst, especially palladium catalysts such as palladium diacetate or bis(triphenylphosphine)palladium(II) dichloride, or nickel catalysts such as nickel(II) acetylacetonate or bis(triphenylphosphine)nickel(II) chloride, preferably at elevated temperature in an organic solvent such as 1,2-dimethoxyethane. The “M” radical here represents, for example, B(OR^{b})(OR^{c}), where the R^{b} and R^{c} radicals are independently, for example, hydrogen or (C₁-C₄)-alkyl, or, if the radicals R^{b} and R^{c} are bonded to one another, together are ethylene or propylene (Scheme 9).

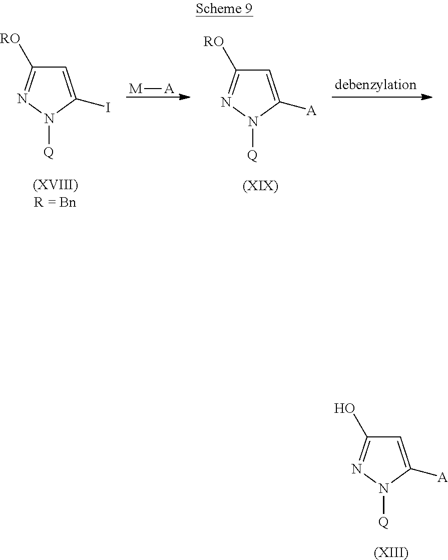

The 5-aminopyrazole of the general formula (XX) can be synthesized by alkylation of the compound of the general formula (XIII) with an alpha-halocarboxylic ester of the general formula (IV) in the presence of a base, by or analogously to methods known to the person skilled in the art (see Scheme 10 below). The base may be a carbonate salt of an alkali metal (for example lithium, sodium, potassium or caesium), and the reaction preferably takes place within a temperature range between room temperature and 150° C. in an appropriate solvent, for example dichloromethane, acetonitrile, N,N-dimethylformamide or ethyl acetate.

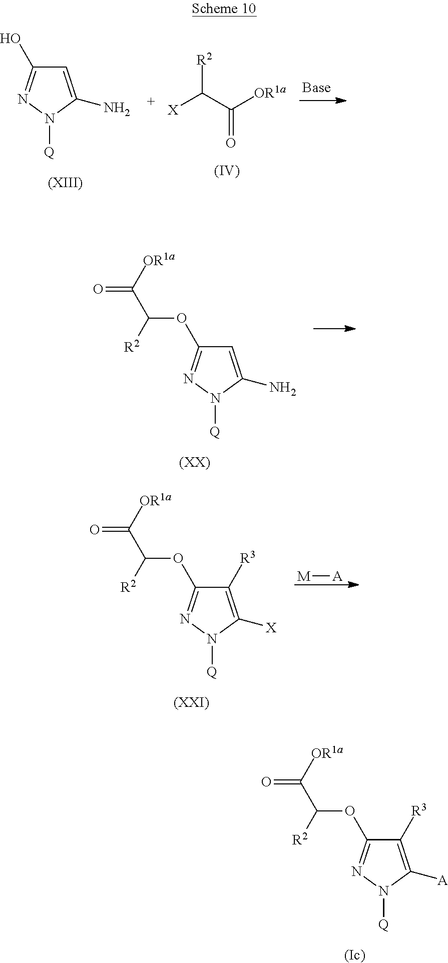

Subsequently, as likewise shown in Scheme 10, 5-halopyrazoles of the general formula (XXI) are synthesized by diazotization of the 5-aminopyrazole of the general formula (XX) by reaction with the customary organic or inorganic nitrites, for example 1,1-dimethylethyl nitrite, tert-butyl nitrite or isoamyl nitrite, in the presence of copper(I) and/or copper(II) bromide, copper(I) and/or copper(II) chloride, or in the presence of copper(I) iodide or elemental iodine. The reaction preferably takes place within the temperature range between 0° C. and 120° C. in an appropriate solvent, for example dichloromethane, acetonitrile, N,N-dimethylformamide or N,N-dimethylacetamide. The “X” radical of the 5-halopyrazoles of the general formula (XXI) is, for example, chlorine, bromine or iodine. The subsequent conversion to the compound of the formula (Ic) is effected by reaction of the 5-halopyrazoles of the general formula (XXI) in a suitable solvent with a (het)aryl derivative A-M with addition of an appropriate amount of a transition metal catalyst, especially palladium catalysts such as palladium diacetate or bis(triphenylphosphine)palladium(II) dichloride, or nickel catalysts such as nickel(II) acetylacetonate or bis(triphenylphosphine)nickel(II) chloride, preferably at elevated temperature in an organic solvent such as 1,2-dimethoxyethane. The “M” radical here represents, for example, Mg-Hal, Zn-Hal, Sn((C₁-C₄)alkyl)₃, lithium, copper or B(OR^{b})(OR^{c}), where the R^{b} and R^{c} radicals are independently, for example, hydrogen, (C₁-C₄)-alkyl, or, when the R^{b} and R^{c} radicals are bonded to one another, they are collectively ethylene or propylene.

Selected detailed synthesis examples for the inventive compounds of the general formula (I) are adduced below. The example numbers given correspond to the numbering given in Table A below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets.

In the case of diastereomer mixtures, what is reported is either the significant signals for each of the two diastereomers or the characteristic signal of the main diastereomer. The abbreviations used for chemical groups have, for example, the following meanings: Me=CH₃, Et=CH₂CH₃, t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### 3-(3,4-Difluorophenyl)prop-2-ynoic acid

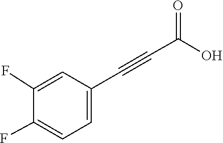 [SMILES: O=C(O)C#Cc1ccc(F)c(F)c1]

Under an argon atmosphere, the following are added successively to 5.00 g (20.83 mmol) of 1,2-difluoro-4-iodobenzene in 30 ml of dry tetrahydrofuran: 1.46 g (20.83 mmol) of propiolic acid, 0.29 g (0.42 mmol) of bis(triphenylphosphine)palladium(11) dichloride, 0.16 g (0.83 mmol) of copper(I)iodide and 7.38 g (72.92 mmol) of diisopropylamine. The mixture is stirred at room temperature for 2 hours, the reaction mixture is added to water, 15.00 ml of 2 N hydrochloric acid is added, and extraction is effected repeatedly with ethyl acetate. The combined organic phases are dried over sodium sulfate and concentrated under reduced pressure. After purification by column chromatography on silica gel with heptane/ethyl acetate (start with heptane/ethyl acetate=95:5, to heptane/ethyl acetate=40:60 within 15 min), 2.89 g (76%) of a product with m/z=183 [M+] is obtained.

¹H NMR (400 MHz, d6-DMSO): δ=7.56 (m, 2H), 7.86 (m, 1H), 13.95 (bs, 1H).

### 3-(3,4-Difluorophenyl)-N′-(3-fluoropyridin-2-yl)prop-2-yne hydrazide

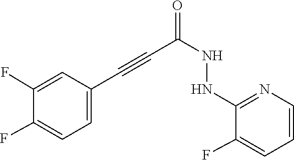 [SMILES: O=C(C#Cc1ccc(F)c(F)c1)NNc1ncccc1F]

To a solution of 2.20 g (12.08 mmol) of 3-(3,4-difluorophenyl)prop-2-ynoic acid, 1.77 g (13.90 mmol) of 2-fluoro-6-hydrazinopyridine and 3.06 g (30.20 mmol) of triethylamine in 180 ml of THF is added dropwise 15.34 g (24.16 mmol) of a 50% propanephosphonic anhydride solution in THF, and this mixture is stirred at room temperature for one hour. For workup, H₂O is added, the organic phase is removed, and the aqueous phase is extracted repeatedly with CH₂Cl₂. The combined organic phase is dried over Na₂SO₄ and concentrated. 3.20 g (72%) of crude product of 80% purity is obtained, which is used without further purification for the next reaction stage.

### 5-(3,4-Difluorophenyl)-1-(3-fluoropyridin-2-yl)-1H-pyrazol-3-ol

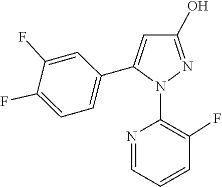 [SMILES: Oc1cc(-c2ccc(F)c(F)c2)n(-c2ncccc2F)n1]

To a solution of 3.20 g (9.89 mmol) of 3-(3,4-difluorophenyl)-N′-(3-fluoropyridin-2-yl)prop-2-yne hydrazide in 50 ml of acetonitrile and 8 ml of DMF is added 151 mg (0.79 mmol) of CuI, and the mixture is refluxed for three hours. This is followed by removal by filtration and concentration, and purification of the crude product by column chromatography using silica gel with heptane/ethyl acetate (3:7). In this way, 1.96 g (67%) of product is obtained in solid form.

¹H NMR (400 MHz, DMSO-d₆): δ 6.15 (s, 1H), 6.95 (m, 1H), 7.30-7.40 (m, 2H), 7.55 (m, 1H), 7.95 (m, 1H), 8.25 (m, 1H).

### Ethyl (2RS)-{[4-bromo-1-(2-fluorophenyl)-5-(6-fluoropyridin-3-yl)-1H-pyrazol-3-yl]oxy}(ethoxy)acetate (I-01)

### Ethyl (2RS)-ethoxy{[1-(2-fluorophenyl)-5-(6-fluoropyridin-3-yl)-1H-pyrazol-3-yl]oxy}acetate

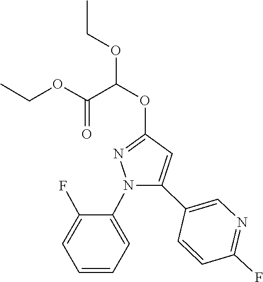 [SMILES: CCOC(=O)C(OCC)Oc1cc(-c2ccc(F)nc2)n(-c2ccccc2F)n1]

To a solution of 0.25 g (0.91 mmol) of 5-1-(2-fluorophenyl)-5-(6-fluoropyridin-3-yl)-1H-pyrazol-3-ol in 10 ml of acetonitrile are successively added 253 g (1.83 mmol) of K₂CO₃ and 186 mg (1.83 mmol) of ethyl (2RS)-chloro(ethoxy)acetate, and then the mixture is stirred under reflux for 4 hours. Thereafter, CH₂Cl₂ and H₂O are added to the reaction mixture (about 10 ml of each). The phases are separated by means of a separator cartridge and the organic phase is then concentrated under reduced pressure. Purification by column chromatography using silica gel with heptane/ethyl acetate gives 237 mg (63% yield) of the target product.

¹H NMR (400 MHz, CDCl3): δ 1.31 (t, 6H), 3.84-4.02 (br m, 2H), 4.30 (m, 2H), 5.94 (s, 1H), 6.16 (s, 1H), 6.87 (dd, 1H), 7.22 (t, 1H), 7.37 (m, 1H), 7.44 (dt, 1H), 7.59 (dt, 1H), 8.08 (m, 1H).

### Ethyl (2RS)-{[4-bromo-1-(2-fluorophenyl)-5-(6-fluoropyridin-3-yl)-1H-pyrazol-3-yl]oxy}(ethoxy)acetate (I-01)

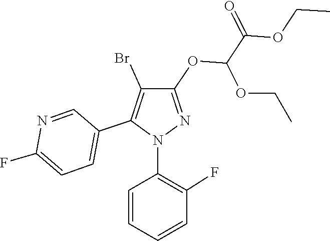 [SMILES: CCOC(=O)C(OCC)Oc1nn(-c2ccccc2F)c(-c2ccc(F)nc2)c1Br]

To a solution of 118 mg (0.29 mmol) of ethyl (2RS)-ethoxy{[1-(2-fluorophenyl)-5-(6-fluoropyridin-3-yl)-1H-pyrazol-3-yl]oxy}acetate in 5 ml acetonitrile is added 156 mg (0.87 mmol) of N-bromosuccinimide, and the mixture is stirred under reflux conditions for 3 hours. Then the reaction mixture is left to stand at room temperature overnight, and CH₂Cl₂ and H₂O are added to the reaction mixture (about 10 ml of each). The phases are separated by means of a separator cartridge and the organic phase is then concentrated under reduced pressure. Column chromatography purification using silica gel with heptane/ethyl acetate gives 141 mg (98%) of ethyl (2RS)-{[4-bromo-1-(2-fluorophenyl)-5-(6-fluoropyridin-3-yl)-1H-pyrazol-3-yl]oxy}(ethoxy)acetate (I-1).

¹H NMR (400 MHz, CDCl3): δ 1.32 (pseudo q, 6H), 3.86-4.06 (br m, 2H), 4.30 (m, 2H), 5.96 (s, 1H), 6.92 (dd, 1H), 7.02 (dt, 1H), 7.22 (dt, 1H), 7.35 (m, 1H), 7.39 (dt, 1H), 7.74 (dt, 1H), 8.10 (m, 1H).

### Methyl (2RS)-{[4-bromo-1-(2-fluorophenyl)-5-(6-fluoropyridin-3-yl)-1H-pyrazol-3-yl]oxy}(methoxy)acetate (I-04)

### Methyl (2RS)-methoxy{[1-(2-fluorophenyl)-5-(6-fluoropyridin-3-yl)-1H-pyrazol-3-yl]oxy}acetate

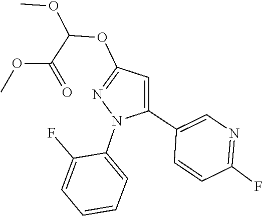 [SMILES: COC(=O)C(OC)Oc1cc(-c2ccc(F)nc2)n(-c2ccccc2F)n1]

To a solution of 0.25 g (0.91 mmol) of 5-1-(2-fluorophenyl)-5-(6-fluoropyridin-3-yl)-1H-pyrazol-3-ol in 10 ml of acetonitrile are successively added 253 mg (1.83 mmol) of K₂CO₃ and 139 mg (1.83 mmol) of methyl (2RS)-chloro(methoxy)acetate, and then the mixture is stirred under reflux for 4 hours. Thereafter, CH₂Cl₂ and H₂O are added to the reaction mixture (about 10 ml of each). The phases are separated by means of a separator cartridge and the organic phase is then concentrated under reduced pressure. Purification by column chromatography using silica gel with heptane/ethyl acetate gives 240 mg (69% yield) of the target product.

¹H NMR (400 MHz, CDCl3): δ 3.66 (s, 3H), 3.85 (s, 3H), 5.93 (s, 1H), 6.17 (s, 1H), 6.85 (dd, 1H), 7.06 (dt, 1H), 7.24 (dt, 1H), 7.35 (m, 1H), 7.44 (dt, 1H), 7.59 (dt, 1H), 8.09 (m, 1H).

### Methyl (2RS)-{[4-bromo-1-(2-fluorophenyl)-5-(6-fluoropyridin-3-yl)-1H-pyrazol-3-yl]oxy}(methoxy)acetate (I-04)

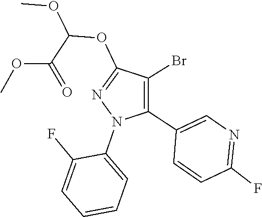 [SMILES: COC(=O)C(OC)Oc1nn(-c2ccccc2F)c(-c2ccc(F)nc2)c1Br]

To a solution of 120 mg (0.32 mmol) of methyl (2RS)-methoxy{[1-(2-fluorophenyl)-5-(6-fluoropyridin-3-yl)-1H-pyrazol-3-yl]oxy}acetate in 5 ml acetonitrile is added 171 mg (0.95 mmol) of N-bromosuccinimide, and the mixture is stirred under reflux conditions for 6 hours. Thereafter, the reaction mixture was cooled to room temperature and then CH₂Cl₂ and H₂O were added (about 10 ml of each). The phases are separated by means of a separator cartridge and the organic phase is then concentrated under reduced pressure. Column chromatography purification using silica gel with heptane/ethyl acetate gives 134 mg (91%) of methyl (2RS)-{[4-bromo-1-(2-fluorophenyl)-5-(6-fluoropyridin-3-yl)-1H-pyrazol-3-yl]oxy}(methoxy)acetate (I-1).

¹H NMR (400 MHz, CDCl3): δ 3.69 (s, 3H), 3.86 (s, 3H), 5.96 (s, 1H), 6.93 (dd, 1H), 7.03 (dt, 1H), 7.21 (dt, 1H), 7.35 (m, 1H), 7.41 (dt, 1H), 7.74 (dt, 1H), 8.11 (m, 1H).

### Methyl ({5-(6-fluoropyridin-3-yl)-4-iodo-1-[3-(methylsulfanyl)pyridin-2-yl]-1H-pyrazol-3-yl}oxy)(methoxy)acetate (I-101) and methyl ({5-(6-fluoropyridin-3-yl)-4-iodo-1-[3-(methylsulfinyl)pyridin-2-yl]-1H-pyrazol-3-yl}oxy)(methoxy)acetate (I-104)

### 3-(6-Fluoropyridin-3-yl)prop-2-ynoic acid

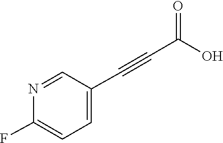 [SMILES: O=C(O)C#Cc1ccc(F)nc1]

Under an argon atmosphere, the following were added successively to 20.00 g (130.05 mmol, 1.0 equiv.) of 2-fluoro-5-iodopyridine in 400 ml of dry tetrahydrofuran: 10.02 g (143.06 mmol, 1.10 equiv.) of propiolic acid, 1.83 g (2.60 mmol, 0.20 equiv.) of bis(triphenylphosphine)palladium(11) dichloride, 0.99 g (5.02 mmol, 0.04 equiv.) of copper(I)iodide and 63.80 ml (455.19 mmol, 3.50 equiv.) of diisopropylamine. The mixture was stirred at room temperature for 2 hours and diluted with ethyl acetate (300 ml), the reaction mixture was added to ice-water (200 ml), 2 N hydrochloric acid was added, and extraction was effected repeatedly with ethyl acetate. The combined organic phases were dried over sodium sulfate and concentrated under reduced pressure. The residue was stirred with a mixture (1:1) of ethyl acetate and n-heptane, and filtered with suction. The filtercake was dried under reduced pressure and used without further purification in the next synthesis stage. 19.96 g (74%, 80% pure) of 3-(6-fluoropyridin-3-yl)prop-2-ynoic acid was obtained in the form of a brown solid.

¹H NMR (400 MHz, d⁶-DMSO δ, ppm) 14.03 (bs, 1H), 8.57 (d, 1H), 8.32 (m, 1H), 7.32 (m, 1H).

### 2-Hydrazino-3-(methylsulfanyl)pyridine

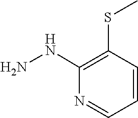

2-Fluoro-3-(methylsulfanyl)pyridine (10.0 g, 69.84 mmol, 1.0 equiv) was suspended in tert-butanol (50 ml), and then hydrazine hydrate (14.61 ml, 300.32 mmol, 4.30 equiv.) and potassium carbonate (8.10 g, 58.61 mmol, 0.83 equiv.) were added. The suspension was heated to boiling overnight. After cooling to room temperature, the mixture was diluted with water (200 ml) and extracted three times with dichloromethane (200 ml each time). The combined organic phases were washed with a saturated sodium chloride solution and dried over sodium sulfate. Then the solvent was removed under reduced pressure. By final purification of the resulting crude product by column chromatography (ethyl acetate/heptane gradient), 2-hydrazino-3-(methylsulfanyl)pyridine was isolated in the form of a beige solid (10.12 g, 88% of theory).

¹H NMR (400 MHz, d⁶-DMSO δ, ppm) 7.98 (m, 1H), 7.47 (m, 1H), 7.01 (bs, 1H), 6.64 (m, 1H), 4.21 (bs, 2H), 2.36 (s, 3H).

### 3-(6-Fluoropyridin-3-yl)-N′-[3-(methylsulfanyl)pyridin-2-yl]prop-2-yne hydrazide

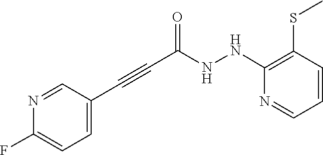 [SMILES: CSc1cccnc1NNC(=O)C#Cc1ccc(F)nc1]

3-(6-Fluoropyridin-3-yl)prop-2-ynoic acid (2.40 g, 14.53 mmol, 1.0 equiv) was dissolved in THF (100 ml), and 2-hydrazino-3-(methylsulfanyl)pyridine (2.48 g, 15.99 mmol, 1.1 equiv.) and triethylamine (6.08 ml, 43.60 mmol, 3.0 equiv.) were added. At 10° C., a 50% T3P solution in THF (17.30 ml, 29.07 mmol, 2.0 equiv.) was added within 20 minutes. The resulting reaction mixture was stirred at room temperature overnight. Then the solvent was removed under reduced pressure, and ethyl acetate (200 ml) and a 1 M pH=4.65 buffer solution (40 ml) was added. The organic phase was washed with a saturated sodium chloride solution (20 ml) and dried over sodium sulfate, and the solvent was removed under reduced pressure. By final purification of the resulting crude product by column chromatography (ethyl acetate/heptane gradient), 3-(6-fluoropyridin-3-yl)-N′-[3-(methylsulfanyl)pyridin-2-yl]prop-2-yne hydrazide was isolated in the form of a brown solid (1.83 g, 39% of theory).

¹H NMR (400 MHz, d⁶-DMSO δ, ppm) 10.60 (bs, 1H), 8.56 (s, 1H), 8.26 (m, 1H), 8.18 (bs, 1H), 7.97 (m, 1H), 7.61 (d, 1H), 7.21 (dd, 1H), 6.81 (m, 1H), 2.46 (s, 3H).

### 5-(6-Fluoropyridin-3-yl)-1-[3-(methylsulfanyl)pyridin-2-yl]-1H-pyrazol-3-ol

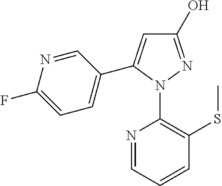 [SMILES: CSc1cccnc1-n1nc(O)cc1-c1ccc(F)nc1]

3-(6-Fluoropyridin-3-yl)-N′-[3-(methylsulfanyl)pyridin-2-yl]prop-2-yne hydrazide (4.00 g, 13.23 mmol, 1.0 equiv.) was dissolved in a mixture of DMF (50 ml) and 1,2-dichloroethane (150 ml). Copper(I) iodide (0.50 g, 2.65 mmol, 0.20 equiv.) was added to the solution. The resulting brown reaction mixture was heated to 90° C. for two days. After cooling to room temperature, the solvent was removed under reduced pressure. By final purification of the resulting crude product by column chromatography (ethyl acetate/heptane gradient), 5-(6-fluoropyridin-3-yl)-1-[3-(methylsulfanyl)pyridin-2-yl]-1H-pyrazol-3-ol was isolated in the form of a brown solid (1.79 g, 42% of theory).

¹H NMR (400 MHz, d⁶-DMSO δ, ppm) 10.33 (bs, 1H), 8.17 (m, 1H), 8.02 (m, 1H), 7.90 (d, 1H), 7.66 (m, 1H), 7.49 (m, 1H), 7.13 (dd, 1H), 6.17 (bs, 1H), 2.43 (s, 3H).

### Methyl ({5-(6-fluoropyridin-3-yl)-1-[3-(methylsulfanyl)pyridin-2-yl]-1H-pyrazol-3-yl}oxy)(methoxy) acetate

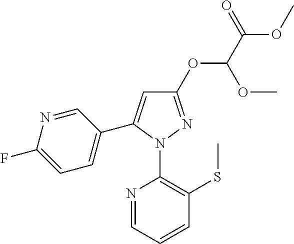 [SMILES: COC(=O)C(OC)Oc1cc(-c2ccc(F)nc2)n(-c2ncccc2SC)n1]

5-(6-Fluoropyridin-3-yl)-1-[3-(methylsulfanyl)pyridin-2-yl]-1H-pyrazol-3-ol (277 mg, 0.92 mmol, 1.0 equiv) was dissolved in acetonitrile (20 ml), and then methyl chloro(methoxy)acetate (190 mg, 1.37 mmol, 1.50 equiv.) and potassium carbonate (380 mg, 2.75 mmol, 3.0 equiv.) were added. The suspension was heated to 90° C. for 3 hours. After cooling, the solids were filtered off by suction, the residue was washed through twice with acetonitrile (4 ml each time), and the solvent was removed under reduced pressure. By final purification of the resulting crude product by column chromatography (ethyl acetate/heptane gradient), methyl ({5-(6-fluoropyridin-3-yl)-1-[3-(methylsulfanyl)pyridin-2-yl]-1H-pyrazol-3-yl}oxy)(methoxy) acetate was isolated in the form of a brown solid (330 mg, 84% of theory).

¹H NMR (400 MHz, CDCl₃ δ, ppm) 8.17 (m, 1H), 8.06 (d, 1H), 7.68-7.61 (m, 21H), 7.30 (dd, 1H), 6.85 (dd, 1H), 6.19 (s, 1H), 5.99 (s, 1H), 3.84 (s, 3H), 3.66 (s, 3H), 2.38 (s, 3H).

### Methyl ({5-(6-fluoropyridin-3-yl)-4-iodo-1-[3-(methylsulfanyl)pyridin-2-yl]-1H-pyrazol-3-yl}oxy)(methoxy)acetate (I-101) and methyl ({5-(6-fluoropyridin-3-yl)-4-iodo-1-[3-(methylsulfinyl)pyridin-2-yl]-1H-pyrazol-3-yl}oxy)(methoxy)acetate (I-104)

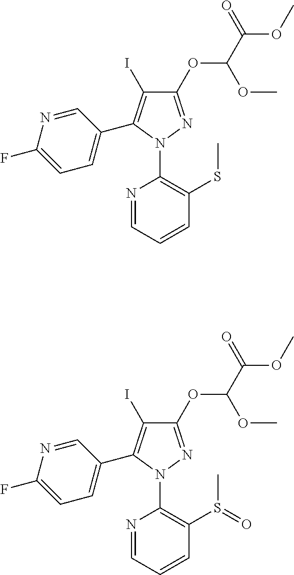 [SMILES: COC(=O)C(OC)Oc1nn(-c2ncccc2SC)c(-c2ccc(F)nc2)c1I ; COC(=O)C(OC)Oc1nn(-c2ncccc2S(C)=O)c(-c2ccc(F)nc2)c1I]

Methyl ({5-(6-fluoropyridin-3-yl)-1-[3-(methylsulfanyl)pyridin-2-yl]-1H-pyrazol-3-yl}oxy)(methoxy)acetate (180 mg, 0.44 mmol, 1.0 equiv) was dissolved in acetonitrile (12 ml), and 1,3-diiodo-5,5-dimethylimidazolidine-2,4-dione (101 mg, 0.27 mmol, 0.6 equiv.) was added at room temperature. The reaction was stirred at room temperature overnight. Then one drop of conc. sulfuric acid was added to the reaction mixture, which was stirred at room temperature overnight. After checking the reaction by thin-film chromatography, another drop of conc. sulfuric acid was added, and the mixture was stirred at room temperature for 3 hours. Thereafter, water (5 ml) and a saturated sodium hydrogencarbonate solution were added to the reaction solution, which was extracted twice with dichloromethane (70 ml). The organic phase was dried over magnesium sulfate and the solvent was removed under reduced pressure. By final purification of the resulting crude product by column chromatography (ethyl acetate/heptane gradient), methyl ({5-(6-fluoropyridin-3-yl)-4-iodo-1-[3-(methylsulfanyl)pyridin-2-yl]-1H-pyrazol-3-yl}oxy)(methoxy)acetate was isolated in the form of a white solid (130 mg, 52% of theory), and methyl ({5-(6-fluoropyridin-3-yl)-4-iodo-1-[3-(methylsulfinyl)pyridin-2-yl]-1H-pyrazol-3-yl}oxy)(methoxy)acetate in the form of a white solid (83 mg, 32% of theory).

I-101: methyl ({5-(6-fluoropyridin-3-yl)-4-iodo-1-[3-(methylsulfanyl)pyridin-2-yl]-1H-pyrazol-3-yl}oxy)(methoxy)acetate ¹H NMR (400 MHz, CDCl₃ δ, ppm) 8.15-8.10 (m, 2H), 7.85 (m, 1H), 7.58 (m, 1H), 7.27 (m, 1H), 6.91 (m, 1H), 5.99 (m, 1H), 3.84 (s, 3H), 3.68 (s, 3H), 2.39 (s, 3H). I-104: methyl ({5-(6-fluoropyridin-3-yl)-4-iodo-1-[3-(methylsulfinyl)pyridin-2-yl]-1H-pyrazol-3-yl}oxy)(methoxy)acetate ¹H NMR (400 MHz, CDCl₃ δ, ppm) 8.15-8.10 (m, 2H), 7.85 (m, 1H), 7.27-7.25 (m, 2H), 6.91 (m, 1H), 5.99 (m, 1H), 3.84 (s, 3H), 3.68 (s, 3H), 2.39 (s, 3H).

### Methyl {[4-chloro-1-(3-chloro-2-fluorophenyl)-5-(5-fluoropyridin-3-yl)-1H-pyrazol-3-yl]oxy}(methoxy)acetate (I-26)

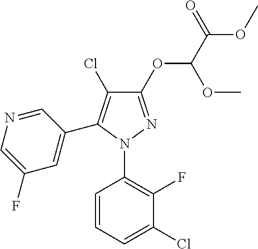 [SMILES: COC(=O)C(OC)Oc1nn(-c2cccc(Cl)c2F)c(-c2cncc(F)c2)c1Cl]

Methyl {[1-(3-chloro-2-fluorophenyl)-5-(5-fluoropyridin-3-yl)-1H-pyrazol-3-yl]oxy}(methoxy)acetate (100 mg, 0.24 mmol, 1.0 equiv) was dissolved in acetonitrile (10 ml), and 1,3-dichloro-5,5-dimethylimidazolidine-2,4-dione (29 mg, 0.14 mmol, 0.6 equiv.) was added at room temperature. The reaction was stirred at room temperature overnight. The reaction mixture was then stirred at room temperature overnight. Thereafter, water (5 ml) and a saturated sodium hydrogencarbonate solution were added to the reaction solution, which was extracted twice with dichloromethane (70 ml). The organic phase was dried over magnesium sulfate and the solvent was removed under reduced pressure. By final purification of the resulting crude product by column chromatography (ethyl acetate/heptane gradient), methyl {[4-chloro-1-(3-chloro-2-fluorophenyl)-5-(5-fluoropyridin-3-yl)-1H-pyrazol-3-yl]oxy}(methoxy)acetate was isolated in the form of a white solid (94 mg, 82% of theory).

¹H NMR (400 MHz, CDCl₃ δ, ppm) 8.49 (d, 1H), 8.26 (d, 1H), 7.45-7.41 (m, 2H), 7.29 (m, 1H), 7.17 (m, 1H), 5.94 (s, 1H), 3.86 (s, 3H), 3.69 (s, 3H).

### Methyl ({4-chloro-5-(6-fluoropyridin-3-yl)-1-[3-(methylsulfinyl)pyridin-2-yl]-1H-pyrazol-3-yl}oxy)(methoxy)acetate (I-103)

and

### methyl {[4-chloro-1-{3-[(chloromethyl)sulfanyl]pyridin-2-yl}-5-(6-fluoropyridin-3-yl)-1H-pyrazol-3-yl]oxy}(methoxy)acetate (I-102)

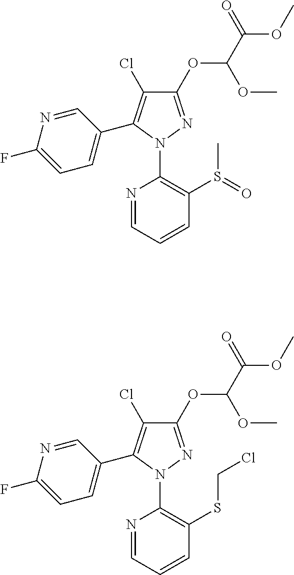 [SMILES: COC(=O)C(OC)Oc1nn(-c2ncccc2S(C)=O)c(-c2ccc(F)nc2)c1Cl ; COC(=O)C(OC)Oc1nn(-c2ncccc2SCCl)c(-c2ccc(F)nc2)c1Cl]

Methyl ({5-(6-fluoropyridin-3-yl)-1-[3-(methylsulfanyl)pyridin-2-yl]-1H-pyrazol-3-yl}oxy)(methoxy)acetate (60 mg, 0.14 mmol, 1.0 equiv) was dissolved in acetonitrile (5 ml), and 1,3-dichloro-5,5-dimethylimidazolidine-2,4-dione (18 mg, 0.08 mmol, 0.6 equiv.) was added at room temperature. The reaction was stirred at room temperature overnight. The reaction mixture was then stirred at room temperature overnight. Thereafter, water (5 ml) and a saturated sodium hydrogencarbonate solution were added to the reaction solution, which was extracted twice with dichloromethane (70 ml). The organic phase was dried over magnesium sulfate and the solvent was removed under reduced pressure. By final purification of the resulting crude product by column chromatography (ethyl acetate/heptane gradient), methyl ({4-chloro-5-(6-fluoropyridin-3-yl)-1-[3-(methylsulfinyl)pyridin-2-yl]-1H-pyrazol-3-yl}oxy)(methoxy)acetate (11 mg, 15% of theory) and methyl {[4-chloro-1-{3-[(chloromethyl)sulfanyl]pyridin-2-yl}-5-(6-fluoropyridin-3-yl)-1H-pyrazol-3-yl]oxy}(methoxy)acetate (20 mg, 27% of theory) were isolated.

I-103: methyl ({4-chloro-5-(6-fluoropyridin-3-yl)-1-[3-(methylsulfinyl)pyridin-2-yl]-1H-pyrazol-3-yl}oxy)(methoxy)acetate ¹H NMR (400 MHz, CDCl₃ δ, ppm) 8.59 (m, 1H), 8.23-8.17 (m, 2H), 7.81 (m, 1H), 7.45 (m, 1H), 6.98 (m, 1H), 5.84 (s, 1H), 3.89 (s, 3H), 3.68 (s, 3H), 2.94 (d, 3H).

I-102: methyl {[4-chloro-1-{3-[(chloromethyl)sulfanyl]pyridin-2-yl}-5-(6-fluoropyridin-3-yl)-1H-pyrazol-3-yl]oxy}(methoxy)acetate ¹H NMR (400 MHz, CDCl₃ δ, ppm) 8.25 (m, 1H), 8.13 (d, 1H), 8.05 (d, 1H), 7.83 (m, 1H), 7.35 (m, 1H), 6.9 (dd, 1H), 5.93 (s, 1H), 4.90 (s, 2H), 3.85 (s, 3H), 3.68 (s, 3H).

### (2RS)-{[4-Bromo-1-(2-fluorophenyl)-5-(6-fluoropyridin-3-yl)-1H-pyrazol-3-yl]oxy}(ethoxy)acetic acid (I-51)

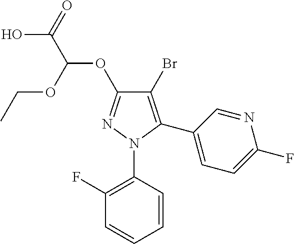 [SMILES: CCOC(Oc1nn(-c2ccccc2F)c(-c2ccc(F)nc2)c1Br)C(=O)O]

To an initial charge of 285.0 mg (0.591 mmol) of ethyl (2RS)-{[4-bromo-1-(2-fluorophenyl)-5-(6-fluoropyridin-3-yl)-1H-pyrazol-3-yl]oxy}(ethoxy)acetate in 3.6 ml of tetrahydrofuran and 1.2 ml of water is added 49.5 mg (1.182 mmol) of lithium hydroxide monohydrate. The reaction mixture is stirred at room temperature for two hours.

Ethyl acetate is added, and the mixture is acidified with 0.6 ml (1.182 mmol) of 2M aqueous hydrochloric acid and extracted repeatedly with ethyl acetate. The combined organic phases are dried and concentrated under reduced pressure. 265.8 mg (94% yield) of a yellowish oil of 95% purity is obtained.

### Methyl 3-{[(2RS)-2-{[4-bromo-1-(2-fluorophenyl)-5-(6-fluoropyridin-3-yl)-1H-pyrazol-3-yl]oxy}-2-ethoxyethanoyl]oxy}propanoate (I-59)

### Methyl 3-{[(2R*)-2-{[4-bromo-1-(2-fluorophenyl)-5-(6-fluoropyridin-3-yl)-1H-pyrazol-3-yl]oxy}-2-ethoxyethanoyl]oxy}propanoate (enantiomer 1, I-64)

### Methyl 3-{[(2R*)-2-{[4-bromo-1-(2-fluorophenyl)-5-(6-fluoropyridin-3-yl)-1H-pyrazol-3-yl]oxy}-2-ethoxyethanoyl]oxy}propanoate (enantiomer 2, I-63)

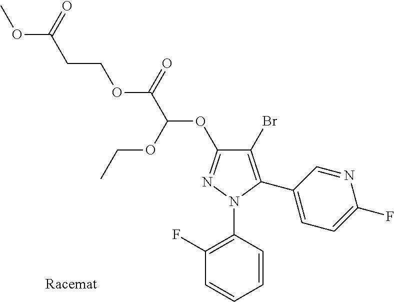 [SMILES: CCOC(Oc1nn(-c2ccccc2F)c(-c2ccc(F)nc2)c1Br)C(=O)OCCC(=O)OC]

To an initial charge of 1000.0 mg (2.202 mmol) of (2RS)-{[4-bromo-1-(2-fluorophenyl)-5-(6-fluoropyridin-3-yl)-1H-pyrazol-3-yl]oxy}(ethoxy)acetic acid in 32.2 ml of tetrahydrofuran are successively added 723.8 mg (6.605 mmol) of methyl 3-hydroxypropanoate, 2101.5 mg (3.302 mmol) of propylphosphonic anhydride (T3P), 2.7 mg (0.022 mmol) of 4-dimethylaminopyridine (DMAP) and 445.6 mg (4.403 mmol) of triethylamine. The reaction mixture is stirred at 50° C. for two hours. Methylene chloride and saturated aqueous ammonium chloride solution are added, and the mixture is extracted repeatedly with methylene chloride. The combined organic phases are separated using a phase separator, dried and concentrated under reduced pressure. The residue is taken up in a little methylene chloride and chromatographed on a Biotage Isolera (column: MN Chromabond RS40, gradient: 10% to 90% EA in 8 runs). After a first-round of 59.4 mg of a colourless oil, which consists of a mixture of unknown components and is discarded, 737.0 mg (59% yield) of methyl 3-{[(2RS)-2-{[4-bromo-1-(2-fluorophenyl)-5-(6-fluoropyridin-3-yl)-1H-pyrazol-3-yl]oxy}-2-ethoxyethanoyl]oxy}propanoate (I-59) is obtained in the form of a colourless oil.

This mixture is then separated into the enantiomers by chiral supercritical fluid chromatography (SFC) by the following method: Chir_C₁_IC_B1_90CO2_MeOH_QDA1. After 2.904 min, 240.8 mg (20% yield) of a colourless oil is obtained (enantiomer 1, I-64)

¹H NMR (400 MHz, CDCl₃): δ=1.30 (t, 3H), 2.70 (t, 2H), 3.65 (s, 3H), 3.85 (m, 1H), 4.05 (m, 1H), 4.50 (t, 2H), 5.95 (s, 1H), 6.95 (dd, 1H), 7.05 (dt, 1H), 7.20 (t, 1H), 7.35 (m, 1H), 7.45 (dt, 1H), 7.75 (dt, 1H), 8.10 (d, 1H)
and
after 2.987 min, 320.9 mg (26% yield) of a colourless oil (enantiomer 2, I-63)

¹H NMR (400 MHz, CDCl₃): δ=1.30 (t, 3H), 2.70 (t, 2H), 3.65 (s, 3H), 3.85 (m, 1H), 4.05 (m, 1H), 4.50 (t, 2H), 5.95 (s, 1H), 6.95 (dd, 1H), 7.05 (dt, 1H), 7.20 (t, 1H), 7.35 (m, 1H), 7.45 (dt, 1H), 7.75 (dt, 1H), 8.10 (d, 1H)

The enantiomerically pure double esters thus obtained are then each hydrolysed separately and then re-esterified separately:

### (2R*)-{[4-Bromo-1-(2-fluorophenyl)-5-(6-fluoropyridin-3-yl)-1H-pyrazol-3-yl]oxy}(ethoxy)acetic acid (enantiomer1)

To an initial charge of 120.4 mg (0.223 mmol) of methyl 3-{[(2R*)-2-{[4-bromo-1-(2-fluorophenyl)-5-(6-fluoropyridin-3-yl)-1H-pyrazol-3-yl]oxy}-2-ethoxyethanoyl]oxy}propanoate (enantiomer1) in 5.0 ml of tetrahydrofuran and 2.0 ml of water is added 18.7 mg (0.446 mmol) of lithium hydroxide monohydrate. The reaction mixture is stirred at room temperature for two hours. Ethyl acetate is added, and the mixture is acidified with 0.22 ml (0.446 mmol) of 2M aqueous hydrochloric acid and extracted repeatedly with ethyl acetate. The combined organic phases are dried and concentrated under reduced pressure. 101.0 mg (97% yield) of a yellowish oil of 98% purity is obtained, which crystallizes later on.

### (2R*)-{[4-Bromo-1-(2-fluorophenyl)-5-(6-fluoropyridin-3-yl)-1H-pyrazol-3-yl]oxy}(ethoxy)acetic acid (enantiomer2, I-65)

To an initial charge of 214.0 mg (0.396 mmol) of methyl 3-{[(2R*)-2-{[4-bromo-1-(2-fluorophenyl)-5-(6-fluoropyridin-3-yl)-1H-pyrazol-3-yl]oxy}-2-ethoxyethanoyl]oxy}propanoate (enantiomer2) in 5.0 ml of tetrahydrofuran and 2.0 ml of water is added 33.2 mg (0.792 mmol) of lithium hydroxide monohydrate. The reaction mixture is stirred at room temperature for two hours.

Ethyl acetate is added, and the mixture is acidified with 0.40 ml (0.792 mmol) of 2M aqueous hydrochloric acid and extracted repeatedly with ethyl acetate. The combined organic phases are dried and concentrated under reduced pressure. 188.9 mg (99% yield) of a yellowish oil of 95% purity is obtained, which crystallizes later on.

### Ethyl (2R*)-{[4-bromo-1-(2-fluorophenyl)-5-(6-fluoropyridin-3-yl)-1H-pyrazol-3-yl]oxy}(ethoxy)acetate (enantiomer1, I-66)

To an initial charge of 101.0 mg (0.222 mmol) of (2R*)-{[4-bromo-1-(2-fluorophenyl)-5-(6-fluoropyridin-3-yl)-1H-pyrazol-3-yl]oxy}(ethoxy)acetic acid (enantiomer1) in 5.0 ml of tetrahydrofuran are successively added 32.3 mg (0.667 mmol) of ethanol, 212.3 mg (0.334 mmol) of propylphosphonic anhydride (T3P), 0.27 mg (0.002 mmol) of 4-dimethylaminopyridine (DMAP) and 45.0 mg (0.445 mmol) of triethylamine. The reaction mixture is stirred at 50° C. for two hours. Methylene chloride and saturated aqueous ammonium chloride solution are added, and the mixture is extracted repeatedly with methylene chloride. The combined organic phases are separated using a phase separator, dried and concentrated under reduced pressure. The residue is taken up in a little methylene chloride and chromatographed on a Biotage Isolera (column: MN Chromabond RS40, gradient: 10% to 90% EA in 8 runs). 40.3 mg (36% yield) of a colourless oil of 98% purity is obtained.

### Ethyl (2R*)-{[4-bromo-1-(2-fluorophenyl)-5-(6-fluoropyridin-3-yl)-1H-pyrazol-3-yl]oxy}(ethoxy)acetate (enantiomer2, I-67)

To an initial charge of 100.0 mg (0,220 mmol) of (2R*)-{[4-bromo-1-(2-fluorophenyl)-5-(6-fluoropyridin-3-yl)-1H-pyrazol-3-yl]oxy}(ethoxy)acetic acid (enantiomer2) in 5.0 ml of tetrahydrofuran are successively added 32.0 mg (0.660 mmol) of ethanol, 210.1 mg (0.330 mmol) of propylphosphonic anhydride (T3P), 0.27 mg (0.002 mmol) of 4-dimethylaminopyridine (DMAP) and 44.6 mg (0.440 mmol) of triethylamine. The reaction mixture is stirred at 50° C. for two hours. Methylene chloride and saturated aqueous ammonium chloride solution are added, and the mixture is extracted repeatedly with methylene chloride. The combined organic phases are separated using a phase separator, dried and concentrated under reduced pressure. The residue is taken up in a little methylene chloride and chromatographed on a Biotage Isolera (column: MN Chromabond RS40, gradient: 10% to 90% EA in 8 runs). 34.8 mg (32% yield) of a colourless oil of 98% purity is obtained.

### Methyl (2RS)-{[4-(difluoromethyl)-1-(2-fluorophenyl)-5-(6-fluoropyridin-3-yl)-1H-pyrazol-3-yl]oxy}(methoxy)acetate (I-120)

### 3-(Benzyloxy)-1-(2-fluorophenyl)-5-(6-fluoropyridin-3-yl)-1H-pyrazole-4-carbaldehyde

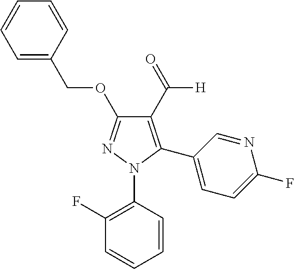 [SMILES: O=Cc1c(OCc2ccccc2)nn(-c2ccccc2F)c1-c1ccc(F)nc1]

6 ml of tetrahydrofuran is added under argon, and the mixture is cooled to −70° C. 0.7 ml (0.971 mmol) of a solution of isopropylmagnesium chloride-lithium chloride complex is added dropwise thereto. Subsequently, 485.0 mg (0.971 mmol) of 5-[3-(benzyloxy)-1-(2-fluorophenyl)-4-iodo-1H-pyrazol-5-yl]-2-fluoropyridine, dissolved in 3 ml of tetrahydrofuran, are added dropwise. After stirring for one hour at −70° C., 177.5 mg (2.429 mmol) of N,N-dimethylformamide are added dropwise and the reaction mixture is stirred at room temperature for two hours.

Saturated aqueous ammonium chloride solution is then added to the reaction mixture, which is extracted twice with ethyl acetate. The organic phases are combined, dried over magnesium sulfate, filtered and concentrated under reduced pressure. The residue is taken up in a little methylene chloride and chromatographed (Biotage Isolera, column: MN Chromabond RS40, 5% to 50% EA in 8 runs). 303.6 mg (79% yield) of an oil of 99% purity is obtained.

¹H NMR (400 MHz, CDCl₃): δ=5.40 (s, 2H), 6.95 (dd, 1H), 7.05 (dt, 1H), 7.25 (dt, 1H), 7.35-7.45 (m, 4H), 7.45-7.55 (m, 3H), 7.85 (dt, 1H), 8.05 (m, 1H), 9.90 (s, 1H)

### Methyl (2RS)-{[4-(difluoromethyl)-1-(2-fluorophenyl)-5-(6-fluoropyridin-3-yl)-1H-pyrazol-3-yl]oxy}(methoxy)acetate (I-120)

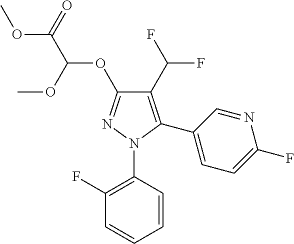 [SMILES: COC(=O)C(OC)Oc1nn(-c2ccccc2F)c(-c2ccc(F)nc2)c1C(F)F]

An initial charge of 248.0 mg (0.604 mmol) of the methyl (2RS)-{[1-(2-fluorophenyl)-5-(6-fluoropyridin-3-yl)-4-formyl-1H-pyrazol-3-yl]oxy}(methoxy)acetate thus prepared in 10.0 ml of methylene chloride is cooled to 0° C. Subsequently, 291.8 mg (1.811 mmol) of diethylaminosulfur trifluoride (DAST) are added dropwise, and the reaction mixture is allowed to come gradually to room temperature. Water is added, and the mixture is extracted repeatedly with methylene chloride. The combined organic phases are removed using a phase separator, dried and concentrated under reduced pressure. The residue is taken up in a little methylene chloride and chromatographed (Biotage Isolera, column: MN Chromabond RS40, 5% to 65% EA in 9 runs). 239.3 mg (88% yield) of a colourless oil of 95% purity are obtained.

### Methyl {[4-cyclopropyl-1-(2,5-difluorophenyl)-5-(5-fluoropyridin-3-yl)-1H-pyrazol-3-yl]oxy}(methoxy)acetate (I-31)

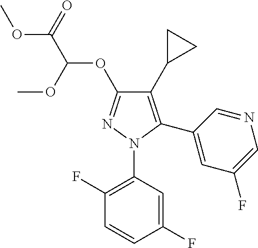 [SMILES: COC(=O)C(OC)Oc1nn(-c2cc(F)ccc2F)c(-c2cncc(F)c2)c1C1CC1]

To 0.530 g (0.970 mmol) of methyl {[1-(2,5-difluorophenyl)-5-(5-fluoropyridin-3-yl)-4-iodo-1H-pyrazol-3-yl]oxy}(methoxy)acetate in 25.0 ml of dioxane under nitrogen were added 0.250 g (2.909 mmol) of cyclopropylboronic acid, 0.295 g (1.939 mmol) of caesium fluoride and 0.079 g (0.097 mmol) of [1,1′-bis(diphenylphosphino)ferrocene]dichloropalladium(II) (complex with dichloromethane), and the mixture was stirred under reflux for 3 h. The reaction mixture was concentrated under reduced pressure, the residue was taken up in dichloromethane and water, the aqueous phase was extracted repeatedly with dichloromethane, the combined organic phases were dried over sodium sulfate, and the solvent was removed under reduced pressure. After purification by column chromatography on silica gel with heptane/ethyl acetate, 0.424 g (96% of theory) of an oil was obtained.

### Methyl {[1-(3-fluoropyridin-2-yl)-5-(6-fluoropyridin-3-yl)-4-(trifluoromethyl)-1H-pyrazol-3-yl]oxy}(methoxy)acetate (I-41)

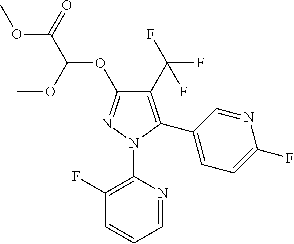 [SMILES: COC(=O)C(OC)Oc1nn(-c2ncccc2F)c(-c2ccc(F)nc2)c1C(F)(F)F]

To 0.500 g (0.996 mmol) of methyl {[1-(3-fluoropyridin-2-yl)-5-(6-fluoropyridin-3-yl)-4-iodo-1H-pyrazol-3-yl]oxy}(methoxy)acetate in 25.0 ml of dimethylacetamide were added 0.956 g (4.978 mmol) of methyl difluoro(fluorosulfonyl)acetate and 0.379 g (1.991 mmol) of copper(I) iodide, and the mixture was stirred at 85° C. for 5 h. Ethyl acetate was added to the reaction mixture, which was filtered, the filtrate was concentrated under reduced pressure, the residue was taken up in dichloromethane and water, the aqueous phase was extracted repeatedly with dichloromethane, the combined organic phases were dried over sodium sulfate, and the solvent was removed under reduced pressure. After purification by column chromatography on silica gel with heptane/ethyl acetate, 0.241 g (52% of theory) of an oil was obtained.

### {[4-Chloro-5-(6-fluoropyridin-3-yl)-1-(pyrazin-2-yl)-1H-pyrazol-3-yl]oxy}(methoxy)acetic acid (I-42)

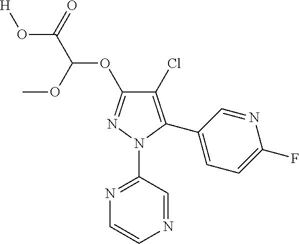 [SMILES: COC(Oc1nn(-c2cnccn2)c(-c2ccc(F)nc2)c1Cl)C(=O)O]

To 0.180 g (0.457 mmol) of methyl {[4-chloro-5-(6-fluoropyridin-3-yl)-1-(pyrazin-2-yl)-1H-pyrazol-3-yl]oxy}(methoxy)acetate in 10.0 ml of tetrahydrofuran was added a solution of 0.027 g (1.143 mmol) of lithium hydroxide in 3 ml of water, and the mixture was stirred at 25° C. for 2 h. The aqueous phase was adjusted to pH=2-3 with 2 M aqueous hydrochloric acid, the solvent was removed under reduced pressure, and the residue was taken up with water and extracted three times with dichloromethane. The combined organic phases were dried over sodium sulfate, and the solvent was removed under reduced pressure. A colourless solid (0.179 g, 96% of theory) was obtained.

The inventive compounds of the formula (I) (and/or salts thereof), referred to collectively as “compounds of the invention” hereinafter, have excellent herbicidal efficacy against a broad spectrum of economically important monocotyledonous and dicotyledonous annual harmful plants.

The present invention therefore also provides a method of controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence.

Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

The compounds of the invention can be selective in crops of useful plants and can also be employed as non-selective herbicides.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants.

Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A)
genetically modified plants having reduced photorespiration, which have higher yields and higher stress tolerance (EP 0305398 A)
transgenic crop plants which produce pharmaceutically or diagnostically important proteins (“molecular pharming”)
transgenic crop plants which feature higher yields or better quality
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. For the connection of the DNA fragments to one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, NY; or Winnacker “Gene und Klone” [Genes and Clones], VCH Weinheim, 2nd edition, 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Obtainable in this way are transgenic plants having properties altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients, or to any desired combinations of these active ingredients.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as maize or soya with the trade name or the designation Optimum™ GAT™ (glyphosate ALS tolerant), for example.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Kuchler, “Chemische Technologie” [Chemical Technology], Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The necessary formulation auxiliaries, such as inert materials, surfactants, solvents and further additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesell., Stuttgart 1976, Winnacker-Kuchler, “Chemische Technologie”, Volume 7, C. Hanser Verlag Munich, 4th ed. 1986.

On the basis of these formulations, it is also possible to produce combinations with other active ingredients, for example insecticides, acaricides, herbicides, fungicides, and also with safeners, fertilizers and/or growth regulators, for example in the form of a finished formulation or as a tank mix. Combination partners usable for the compounds of the invention in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as known, for example, from Weed Research 26 (1986) 441-445 or “The Pesticide Manual”, 16th edition, The British Crop Protection Council and the Royal Soc. of Chemistry, 2006, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Examples of such herbicidal mixing partners are:

acetochlor, acifluorfen, acifluorfen-methyl, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, aminopyralid-dimethylammonium, aminopyralid-tripromine, amitrol, ammonium sulfamat, anilofos, asulam, asulam-potassium, asulam-sodium, atrazin, azafenidin, azimsulfuron, beflubutamid, (S)-(−)-beflubutamid, beflubutamid-M, benazolin, benazolin-ethyl, benazolin-dimethylammonium, benazolin-potassium, benfluralin, benfuresat, bensulfuron, bensulfuron-methyl, bensulid, bentazon, bentazon-sodium, benzobicyclon, benzofenap, bicyclopyron, bifenox, bilanafos, bilanafos-sodium, bipyrazon, bispyribac, bispyribac-sodium, bixlozon, bromacil, bromacil-lithium, bromacil-sodium, bromobutid, bromofenoxim, bromoxynil, bromoxynil-butyrate, -potassium, -heptanoate and -octanoate, busoxinon, butachlor, butafenacil, butamifos, butenachlor, butralin, butroxydim, butylat, cafenstrol, cambendichlor, carbetamid, carfentrazon, carfentrazon-ethyl, chloramben, chloramben-ammonium, chloramben-diolamine, chlroamben-methyl, chloramben-methylammonium, chloramben-sodium, chlorbromuron, chlorfenac, chlorfenac-ammonium, chlorfenac-sodium, chlorfenprop, chlorfenprop-methyl, chlorflurenol, chlorflurenol-methyl, chloridazon, chlorimuron, chlorimuron-ethyl, chlorophthalim, chlorotoluron, chlorsulfuron, chlorthal, chlorthal-dimethyl, chlorthal-monomethyl, cinidon, cinidon-ethyl, cinmethylin, exo-(+)-cinmethylin, i.e. (1R,2S,4S)-4-isopropyl-1-methyl-2-[(2-methylbenzyl)oxy]-7-oxabicyclo[2.2.1]-heptane, exo-(−)-cinmethylin, i.e. (1R,2S,4S)-4-isopropyl-1-methyl-2-[(2-methylbenzyl)oxy]-7-oxabicyclo[2.2.1]heptane, cinosulfuron, clacyfos, clethodim, clodinafop, clodinafop-ethyl, clodinafop-propargyl, clomazone, clomeprop, clopyralid, clopyralid-methyl, clopyralid-olamine, clopyralid-potassium, clopyralid-tripomine, cloransulam, cloransulam-methyl, cumyluron, cyanamid, cyanazin, cycloat, cyclopyranil, cyclopyrimorat, cyclosulfamuron, cycloxydim, cyhalofop, cyhalofop-butyl, cyprazin, 2,4-D (including the -theammonium, -butotyl, -butyl, -choline, -diethylammonium, -dimethylammonium, -diolamine, -doboxyl, -dodecylammonium, -etexyl, -ethyl, -2-ethylhexyl, -heptylammonium, -isobutyl, -isooctyl, -isopropyl, -isopropylammonium, -lithium, -meptyl, -methyl, -potassium, -tetradecylammonium, -triethylammonium, -triisopropanolammonium, -tripromine and -trolamine salt thereof), 2,4-DB, 2,4-DB-butyl, -dimethylammonium, -isooctyl, -potassium and -sodium, daimuron (dymron), dalapon, dalapon-calcium, dalapon-Magnesium, dalapon-sodium, dazomet, dazomet-sodium, n-decanol, 7-deoxy-d-sedoheptulose, desmedipham, detosyl-pyrazolate (dTP), dicamba and salts thereof, e.g. dicamba-biproamine, dicamba-N,N-Bis(3-aminopropyl)methylamine, dicamba-butotyl, dicamba-choline, dicamba-diglycolamine, dicamba-dimethylammonium, dicamba-diethanolaminemmonium, dicamba-diethylammonium, dicamba-isopropylammonium, dicamba-methyl, dicamba-monoethanolamine, dicamba-olamine, dicamba-potassium, dicamba-sodium, dicamba-triethanolamine, dichlobenil, 2-(2,5-dichlorobenzyl)-4,4-dimethyl-1,2-oxazolidin-3-one, dichlorprop, dichlorprop-butotyl, dichlorprop-dimethylammonium, dichlorprop-etexyl, dichlorprop-ethylammonium, dichlorprop-isoctyl, dichlorprop-methyl, dichlorprop-potassium, dichlorprop-sodium, dichlorprop-P, dichlorprop-P-dimethylammonium, dichlorprop-P-etexyl, dichlorprop-P-potassium, dichlorprop-sodium, diclofop, diclofop-methyl, diclofop-P, diclofop-P-methyl, diclosulam, difenzoquat, difenzoquat-metilsulfate, diflufenican, diflufenzopyr, diflufenzopyr-sodium, dimefuron, dimepiperate, dimesulfazet, dimethachlor, dimethametryn, dimethenamid, dimethenamid-P, dimetrasulfuron, dinitramine, dinoterb, dinoterb-acetate, diphenamid, diquat, diquat-dibromid, diquat-dichloride, dithiopyr, diuron, DNOC, DNOC-ammonium, DNOC-potassium, DNOC-sodium, endothal, endothal-diammonium, endothal-dipotassium, endothal-disodium, epyrifenacil (S-3100), ePTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-5231, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenpyrazone, fenquinotrione, fentrazamid, flamprop, flamprop-isoproyl, flamprop-methyl, flamprop-M-isopropyl, flamprop-M-methyl, flazasulfuron, florasulam, florpyrauxifen, florpyrauxifen-benzyl, fluazifop, fluazifop-butyl, fluazifop-methyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanat, flupropanat-sodium, flupyrsulfuron, flupyrsulfuron-methyl, flupyrsulfuron-methyl-sodium, fluridon, flurochloridon, fluroxypyr, fluroxypyr-butometyl, fluroxypyr-meptyl, flurtamon, fluthiacet, fluthiacet-methyl, fomesafen, fomesafen-sodium, foramsulfuron, foramsulfuron-sodium, fosamine, fosamine-ammonium, glufosinate, glufosinate-ammonium, glufosinate-sodium, L-glufosinate-ammonium, L-glufosinate-sodium, glufosinate-P-sodium, glufosinate-P-ammonium, glyphosate, glyphosate-ammonium, -isopropylammonium, -diammonium, -dimethylammonium, -potassium, -sodium, glyphosate-sesquisodium and -trimesium, H-9201, i.e. O-(2,4-dimethyl-6-nitrophenyl)-O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, haloxifop-sodium, hexazinon, HNPC-A8169, i.e. prop-2-yn-1-yl (2S)-2-{3-[(5-tert-butylpyridin-2-yl)oxy]phenoxy}propanoate, HW-02, i.e. 1-(dimethoxyphosphoryl)ethyl (2,4-dichlorophenoxy)acetate, hydantocidin, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazaquin-methyl, imazethapyr, imazethapyr-ammonium, imazosulfuron, indanofan, indaziflam, iodosulfuron, iodosulfuron-methyl, iodosulfuron-methyl-sodium, ioxynil, ioxynil-lithium, -octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, ketospiradox-potassium, lactofen, lenacil, linuron, MCPA, MCPA-butotyl, -butyl, -dimethylammonium, -diolamine, -2-ethylhexyl, -ethyl, -isobutyl, -isoctyl, -isopropyl, -isopropylammonium, -methyl, -olamine, -potassium, -sodium and -trolamine, MCPB, MCPB-methyl, -ethyl and -sodium, mecoprop, mecoprop-butotyl, mecoprop-dimethylammonium, mecoprop-diolamine, mecoprop-etexyl, mecoprop-ethadyl, mecoprop-isoctyl, mecoprop-methyl, mecoprop-potassium, mecoprop-sodium, and mecoprop-trolamine, mecoprop-P, mecoprop-P-butotyl, -dimethylammonium, -2-ethylhexyl and -potassium, mefenacet, mefluidid, mefluidid-diolamine, mefluidid-potassium, mesosulfuron, mesosulfuron-methyl, mesosulfuron-sodium, mesotrion, methabenzthiazuron, metam, metamifop, metamitron, metazachlor, metazosulfuron, methabenzthiazuron, methiopyrsulfuron, methiozolin, methyl isothiocyanat, metobromuron, metolachlor, S-metolachlor, metosulam, metoxuron, metribuzin, metsulfuron, metsulfuron-methyl, molinat, monolinuron, monosulfuron, monosulfuron-methyl, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, NC-656, i.e. 3-[(isopropylsulfonyl)methyl]-N-(5-methyl-1,3,4-oxadiazol-2-yl)-5-(trifluoromethyl)[1,2,4]triazolo-[4,3-a]pyridine-8-carboxamide, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefone, oxyfluorfen, paraquat, paraquat-dichloride, paraquat-dimethylsulfate, pebulate, pendimethalin, penoxsulam, pentachlorophenol, pentoxazone, pethoxamid, petroleum oil, phenmedipham, phenmedipham-ethyl, picloram, picloram-dimethylammonium, picloram-etexyl, picloram-isoctyl, picloram-methyl, picloram-olamine, picloram-potassium, picloram-triethylammonium, picloram-tripromine, picloram-trolamine, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamid, prosulfocarb, prosulfuron, pyraclonil, pyraflufen, pyraflufen-ethyl, pyrasulfotol, pyrazolynat (pyrazolat), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridat, pyriftalid, pyriminobac, pyriminobac-methyl, pyrimisulfan, pyrithiobac, pyrithiobac-sodium, pyroxasulfon, pyroxsulam, quinclorac, quinclorac-dimethylammonium, quinclorac-methyl, quinmerac, quinoclamin, quizalofop, quizalofop-ethyl, quizalofop-P, quizalofop-P-ethyl, quizalofop-P-tefuryl, QYM201, i.e. 1-{2-chloro-3-[(3-cyclopropyl-5-hydroxy-1-methyl-1H-pyrazol-4-yl)carbonyl]-6-(trifluoromethyl)phenyl}piperidin-2-one, rimsulfuron, saflufenacil, sethoxydim, siduron, simazine, simetryn, SL-261, sulcotrione, sulfentrazone, sulfometuron, sulfometuron-methyl, sulfosulfuron, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid) and salts thereof, e.g. TCA-ammonium, TCA-calcium, TCA-ethyl, TCA-magnesium, TCA-sodium, tebuthiuron, tefuryltrione, tembotrion, tepraloxydim, terbacil, terbucarb, terbumeton, terbuthylazine, terbutryn, tetflupyrolimet, thaxtomin, thenylchlor, thiazopyr, thiencarbazone, thiencarbazon-methyl, thifensulfuron, thifensulfuron-methyl, thiobencarb, tiafenacil, tolpyralat, topramezon, tralkoxydim, triafamon, tri-allate, triasulfuron, triaziflam, tribenuron, tribenuron-methyl, triclopyr, triclopyr-butotyl, triclopyr-choline, triclopyr-ethyl, triclopyr-triethylammonium, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, 3-(2-chloro-4-fluoro-5-(3-methyl-2,6-dioxo-4-trifluoromethyl-3,6-dihydropyrimidin-1 (2H)-yl)phenyl)-5-methyl-4,5-dihydroisoxazole-5-carboxylic acid ethyl ester, 3-chloro-2-[3-(difluoromethyl)isoxazolyl-5-yl]phenyl 5-chloropyrimidin-2-yl ether, 2-(3,4-dimethoxyphenyl)-4-[(2-hydroxy-6-oxocyclohex-1-en-1-yl)carbonyl]-6-methylpyridazin-3(2H)-one, 2-({2-[(2-methoxyethoxy)methyl]-6-methylpyridin-3-yl}carbonyl)cyclohexane-1,3-dione, (5-hydroxy-1-methyl-1H-pyrazol-4-yl)(3,3,4-trimethyl-1,1-dioxido-2,3-dihydro-1-benzothiophen-5-yl)methanone, 1-methyl-4-[(3,3,4-trimethyl-1,1-dioxido-2,3-dihydro-1-benzothiophen-5-yl)carbonyl]-1H-pyrazol-5-yl propane-1-sulfonate, 4-{2-chloro-3-[(3,5-dimethyl-1H-pyrazol-1-yl)methyl]-4-(methylsulfonyl)benzoyl}-1-methyl-1H-pyrazol-5-yl 1,3-dimethyl-1H-pyrazole-4-carboxylate, cyanomethyl 4-amino-3-chloro-5-fluoro-6-(7-fluoro-1H-indol-6-yl)pyridine-2-carboxylate, prop-2-yn-1-yl 4-amino-3-chloro-5-fluoro-6-(7-fluoro-1H-indol-6-yl)pyridine-2-carboxylate, methyl 4-amino-3-chloro-5-fluoro-6-(7-fluoro-1H-indol-6-yl)pyridine-2-carboxylate, 4-amino-3-chloro-5-fluoro-6-(7-fluoro-1H-indol-6-yl)pyridine-2-carboxylic acid, benzyl 4-amino-3-chloro-5-fluoro-6-(7-fluoro-1H-indol-6-yl)pyridine-2-carboxylate, ethyl 4-amino-3-chloro-5-fluoro-6-(7-fluoro-1H-indol-6-yl)pyridine-2-carboxylate, methyl 4-amino-3-chloro-5-fluoro-6-(7-fluoro-1-isobutyryl-1H-indol-6-yl)pyridine-2-carboxylate, methyl 6-(1-acetyl-7-fluoro-1H-indol-6-yl)-4-amino-3-chloro-5-fluoropyridine-2-carboxylate, methyl 4-amino-3-chloro-6-[1-(2,2-dimethylpropanoyl)-7-fluoro-1H-indol-6-yl]-5-fluoropyridine-2-carboxylate, methyl 4-amino-3-chloro-5-fluoro-6-[7-fluoro-1-(methoxyacetyl)-1H-indol-6-yl]pyridine-2-carboxylate, potassium 4-amino-3-chloro-5-fluoro-6-(7-fluoro-1H-indol-6-yl)pyridine-2-carboxylate, sodium 4-amino-3-chloro-5-fluoro-6-(7-fluoro-1H-indol-6-yl)pyridine-2-carboxylate, butyl 4-amino-3-chloro-5-fluoro-6-(7-fluoro-1H-indol-6-yl)pyridine-2-carboxylate, 4-hydroxy-1-methyl-3-[4-(trifluoromethyl)pyridin-2-yl]imidazolidin-2-one, 3-(5-tert-butyl-1,2-oxazol-3-yl)-4-hydroxy-1-methylimidazolidin-2-one.

Examples of plant growth regulators as possible mixing partners are:

Abscisic acid, acibenzolar, acibenzolar-S-methyl, 1-aminocyclopro-1-ylcarboxylic acid and derivatives thereof, 5-aminolevulinic acid, ancymidol, 6-benzylaminopurine, brassinolide, brassinolide-ethyl, catechin, chitooligosaccharides (CO; COs differ from LCOs in that they have no pendant fatty acid chain, which is characteristic of LCOs. COs, sometimes also referred to as N-acetylchitooligosaccharides, likewise consist of GlcNAc radicals, but have side-chain decorations which distinguish them from chitin molecules [(C₈H₁₃NO₅)ₙ, CAS No. 1398-61-4] and chitosan molecules [(C₈H₁₁NO₄)ₙ, CAS No. 9012-76-4], chitin compounds, chlormequat chloride, cloprop, cyclanilide, 3-(cycloprop-1-enyl)propionic acid, daminozide, dazomet, dazomet-sodium, n-decanol, dikegulac, dikegulac-sodium, endothal, endothal-dipotassium, -disodium and -mono(N,N-dimethylalkylammonium), ethephon, flumetralin, flurenol, flurenol-butyl, flurenol-methyl, flurprimidol, forchlorfenuron, gibberellic acid, inabenfid, indole-3-acetic acid (IAA), 4-indol-3-ylbutyric acid, isoprothiolane, probenazole, jasmonic acid, jasmonic acid or derivatives thereof (e.g. methyl jasmonate), lipo-chitooligosaccharides (LCO, sometimes also referred to as symbiotic nodulation (Nod) signals (or Nod factors) or as Myc factors), and consist of an oligosaccharide skeleton of $1,4-linked N-acetyl-D-glucosamine (“GlcNAc”) residues having an N-linked fatty acyl chain condensed at the non-reducing end. As is known to the person skilled in the art, LCOs differ in the number of GlcNAc radicals in their skeleton, in their length and in the degree of saturation of the fatty acyl chain and in the substitutions of reducing and non-reducing sugar moieties), linoleic acid or derivatives thereof, linolenic acid or derivatives thereof, maleic hydrazide, mepiquat chloride, mepiquat pentaborate, 1-methylcyclopropene, 3′-methylabscisic acid, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, 4-phenylbutyric acid, N-phenylphthalamic acid, prohexadione, prohexadione-calcium, prohydrojasmone, salicylic acid, methyl salicylate, strigolactone, tecnazene, thidiazuron, triacontanol, trinexapac, trinexapac-ethyl, tsitodef, uniconazole, uniconazole-P, 2-fluoro-N-(3-methoxyphenyl)-9H-purin-6-amine.

Safeners which can be used in combination with the inventive compounds of the formula (I) and optionally in combinations with further active ingredients such as insecticides, acaricides, herbicides, fungicides as listed above are preferably selected from the group consisting of:
S1) Compounds of the formula (S1)

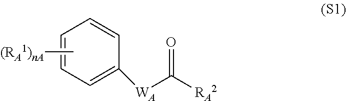

where the symbols and indices are defined as follows:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably
a radical from the group of (W_{A}¹) to (W_{A}⁴),

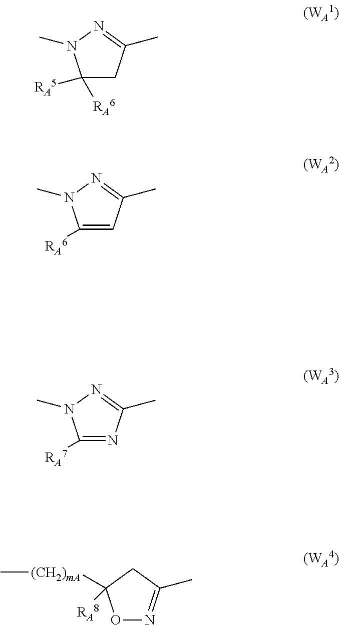

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹, where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
preferably:
a) compounds of the dichlorophenylpyrazoline-3-carboxylic acid type (Sla), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S16), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

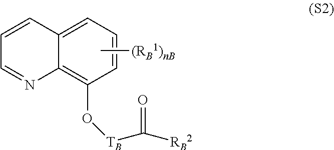

where the symbols and indices have the meanings below:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated
or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbon radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2a), preferably
1-methylhexyl (5-chloro-8-quinolinoxy)acetate (“cloquintocet-mexyl”) (S2-1),
(1,3-dimethylbut-1-yl) (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
   2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;
   b) compounds of the (5-chloro-8-quinolinoxy)malonic acid type (S26), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
   S3) Compounds of the formula (S3)

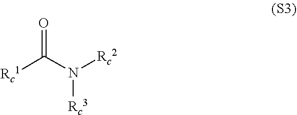

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
preferably:
   active ingredients of the dichloroacetamide type, which are frequently used as pre-emergence safeners (soil-acting safeners), for example
“dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (S3-6),
“AD-67” or “MON 4660” (3-dichloroacetyl-1-oxa-3-azaspiro[4.5]decane) from Nitrokemia or Monsanto (S3-7),
“TI-35” (1-dichloroacetylazepane) from TRI-Chemical RT (S3-8),
“diclonon” (dicyclonon) or “BAS145138” or “LAB145138” (S3-9)
((RS)-1-dichloroacetyl-3,3,8a-trimethylperhydropyrrolo[1,2-a]pyrimidin-6-one) from BASF,
“furilazole” or “MON 13900” ((RS)-3-dichloroacetyl-5-(2-furyl)-2,2-dimethyloxazolidine) (S3-10); and the (R) isomer thereof (S3-11).
S4)N-acylsulfonamides of the formula (S4) and salts thereof,

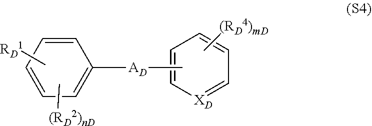

in which the symbols and indices are defined as follows:
A_{D} is SO₂—NR_{D}³—CO or CO—NR_{D}³-SO₂
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₈-C₆)-cycloalkenyl, phenyl or 3- to 6-membered heterocyclyl containing v_{D} heteroatoms from the group consisting of nitrogen, oxygen and sulfur, where the seven latter radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxyl, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
among these, preference is given to compounds of the N-acylsulfonamide type, for example of the formula (S4^{a}) below, which are known, for example, from WO-A-97/45016

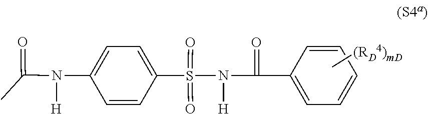

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃;
m_{D} is 1 or 2;
v_{D} is 0, 1, 2 or 3;
and also
acylsulfamoylbenzamides, for example of the formula (S4^{b}) below, which are known, for example, from WO-A-99/16744,

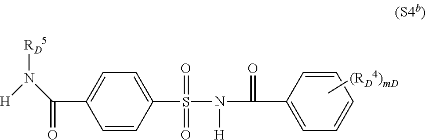

e.g. those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
and also
compounds of the N-acylsulfamoylphenylurea type of the formula (S4), which are known, for example, from EP-A-365484,

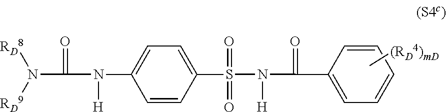

in which
R_{D}⁸ and R_{D}⁹ are independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃,
m_{D} Is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
   and also
   N-phenylsulfonylterephthalamides of the formula (S4d), which are known, for example, from CN 101838227,

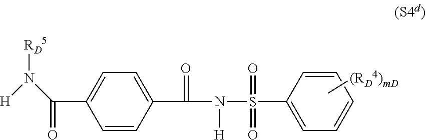

e.g. those in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₈-C₆)-cycloalkenyl.
S5) Active ingredients from the class of the hydroxyaromatics and the aromatic-aliphatic carboxylic acid derivatives (S5), for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example
1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,
in which the symbols and indices are defined as follows:

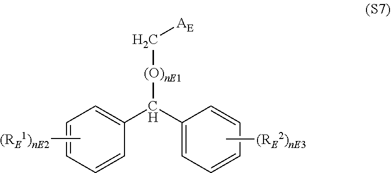

R_{E}¹, R_{E}² are independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴
R_{E}³, R_{E}⁴ are independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 Or 1
n_{E}², n_{E} are independently 0, 1 or 2, preferably:
diphenylmethoxyacetic acid,
ethyl diphenylmethoxyacetate,
methyl diphenylmethoxyacetate (CAS reg. no. 41858-19-9) (S7-1).
S8) Compounds of the formula (S8), as described in WO-A-98/27049,

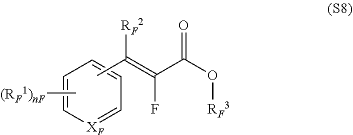

in which
X_{F} is CH or N,
n_{F} in the case that X_{F}=N is an integer from 0 to 4 and in the case that X_{F}=CH is an integer from 0 to 5,
R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,
preferably compounds in which
X_{F} is CH,
n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy,
or salts thereof.
S9) Active ingredients from the class of the 3-(5-tetrazolylcarbonyl)-2-quinolones (S9), for example
1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10a) or (S106)
   as described in WO-A-2007/023719 and WO-A-2007/023764

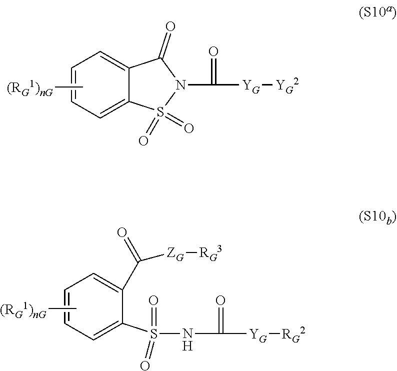

in which
R_{G}¹ is halogen, (C₁-C₄)-alkyl, methoxy, nitro, cyano, CF₃, OCF₃,
Y_{G}, Z_{G} independently of one another represent O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active ingredients of the oxyimino compounds type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
“fluxofenim” (1-(4-chlorophenyl)-2,2,2-trifluoro-1-ethanone 0-(1,3-dioxolan-2-ylmethyl)oxime) (S11-2), which is known as a seed-dressing safener for millet/sorghum against metolachlor damage, and
“cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
“naphthalic anhydride” (1,8-naphthalenedicarboxylic anhydride) (S13-1), which is known as a seed-dressing safener for maize against thiocarbamate herbicide damage,
“fenclorim” (4,6-dichloro-2-phenylpyrimidine) (S13-2), which is known as a safener for pretilachlor in sown rice,
“flurazole” (benzyl 2-chloro-4-trifluoromethyl-1,3-thiazole-5-carboxylate) (S13-3), which is known as a seed-dressing safener for millet/sorghum against alachlor and metolachlor damage,
“CL 304415” (CAS Reg. No. 31541-57-8) (4-carboxy-3,4-dihydro-2H-1-benzopyran-4-acetic acid) (S13-4) from American Cyanamid, which is known as a safener for corn against damage by imidazolinones,
“MG 191” (CAS Reg. No. 96420-72-3) (2-dichloromethyl-2-methyl-1,3-dioxolane) (S13-5) from Nitrokemia, which is known as a safener for maize,
“MG 838” (CAS Reg. No. 133993-74-5) (2-propenyl 1-oxa-4-azaspiro[4.5]decane-4-carbodithioate) (S13-6) from Nitrokemia,
“disulfoton” (0,0-diethyl S-2-ethylthioethyl phosphorodithioate) (S13-7),
“dietholate” (0,0-diethyl O-phenyl phosphorothioate) (S13-8),
“mephenate” (4-chlorophenyl methylcarbamate) (S13-9).
S14) Active ingredients which, in addition to herbicidal action against harmful plants, also have safener action on crop plants such as rice, for example
“dimepiperate” or “MY 93” (S-1-methyl 1-phenylethylpiperidine-1-carbothioate), which is known as a safener for rice against damage by the herbicide molinate,
“daimuron” or “SK 23” (1-(1-methyl-1-phenylethyl)-3-p-tolylurea), which is known as a safener for rice against damage by the herbicide imazosulfuron,
“cumyluron”=“JC 940” (3-(2-chlorophenylmethyl)-1-(1-methyl-1-phenylethyl)urea, see JP-A-60087254), which is known as safener for rice against damage by some herbicides,
“methoxyphenone” or “NK 049” (3,3′-dimethyl-4-methoxybenzophenone), which is known as a safener for rice against damage by some herbicides,
“CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

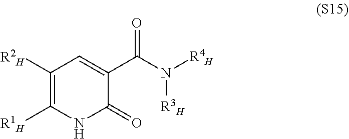

as described in WO-A-2008/131861 and WO-A-2008/131860 in which
R_{H}¹ is a (C₁-C₆)-haloalkyl radical and
R_{H}² is hydrogen or halogen and
R_{H}³, R_{H}⁴ are independently hydrogen, (C₁-C₁₆)-alkyl, (C₂-C₁₆)-alkenyl or (C₂-C₁₆)-alkynyl,
where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
where each of the 4 latter radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichloro-ethyl).

Particularly preferred safeners are mefenpyr-diethyl, cyprosulfamide, isoxadifen-ethyl, cloquintocet-mexyl, dichlormid and metcamifen.

Wettable powders are preparations uniformly dispersible in water which, in addition to the active ingredient and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57. For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103. The agrochemical preparations contain generally 0.1% to 99% by weight, especially 0.1% to 95% by weight, of compounds of the invention. In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partially on whether the active ingredient is in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The required application rate of the compounds of the formula (I) and their salts varies according to the external conditions such as, inter alia, temperature, humidity and the type of herbicide used. It can vary within wide limits, for example between 0.001 and 10.0 kg/ha or more of active substance, but it is preferably between 0.005 and 5 kg/ha, more preferably in the range of from 0.01 to 1.5 kg/ha, more preferably in the range of from 0.05 to 1 kg/ha. This applies both to pre-emergence and to post-emergence application.

A carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, maize cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins, and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions of the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active ingredients can be combined with any solid or liquid additive commonly used for formulation purposes. In general, the compositions and formulations of the invention contain between 0.05% and 99% by weight, 0.01% and 98% by weight, preferably between 0.1% and 95% by weight, more preferably between 0.5% and 90% active ingredient, most preferably between 10 and 70 percent by weight. The active ingredients or compositions of the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, sprayable powders, soluble powders, dusts and granules, water-soluble granules or tablets, water-soluble powders for the treatment of seed, wettable powders, natural products and synthetic substances impregnated with active ingredient, and also microencapsulations in polymeric substances and in coating materials for seed, and also ULV cold-fogging and warm-fogging formulations.

The formulations mentioned can be produced in a manner known per se, for example by mixing the active ingredients with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions of the invention include not only formulations which are already ready for use and can be deployed with a suitable apparatus onto the plant or the seed, but also commercial concentrates which have to be diluted with water prior to use.

The active ingredients of the invention may be present as such or in their (commercial standard) formulations, or else in the use forms prepared from these formulations as a mixture with other (known) active ingredients, such as insecticides, attractants, sterilants, bactericides, acaricides, nematicides, fungicides, growth regulators, herbicides, fertilizers, safeners or semiochemicals.

The inventive treatment of the plants and plant parts with the active ingredients or compositions is effected directly or by action on their surroundings, habitat or storage space by the customary treatment methods, for example by dipping, spraying, atomizing, irrigating, evaporating, dusting, fogging, broadcasting, foaming, painting, spreading-on, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

As also described below, the treatment of transgenic seed with the active ingredients or compositions of the invention is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition of the invention and/or further additives applied to the seed is chosen such that the germination of the seed is not impaired and the plant which arises therefrom is not damaged. This has to be ensured particularly in the case of active ingredients which can exhibit phytotoxic effects at certain application rates.

The compositions of the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417 A, 4,245,432 A, 4,808,430, 5,876,739, US 2003/0176428 A1, WO 2002/080675 A1, WO 2002/028186 A2.

The active ingredients of the invention can be converted to the customary seed-dressing formulations, such as solutions, emulsions, suspensions, powders, foams, slurries or other coating compositions for seed, and also ULV formulations.

These formulations are produced in a known manner, by mixing the active ingredients with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active ingredients. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active ingredients. Preference can be given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active ingredients. Silicone antifoams and magnesium stearate can be used with preference.

Preservatives which may be present in the seed-dressing formulations usable in accordance with the invention are all substances usable for such purposes in agrochemical compositions. Examples include dichlorophen and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active ingredients of the invention, given good plant compatibility, favorable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

Plants which can be treated in accordance with the invention include the following main crop plants: maize, soybean, cotton, Brassica oil seeds such as Brassica napus (e.g. Canola), Brassica rapa, B. juncea (e.g. (field) mustard) and Brassica carinata, rice, wheat, sugar beet, sugar cane, oats, rye, barley, millet and sorghum, triticale, flax, grapes and various fruit and vegetables from various botanic taxa, for example Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes, potatoes, peppers, aubergines), Liliaceae sp., Compositae sp. (for example lettuce, artichokes and chicory—including root chicory, endive or common chicory), Umbelliferae sp. (for example carrots, parsley, celery and celeriac), Cucurbitaceae sp. (for example cucumbers—including gherkins, pumpkins, watermelons, calabashes and melons), Alliaceae sp. (for example leeks and onions), Cruciferae sp. (for example white cabbage, red cabbage, broccoli, cauliflower, Brussels sprouts, pak choi, kohlrabi, radishes, horseradish, cress and chinese cabbage), Leguminosae sp. (for example peanuts, peas, and beans—for example common beans and broad beans), Chenopodiaceae sp. (for example Swiss chard, fodder beet, spinach, beetroot), Malvaceae (for example okra), Asparagaceae (for example asparagus); useful plants and ornamental plants in the garden and woods; and in each case genetically modified types of these plants.

As mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) which have been grown by conventional breeding, by mutagenesis or by recombinant DNA techniques. They may be cultivars, varieties, biotypes and genotypes.

The treatment method of the invention can be used for the treatment of genetically modified organisms (GMOs), e.g. plants or seeds. Genetically modified plants (or transgenic plants) are plants in which a heterologous gene has been stably integrated into the genome. The term “heterologous gene” means essentially a gene which is provided or assembled outside the plant and which, upon introduction into the nuclear genome, the chloroplast genome or the mitochondrial genome, imparts to the transformed plant novel or improved agronomical or other traits because it expresses a protein or polypeptide of interest or another gene which is present in the plant, or other genes which are present in the plant are down-regulated or switched off (for example by means of antisense technology, co-suppression technology or RNAi technology [RNA interference]). A heterologous gene that is located in the genome is also called a transgene. A transgene that is defined by its specific presence in the plant genome is called a transformation or transgenic event.

Depending on the plant species or plant cultivars, their location and growth conditions (soils, climate, vegetation period, diet), the inventive treatment may also result in superadditive (“synergistic”) effects. For example, the

following effects which exceed the effects actually to be expected are possible: reduced application rates and/or widened spectrum of activity and/or increased efficacy of the active ingredients and compositions which can be used in accordance with the invention, better plant growth, increased tolerance to high or low temperatures, increased tolerance to drought or to water or soil salinity, increased flowering performance, easier harvesting, accelerated maturation, higher harvest yields, bigger fruits, greater plant height, greener leaf color, earlier flowering, higher quality and/or a higher nutritional value of the harvested products, higher sugar concentration within the fruits, better storage stability and/or processability of the harvested products.

Plants and plant cultivars which are preferably treated in accordance with the invention include all plants which have genetic material which imparts particularly advantageous, useful traits to these plants (whether obtained by breeding and/or biotechnological means).

Examples of nematode-resistant plants are described, for example, in the following U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 and 12/497,221.

Plants that may be treated according to the invention are hybrid plants that already express the characteristics of heterosis, or hybrid effect, which results in generally higher yield, vigor, better health and resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (e.g. in maize) be produced by detasselling (i.e. the mechanical removal of the male reproductive organs or male flowers) but, more typically, male sterility is the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate by various methods. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science, 221, 370-371), the CP4 gene of the bacterium Agrobacterium sp. (Barry et al., 1992, Curr. Topics Plant Physiol. 7, 139-145), the genes encoding a petunia EPSPS (Shah et al., 1986, Science 233, 478-481), a tomato EPSPS (Gasser et al., 1988, J. Biol. Chem. 263, 4280-4289) or an Eleusine EPSPS (WO 01/66704). It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes. Plants which express EPSPS genes which impart glyphosate tolerance have been described. Plants which express other genes which impart glyphosate tolerance, for example decarboxylase genes, have been described.

Other herbicide-resistant plants are for example plants made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyse the reaction in which para-hydroxyphenylpyruvate (HPP) is converted to homogentisate. Plants tolerant to HPPD inhibitors can be transformed with a gene encoding a naturally-occurring resistant HPPD enzyme, or a gene encoding a mutated or chimeric HPPD enzyme, as described in WO 96/38567, WO 99/24585, WO 99/24586, WO 2009/144079, WO 2002/046387 or U.S. Pat. No. 6,768,044. Tolerance to HPPD inhibitors can also be obtained by transforming plants with genes encoding certain enzymes enabling the formation of homogentisate despite inhibition of the native HPPD enzyme by the HPPD inhibitor. Such plants are described in WO 99/34008 and WO 02/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928. In addition, plants can be made more tolerant to HPPD inhibitors by inserting into the genome thereof a gene which encodes an enzyme which metabolizes or degrades HPPD inhibitors, for example CYP450 enzymes (see WO 2007/103567 and WO 2008/150473).

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright (Weed Science 2002, 50, 700-712). The production of sulfonylurea-tolerant plants and imidazolinone-tolerant plants has been described. Further sulfonylurea- and imidazolinone-tolerant plants have also been described.

Further plants tolerant to imidazolinones and/or sulfonylureas can be obtained by induced mutagenesis, by selection in cell cultures in the presence of the herbicide or by mutation breeding (cf., for example, for soya beans U.S. Pat. No. 5,084,082, for rice WO 97/41218, for sugar beet U.S. Pat. No. 5,773,702 and WO 99/057965, for lettuce U.S. Pat. No. 5,198,599 or for sunflower WO 01/065922).

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:
a. plants which contain a transgene capable of reducing the expression and/or the activity of the poly(ADP-ribose) polymerase (PARP) gene in the plant cells or plants;
b. plants which contain a stress tolerance-enhancing transgene capable of reducing the expression and/or the activity of the PARG-encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage stability of the harvested product and/or altered properties of specific components of the harvested product such as, for example:
1) Transgenic plants which synthesize a modified starch which, in its physicochemical characteristics, in particular the amylose content or the amylose/amylopectin ratio, the degree of branching, the average chain length, the side chain distribution, the viscosity behaviour, the gelling strength, the starch granule size and/or the starch granule morphology, is changed in comparison with the synthesized starch in wild-type plant cells or plants, so that this modified starch is better suited to specific applications.
2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.
3) Transgenic plants which produce hyaluronan.
4) Transgenic plants or hybrid plants such as onions with particular properties, such as “high soluble solids content”, “low pungency” (LP) and/or “long storage” (LS).

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:
a) plants, such as cotton plants, containing an altered form of cellulose synthase genes;
b) plants, such as cotton plants, which contain an altered form of rsw2 or rsw3 homologous nucleic acids, such as cotton plants with an increased expression of sucrose phosphate synthase;
c) plants, such as cotton plants, with increased expression of sucrose synthase;
d) plants, such as cotton plants, wherein the timing of the plasmodesmatal gating at the basis of the fibre cell is altered, for example through downregulation of fibre-selective β-1,3-glucanase;
e) plants, such as cotton plants, which have fibres with altered reactivity, for example through expression of the N-acetylglucosaminetransferase gene, including nodC, and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:
a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Plants or plant cultivars (which can be obtained by plant biotechnology methods such as genetic engineering) which may also be treated according to the invention are plants such as potatoes which are virus-resistant, for example to the potato virus Y (SY230 and SY233 events from Tecnoplant, Argentina), or which are resistant to diseases such as potato late blight (e.g. RB gene), or which exhibit reduced cold-induced sweetness (which bear the genes Nt-Inh, II-INV) or which exhibit the dwarf phenotype (A-20 oxidase gene).

Plants or plant cultivars (obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants containing a mutation imparting such altered characteristics, and include plants such as oilseed rape with retarded or reduced seed shattering.

Particularly useful transgenic plants which can be treated according to the invention are plants with transformation events or combinations of transformation events which are the subject of granted or pending petitions for nonregulated status in the USA at the Animal and Plant Health Inspection Service (APHIS) of the United States Department of Agriculture (USDA). Information relating to this is available at any time from APHIS (4700 River Road Riverdale, MD 20737, USA), for example via the website http://www.aphis.usda.gov/brs/not_reg.html. At the filing date of this application, the petitions with the following information were either granted or pending at APHIS:
Petition: Identification number of the petition. The technical description of the transformation event can be found in the specific petition document available from APHIS on the website via the petition number. These descriptions are hereby disclosed by reference.
Extension of a petition: Reference to an earlier petition for which an extension of scope or term is being requested.
Institution: Name of the person submitting the petition.
Regulated article: The plant species in question.
Transgenic phenotype: The trait imparted to the plant by the transformation event.
Transformation event or line: The name of the event(s) (sometimes also referred to as line(s)) for which nonregulated status is being requested.
APHIS documents: Various documents which have been published by APHIS with regard to the petition or can be obtained from APHIS on request.

Particularly useful transgenic plants which can be treated in accordance with the invention are plants which comprise one or more genes which code for one or more toxins, for example the transgenic plants which are sold under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned include maize varieties, cotton varieties and soya bean varieties which are available under the following trade names: Roundup Ready® (tolerance to glyphosates, for example maize, cotton, soya beans), Liberty Link® (tolerance to phosphinothricin, for example oilseed rape), IMI® (tolerance to imidazolinone) and SCS® (tolerance to sulfonylurea), for example maize. Herbicide-resistant plants (plants bred in a conventional manner for herbicide tolerance) which may be mentioned include the varieties sold under the name Clearfield® (for example maize).

NMR data of selected examples: The ¹H NMR data of selected examples of compounds of the general formula (I) are stated in two different ways, namely (a) conventional NMR evaluation and interpretation or (b) in the form of ¹H NMR peak lists according to the method described below.

### a) Conventional NMR Interpretation

### Example No. I-10

¹H NMR (CDCl₃ δ, ppm): 1.20 (d, 3H), 2.85 (sext, 1H), 3.65 (d, 3H), 3.70 (s, 3H), 4.30 (dd, 1H), 4.45 (dd, 1H), 5.90 (s, 1H), 6.95 (m, 1H), 7.05 (t, 1H), 7.20 (t, 1H), 7.35 (m, 1H), 7.45 (t, 1H), 7.75 (m, 1H), 8.10 (s, 1H).

### Example No. I-12

¹H NMR (d6-DMSO: δ, ppm): 3.55 (s, 3H), 5.80 (s, 1H), 7.25-7.35 (m, 3H), 7.50 (m, 1H), 7.60 (t, 1H), 7.95 (m, 1H), 8.20 (d, 1H).

### Example No. I-13

¹H NMR (CDCl₃ δ, ppm): 3.70 (s, 3H), 3.85 (s, 3H), 5.95 (s, 1H), 7.15 (t, 1H), 7.30 (m, 1H), 7.45 (m, 2H), 8.30 (m, 1H), 8.50 (m, 1H).

### Example No. I-18

¹H NMR (CDCl₃ δ, ppm): 3.70 (s, 3H), 3.85 (s, 3H), 5.95 (s, 1H), 7.15 (t, 1H), 7.30 (m, 1H), 7.45 (m, 2H), 8.30 (m, 1H), 8.50 (m, 1H).

### Example No. I-63

¹H NMR (CDCl₃ δ, ppm): 1.30 (t, 3H), 2.70 (t, 2H), 3.65 (s, 3H), 3.85 (m, 1H), 4.05 (m, 1H), 4.50 (t, 2H), 5.95 (s, 1H), 6.95 (dd, 1H), 7.05 (dt, 1H), 7.20 (t, 1H), 7.35 (m, 1H), 7.45 (dt, 1H), 7.75 (dt, 1H), 8.10 (d, 1H).

### Example No. I-64

¹H NMR (CDCl₃ δ, ppm): 1.30 (t, 3H), 2.70 (t, 2H), 3.65 (s, 3H), 3.85 (m, 1H), 4.05 (m, 1H), 4.50 (t, 2H), 5.95 (s, 1H), 6.95 (dd, 1H), 7.05 (dt, 1H), 7.20 (t, 1H), 7.35 (m, 1H), 7.45 (dt, 1H), 7.75 (dt, 1H), 8.10 (d, 1H).

### b) NMR Peak List Method

The 1H NMR data of selected examples are noted in the form of 1H NMR peak lists. For each signal peak, first the δ value in ppm and then the signal intensity in round brackets are listed. The δ value-signal intensity number pairs for different signal peaks are listed with separation from one another by semicolons.

The peak list for one example therefore takes the form of:
δ₁ (intensity₁); δ₂ (intensity₂); . . . ; δᵢ(intensityᵢ); . . . ; δₙ(intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in this case to identify reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in the Research Disclosure Database Number 564025.

I-01: ¹H NMR(400.6 MHz, CDCl3):
δ = 8.1084 (1.7); 8.1064 (1.4); 8.1042 (1.4); 8.1022 (1.9); 7.7670 (0.9); 7.7607 (0.9); 7.7483 (1.0); 7.7457 (1.1); 7.7420 (1.1);
7.7395 (1.1); 7.7270 (1.0); 7.7208 (1.0); 7.4214 (0.7); 7.4170 (0.8); 7.4022 (1.2); 7.3978 (1.4); 7.3833 (0.8); 7.3789 (1.0);
7.3759 (0.5); 7.3591 (0.5); 7.3569 (0.7); 7.3551 (0.6); 7.3524 (0.6); 7.3506 (0.6); 7.3447 (0.8); 7.3430 (0.6); 7.3402 (0.6);
7.3384 (0.6); 7.3362 (0.7); 7.3317 (0.6); 7.3240 (0.6); 7.3195 (0.5); 7.2614 (26.1); 7.2282 (0.8); 7.2267 (0.9); 7.2247 (0.9);
7.2053 (1.3); 7.1897 (0.5); 7.1881 (0.6); 7.1864 (0.5); 7.0496 (0.9); 7.0464 (0.9); 7.0288 (0.9); 7.0250 (1.4); 7.0212 (0.9);
7.0037 (0.8); 7.0005 (0.8); 6.9465 (1.2); 6.9450 (1.3); 6.9391 (1.2); 6.9376 (1.2); 6.9254 (1.2); 6.9238 (1.2); 6.9179 (1.2);
6.9164 (1.1); 5.9638 (10.0); 4.3350 (1.0); 4.3323 (1.0); 4.3171 (3.2); 4.3146 (3.2); 4.2993 (3.3); 4.2968 (3.2); 4.2814 (1.1);
4.2791 (1.0); 4.0589 (1.4); 4.0528 (0.6); 4.0413 (1.4); 4.0351 (1.8); 4.0174 (1.8); 3.9997 (0.5); 3.9172 (0.5); 3.8996 (1.7);
3.8819 (1.8); 3.8757 (1.3); 3.8644 (0.6); 3.8581 (1.3); 1.7270 (3.8); 1.5555 (15.4); 1.3509 (6.2); 1.3332 (14.5); 1.3142 (16.0);
1.2963 (6.6); 0.0079 (1.1); −0.0002 (39.4); −0.0085 (1.1)
I-02: ¹H NMR(400.6 MHz, CDCl3):
δ = 8.2765 (1.2); 8.2747 (1.3); 8.2727 (1.3); 8.2710 (1.3); 8.2649 (1.3); 8.2631 (1.3); 8.2611 (1.3); 8.2595 (1.2); 7.4813 (0.8);
7.4776 (0.9); 7.4607 (1.3); 7.4586 (1.2); 7.4570 (1.4); 7.4550 (1.0); 7.4380 (1.2); 7.4342 (1.2); 7.3492 (1.2); 7.3401 (1.4);
7.3376 (1.3); 7.3285 (2.1); 7.3195 (1.0); 7.3169 (1.3); 7.3114 (3.1); 7.3101 (2.3); 7.3079 (1.3); 7.3061 (1.3); 7.2949 (1.8);
7.2895 (6.6); 7.2845 (1.2); 7.2606 (54.0); 7.2570 (8.0); 7.2512 (1.8); 7.2403 (1.1); 7.2349 (2.9); 7.2271 (0.6); 6.0342 (9.2);
4.3190 (1.1); 4.3172 (1.1); 4.3011 (3.5); 4.2994 (3.3); 4.2832 (3.6); 4.2817 (3.3); 4.2653 (1.2); 4.0504 (1.4); 4.0443 (0.6);
4.0327 (1.4); 4.0265 (1.8); 4.0088 (1.8); 3.9912 (0.5); 3.9116 (0.6); 3.8941 (1.6); 3.8764 (1.6); 3.8702 (1.2); 3.8589 (0.5);
3.8525 (1.2); 1.5459 (11.7); 1.3375 (5.8); 1.3198 (13.4); 1.3182 (10.4); 1.3019 (7.7); 1.3003 (16.0); 1.2825 (6.6); 0.9234
(0.5); 0.9066 (0.5); 0.0080 (1.9); −0.0002 (77.3); −0.0085 (2.3)
I-03: ¹H NMR(400.6 MHz, CDCl3):
δ = 8.2867 (0.6); 8.2848 (0.7); 8.2829 (0.7); 8.2812 (0.6); 8.2751 (0.6); 8.2733 (0.7); 8.2713 (0.7); 8.2697 (0.6); 7.4798 (0.5);
7.4629 (0.7); 7.4608 (0.6); 7.4591 (0.8); 7.4571 (0.6); 7.4402 (0.7); 7.4364 (0.7); 7.3555 (0.7); 7.3464 (0.8); 7.3439 (0.7);
7.3348 (1.2); 7.3257 (0.5); 7.3233 (0.6); 7.3160 (1.8); 7.3145 (1.5); 7.3106 (0.7); 7.2996 (0.9); 7.2941 (3.6); 7.2889 (0.6);
7.2608 (27.0); 7.2577 (3.9); 7.2520 (1.0); 7.2410 (0.7); 7.2368 (1.2); 7.2357 (1.7); 6.0273 (4.8); 3.8444 (14.4); 3.6785 (15.7);
2.9650 (5.6); 2.7764 (0.7); 1.5444 (16.0); 0.0079 (1.0); −0.0002 (40.5); −0.0085 (1.1)
I-04: ¹H NMR(400.6 MHz, CDCl3):
δ = 8.1127 (0.9); 8.1106 (0.7); 8.1085 (0.7); 8.1065 (0.9); 7.7491 (0.5); 7.7465 (0.6); 7.7429 (0.6); 7.7403 (0.6); 7.7279 (0.5);
7.4115 (0.6); 7.4072 (0.7); 7.3883 (0.5); 7.2606 (29.5); 7.2294 (0.5); 7.2097 (0.6); 7.0296 (0.7); 6.9501 (0.6); 6.9486 (0.6);
6.9427 (0.6); 6.9411 (0.6); 6.9290 (0.6); 6.9274 (0.6); 6.9215 (0.6); 6.9199 (0.6); 5.9592 (5.0); 3.8576 (14.6); 3.6882 (16.0);
1.9660 (1.0); 1.7396 (0.9); 1.5425 (12.4); 0.0079 (1.2); −0.0002 (43.4); −0.0085 (1.2)
I-05: ¹H NMR(400.6 MHz, CDCl3):
δ = 8.2427 (0.6); 8.2410 (0.7); 8.2391 (0.7); 8.2373 (0.6); 8.2311 (0.7); 8.2294 (0.7); 8.2275 (0.7); 8.2257 (0.7); 8.1461 (0.8);
8.1441 (0.7); 8.1419 (0.7); 8.1398 (1.0); 7.8635 (0.5); 7.8511 (0.5); 7.8486 (0.6); 7.8448 (0.6); 7.8423 (0.7); 7.8299 (0.5);
7.8236 (0.6); 7.5245 (0.5); 7.5075 (0.7); 7.5049 (0.7); 7.5039 (0.8); 7.5014 (0.6); 7.4844 (0.7); 7.4807 (0.7); 7.3664 (0.6);
7.3574 (0.7); 7.3548 (0.7); 7.3458 (1.1); 7.3367 (0.5); 7.3341 (0.6); 7.2614 (18.2); 6.9702 (0.6); 6.9686 (0.7); 6.9627 (0.6);
6.9611 (0.7); 6.9490 (0.6); 6.9473 (0.7); 6.9415 (0.6); 6.9399 (0.6); 6.0108 (5.2); 3.8525 (14.3); 3.6874 (16.0); 2.9651 (9.0);
2.7762 (0.9); 1.5513 (1.1); 0.0080 (0.6); −0.0002 (24.4); −0.0085 (0.8)
I-06: ¹H NMR(400.6 MHz, CDCl3):
δ = 8.2927 (0.7); 8.2910 (0.8); 8.2890 (0.8); 8.2873 (0.7); 8.2811 (0.7); 8.2794 (0.8); 8.2774 (0.8); 8.2758 (0.7); 7.4475 (0.5);
7.4437 (0.5); 7.4268 (0.8); 7.4248 (0.7); 7.4230 (0.8); 7.4212 (0.6); 7.4042 (0.7); 7.4005 (0.7); 7.3381 (1.3); 7.3360 (1.2);
7.3341 (1.3); 7.3317 (0.8); 7.3290 (1.2); 7.3248 (1.2); 7.3210 (3.3); 7.3186 (3.0); 7.3137 (3.8); 7.3105 (2.5); 7.3066 (1.7);
7.3017 (2.1); 7.2927 (0.7); 7.2606 (16.9); 6.0485 (5.0); 3.8463 (14.9); 3.6839 (16.0); 1.5440 (2.2); 0.0080 (0.8); −0.0002
(27.4); −0.0085 (0.8)
I-06: ¹H NMR(400.6 MHz, CDCl3):
δ = 8.2924 (0.6); 8.2906 (0.7); 8.2886 (0.7); 8.2869 (0.6); 8.2808 (0.6); 8.2790 (0.7); 8.2770 (0.7); 8.2753 (0.6); 7.4436 (0.5);
7.4267 (0.8); 7.4247 (0.6); 7.4229 (0.8); 7.4210 (0.6); 7.4041 (0.7); 7.4003 (0.7); 7.3379 (1.1); 7.3357 (1.0); 7.3340 (1.1);
7.3315 (0.6); 7.3287 (1.1); 7.3247 (1.0); 7.3207 (3.0); 7.3183 (2.7); 7.3135 (3.5); 7.3102 (2.2); 7.3064 (1.5); 7.3015 (1.9);
7.2926 (0.6); 7.2608 (9.0); 6.0484 (4.8); 3.8459 (14.7); 3.6837 (16.0); 1.5521 (1.3); −0.0002 (14.2)
I-07: ¹H NMR(400.6 MHz, CDCl3):
δ = 8.1369 (0.6); 8.1351 (0.7); 8.1332 (0.7); 8.1315 (0.7); 8.1252 (0.7); 8.1234 (0.8); 8.1215 (0.7); 8.1199 (0.7); 7.5255 (0.6);
7.5218 (0.6); 7.5049 (0.7); 7.5015 (1.0); 7.4982 (0.6); 7.4813 (0.7); 7.4776 (0.7); 7.4227 (0.6); 7.4071 (0.6); 7.3038 (0.7);
7.2950 (0.8); 7.2922 (0.7); 7.2833 (1.2); 7.2744 (0.6); 7.2716 (0.6); 7.2608 (17.5); 6.9259 (0.6); 6.7674 (0.5); 6.0087 (5.0);
3.8530 (14.6); 3.6917 (16.0); 2.7761 (4.8); 1.5462 (4.6); 0.0079 (0.7); −0.0002 (26.1); −0.0085 (0.8)
I-08: ¹H NMR(400.6 MHz, CDCl3):
δ = 8.1254 (1.2); 8.1237 (1.3); 8.1218 (1.3); 8.1201 (1.2); 8.1138 (1.2); 8.1121 (1.4); 8.1102 (1.4); 8.1086 (1.2); 7.5234 (1.0);
7.5196 (1.1); 7.5027 (1.3); 7.4994 (1.8); 7.4961 (1.1); 7.4792 (1.2); 7.4754 (1.2); 7.4472 (0.7); 7.4315 (0.8); 7.4260 (1.1);
7.4103 (1.1); 7.4057 (0.9); 7.3899 (0.8); 7.2971 (1.2); 7.2883 (1.3); 7.2855 (1.2); 7.2766 (2.1); 7.2677 (1.1); 7.2649 (1.2);
7.2607 (26.9); 7.2561 (1.5); 6.9517 (0.5); 6.9492 (0.6); 6.9454 (0.6); 6.9430 (0.6); 6.9319 (0.7); 6.9297 (1.0); 6.9275 (0.8);
6.9256 (0.8); 6.9235 (1.0); 6.9103 (0.5); 6.9077 (0.5); 6.9040 (0.6); 6.9016 (0.5); 6.7860 (0.8); 6.7798 (0.8); 6.7640 (0.9);
6.7612 (1.0); 6.7579 (0.9); 6.7551 (0.9); 6.7393 (0.8); 6.7332 (0.7); 6.0211 (9.4); 4.3263 (1.4); 4.3086 (4.5); 4.2908 (4.8);
4.2730 (1.6); 4.0671 (1.3); 4.0610 (0.6); 4.0495 (1.4); 4.0432 (1.8); 4.0255 (1.8); 4.0079 (0.6); 3.9057 (1.6); 3.8881 (1.7);
3.8818 (1.3); 3.8705 (0.6); 3.8641 (1.3); 1.5467 (11.0); 1.3445 (6.0); 1.3267 (15.5); 1.3083 (16.0); 1.2903 (6.3); 0.0080
(1.0); −0.0002 (38.1); −0.0085 (1.2)
I-09: ¹H NMR(400.6 MHz, CDCl3):
δ = 7.2716 (0.9); 7.2602 (36.6); 7.2521 (0.5); 7.0626 (0.5); 7.0384 (0.5); 5.9346 (2.7); 4.3107 (1.6); 4.2929 (1.7); 4.2751 (0.6);
4.0317 (0.5); 3.8935 (0.5); 3.8759 (0.5); 1.5358 (16.0); 1.3462 (1.8); 1.3284 (5.3); 1.3104 (5.3); 1.2924 (1.8); 0.0080 (1.8);
0.0057 (0.7); −0.0002 (58.0); −0.0084 (1.5)
I-11: ¹H NMR(400.6 MHz, CDCl3):
δ = 7.3085 (0.5); 7.3029 (0.8); 7.2928 (0.6); 7.2880 (0.8); 7.2822 (1.5); 7.2721 (0.9); 7.2667 (0.8); 7.2604 (18.8); 7.1597 (0.8);
7.1567 (0.8); 7.1542 (0.8); 7.1514 (0.8); 7.1383 (0.6); 7.1354 (0.6); 7.1327 (0.6); 7.1299 (0.6); 7.0678 (1.2); 7.0623 (1.0);
7.0437 (1.2); 7.0381 (1.1); 6.9269 (0.7); 6.9207 (0.7); 6.8368 (0.6); 6.8306 (0.5); 6.8148 (0.7); 6.8124 (0.7); 6.8087 (0.6);
6.8064 (0.6); 6.7906 (0.6); 5.9307 (5.4); 3.8537 (15.6); 3.6829 (16.0); 1.7268 (1.3); 1.5371 (6.3); 0.0078 (1.0); −0.0002
(28.7); −0.0085 (0.8)
I-14: ¹H NMR(400.6 MHz, CDCl3):
δ = 8.2440 (0.7); 8.2421 (0.8); 8.2403 (0.8); 8.2386 (0.7); 8.2323 (0.7); 8.2306 (0.8); 8.2287 (0.8); 8.2270 (0.7); 8.1452 (1.0);
8.1432 (0.7); 8.1409 (0.8); 8.1390 (1.0); 7.8699 (0.5); 7.8637 (0.5); 7.8512 (0.6); 7.8487 (0.6); 7.8450 (0.6); 7.8425 (0.6);
7.8301 (0.6); 7.8238 (0.6); 7.5251 (0.6); 7.5214 (0.6); 7.5044 (0.8); 7.5009 (0.8); 7.4984 (0.6); 7.4814 (0.7); 7.4776 (0.7);
7.3649 (0.7); 7.3559 (0.8); 7.3533 (0.7); 7.3443 (1.2); 7.3352 (0.6); 7.3326 (0.6); 7.3236 (0.5); 7.2616 (13.1); 6.9691 (0.7);
6.9676 (0.7); 6.9616 (0.7); 6.9601 (0.7); 6.9479 (0.7); 6.9463 (0.7); 6.9404 (0.7); 6.9389 (0.7); 5.9799 (5.5); 4.3373 (0.6);
4.3361 (0.6); 4.3194 (2.1); 4.3184 (2.0); 4.3015 (2.2); 4.3007 (2.1); 4.2835 (0.8); 3.6874 (16.0); 1.5584 (2.2); 1.3368 (3.7);
1.3190 (7.8); 1.3011 (3.6); −0.0002 (16.6)
I-15: ¹H NMR(400.6 MHz, CDCl3):
δ = 8.1990 (1.6); 8.1948 (1.3); 8.1930 (1.4); 8.0201 (1.4); 8.0184 (1.4); 8.0170 (1.3); 8.0147 (1.3); 8.0132 (1.3); 8.0115 (1.2);
7.7431 (0.6); 7.7369 (0.6); 7.7246 (0.9); 7.7220 (0.9); 7.7184 (0.8); 7.7158 (0.7); 7.7035 (0.7); 7.6973 (0.6); 7.6456 (0.6);
7.6385 (0.6); 7.6289 (0.8); 7.6237 (0.9); 7.6221 (0.9); 7.6167 (0.7); 7.6071 (0.7); 7.6000 (0.6); 7.2628 (5.5); 7.0377 (1.0);
7.0315 (0.9); 7.0302 (0.9); 7.0165 (1.0); 7.0103 (0.8); 7.0089 (0.8); 6.9499 (1.0); 6.9415 (1.0); 6.9280 (1.0); 6.9208 (0.9);
6.9197 (0.9); 5.9424 (5.1); 5.3007 (2.1); 3.8696 (15.2); 3.6906 (16.0); 1.5591 (0.9); 0.0699 (2.9); −0.0002 (7.3)
I-16: ¹H NMR(400.6 MHz, CDCl3):
δ = 8.4858 (1.7); 8.4789 (1.7); 8.2703 (0.9); 8.2667 (1.6); 8.2632 (0.9); 7.4544 (0.5); 7.4502 (0.6); 7.4472 (0.7); 7.4427 (0.7);
7.4403 (0.8); 7.4380 (0.7); 7.4358 (0.9); 7.4339 (1.2); 7.4299 (0.7); 7.4252 (0.7); 7.4207 (0.7); 7.4180 (1.1); 7.4136 (1.1);
7.3090 (0.5); 7.3048 (1.1); 7.3007 (0.6); 7.2888 (0.7); 7.2846 (0.6); 7.2620 (6.3); 7.1818 (0.7); 7.1780 (0.7); 7.1615 (1.1);
7.1577 (1.2); 5.9423 (5.1); 3.8614 (14.7); 3.6875 (16.0); 1.5650 (0.9); 1.5022 (1.8); 0.0698 (0.9); −0.0002 (8.4)
I-17: ¹H NMR(400.6 MHz, CDCl3):
δ = 8.4859 (1.9); 8.4790 (1.9); 8.2755 (1.1); 8.2720 (1.8); 8.2684 (1.0); 7.4547 (0.7); 7.4501 (0.8); 7.4476 (1.0); 7.4432 (1.0);
7.4327 (0.8); 7.4281 (1.2); 7.4259 (1.8); 7.4217 (1.2); 7.4096 (0.6); 7.4054 (0.6); 7.3144 (0.5); 7.3102 (0.5); 7.2983 (0.6);
7.2942 (1.2); 7.2901 (0.6); 7.2782 (0.7); 7.2740 (0.6); 7.2618 (5.7); 7.1726 (0.8); 7.1688 (0.7); 7.1524 (1.2); 7.1486 (1.1);
7.1321 (0.5); 5.9415 (5.1); 5.3000 (0.9); 3.8600 (14.8); 3.6864 (16.0); 1.5647 (0.8); 0.0698 (1.3); −0.0002 (7.8)
I-19: ¹H NMR(400.6 MHz, CDCl3):
δ = 8.4835 (1.8); 8.4766 (1.7); 8.2772 (1.0); 8.2735 (1.7); 8.2699 (0.9); 7.4388 (0.6); 7.4343 (0.7); 7.4312 (0.9); 7.4270 (1.0);
7.4169 (0.7); 7.4124 (1.0); 7.4102 (1.7); 7.4060 (1.1); 7.3940 (0.6); 7.3898 (0.6); 7.2921 (0.5); 7.2880 (0.5); 7.2761 (0.6);
7.2719 (1.2); 7.2677 (0.8); 7.2617 (6.4); 7.2559 (0.7); 7.2517 (0.6); 7.1566 (0.7); 7.1528 (0.7); 7.1364 (1.2); 7.1326 (1.2);
5.9330 (5.2); 5.3000 (0.9); 3.8581 (15.1); 3.6894 (0.5); 3.6842 (16.0); 1.5625 (1.2); 0.0696 (3.2); −0.0002 (8.9)
I-20: ¹H NMR(400.6 MHz, CDCl3):
δ = 8.1220 (0.8); 7.7551 (0.7); 7.7528 (0.7); 7.7489 (0.7); 7.7467 (0.6); 7.7341 (0.5); 7.4540 (0.5); 7.4390 (0.9); 7.4351 (0.9);
7.4202 (0.6); 7.4159 (0.6); 7.3485 (0.5); 7.2617 (8.7); 7.2359 (0.7); 7.2343 (0.7); 7.2160 (1.0); 7.0503 (0.6); 7.0471 (0.6);
7.0294 (0.6); 7.0256 (1.0); 7.0219 (0.6); 7.0044 (0.6); 6.9492 (0.5); 6.9425 (0.5); 6.9280 (0.5); 5.9063 (5.5); 5.3001 (0.7);
4.5320 (2.0); 4.5157 (4.3); 4.4995 (2.1); 3.6783 (16.0); 3.6596 (14.7); 2.7353 (1.9); 2.7190 (3.9); 2.7028 (1.8); 1.5563
(0.8); −0.0002 (12.2); −0.0029 (0.6)
I-21: ¹H NMR(600.3 MHz, CDCl3):
δ = 8.1215 (3.4); 8.1173 (3.9); 8.1118 (1.0); 7.7659 (1.3); 7.7617 (1.4); 7.7534 (1.7); 7.7518 (1.8); 7.7492 (1.8); 7.7476 (2.0);
7.7431 (0.6); 7.7393 (1.5); 7.7351 (1.4); 7.7290 (0.4); 7.4570 (0.3); 7.4542 (0.4); 7.4504 (1.2); 7.4475 (1.4); 7.4443 (0.7);
7.4414 (0.8); 7.4375 (2.2); 7.4348 (2.3); 7.4287 (0.5); 7.4249 (1.4); 7.4220 (1.4); 7.3698 (0.8); 7.3669 (0.8); 7.3645 (0.4);
7.3617 (1.0); 7.3587 (1.0); 7.3571 (1.3); 7.3561 (1.4); 7.3544 (1.4); 7.3490 (1.3); 7.3479 (1.3); 7.3462 (1.2); 7.3450 (1.1);
7.3434 (1.1); 7.3405 (1.0); 7.3353 (0.9); 7.3324 (0.8); 7.2615 (32.0); 7.2359 (0.4); 7.2298 (1.5); 7.2286 (1.6); 7.2229 (0.8);
7.2157 (2.5); 7.2101 (0.4); 7.2041 (1.1); 7.2028 (1.1); 7.0503 (0.4); 7.0483 (0.4); 7.0423 (1.5); 7.0402 (1.5); 7.0365 (0.5);
7.0339 (0.7); 7.0314 (0.6); 7.0284 (1.5); 7.0259 (2.5); 7.0235 (1.6); 7.0198 (0.5); 7.0176 (0.4); 7.0117 (1.4); 7.0096 (1.3);
6.9476 (0.6); 6.9432 (2.5); 6.9386 (2.1); 6.9340 (0.6); 6.9290 (2.4); 6.9245 (2.0); 5.9063 (19.2); 5.3001 (3.2); 4.5265 (5.5);
4.5157 (12.1); 4.5048 (5.8); 3.8707 (0.3); 3.6782 (45.8); 3.6599 (50.0); 3.2490 (0.3); 2.7298 (6.1); 2.7190 (12.4); 2.7082
(5.9); 1.5553 (2.6); 1.2552 (0.4); 0.1573 (0.4); 0.1265 (0.4); −0.0001 (3.6)
I-22: ¹H NMR(400.6 MHz, CDCl3):
δ = 8.1122 (1.1); 8.1060 (1.2); 7.7690 (0.5); 7.7628 (0.5); 7.7503 (0.6); 7.7479 (0.7); 7.7441 (0.6); 7.7416 (0.6); 7.7292 (0.6);
7.7229 (0.5); 7.4144 (0.8); 7.4101 (0.9); 7.3956 (0.5); 7.3912 (0.6); 7.2607 (12.8); 7.2276 (0.6); 7.2080 (0.8); 7.0509 (0.6);
7.0478 (0.5); 7.0301 (0.5); 7.0263 (0.9); 7.0226 (0.6); 6.9476 (0.8); 6.9414 (0.8); 6.9276 (0.7); 6.9201 (0.7); 5.9187 (5.5);
5.2999 (0.6); 4.3424 (0.8); 4.3248 (2.6); 4.3069 (2.7); 4.2891 (0.9); 3.6886 (16.0); 1.5438 (4.2); 1.3406 (3.7); 1.3228 (7.7);
1.3050 (3.6); 0.0080 (0.6); −0.0002 (19.2); −0.0085 (0.5)
I-23: ¹H NMR (400.6 MHz, CDCl3):
δ = 8.1121 (1.1); 8.1058 (1.1); 7.7691 (0.5); 7.7628 (0.5); 7.7503 (0.6); 7.7478 (0.6); 7.7441 (0.6); 7.7416 (0.6); 7.7291 (0.6);
7.7229 (0.5); 7.4143 (0.7); 7.4101 (0.8); 7.3955 (0.5); 7.3912 (0.6); 7.2607 (13.0); 7.2292 (0.5); 7.2275 (0.5); 7.2080 (0.8);
7.0509 (0.5); 7.0476 (0.5); 7.0301 (0.5); 7.0263 (0.8); 7.0225 (0.6); 6.9490 (0.7); 6.9475 (0.7); 6.9415 (0.7); 6.9400 (0.7);
6.9277 (0.7); 6.9262 (0.7); 6.9202 (0.7); 6.9188 (0.6); 5.9187 (5.6); 5.3000 (0.6); 4.3424 (0.8); 4.3246 (2.4); 4.3068 (2.6);
4.2890 (0.9); 3.6886 (16.0); 1.5438 (4.6); 1.3406 (3.7); 1.3228 (7.8); 1.3050 (3.6); 0.0080 (0.5); −0.0002 (19.4); −0.0085 (0.6)
I-24: ¹H-NMR(400.6 MHz, d₆-DMSO):
δ = 8.2143 (1.3); 8.2081 (1.4); 7.9769 (0.5); 7.9707 (0.5); 7.9558 (0.7); 7.9495 (0.7); 7.9364 (0.6); 7.9301 (0.5); 7.6087 (0.5);
7.5937 (0.9); 7.5893 (1.0); 7.5741 (0.6); 7.5698 (0.6); 7.3467 (0.6); 7.3438 (0.7); 7.3277 (0.9); 7.3243 (1.4); 7.3199 (0.8);
7.3051 (0.6); 7.2995 (1.4); 7.2941 (1.6); 7.2782 (0.9); 7.2729 (1.2); 7.2711 (1.1); 5.7921 (7.4); 3.6178 (1.3); 3.6156 (0.7);
3.6116 (0.7); 3.6094 (0.6); 3.6075 (1.0); 3.6012 (2.9); 3.5951 (1.0); 3.5909 (0.7); 3.5871 (1.2); 3.5846 (1.3); 3.5544 (16.0);
3.3210 (5.2); 2.5242 (0.7); 2.5195 (1.1); 2.5108 (13.4); 2.5062 (29.6); 2.5016 (41.2); 2.4970 (29.0); 2.4924 (12.7); 1.7761
(1.2); 1.7685 (1.0); 1.7642 (0.6); 1.7595 (3.5); 1.7548 (0.6); 1.7506 (1.0); 1.7430 (1.1); 1.3556 (2.5); −0.0002 (17.7); −0.0085
(0.5)
I-25: ¹H-NMR(400.6 MHz, d₆-DMSO):
δ = 8.2145 (1.5); 8.2082 (1.5); 7.9770 (0.6); 7.9708 (0.6); 7.9560 (0.8); 7.9497 (0.8); 7.9364 (0.6); 7.9301 (0.6); 7.6088 (0.6);
7.5937 (1.0); 7.5894 (1.2); 7.5742 (0.6); 7.5699 (0.6); 7.5040 (0.5); 7.4988 (0.5); 7.4911 (0.5); 7.4839 (0.5); 7.3469 (0.7);
7.3439 (0.8); 7.3278 (1.0); 7.3244 (1.5); 7.3202 (0.9); 7.3051 (0.7); 7.2997 (1.6); 7.2941 (1.7); 7.2785 (1.0); 7.2729 (1.4);
5.7924 (6.8); 3.6178 (1.2); 3.6156 (0.8); 3.6115 (0.8); 3.6075 (1.0); 3.6012 (2.7); 3.5951 (0.9); 3.5909 (0.7); 3.5870 (1.0);
3.5846 (1.1); 3.5546 (16.0); 3.3205 (4.3); 2.5241 (0.9); 2.5194 (1.3); 2.5108 (14.5); 2.5062 (31.4); 2.5016 (43.5); 2.4970
(30.4); 2.4925 (13.4); 1.7760 (1.1); 1.7683 (1.1); 1.7595 (3.3); 1.7546 (0.7); 1.7507 (1.0); 1.7430 (1.0); 1.3556 (2.7); −0.0002
(14.5)
I-26: ¹H NMR(400.6 MHz, CDCl3):
δ = 8.1826 (1.1); 8.1763 (1.0); 7.6998 (0.6); 7.6936 (0.5); 7.6811 (0.6); 7.6786 (0.7); 7.6749 (0.6); 7.6724 (0.6); 7.6599 (0.6);
7.6537 (0.5); 7.3269 (1.7); 7.3229 (0.7); 7.3190 (0.6); 7.3126 (1.0); 7.3085 (2.4); 7.3051 (1.6); 7.3007 (1.0); 7.2902 (0.7);
7.2875 (0.6); 7.2604 (15.6); 7.1577 (1.6); 7.1531 (1.6); 7.1476 (0.5); 7.1419 (0.7); 7.1369 (1.5); 7.1334 (1.4); 6.9657 (0.7);
6.9596 (0.7); 6.9445 (0.7); 6.9383 (0.7); 5.9867 (5.1); 3.8675 (15.0); 3.6982 (16.0); 1.5414 (5.3); 0.0079 (0.9); −0.0002
(23.8); −0.0085 (0.6)
I-27: ¹H NMR(400.6 MHz, CDCl3):
δ = 7.2605 (10.2); 7.1919 (0.5); 7.1897 (0.6); 7.1464 (1.0); 7.1295 (0.8); 7.1268 (1.3); 7.1218 (1.0); 7.1049 (0.7); 7.1006 (0.7);
7.0800 (0.5); 7.0752 (0.7); 7.0737 (0.7); 7.0549 (0.9); 5.9572 (4.9); 5.2998 (2.3); 3.8510 (14.8); 3.6805 (16.0); 2.1997 (3.4);
2.1943 (3.4); 1.5424 (4.4); −0.0002 (14.9)
I-28: ¹H NMR(400.6 MHz, CDCl3):
δ = 8.4852 (1.9); 8.4783 (1.9); 8.2941 (1.1); 8.2904 (1.8); 8.2866 (1.0); 7.4349 (0.6); 7.4304 (0.7); 7.4280 (0.7); 7.4236 (0.6);
7.4130 (0.6); 7.4085 (0.7); 7.4061 (0.6); 7.4017 (0.6); 7.2612 (7.8); 7.1937 (0.5); 7.1802 (0.7); 7.1737 (0.5); 7.0494 (0.5);
7.0399 (0.6); 7.0057 (0.6); 6.9938 (0.6); 6.9828 (0.9); 6.9710 (0.9); 5.9357 (5.2); 3.8652 (15.3); 3.6867 (16.0); 1.5644 (0.8);
0.8818 (0.6); −0.0002 (12.1)
I-29: ¹H NMR(400.6 MHz, CDCl3):
δ = 8.4874 (1.6); 8.4805 (1.6); 8.2941 (0.9); 8.2903 (1.6); 8.2867 (0.9); 7.4498 (0.6); 7.4454 (0.6); 7.4429 (0.7); 7.4385 (0.6);
7.4278 (0.6); 7.4234 (0.7); 7.4210 (0.6); 7.4165 (0.6); 7.2615 (6.7); 7.2011 (0.6); 7.0195 (0.5); 7.0077 (0.5); 6.9966 (0.8);
6.9849 (0.8); 5.9444 (5.1); 3.8668 (15.2); 3.6889 (16.0); −0.0002 (10.0)
I-30: ¹H NMR(400.6 MHz, CDCl3):
δ = 8.1876 (1.0); 8.1856 (0.8); 8.1834 (0.8); 8.1813 (1.0); 7.7057 (0.5); 7.6995 (0.5); 7.6871 (0.6); 7.6845 (0.6); 7.6808 (0.6);
7.6783 (0.6); 7.6659 (0.6); 7.6596 (0.6); 7.3192 (1.6); 7.3152 (0.7); 7.3112 (0.6); 7.3049 (0.8); 7.3008 (2.2); 7.2974 (1.6);
7.2930 (1.0); 7.2826 (0.7); 7.2799 (0.5); 7.2615 (5.8); 7.1472 (1.6); 7.1425 (1.5); 7.1314 (0.7); 7.1263 (1.4); 7.1228 (1.4);
6.9655 (0.7); 6.9641 (0.7); 6.9580 (0.7); 6.9565 (0.7); 6.9443 (0.6); 6.9429 (0.7); 6.9368 (0.7); 6.9353 (0.6); 5.9882 (5.2);
3.8665 (15.2); 3.6975 (16.0); 1.5569 (2.0); −0.0002 (8.2)
I-31: ¹H NMR(400.6 MHz, CDCl3):
δ = 8.4479 (1.6); 8.4412 (1.6); 8.3067 (1.8); 7.3483 (0.7); 7.3425 (0.8); 7.3372 (0.6); 7.3260 (0.7); 7.3207 (0.8); 7.3148 (0.6);
7.2609 (10.1); 7.1618 (0.6); 7.1550 (0.6); 7.1478 (1.0); 7.1411 (0.6); 7.1342 (0.5); 7.1267 (0.5); 7.0040 (0.6); 6.9942 (0.8);
6.9822 (1.0); 6.9694 (0.8); 6.9588 (1.0); 6.9467 (0.9); 5.9490 (5.2); 3.8495 (15.4); 3.6600 (16.0); 1.6010 (0.6); 1.5940 (0.6);
1.5801 (1.1); 1.5672 (1.1); 1.5599 (0.9); 0.8140 (1.6); 0.8066 (1.2); 0.7930 (1.5); 0.7829 (1.4); 0.7752 (1.6); 0.7689 (1.8);
0.7625 (1.5); 0.7577 (1.2); −0.0002 (15.6); −0.0085 (0.5)
I-32: ¹H NMR(400.6 MHz, CDCl3):
δ = 8.5250 (2.1); 8.5186 (2.0); 8.3206 (2.4); 7.4933 (0.9); 7.4886 (1.2); 7.4826 (0.8); 7.4716 (0.9); 7.4670 (1.2); 7.4610 (0.8);
7.2609 (6.8); 7.2280 (0.6); 7.2203 (0.7); 7.2137 (0.7); 7.2074 (1.1); 7.2005 (0.8); 7.1940 (0.7); 7.1863 (0.7); 7.0830 (0.6);
7.0775 (0.7); 7.0697 (0.8); 7.0602 (0.8); 7.0515 (0.8); 7.0215 (0.8); 7.0097 (0.8); 6.9985 (1.2); 6.9867 (1.2); 6.9763 (0.5);
5.9588 (6.2); 5.2998 (0.6); 3.7174 (16.0); 1.4321 (2.4); 0.8818 (0.5); −0.0002 (10.4); −0.0007 (10.6)
I-33: ¹H NMR(400.6 MHz, CDCl3):
δ = 8.4858 (1.9); 8.4791 (2.0); 8.2903 (2.3); 7.4508 (0.7); 7.4441 (1.0); 7.4398 (0.8); 7.4288 (0.7); 7.4244 (1.0); 7.4220 (1.0);
7.4179 (0.8); 7.2610 (9.2); 7.2259 (0.5); 7.2184 (0.6); 7.2119 (0.6); 7.2046 (1.0); 7.1986 (0.7); 7.1917 (0.6); 7.1840 (0.6);
7.0623 (0.8); 7.0522 (0.7); 7.0426 (0.6); 7.0157 (0.7); 7.0038 (0.7); 6.9927 (1.0); 6.9809 (1.0); 5.9037 (5.4); 4.3508 (0.9);
4.3330 (2.8); 4.3152 (2.8); 4.2977 (1.0); 3.6896 (16.0); 1.5662 (0.8); 1.3504 (3.8); 1.3326 (7.9); 1.3148 (3.8); 0.0077
(0.5); −0.0002 (13.9)
I-34: ¹H-NMR(400.6 MHz, d₆-DMSO):
δ = 13.4162 (0.7); 8.6184 (2.3); 8.6115 (2.4); 8.3661 (1.3); 8.3619 (2.4); 8.3580 (1.2); 7.7899 (0.7); 7.7855 (0.8); 7.7830 (0.8);
7.7786 (0.7); 7.7660 (0.7); 7.7616 (0.8); 7.7591 (0.7); 7.7547 (0.7); 7.4690 (0.7); 7.4650 (0.6); 7.3446 (0.5); 7.3380 (1.1);
7.3331 (0.6); 7.3262 (1.4); 7.3221 (1.1); 7.3174 (0.9); 7.3052 (0.8); 5.7460 (7.0); 3.5314 (16.0); 3.3209 (2.5); 2.5413 (1.6);
2.5197 (0.6); 2.5109 (10.3); 2.5064 (22.9); 2.5018 (32.4); 2.4971 (23.0); 2.4926 (10.3); 1.6916 (0.5); 1.6835 (0.6); 1.6706
(1.1); 1.6575 (0.6); 1.6496 (0.6); 0.7690 (0.5); 0.7648 (0.6); 0.7580 (1.8); 0.7541 (0.8); 0.7482 (0.8); 0.7436 (0.5); 0.7369
(1.8); 0.7333 (0.8); 0.7279 (0.6); 0.6347 (0.8); 0.6303 (0.6); 0.6216 (0.8); 0.6172 (0.6); 0.6006 (0.7); 0.5951 (0.8); 0.5877
(0.7); 0.5822 (0.7); 0.0081 (0.5); −0.0002 (18.1); −0.0085 (0.5)
I-35: ¹H NMR(400.6 MHz, CDCl3):
δ = 9.0565 (2.1); 9.0530 (2.0); 8.4051 (2.0); 8.3988 (2.0); 8.2444 (1.3); 8.2382 (1.2); 8.0212 (1.4); 8.0175 (1.4); 8.0148 (1.4);
8.0112 (1.2); 7.8671 (0.6); 7.8609 (0.6); 7.8482 (0.7); 7.8460 (0.7); 7.8421 (0.7); 7.8399 (0.7); 7.8272 (0.6); 7.8211 (0.6);
7.2606 (24.7); 7.0383 (0.8); 7.0310 (0.8); 7.0172 (0.8); 7.0098 (0.8); 6.0073 (5.2); 3.8961 (15.3); 3.7202 (16.0); 1.5428 (6.9);
0.0078 (1.2); −0.0002 (35.2); −0.0086 (1.1)
I-36: ¹H NMR(400.6 MHz, CDCl3):
δ = 9.0570 (1.9); 9.0535 (1.9); 8.4020 (1.9); 8.3956 (1.9); 8.2350 (1.2); 8.2288 (1.2); 8.0138 (1.3); 8.0102 (1.4); 8.0075 (1.4);
8.0038 (1.3); 7.8608 (0.6); 7.8546 (0.6); 7.8421 (0.7); 7.8398 (0.7); 7.8359 (0.6); 7.8336 (0.7); 7.8210 (0.6); 7.8148 (0.6);
7.2622 (7.3); 7.0350 (0.8); 7.0289 (0.8); 7.0277 (0.8); 7.0139 (0.8); 7.0078 (0.7); 7.0065 (0.7); 6.0044 (5.4); 3.8959 (15.4);
3.7191 (16.0); 1.5638 (1.2); −0.0002 (10.1)
I-37: ¹H NMR(400.6 MHz, CDCl3):
δ = 8.4468 (1.6); 8.4400 (1.6); 8.3115 (1.0); 8.3077 (1.6); 8.3040 (0.9); 7.3500 (0.6); 7.3456 (0.7); 7.3432 (0.6); 7.3389 (0.6);
7.3276 (0.6); 7.3232 (0.7); 7.3208 (0.6); 7.3164 (0.6); 7.2615 (7.8); 7.1535 (0.8); 7.0014 (0.5); 6.9916 (0.7); 6.9833 (0.5);
6.9811 (0.6); 6.9787 (0.7); 6.9665 (0.7); 6.9558 (0.8); 6.9437 (0.8); 5.9131 (5.4); 4.3363 (0.6); 4.3317 (0.6); 4.3184 (1.7);
4.3139 (1.7); 4.3005 (1.8); 4.2962 (1.7); 4.2827 (0.6); 4.2784 (0.5); 3.6605 (16.0); 1.5964 (0.6); 1.5876 (0.5); 1.5798 (1.3);
1.5741 (0.7); 1.5672 (0.5); 1.5621 (0.7); 1.3434 (3.8); 1.3255 (7.9); 1.3077 (3.7); 0.8105 (1.6); 0.7909 (3.6); 0.7839 (0.9);
0.7813 (0.8); 0.7777 (2.7); −0.0002 (11.1)
I-38: ¹H NMR(400.6 MHz, CDCl3):
δ = 8.2474 (0.6); 8.2457 (0.7); 8.2438 (0.8); 8.2421 (0.7); 8.2358 (0.7); 8.2340 (0.8); 8.2322 (0.8); 8.2305 (0.7); 8.1388 (1.0);
8.1369 (0.8); 8.1347 (0.8); 8.1325 (1.0); 7.8627 (0.5); 7.8565 (0.5); 7.8441 (0.6); 7.8415 (0.6); 7.8378 (0.6); 7.8353 (0.6);
7.8228 (0.6); 7.8166 (0.6); 7.5366 (0.5); 7.5329 (0.5); 7.5158 (0.7); 7.5123 (0.9); 7.5098 (0.6); 7.4927 (0.7); 7.4890 (0.7);
7.3722 (0.7); 7.3632 (0.8); 7.3606 (0.7); 7.3515 (1.2); 7.3425 (0.6); 7.3399 (0.6); 7.3309 (0.5); 7.2619 (8.5); 6.9749 (0.6);
6.9734 (0.7); 6.9674 (0.7); 6.9660 (0.7); 6.9537 (0.6); 6.9522 (0.7); 6.9462 (0.7); 6.9447 (0.7); 6.0115 (5.1); 3.8538 (14.7);
3.6887 (16.0); 1.5594 (0.7); −0.0002 (12.2)
I-39: ¹H NMR(400.6 MHz, CDCl3):
δ = 8.2306 (0.6); 8.2288 (0.7); 8.2269 (0.7); 8.2252 (0.6); 8.2189 (0.6); 8.2171 (0.7); 8.2153 (0.7); 8.2136 (0.6); 8.1440 (0.9);
8.1419 (0.7); 8.1398 (0.8); 8.1377 (1.0); 7.8586 (0.5); 7.8399 (0.6); 7.8375 (0.6); 7.8337 (0.6); 7.8312 (0.6); 7.8188 (0.5);
7.8125 (0.5); 7.5147 (0.5); 7.5110 (0.5); 7.4940 (0.7); 7.4915 (0.7); 7.4904 (0.8); 7.4879 (0.6); 7.4709 (0.7); 7.4672 (0.6);
7.3518 (0.6); 7.3428 (0.7); 7.3402 (0.7); 7.3311 (1.1); 7.3221 (0.5); 7.3195 (0.5); 7.2614 (12.9); 6.9671 (0.6); 6.9656 (0.7);
6.9597 (0.7); 6.9581 (0.6); 6.9460 (0.6); 6.9444 (0.6); 6.9385 (0.6); 6.9369 (0.6); 6.0029 (5.0); 3.8507 (14.2); 3.6849 (16.0);
2.0454 (1.0); 1.5523 (2.4); 1.2596 (0.7); 0.0080 (0.5); −0.0002 (20.1); −0.0085 (0.5)
I-40: ¹H NMR(400.6 MHz, CDCl3):
δ = 8.2019 (0.6); 8.2001 (0.7); 8.1983 (0.7); 8.1965 (0.7); 8.1902 (0.6); 8.1884 (0.8); 8.1866 (0.7); 8.1849 (0.6); 8.1449 (1.0);
8.1430 (0.8); 8.1407 (0.8); 8.1387 (1.0); 7.7815 (0.5); 7.7753 (0.5); 7.7625 (0.6); 7.7605 (0.6); 7.7563 (0.6); 7.7543 (0.6);
7.7415 (0.5); 7.7353 (0.5); 7.4822 (0.5); 7.4785 (0.5); 7.4616 (0.7); 7.4582 (1.0); 7.4551 (0.6); 7.4382 (0.7); 7.4345 (0.6);
7.2941 (0.7); 7.2852 (0.7); 7.2824 (0.7); 7.2735 (1.2); 7.2618 (10.4); 7.2529 (0.5); 6.9261 (0.7); 6.9247 (0.7); 6.9187 (0.7);
6.9172 (0.7); 6.9050 (0.6); 6.9036 (0.7); 6.8976 (0.6); 6.8961 (0.6); 6.0191 (5.3); 3.8339 (14.8); 3.6578 (16.0); 2.3244 (0.5);
1.5795 (0.8); 1.5627 (1.6); 0.7921 (1.4); 0.7865 (0.7); 0.7745 (2.4); 0.7718 (2.4); 0.7692 (1.7); 0.7652 (0.9); 0.7611 (1.4);
0.7584 (0.9); 0.7561 (0.7); 0.7539 (0.6); 0.7522 (0.6); −0.0002 (14.2)
I-41: ¹H NMR(400.6 MHz, CDCl3):
δ = 8.2448 (0.6); 8.2432 (0.6); 8.2412 (0.7); 8.2397 (0.6); 8.2332 (0.6); 8.2316 (0.7); 8.2297 (0.7); 8.2282 (0.6); 8.1268 (0.8);
8.1207 (0.9); 7.5245 (0.5); 7.5073 (0.7); 7.5056 (0.7); 7.5037 (0.8); 7.5019 (0.6); 7.4848 (0.7); 7.4811 (0.7); 7.3925 (0.6);
7.3832 (0.7); 7.3809 (0.6); 7.3717 (1.0); 7.2624 (6.2); 6.9551 (0.6); 6.9535 (0.6); 6.9476 (0.7); 6.9461 (0.6); 6.9339 (0.6);
6.9323 (0.6); 6.9264 (0.6); 6.9249 (0.6); 6.0131 (4.4); 3.8462 (14.7); 3.6760 (16.0); 1.5629 (1.0); −0.0002 (8.8)
I-42: ¹H NMR(400.6 MHz, CDCl3):
δ = 9.0861 (1.8); 9.0828 (1.7); 8.4096 (1.9); 8.4030 (2.0); 8.2512 (1.1); 8.2453 (1.2); 8.0492 (1.1); 8.0455 (1.2); 8.0429 (1.2);
8.0392 (1.1); 7.8648 (0.6); 7.8586 (0.6); 7.8461 (0.6); 7.8436 (0.7); 7.8399 (0.7); 7.8374 (0.8); 7.8250 (0.7); 7.8188 (0.6);
7.5186 (0.8); 7.2873 (0.9); 7.2666 (0.7); 7.2658 (0.8); 7.2602 (146.0); 7.2552 (2.3); 7.0434 (0.8); 7.0345 (0.8); 7.0207 (0.8);
7.0147 (0.8); 6.9965 (0.8); 5.9787 (3.4); 5.3002 (1.1); 3.7659 (16.0); 2.2717 (0.7); 1.4322 (7.4); 1.2549 (1.3); 0.1457 (0.6);
0.0269 (1.1); 0.0079 (4.8); −0.0002 (195.7); −0.0053 (3.4); −0.0085 (6.2); −0.1492 (0.6)
I-43: ¹H-NMR(400.6 MHz, d₆-DMSO):
δ = 9.0093 (1.7); 9.0086 (1.7); 9.0058 (1.8); 9.0051 (1.8); 8.5383 (1.7); 8.5376 (1.7); 8.5319 (1.8); 8.3544 (1.0); 8.3481 (1.0);
8.2087 (1.4); 8.2051 (1.5); 8.2023 (1.5); 8.1987 (1.4); 8.1067 (0.6); 8.1004 (0.6); 7.3341 (0.6); 7.3285 (0.6); 7.3128 (0.6);
7.3072 (0.6); 5.9707 (4.6); 3.5952 (11.6); 3.3200 (16.0); 2.5237 (1.3); 2.5191 (1.7); 2.5103 (24.6); 2.5057 (54.4); 2.5011
(77.0); 2.4966 (54.2); 2.4920 (24.8); 0.0079 (2.0); 0.0054 (0.6); 0.0046 (0.7); −0.0002 (71.0); −0.0059 (1.0); −0.0068
(0.9); −0.0085 (2.2)
I-44: ¹H NMR(400.6 MHz, CDCl3):
δ = 8.2982 (0.7); 8.2965 (0.8); 8.2945 (0.8); 8.2929 (0.8); 8.2865 (0.7); 8.2849 (0.8); 8.2829 (0.8); 8.2813 (0.7); 8.1484 (1.0);
8.1465 (0.8); 8.1442 (0.8); 8.1422 (1.1); 7.8667 (0.6); 7.8604 (0.5); 7.8481 (0.6); 7.8454 (0.7); 7.8418 (0.6); 7.8391 (0.6);
7.8268 (0.6); 7.8205 (0.6); 7.5463 (0.6); 7.5426 (0.6); 7.5255 (0.8); 7.5231 (0.8); 7.5219 (0.9); 7.5195 (0.8); 7.5025 (0.8);
7.4988 (0.7); 7.4009 (0.7); 7.3918 (0.8); 7.3892 (0.8); 7.3801 (1.2); 7.3710 (0.6); 7.3685 (0.6); 7.3594 (0.5); 7.2609 (26.2);
6.9873 (0.7); 6.9857 (0.7); 6.9799 (0.8); 6.9784 (0.7); 6.9661 (0.7); 6.9645 (0.7); 6.9586 (0.7); 6.9571 (0.7); 5.9988 (6.6);
3.7189 (16.0); 1.2640 (0.7); 0.8820 (1.5); 0.8643 (0.6); 0.0080 (1.0); 0.0023 (1.2); −0.0002 (34.1); −0.0085 (0.9)
I-45: ¹H NMR(400.6 MHz, CDCl3):
δ = 8.3047 (0.8); 8.3030 (0.9); 8.3010 (0.9); 8.2995 (0.8); 8.2930 (0.9); 8.2914 (1.0); 8.2893 (0.9); 8.2879 (0.8); 8.1565 (1.2);
8.1545 (0.8); 8.1521 (1.0); 8.1503 (1.2); 7.8749 (0.6); 7.8686 (0.6); 7.8563 (0.7); 7.8537 (0.7); 7.8501 (0.7); 7.8474 (0.7);
7.8351 (0.6); 7.8288 (0.6); 7.5365 (0.6); 7.5328 (0.6); 7.5157 (0.9); 7.5133 (0.9); 7.5122 (0.9); 7.5099 (0.6); 7.4928 (0.8);
7.4891 (0.7); 7.3980 (0.8); 7.3888 (0.9); 7.3863 (0.8); 7.3772 (1.3); 7.3681 (0.6); 7.3656 (0.6); 7.3564 (0.5); 7.2615 (13.5);
6.9835 (0.8); 6.9821 (0.8); 6.9762 (0.8); 6.9748 (0.7); 6.9623 (0.8); 6.9608 (0.8); 6.9550 (0.8); 6.9535 (0.7); 5.9985 (7.0);
5.3004 (0.7); 3.7137 (16.0); −0.0002 (18.0); −0.0085 (0.5)
I-46: ¹H NMR(400.6 MHz, CDCl3):
δ = 9.0617 (1.6); 9.0611 (1.6); 9.0582 (1.6); 8.4015 (1.5); 8.4008 (1.5); 8.3951 (1.6); 8.3945 (1.6); 8.2443 (1.0); 8.2422 (0.8);
8.2401 (0.8); 8.2381 (1.0); 8.2361 (0.6); 8.0190 (1.3); 8.0153 (1.3); 8.0126 (1.3); 8.0090 (1.2); 7.8681 (0.5); 7.8619 (0.5);
7.8494 (0.6); 7.8469 (0.6); 7.8432 (0.6); 7.8408 (0.6); 7.8282 (0.6); 7.8220 (0.6); 7.2623 (9.0); 7.0389 (0.7); 7.0374 (0.7);
7.0314 (0.7); 7.0299 (0.7); 7.0177 (0.7); 7.0162 (0.6); 7.0103 (0.7); 7.0088 (0.6); 5.9706 (5.4); 4.3786 (0.5); 4.3644 (1.6);
4.3608 (1.6); 4.3466 (1.7); 4.3430 (1.6); 4.3287 (0.6); 3.7204 (16.0); 1.5652 (1.5); 1.3814 (3.6); 1.3635 (7.7); 1.3457
(3.5); −0.0002 (11.6)
I-47: ¹H NMR(400.6 MHz, CDCl3):
δ = 9.0626 (1.7); 9.0617 (1.6); 9.0590 (1.7); 9.0582 (1.6); 8.3981 (1.7); 8.3918 (1.8); 8.3908 (1.6); 8.2346 (1.0); 8.2324 (0.8);
8.2303 (0.8); 8.2285 (1.0); 8.0106 (1.3); 8.0070 (1.4); 8.0042 (1.3); 8.0006 (1.3); 7.8609 (0.5); 7.8547 (0.5); 7.8422 (0.6);
7.8397 (0.6); 7.8359 (0.6); 7.8336 (0.6); 7.8211 (0.6); 7.8149 (0.6); 7.2616 (13.4); 7.0358 (0.7); 7.0343 (0.7); 7.0284 (0.7);
7.0270 (0.6); 7.0147 (0.7); 7.0132 (0.7); 7.0073 (0.7); 7.0057 (0.6); 5.9675 (5.5); 4.3784 (0.5); 4.3646 (1.6); 4.3606 (1.6);
4.3467 (1.6); 4.3429 (1.6); 4.3288 (0.5); 3.7196 (16.0); 1.5566 (5.0); 1.3816 (3.6); 1.3638 (7.7); 1.3460 (3.5); −0.0002 (17.9)
I-48: ¹H NMR(400.6 MHz, CDCl3):
δ = 8.2487 (0.7); 8.2469 (0.8); 8.2451 (0.8); 8.2434 (0.7); 8.2371 (0.8); 8.2353 (0.8); 8.2334 (0.8); 8.2318 (0.7); 8.1376 (1.0);
8.1355 (0.8); 8.1333 (0.9); 8.1314 (1.0); 7.8630 (0.5); 7.8568 (0.5); 7.8443 (0.6); 7.8418 (0.7); 7.8381 (0.6); 7.8355 (0.6);
7.8231 (0.6); 7.8169 (0.6); 7.5335 (0.5); 7.5298 (0.5); 7.5128 (0.8); 7.5093 (0.8); 7.5067 (0.6); 7.4897 (0.7); 7.4860 (0.7);
7.3707 (0.7); 7.3617 (0.8); 7.3591 (0.7); 7.3501 (1.2); 7.3410 (0.6); 7.3385 (0.6); 7.3295 (0.5); 7.2617 (11.1); 6.9740 (0.7);
6.9725 (0.7); 6.9665 (0.8); 6.9650 (0.7); 6.9528 (0.7); 6.9513 (0.7); 6.9453 (0.7); 6.9438 (0.6); 5.9801 (5.5); 4.3383 (0.7);
4.3204 (2.2); 4.3025 (2.4); 4.2846 (0.8); 3.6883 (16.0); 1.5581 (4.5); 1.3374 (3.6); 1.3196 (7.6); 1.3017 (3.6); −0.0002 (14.0)
I-49: ¹H NMR(400.6 MHz, CDCl3):
δ = 8.1092 (1.0); 8.1071 (0.7); 8.1048 (0.8); 8.1030 (1.0); 7.7385 (0.6); 7.7361 (0.6); 7.7323 (0.6); 7.7299 (0.6); 7.7175 (0.5);
7.3929 (0.7); 7.3885 (0.8); 7.3698 (0.6); 7.2618 (8.2); 7.2173 (0.5); 7.2156 (0.5); 7.1961 (0.7); 7.0422 (0.5); 7.0176 (0.8);
7.0139 (0.5); 6.9477 (0.7); 6.9462 (0.7); 6.9403 (0.7); 6.9387 (0.6); 6.9265 (0.7); 6.9249 (0.7); 6.9191 (0.7); 6.9175 (0.6);
5.9525 (5.0); 5.3002 (0.6); 3.8555 (14.9); 3.6857 (16.0); 1.5577 (1.8); −0.0002 (9.9)
I-50: ¹H NMR(400.6 MHz, CDCl3):
δ = 8.1055 (1.0); 8.1036 (1.5); 8.1015 (1.2); 8.0994 (1.2); 8.0973 (1.6); 8.0954 (1.1); 7.7613 (0.8); 7.7550 (0.8); 7.7426 (1.0);
7.7400 (1.0); 7.7363 (1.0); 7.7338 (1.0); 7.7213 (0.9); 7.7151 (0.9); 7.4301 (0.6); 7.4258 (0.7); 7.4109 (1.1); 7.4065 (1.3);
7.3921 (0.7); 7.3877 (0.9); 7.3642 (0.6); 7.3623 (0.5); 7.3598 (0.5); 7.3579 (0.5); 7.3520 (0.7); 7.3502 (0.5); 7.3475 (0.6);
7.3457 (0.5); 7.3435 (0.6); 7.3390 (0.5); 7.3313 (0.6); 7.2624 (11.2); 7.2360 (0.7); 7.2343 (0.8); 7.2326 (0.8); 7.2311 (0.7);
7.2145 (1.1); 7.2129 (1.2); 7.1957 (0.5); 7.1940 (0.5); 7.0577 (0.8); 7.0545 (0.8); 7.0369 (0.8); 7.0331 (1.3); 7.0293 (0.8);
7.0118 (0.7); 7.0085 (0.7); 6.9514 (1.1); 6.9498 (1.1); 6.9439 (1.1); 6.9423 (1.1); 6.9301 (1.0); 6.9285 (1.1); 6.9226 (1.0);
6.9210 (1.1); 5.9647 (8.7); 4.3359 (0.8); 4.3333 (0.9); 4.3180 (2.8); 4.3155 (2.9); 4.3001 (2.9); 4.2978 (2.9); 4.2823 (1.0);
4.2800 (0.9); 4.0593 (1.2); 4.0416 (1.2); 4.0354 (1.6); 4.0177 (1.6); 3.9017 (1.6); 3.8841 (1.6); 3.8779 (1.2); 3.8665 (0.5);
3.8602 (1.2); 3.7629 (0.8); 3.7526 (0.6); 3.7463 (2.1); 3.7401 (0.6); 3.7297 (0.9); 1.8696 (0.8); 1.8620 (0.7); 1.8530 (2.5);
1.8441 (0.7); 1.8364 (0.8); 1.5681 (5.2); 1.4322 (1.1); 1.3491 (5.6); 1.3316 (14.5); 1.3144 (16.0); 1.2967 (6.0); 0.0023
(0.6); −0.0002 (14.1)
I-51: ¹H NMR(400.6 MHz, CDCl3):
δ = 8.1253 (2.5); 8.1234 (1.9); 8.1209 (2.0); 8.1190 (2.6); 7.7718 (1.3); 7.7656 (1.3); 7.7532 (1.4); 7.7506 (1.5); 7.7470 (1.4);
7.7444 (1.4); 7.7320 (1.3); 7.7257 (1.2); 7.4368 (1.0); 7.4324 (1.2); 7.4175 (1.7); 7.4132 (2.0); 7.3987 (1.2); 7.3944 (1.4);
7.3899 (0.7); 7.3855 (0.6); 7.3777 (0.7); 7.3731 (0.7); 7.3710 (1.0); 7.3692 (0.9); 7.3666 (0.9); 7.3648 (0.8); 7.3587 (1.0);
7.3571 (0.8); 7.3543 (0.9); 7.3525 (0.8); 7.3503 (0.9); 7.3458 (0.8); 7.3381 (0.8); 7.3337 (0.7); 7.2619 (43.9); 7.2407 (1.1);
7.2392 (1.2); 7.2373 (1.2); 7.2362 (1.3); 7.2196 (1.8); 7.2179 (1.8); 7.2021 (0.7); 7.2005 (0.8); 7.1988 (0.7); 7.0612 (1.2);
7.0580 (1.2); 7.0404 (1.2); 7.0366 (2.0); 7.0328 (1.2); 7.0153 (1.1); 7.0121 (1.0); 6.9586 (1.7); 6.9571 (1.7); 6.9512 (1.8);
6.9497 (1.6); 6.9373 (1.6); 6.9358 (1.7); 6.9299 (1.6); 6.9284 (1.6); 6.0122 (14.4); 4.1508 (0.5); 4.1329 (1.5); 4.1151 (1.6);
4.0972 (0.6); 4.0888 (1.6); 4.0826 (0.7); 4.0711 (1.7); 4.0649 (2.2); 4.0534 (0.6); 4.0472 (2.2); 4.0295 (0.6); 3.9401 (0.6);
3.9224 (2.1); 3.9162 (0.6); 3.9048 (2.2); 3.8985 (1.7); 3.8872 (0.7); 3.8809 (1.7); 3.8633 (0.5); 2.1131 (1.6); 2.0479 (7.6);
1.4321 (1.3); 1.3631 (7.6); 1.3455 (16.0); 1.3278 (7.4); 1.2773 (2.2); 1.2595 (4.6); 1.2417 (2.4); 1.2215 (0.5); 0.0080
(0.7); −0.0002 (25.4); −0.0085 (0.7)
I-52: ¹H NMR(400.6 MHz, CDCl3):
δ = 8.1195 (1.4); 8.1135 (1.5); 7.7661 (0.8); 7.7598 (0.7); 7.7474 (0.9); 7.7448 (0.9); 7.7412 (0.9); 7.7386 (0.9); 7.7263 (0.8);
7.7200 (0.7); 7.4458 (0.6); 7.4415 (0.7); 7.4265 (1.1); 7.4224 (1.3); 7.4078 (0.8); 7.4034 (0.9); 7.3771 (0.6); 7.3753 (0.6);
7.3728 (0.6); 7.3649 (0.7); 7.3605 (0.6); 7.3586 (0.5); 7.3566 (0.6); 7.3443 (0.5); 7.2623 (10.9); 7.2473 (0.7); 7.2458 (0.8);
7.2441 (0.8); 7.2248 (1.2); 7.2073 (0.5); 7.0681 (0.8); 7.0649 (0.8); 7.0473 (0.8); 7.0435 (1.3); 7.0397 (0.8); 7.0222 (0.7);
7.0190 (0.7); 6.9620 (1.0); 6.9549 (1.0); 6.9408 (0.9); 6.9336 (0.9); 6.0167 (7.7); 4.1510 (1.1); 4.1332 (3.4); 4.1153 (3.5);
4.0975 (1.2); 4.0897 (1.0); 4.0720 (1.0); 4.0659 (1.4); 4.0482 (1.3); 3.9254 (1.3); 3.9078 (1.3); 3.9015 (1.0); 3.8839 (1.0);
2.1119 (4.6); 2.0481 (16.0); 1.4322 (0.9); 1.3623 (4.8); 1.3447 (9.9); 1.3270 (4.6); 1.2775 (4.4); 1.2597 (8.8); 1.2419
(4.2); −0.0002 (14.1)
I-53: ¹H NMR(400.6 MHz, CDCl3):
δ = 8.1147 (1.2); 8.1126 (0.9); 8.1104 (0.9); 8.1083 (1.1); 8.1063 (0.8); 7.7659 (0.7); 7.7596 (0.6); 7.7472 (0.8); 7.7446 (0.8);
7.7410 (0.7); 7.7384 (0.7); 7.7260 (0.7); 7.7197 (0.6); 7.4533 (0.6); 7.4385 (0.8); 7.4341 (0.9); 7.4197 (0.6); 7.4153 (0.6);
7.2618 (38.9); 7.2431 (0.5); 7.2415 (0.5); 7.2397 (0.6); 7.2384 (0.5); 7.2232 (0.8); 7.2200 (0.8); 7.0567 (0.6); 7.0534 (0.6);
7.0358 (0.6); 7.0320 (0.9); 7.0282 (0.6); 7.0107 (0.5); 6.9507 (0.8); 6.9492 (0.9); 6.9433 (0.8); 6.9417 (0.8); 6.9296 (0.8);
6.9280 (0.9); 6.9221 (0.7); 6.9205 (0.7); 5.9538 (6.3); 4.5264 (1.1); 4.5241 (1.2); 4.5100 (2.5); 4.5080 (2.6); 4.4936 (1.2);
4.4919 (1.2); 4.0494 (0.8); 4.0317 (0.9); 4.0256 (1.1); 4.0079 (1.1); 3.8899 (1.1); 3.8723 (1.1); 3.8662 (0.8); 3.8485 (0.8);
3.7009 (0.7); 3.6897 (0.6); 3.6499 (16.0); 2.7295 (1.8); 2.7132 (3.7); 2.6969 (1.8); 1.3397 (3.9); 1.3221 (8.3); 1.3044 (3.7);
0.0080 (0.7); 0.0024 (0.9); −0.0002 (23.7); −0.0084 (0.6)
I-54: ¹H NMR(400.6 MHz, CDCl3):
δ = 8.1117 (2.2); 8.1054 (2.4); 7.7656 (1.0); 7.7593 (1.2); 7.7468 (1.2); 7.7444 (1.3); 7.7406 (1.4); 7.7382 (1.4); 7.7257 (1.1);
7.7194 (1.2); 7.4538 (0.7); 7.4508 (0.6); 7.4350 (1.5); 7.4196 (0.6); 7.4156 (0.9); 7.4127 (0.8); 7.3839 (0.5); 7.3794 (0.5);
7.3717 (0.5); 7.3648 (0.9); 7.3604 (0.8); 7.3527 (0.9); 7.3483 (0.8); 7.3443 (0.9); 7.3398 (0.7); 7.3321 (0.8); 7.3277 (0.6);
7.2615 (85.3); 7.2376 (1.4); 7.2183 (1.8); 7.1991 (0.8); 7.0555 (1.1); 7.0522 (1.1); 7.0347 (1.1); 7.0308 (1.9); 7.0271 (1.3);
7.0095 (1.0); 7.0063 (1.0); 6.9978 (0.5); 6.9506 (1.6); 6.9492 (1.6); 6.9431 (1.7); 6.9418 (1.5); 6.9294 (1.6); 6.9280 (1.6);
6.9219 (1.6); 6.9205 (1.5); 6.0064 (0.6); 5.9656 (5.7); 5.9622 (6.0); 5.3004 (0.6); 4.4568 (1.0); 4.4461 (1.0); 4.4392 (1.0);
4.4295 (1.9); 4.4190 (1.4); 4.4121 (1.4); 4.4017 (1.5); 4.3258 (1.3); 4.3105 (2.5); 4.2986 (1.1); 4.2955 (1.5); 4.2834 (1.8);
4.2684 (1.0); 4.0478 (1.4); 4.0418 (0.7); 4.0301 (1.4); 4.0241 (1.8); 4.0064 (1.8); 3.9887 (0.6); 3.9064 (0.5); 3.8888 (1.6);
3.8711 (1.6); 3.8651 (1.2); 3.8534 (0.6); 3.8498 (2.0); 3.8476 (1.4); 3.6982 (0.6); 3.6949 (0.7); 3.6866 (0.7); 3.6829 (0.8);
3.6477 (15.8); 3.6363 (16.0); 2.8825 (0.8); 2.8651 (1.4); 2.8483 (1.4); 2.8313 (0.8); 1.5559 (1.4); 1.3499 (0.7); 1.3398 (7.5);
1.3323 (1.6); 1.3221 (15.1); 1.3146 (1.1); 1.3045 (7.0); 1.2651 (0.6); 1.2553 (1.3); 1.2246 (0.5); 1.2204 (0.6); 1.2025 (0.7);
1.1966 (6.9); 1.1917 (6.8); 1.1787 (6.9); 1.1738 (6.8); 0.0079 (1.2); −0.0002 (50.1); −0.0085 (1.5)
I-55: ¹H NMR(400.6 MHz, CDCl3):
δ = 8.2456 (0.9); 8.2438 (0.9); 8.2341 (0.9); 8.2322 (0.9); 8.1243 (1.1); 8.1186 (1.2); 7.8192 (0.6); 7.5205 (0.5); 7.5033 (0.8);
7.4998 (0.9); 7.4809 (0.7); 7.4773 (0.7); 7.3910 (0.6); 7.3817 (0.8); 7.3795 (0.7); 7.3702 (1.1); 7.3609 (0.6); 7.3586 (0.6);
7.2612 (17.2); 6.9533 (0.8); 6.9461 (0.8); 6.9323 (0.7); 6.9249 (0.8); 5.9868 (4.4); 4.3308 (0.6); 4.3287 (0.6); 4.3128 (1.9);
4.3110 (1.8); 4.2949 (2.0); 4.2932 (1.8); 4.2771 (0.7); 3.6768 (13.7); 2.0456 (1.2); 1.5503 (16.0); 1.3258 (3.2); 1.3080 (6.7);
1.2902 (3.2); 1.2597 (0.9); 0.0079 (0.8); −0.0002 (24.0); −0.0084 (0.8)
I-56: ¹H NMR(400.6 MHz, CDCl3):
δ = 8.2519 (0.7); 8.2501 (0.8); 8.2482 (0.8); 8.2465 (0.7); 8.2402 (0.7); 8.2384 (0.8); 8.2365 (0.8); 8.2348 (0.7); 8.1605 (1.0);
8.1563 (0.8); 8.1543 (1.1); 7.7884 (0.5); 7.7822 (0.5); 7.7695 (0.6); 7.7673 (0.6); 7.7633 (0.7); 7.7611 (0.6); 7.7484 (0.6);
7.7422 (0.6); 7.5078 (0.6); 7.5040 (0.6); 7.4871 (0.8); 7.4837 (1.0); 7.4806 (0.6); 7.4637 (0.7); 7.4599 (0.7); 7.3349 (0.7);
7.3259 (0.8); 7.3233 (0.7); 7.3142 (1.2); 7.3052 (0.6); 7.3026 (0.6); 7.2936 (0.6); 7.2614 (22.0); 6.9452 (0.7); 6.9437 (0.7);
6.9379 (0.8); 6.9363 (0.7); 6.9241 (0.7); 6.9226 (0.7); 6.9167 (0.7); 6.9152 (0.7); 5.9386 (6.2); 3.6921 (16.0); 2.3457 (0.6);
1.5723 (0.8); 1.5513 (0.6); 1.4322 (0.7); 1.2643 (0.8); 0.8820 (1.7); 0.8643 (0.6); 0.8067 (0.7); 0.8030 (1.1); 0.8012 (0.9);
0.7940 (0.9); 0.7863 (1.1); 0.7829 (0.9); 0.7810 (1.1); 0.7732 (0.9); 0.7701 (0.5); 0.7535 (1.0); 0.7438 (1.3); 0.7399 (1.7);
0.7321 (1.2); 0.7270 (1.1); 0.0080 (0.8); −0.0002 (32.6); −0.0085 (0.9)
I-57: ¹H NMR(400.6 MHz, CDCl3):
δ = 8.2883 (0.8); 8.2868 (0.9); 8.2847 (0.9); 8.2833 (0.8); 8.2767 (0.8); 8.2752 (0.9); 8.2731 (0.9); 8.1342 (1.1); 8.1282 (1.1);
7.8345 (0.5); 7.5348 (0.6); 7.5311 (0.6); 7.5139 (0.9); 7.5124 (0.8); 7.5102 (0.9); 7.5088 (0.7); 7.4915 (0.8); 7.4878 (0.8);
7.4183 (0.8); 7.4090 (1.0); 7.4068 (0.8); 7.3975 (1.3); 7.3882 (0.6); 7.3859 (0.6); 7.3766 (0.5); 7.2616 (11.9); 6.9686 (0.8);
6.9671 (0.8); 6.9613 (0.8); 6.9599 (0.8); 6.9474 (0.8); 6.9459 (0.8); 6.9401 (0.8); 6.9386 (0.8); 6.0169 (5.8); 3.7026 (16.0);
1.4322 (2.2); 1.2626 (0.6); 1.2549 (0.6); 0.8819 (0.9); −0.0002 (17.7); −0.0085 (0.5)
I-58: ¹H NMR(400.6 MHz, CDCl3):
δ = 8.2034 (0.7); 8.2017 (0.8); 8.1999 (0.8); 8.1982 (0.8); 8.1918 (0.7); 8.1900 (0.8); 8.1883 (0.8); 8.1866 (0.7); 8.1446 (1.1);
8.1384 (1.2); 7.7818 (0.5); 7.7756 (0.5); 7.7628 (0.6); 7.7608 (0.7); 7.7566 (0.6); 7.7546 (0.7); 7.7418 (0.6); 7.7356 (0.6);
7.4794 (0.5); 7.4757 (0.6); 7.4587 (0.7); 7.4554 (1.0); 7.4523 (0.6); 7.4353 (0.7); 7.4316 (0.7); 7.2926 (0.7); 7.2837 (0.8);
7.2810 (0.7); 7.2720 (1.2); 7.2615 (15.8); 7.2515 (0.6); 6.9243 (0.8); 6.9181 (0.8); 6.9168 (0.8); 6.9031 (0.8); 6.8957 (0.7);
5.9956 (5.5); 4.3199 (0.5); 4.3163 (0.6); 4.3020 (1.7); 4.2985 (1.7); 4.2841 (1.8); 4.2808 (1.7); 4.2663 (0.6); 4.2630 (0.6);
3.6579 (16.0); 1.5957 (0.5); 1.5788 (0.8); 1.5569 (14.9); 1.3286 (3.7); 1.3108 (7.7); 1.2929 (3.6); 0.7884 (3.5); 0.7715 (9.1);
0.0080 (0.6); −0.0002 (21.3); −0.0085 (0.6)
I-59: ¹H NMR(400.6 MHz, CDCl3):
δ = 8.1205 (1.2); 8.1184 (1.0); 8.1163 (1.0); 8.1143 (1.2); 7.7723 (0.6); 7.7660 (0.6); 7.7536 (0.7); 7.7511 (0.8); 7.7474 (0.7);
7.7448 (0.7); 7.7324 (0.6); 7.7261 (0.6); 7.4472 (0.5); 7.4322 (0.8); 7.4279 (0.9); 7.4134 (0.6); 7.4091 (0.6); 7.2621 (28.0);
7.2588 (0.5); 7.2345 (0.6); 7.2324 (0.6); 7.2310 (0.5); 7.2127 (0.8); 7.0486 (0.6); 7.0453 (0.6); 7.0278 (0.6); 7.0239 (1.0);
7.0202 (0.6); 7.0026 (0.5); 6.9990 (0.6); 6.9463 (0.8); 6.9447 (0.9); 6.9388 (0.8); 6.9373 (0.8); 6.9250 (0.8); 6.9234 (0.9);
6.9175 (0.8); 6.9160 (0.7); 5.9527 (6.2); 4.5254 (1.1); 4.5226 (1.1); 4.5090 (2.5); 4.5064 (2.4); 4.4926 (1.2); 4.4903 (1.2);
4.0491 (0.8); 4.0314 (0.8); 4.0253 (1.1); 4.0076 (1.1); 3.8880 (1.1); 3.8704 (1.1); 3.8642 (0.8); 3.8466 (0.8); 3.7013 (0.6);
3.6902 (0.5); 3.6497 (16.0); 2.7292 (1.8); 2.7129 (3.8); 2.6966 (1.9); 1.3414 (3.8); 1.3237 (8.1); 1.3061 (3.7); −0.0002 (17.6)
I-60: ¹H NMR(400.6 MHz, CDCl3):
δ = 8.0517 (0.9); 8.0462 (1.0); 7.3871 (0.5); 7.3721 (0.6); 7.3679 (1.4); 7.3611 (0.6); 7.3499 (0.9); 7.3483 (1.1); 7.2612 (13.4);
7.2036 (0.5); 7.2019 (0.6); 7.2004 (0.6); 7.1826 (0.9); 7.1811 (0.8); 7.0378 (0.5); 7.0170 (0.5); 7.0132 (0.8); 6.9188 (0.8);
6.9175 (0.7); 6.9114 (0.8); 6.8976 (0.8); 6.8962 (0.7); 6.8901 (0.8); 5.9847 (4.7); 3.8401 (15.5); 3.6541 (16.0); 1.5485 (7.4);
0.0079 (0.5); −0.0002 (19.0); −0.0029 (0.8); −0.0036 (0.5); −0.0085 (0.6)
I-61: ¹H NMR(400.6 MHz, CDCl3):
δ = 8.1234 (1.1); 8.1172 (1.1); 7.6793 (0.5); 7.6603 (0.6); 7.6583 (0.6); 7.6541 (0.6); 7.6521 (0.6); 7.6393 (0.5); 7.6331 (0.5);
7.3783 (0.8); 7.3740 (0.9); 7.3594 (0.5); 7.3550 (0.6); 7.2652 (0.5); 7.2616 (12.9); 7.1905 (0.5); 7.1884 (0.6); 7.1716 (0.8);
7.1687 (0.8); 7.0196 (0.6); 7.0163 (0.5); 6.9988 (0.5); 6.9948 (0.8); 6.9909 (0.6); 6.9045 (0.7); 6.9031 (0.8); 6.8971 (0.7);
6.8956 (0.7); 6.8834 (0.7); 6.8819 (0.7); 6.8759 (0.7); 6.8745 (0.7); 5.9242 (5.5); 5.3002 (0.6); 4.5183 (1.9); 4.5020 (4.3);
4.4857 (2.0); 3.6784 (0.6); 3.6677 (14.5); 3.6597 (0.7); 3.6458 (16.0); 2.7284 (1.7); 2.7121 (3.6); 2.6958 (1.7); 1.5716 (0.7);
1.5694 (0.7); 1.5628 (1.2); 1.5552 (1.4); 1.5498 (0.7); 1.5375 (0.6); 0.7819 (0.8); 0.7799 (1.5); 0.7761 (0.9); 0.7731 (0.8);
0.7594 (3.2); 0.7541 (1.4); 0.7488 (0.9); 0.7449 (1.8); 0.7411 (1.1); −0.0002 (16.2)
I-62: ¹H NMR(400.6 MHz, CDCl3):
δ = 8.0850 (0.9); 8.0791 (0.9); 7.4276 (0.6); 7.4234 (0.7); 7.4046 (0.5); 7.2613 (14.2); 7.2232 (0.5); 7.2035 (0.8); 7.0440 (0.5);
7.0231 (0.5); 7.0195 (0.9); 7.0159 (0.5); 6.9980 (0.5); 6.9393 (0.7); 6.9378 (0.7); 6.9319 (0.7); 6.9303 (0.6); 6.9181 (0.7);
6.9166 (0.7); 6.9106 (0.7); 6.9091 (0.6); 5.9209 (5.1); 4.5212 (1.5); 4.5052 (3.6); 4.4892 (1.7); 3.8499 (0.7); 3.6744 (0.8);
3.6667 (16.0); 3.6493 (14.0); 2.7187 (1.6); 2.7027 (3.2); 2.6867 (1.5); 1.5730 (0.9); 0.0080 (0.7); −0.0002 (23.0); −0.0085 (0.6)
I-65: ¹H NMR(400.6 MHz, CDCl3):
δ = 8.1246 (1.4); 8.1184 (1.5); 7.7712 (0.6); 7.7650 (0.6); 7.7526 (0.7); 7.7500 (0.8); 7.7464 (0.7); 7.7437 (0.7); 7.7314 (0.6);
7.7251 (0.6); 7.4371 (0.5); 7.4328 (0.6); 7.4179 (0.9); 7.4136 (1.1); 7.3991 (0.6); 7.3947 (0.8); 7.3708 (0.6); 7.3585 (0.6);
7.3501 (0.5); 7.2620 (9.8); 7.2404 (0.6); 7.2389 (0.7); 7.2372 (0.7); 7.2177 (1.0); 7.0608 (0.7); 7.0576 (0.6); 7.0400 (0.6);
7.0362 (1.0); 7.0324 (0.7); 7.0149 (0.6); 7.0117 (0.5); 6.9580 (0.9); 6.9567 (1.0); 6.9507 (0.9); 6.9494 (0.9); 6.9368 (0.8);
6.9354 (0.9); 6.9294 (0.8); 6.9281 (0.8); 6.0116 (6.3); 4.0877 (0.9); 4.0700 (0.9); 4.0639 (1.2); 4.0461 (1.2); 3.9214 (1.2);
3.9037 (1.4); 3.8975 (0.9); 3.8799 (0.9); 2.0087 (16.0); 1.4321 (2.4); 1.3626 (4.0); 1.3449 (8.4); 1.3273 (3.9); −0.0002 (14.0)
I-66: ¹H NMR(400.0 MHz, CDCl3):
δ = 8.1085 (2.0); 8.1023 (2.0); 7.7666 (0.9); 7.7603 (0.9); 7.7477 (1.1); 7.7454 (1.2); 7.7416 (1.1); 7.7391 (1.1); 7.7266 (0.9);
7.7203 (0.9); 7.4209 (0.8); 7.4165 (0.9); 7.4018 (1.4); 7.3973 (1.6); 7.3828 (0.9); 7.3783 (1.1); 7.3631 (0.5); 7.3564 (0.8);
7.3438 (0.8); 7.3358 (0.7); 7.3312 (0.6); 7.3234 (0.7); 7.3190 (0.5); 7.2607 (30.8); 7.2240 (1.0); 7.2047 (1.5); 7.1878 (0.6);
7.0494 (1.0); 7.0461 (1.0); 7.0285 (1.0); 7.0248 (1.6); 7.0035 (0.8); 7.0004 (0.8); 6.9452 (1.4); 6.9379 (1.4); 6.9237 (1.3);
6.9161 (1.2); 5.9638 (8.0); 4.3323 (1.0); 4.3168 (3.0); 4.3145 (3.0); 4.2989 (3.1); 4.2968 (3.0); 4.2792 (1.1); 4.0586 (1.2);
4.0410 (1.2); 4.0347 (1.6); 4.0170 (1.6); 3.9993 (0.5); 3.9174 (0.6); 3.8998 (1.6); 3.8820 (1.6); 3.8758 (1.2); 3.8643 (0.6);
3.8581 (1.2); 1.5451 (16.0); 1.3507 (5.6); 1.3327 (14.5); 1.3142 (14.2); 1.2961 (5.7); 1.2547 (1.1); 0.0079 (1.6); −0.0002
(44.1); −0.0084 (1.5)
I-67: ¹H NMR(400.0 MHz, CDCl3):
δ = 8.1085 (2.0); 8.1021 (2.0); 7.7666 (0.9); 7.7603 (0.9); 7.7476 (1.1); 7.7453 (1.1); 7.7413 (1.1); 7.7266 (1.0); 7.7203 (0.9);
7.4208 (0.8); 7.4164 (0.9); 7.4015 (1.4); 7.3973 (1.6); 7.3827 (1.0); 7.3783 (1.1); 7.3629 (0.5); 7.3564 (0.8); 7.3436 (0.8);
7.3359 (0.8); 7.3311 (0.6); 7.3234 (0.7); 7.3190 (0.5); 7.2606 (33.0); 7.2242 (1.0); 7.2070 (1.5); 7.1866 (0.6); 7.0494 (1.0);
7.0462 (0.9); 7.0284 (1.0); 7.0247 (1.6); 7.0209 (1.0); 7.0033 (0.9); 7.0001 (0.8); 6.9451 (1.4); 6.9377 (1.4); 6.9234 (1.3);
6.9165 (1.2); 5.9637 (8.3); 4.3347 (1.1); 4.3322 (1.0); 4.3168 (3.1); 4.3145 (3.0); 4.2989 (3.2); 4.2968 (3.0); 4.2809 (1.1);
4.0587 (1.2); 4.0524 (0.6); 4.0409 (1.3); 4.0347 (1.6); 4.0171 (1.6); 3.9994 (0.5); 3.8996 (1.6); 3.8934 (0.5); 3.8820 (1.7);
3.8758 (1.2); 3.8644 (0.6); 3.8582 (1.2); 1.5436 (16.0); 1.3507 (5.7); 1.3327 (14.6); 1.3142 (14.4); 1.2961 (5.8); 1.2552 (1.0);
0.0079 (1.8); −0.0002 (47.1); −0.0084 (1.4)
I-68: ¹H NMR(400.6 MHz, CDCl3):
δ = 8.4582 (1.8); 8.4514 (1.9); 8.3447 (1.1); 8.3411 (1.9); 8.3376 (1.2); 8.2268 (1.0); 8.2249 (1.1); 8.2168 (0.9); 8.2152 (1.0);
8.2132 (1.0); 7.5034 (0.7); 7.4998 (0.7); 7.4827 (0.9); 7.4794 (1.3); 7.4762 (0.9); 7.4739 (0.9); 7.4695 (0.8); 7.4670 (0.8);
7.4626 (0.8); 7.4593 (0.9); 7.4556 (0.9); 7.4517 (0.8); 7.4473 (0.8); 7.4448 (0.8); 7.4405 (0.7); 7.3243 (0.8); 7.3153 (0.9);
7.3127 (0.9); 7.3036 (1.3); 7.2947 (0.7); 7.2920 (0.7); 7.2830 (0.6); 7.2626 (7.7); 5.9646 (5.6); 3.7037 (0.8); 3.6819 (16.0);
1.6186 (0.5); 1.6116 (0.6); 1.5981 (0.9); 1.5843 (0.5); 1.5774 (0.6); 1.4321 (0.5); 0.8278 (0.7); 0.8252 (0.9); 0.8222 (1.6);
0.8194 (1.2); 0.8145 (1.3); 0.8044 (0.8); 0.8008 (1.3); 0.7978 (1.2); 0.7940 (1.2); 0.7900 (1.4); 0.7820 (1.3); 0.7763 (1.7);
0.7715 (1.2); 0.7700 (1.2); 0.7643 (1.0); −0.0002 (11.5)
I-69: ¹H NMR(400.6 MHz, CDCl3):
δ = 8.1421 (1.7); 8.1362 (1.7); 7.6879 (0.6); 7.6817 (0.6); 7.6688 (0.9); 7.6668 (0.9); 7.6627 (0.9); 7.6607 (0.8); 7.6479 (0.6);
7.6418 (0.6); 7.3852 (0.6); 7.3810 (0.7); 7.3659 (1.1); 7.3620 (1.3); 7.3471 (1.0); 7.3430 (0.9); 7.3324 (0.7); 7.3281 (0.6);
7.3202 (0.7); 7.3157 (0.6); 7.3139 (0.6); 7.3120 (0.6); 7.2622 (7.7); 7.2023 (0.9); 7.2006 (0.9); 7.1826 (1.3); 7.1638 (0.5);
7.0491 (0.8); 7.0462 (0.7); 7.0281 (0.8); 7.0245 (1.2); 7.0209 (0.8); 7.0030 (0.7); 7.0002 (0.6); 6.9242 (1.0); 6.9169 (1.1);
6.9031 (1.0); 6.8958 (1.0); 5.8716 (5.5); 5.3003 (9.8); 3.7155 (0.7); 3.6974 (16.0); 1.5859 (0.6); 1.5782 (0.6); 1.5650 (1.1);
1.5517 (0.6); 1.5440 (0.6); 1.4322 (0.7); 0.8193 (0.5); 0.8060 (1.2); 0.8025 (1.8); 0.7929 (1.2); 0.7857 (1.2); 0.7810 (1.5);
0.7721 (0.9); 0.7319 (0.9); 0.7223 (1.7); 0.7185 (2.2); 0.7140 (1.1); 0.7097 (1.6); 0.7054 (1.7); −0.0002 (10.8)
I-70: ¹H NMR(400.6 MHz, CDCl3):
δ = 8.0643 (0.8); 8.0582 (0.8); 7.3963 (0.8); 7.3812 (1.0); 7.3763 (1.2); 7.3626 (1.0); 7.3614 (0.9); 7.3578 (0.6); 7.3570 (0.6);
7.2620 (10.7); 7.2009 (0.8); 7.1991 (0.8); 7.0264 (0.8); 6.9425 (0.6); 6.9409 (0.7); 6.9351 (0.6); 6.9335 (0.6); 6.9213 (0.7);
6.9197 (0.7); 6.9138 (0.6); 6.9123 (0.7); 5.9847 (0.5); 5.9737 (4.5); 3.8555 (0.8); 3.8498 (15.0); 3.8400 (1.8); 3.6858 (0.8);
3.6744 (16.0); 3.6541 (1.8); −0.0002 (12.1)
I-71: ¹H NMR(400.6 MHz, CDCl3):
δ = 8.1195 (0.9); 8.1133 (1.0); 7.6575 (0.5); 7.6554 (0.6); 7.6513 (0.6); 7.6492 (0.6); 7.3618 (0.7); 7.3574 (0.8); 7.3384 (0.5);
7.2645 (3.9); 7.1673 (0.7); 7.1646 (0.7); 6.9978 (0.7); 6.9939 (0.5); 6.9056 (0.6); 6.9043 (0.6); 6.8982 (0.6); 6.8968 (0.6);
6.8845 (0.6); 6.8831 (0.6); 6.8770 (0.6); 6.8757 (0.6); 5.9711 (5.1); 5.2999 (6.6); 3.8569 (0.6); 3.8397 (15.1); 3.6880 (0.6);
3.6599 (16.0); 1.5936 (1.8); 1.5665 (0.8); 0.7901 (0.6); 0.7870 (1.3); 0.7841 (0.9); 0.7798 (1.0); 0.7710 (0.5); 0.7695 (0.6);
0.7655 (1.1); 0.7630 (1.7); 0.7597 (1.5); 0.7557 (1.0); 0.7541 (0.9); 0.7511 (0.8); 0.7481 (1.2); 0.7439 (1.3); 0.7405 (0.7);
0.7379 (0.7); 0.7366 (0.7); −0.0002 (4.4)
I-72: ¹H NMR(400.6 MHz, CDCl3):
δ = 8.4563 (1.4); 8.4494 (1.4); 8.3117 (0.8); 8.3080 (1.4); 8.3043 (0.9); 8.2158 (0.7); 8.2141 (0.8); 8.2122 (0.8); 8.2106 (0.8);
8.2042 (0.7); 8.2024 (0.8); 8.2006 (0.8); 8.1989 (0.8); 7.5288 (0.6); 7.5251 (0.6); 7.5196 (0.8); 7.5153 (0.7); 7.5128 (0.7);
7.5082 (1.3); 7.5047 (1.0); 7.5020 (0.8); 7.4976 (0.7); 7.4932 (0.7); 7.4907 (0.7); 7.4852 (0.9); 7.4813 (0.7); 7.3536 (0.7);
7.3446 (0.8); 7.3420 (0.8); 7.3329 (1.2); 7.3239 (0.6); 7.3213 (0.6); 7.3123 (0.6); 7.2609 (21.6); 6.0000 (5.1); 3.8533 (15.1);
3.6875 (16.0); 2.0454 (0.5); 1.5556 (5.6); 0.0080 (0.9); 0.0042 (0.6); −0.0002 (32.1); −0.0085 (1.1)
I-73: ¹H NMR(400.6 MHz, CDCl3):
δ = 8.4181 (1.4); 8.4113 (1.4); 8.3210 (0.8); 8.3172 (1.5); 8.3135 (0.9); 8.1912 (0.6); 8.1893 (0.8); 8.1875 (0.8); 8.1858 (0.8);
8.1795 (0.7); 8.1776 (0.8); 8.1758 (0.8); 8.1742 (0.7); 7.4996 (0.6); 7.4959 (0.6); 7.4790 (0.7); 7.4757 (1.0); 7.4724 (0.7);
7.4555 (0.7); 7.4518 (0.7); 7.4347 (0.6); 7.4303 (0.6); 7.4278 (0.7); 7.4234 (0.6); 7.4121 (0.6); 7.4077 (0.7); 7.4052 (0.6);
7.4008 (0.6); 7.2998 (0.7); 7.2909 (0.8); 7.2882 (0.8); 7.2792 (1.2); 7.2703 (0.6); 7.2675 (0.7); 7.2625 (7.8); 7.2588 (0.9);
6.0153 (5.2); 3.8549 (0.8); 3.8360 (14.8); 3.6898 (0.8); 3.6597 (16.0); 2.0454 (0.7); 1.6227 (0.6); 1.6163 (0.5); 1.6084 (1.1);
1.6025 (0.6); 1.5958 (0.5); 1.5927 (0.5); 1.5888 (0.6); 1.2596 (0.6); 0.8819 (0.6); 0.8235 (0.7); 0.8192 (1.2); 0.8181 (1.2);
0.8165 (1.2); 0.8125 (0.8); 0.7974 (2.6); 0.7931 (1.7); 0.7847 (1.3); 0.7836 (1.3); 0.7812 (1.0); 0.7800 (0.9); 0.7767 (0.8);
0.7741 (0.6); −0.0002 (10.6)
I-76: ¹H NMR(400.6 MHz, CDCl3):
δ = 8.4184 (1.3); 8.4116 (1.4); 8.3223 (0.8); 8.3186 (1.4); 8.3149 (0.8); 8.1930 (0.7); 8.1911 (0.8); 8.1893 (0.8); 8.1875 (0.7);
8.1814 (0.7); 8.1795 (0.8); 8.1777 (0.8); 8.1760 (0.7); 7.4995 (0.6); 7.4958 (0.6); 7.4789 (0.7); 7.4755 (1.0); 7.4723 (0.6);
7.4554 (0.7); 7.4517 (0.7); 7.4346 (0.6); 7.4303 (0.6); 7.4277 (0.6); 7.4234 (0.6); 7.4121 (0.6); 7.4077 (0.7); 7.4051 (0.6);
7.4008 (0.6); 7.3005 (0.7); 7.2916 (0.8); 7.2888 (0.7); 7.2799 (1.2); 7.2710 (0.6); 7.2682 (0.7); 7.2625 (9.4); 7.2595 (0.8);
5.9766 (5.9); 4.5173 (1.9); 4.5008 (4.2); 4.4844 (2.0); 3.6815 (14.3); 3.6454 (16.0); 2.7325 (1.7); 2.7161 (3.6); 2.6996 (1.6);
1.6070 (0.5); 1.5997 (1.4); 1.5929 (0.9); 1.5869 (0.6); 1.5848 (0.6); 1.5804 (0.5); 0.8183 (0.6); 0.8128 (1.5); 0.8094 (1.2);
0.8025 (1.2); 0.7985 (2.0); 0.7957 (1.7); 0.7928 (2.2); 0.7899 (1.6); 0.7874 (1.7); 0.7850 (1.9); 0.7822 (0.8); 0.7805
(0.8); −0.0002 (13.8)
I-77: ¹H NMR(400.6 MHz, CDCl3):
δ = 8.4186 (1.0); 8.4119 (1.0); 8.3187 (1.2); 8.1929 (0.7); 8.1911 (0.8); 8.1893 (0.8); 8.1877 (0.7); 8.1812 (0.8); 8.1795 (0.8);
8.1777 (0.8); 7.4994 (0.5); 7.4957 (0.6); 7.4787 (0.7); 7.4754 (1.0); 7.4722 (0.6); 7.4552 (0.7); 7.4516 (0.7); 7.4344 (0.6);
7.4300 (0.6); 7.4275 (0.6); 7.4232 (0.5); 7.4118 (0.6); 7.4075 (0.6); 7.4049 (0.6); 7.4006 (0.5); 7.3004 (0.7); 7.2914 (0.8);
7.2888 (0.7); 7.2798 (1.2); 7.2708 (0.6); 7.2681 (0.6); 7.2623 (9.6); 7.2595 (0.7); 5.9766 (5.6); 4.5172 (1.9); 4.5008 (4.3);
4.4843 (2.0); 3.6815 (14.6); 3.6454 (16.0); 2.7325 (1.8); 2.7160 (3.7); 2.6996 (1.7); 1.5996 (1.1); 1.5925 (0.8); 1.5866 (1.2);
1.5848 (1.2); 1.5806 (0.9); 0.8182 (0.7); 0.8128 (1.5); 0.8093 (1.2); 0.8024 (1.3); 0.7984 (2.1); 0.7957 (1.8); 0.7927 (2.3);
0.7899 (1.6); 0.7873 (1.8); 0.7850 (1.9); 0.7821 (0.8); 0.7805 (0.8); −0.0002 (14.8)
I-78: ¹H NMR(400.0 MHz, CDCl3):
δ = 8.4447 (0.6); 8.3152 (0.6); 7.3493 (0.7); 7.3287 (0.7); 7.2610 (16.2); 7.1937 (0.5); 7.1866 (0.6); 7.1795 (0.9); 7.1732 (0.5);
7.1661 (0.5); 7.0039 (0.6); 6.9945 (0.7); 6.9846 (0.6); 6.9792 (0.8); 6.9674 (0.7); 6.9566 (0.9); 6.9446 (0.9); 5.9047 (5.6);
4.5248 (2.1); 4.5087 (4.5); 4.4925 (2.2); 3.6749 (15.6); 3.6460 (16.0); 2.7315 (2.1); 2.7154 (4.2); 2.6992 (2.0); 1.5851 (0.9);
1.5688 (3.0); 1.5513 (0.8); 0.8101 (1.3); 0.8069 (1.3); 0.7983 (1.1); 0.7853 (2.4); 0.7781 (1.5); 0.7733 (1.2); 0.7690 (1.6);
0.7644 (1.7); 0.7598 (0.9); 0.0079 (0.8); −0.0002 (24.3); −0.0084 (0.8)
I-79: ¹H NMR(400.6 MHz, CDCl3):
δ = 7.3288 (0.5); 7.2611 (18.8); 7.1796 (0.7); 6.9945 (0.5); 6.9798 (0.6); 6.9677 (0.6); 6.9569 (0.7); 6.9449 (0.7); 5.9047 (5.8);
4.5250 (1.8); 4.5089 (4.1); 4.4928 (2.0); 3.6750 (14.8); 3.6461 (16.0); 2.7319 (1.8); 2.7157 (3.7); 2.6996 (1.7); 1.5712 (0.7);
1.5681 (0.6); 1.5552 (5.3); 0.8102 (1.0); 0.8068 (1.0); 0.7980 (0.8); 0.7850 (1.8); 0.7776 (1.1); 0.7730 (1.0); 0.7689 (1.2);
0.7641 (1.3); 0.7598 (0.7); 0.0080 (0.8); −0.0002 (28.8); −0.0085 (0.8)
I-80: ¹H NMR(400.6 MHz, CDCl3):
δ = 8.2045 (0.6); 8.2026 (0.8); 8.2009 (0.8); 8.1991 (0.7); 8.1929 (0.7); 8.1910 (0.8); 8.1892 (0.8); 8.1875 (0.7); 8.1455 (1.0);
8.1394 (1.1); 7.7817 (0.5); 7.7627 (0.6); 7.7606 (0.6); 7.7565 (0.6); 7.7544 (0.6); 7.7417 (0.5); 7.7355 (0.5); 7.4824 (0.6);
7.4787 (0.6); 7.4618 (0.7); 7.4584 (1.0); 7.4552 (0.6); 7.4384 (0.7); 7.4346 (0.7); 7.2956 (0.7); 7.2867 (0.8); 7.2840 (0.7);
7.2750 (1.3); 7.2659 (0.7); 7.2618 (21.3); 7.2544 (0.6); 6.9268 (0.7); 6.9254 (0.7); 6.9194 (0.7); 6.9180 (0.7); 6.9058 (0.7);
6.9043 (0.7); 6.8983 (0.7); 6.8968 (0.6); 5.9794 (5.8); 4.5157 (1.9); 4.4992 (4.2); 4.4828 (1.9); 3.6808 (14.3); 3.6434 (16.0);
2.7322 (1.7); 2.7158 (3.6); 2.6993 (1.6); 1.5697 (1.5); 1.5618 (12.9); 1.5508 (0.6); 0.7927 (0.6); 0.7875 (1.5); 0.7842 (1.2);
0.7786 (1.3); 0.7749 (1.8); 0.7722 (1.3); 0.7699 (1.4); 0.7668 (2.0); 0.7630 (2.2); 0.0080 (0.9); −0.0002 (33.4); −0.0085 (0.8)
I-81: ¹H NMR(400.6 MHz, CDCl3):
δ = 8.2046 (0.6); 8.2027 (0.8); 8.2010 (0.8); 8.1992 (0.7); 8.1930 (0.7); 8.1911 (0.8); 8.1893 (0.8); 8.1876 (0.7); 8.1456 (1.0);
8.1415 (0.8); 8.1395 (1.1); 7.7757 (0.5); 7.7629 (0.6); 7.7608 (0.6); 7.7567 (0.6); 7.7546 (0.6); 7.7418 (0.5); 7.7356 (0.5);
7.4828 (0.6); 7.4791 (0.6); 7.4622 (0.7); 7.4588 (1.0); 7.4556 (0.6); 7.4388 (0.7); 7.4350 (0.7); 7.2960 (0.7); 7.2871 (0.8);
7.2844 (0.7); 7.2754 (1.3); 7.2664 (0.7); 7.2626 (12.3); 7.2549 (0.6); 6.9271 (0.7); 6.9256 (0.7); 6.9197 (0.7); 6.9182 (0.7);
6.9060 (0.6); 6.9045 (0.7); 6.8986 (0.7); 6.8970 (0.7); 5.9795 (5.8); 4.5157 (2.0); 4.4992 (4.3); 4.4828 (2.0); 3.6808 (14.4);
3.6772 (1.7); 3.6434 (16.0); 2.7323 (1.7); 2.7159 (3.6); 2.6994 (1.6); 1.5851 (0.6); 1.5745 (5.5); 0.7927 (0.6); 0.7875 (1.4);
0.7842 (1.2); 0.7786 (1.3); 0.7749 (1.7); 0.7722 (1.4); 0.7700 (1.4); 0.7668 (2.0); 0.7653 (2.1); 0.7631 (2.2); 0.7568
(0.7); −0.0002 (19.1); −0.0085 (0.6)
I-82: ¹H NMR(400.6 MHz, CDCl3):
δ = 8.4181 (1.8); 8.4112 (1.8); 8.3221 (1.0); 8.3182 (1.8); 8.3144 (1.0); 8.1929 (0.8); 8.1911 (0.9); 8.1893 (0.9); 8.1876 (0.8);
8.1813 (0.8); 8.1795 (0.9); 8.1777 (0.9); 8.1761 (0.7); 7.4993 (0.6); 7.4956 (0.6); 7.4786 (0.8); 7.4754 (1.1); 7.4721 (0.6);
7.4551 (0.7); 7.4515 (0.7); 7.4336 (0.7); 7.4292 (0.7); 7.4267 (0.7); 7.4224 (0.6); 7.4110 (0.7); 7.4066 (0.7); 7.4041 (0.7);
7.3998 (0.6); 7.3003 (0.8); 7.2913 (0.8); 7.2886 (0.8); 7.2797 (1.3); 7.2707 (0.7); 7.2680 (0.7); 7.2626 (8.9); 7.2592 (0.8);
5.9767 (5.8); 4.5173 (2.2); 4.5008 (4.4); 4.4844 (2.0); 3.6815 (14.9); 3.6454 (16.0); 2.7325 (1.8); 2.7161 (3.8); 2.6996 (1.7);
2.1720 (2.3); 1.5999 (1.0); 1.5816 (1.5); 1.2647 (0.7); 0.8819 (1.3); 0.8642 (0.5); 0.8181 (0.8); 0.8128 (1.6); 0.8093 (1.3);
0.8025 (1.3); 0.7987 (2.1); 0.7961 (1.8); 0.7926 (2.4); 0.7874 (1.9); 0.7854 (2.0); −0.0002 (13.2)
I-83: ¹H NMR(400.6 MHz, CDCl3):
δ = 8.4468 (1.3); 8.4400 (1.4); 8.3163 (0.8); 8.3125 (1.4); 8.3088 (0.8); 7.3538 (0.6); 7.3494 (0.6); 7.3469 (0.6); 7.3425 (0.6);
7.3314 (0.6); 7.3270 (0.7); 7.3245 (0.6); 7.3202 (0.6); 7.2610 (19.7); 7.1795 (0.7); 6.9942 (0.6); 6.9796 (0.6); 6.9675 (0.6);
6.9567 (0.7); 6.9447 (0.7); 5.9048 (5.7); 4.5250 (1.7); 4.5088 (3.9); 4.4927 (1.8); 3.6750 (14.0); 3.6461 (16.0); 2.7318 (1.6);
2.7156 (3.4); 2.6995 (1.6); 1.5713 (0.7); 1.5679 (0.6); 1.5521 (6.2); 0.8133 (0.6); 0.8101 (1.0); 0.8067 (1.1); 0.8016 (0.7);
0.7980 (0.9); 0.7885 (0.8); 0.7850 (1.8); 0.7824 (1.3); 0.7778 (1.2); 0.7733 (1.0); 0.7691 (1.1); 0.7642 (1.4); 0.7600 (0.7);
0.0080 (0.9); −0.0002 (29.9); −0.0085 (0.8)
I-84: ¹H NMR(400.6 MHz, CDCl3):
δ = 8.4578 (1.9); 8.4509 (1.9); 8.3401 (1.1); 8.3364 (1.9); 8.3328 (1.1); 8.2419 (0.8); 8.2403 (0.9); 8.2384 (1.0); 8.2302 (0.9);
8.2286 (1.0); 8.2266 (1.0); 7.5021 (0.6); 7.4985 (0.6); 7.4814 (0.8); 7.4782 (1.2); 7.4750 (0.7); 7.4607 (0.8); 7.4578 (1.1);
7.4565 (1.0); 7.4541 (1.4); 7.4496 (0.7); 7.4385 (0.7); 7.4342 (0.8); 7.4317 (0.7); 7.4273 (0.6); 7.3247 (0.8); 7.3156 (0.8);
7.3130 (0.8); 7.3040 (1.3); 7.2950 (0.6); 7.2924 (0.6); 7.2834 (0.6); 7.2626 (7.9); 5.9655 (5.7); 3.6829 (16.0); 1.6076 (0.6);
1.5941 (0.8); 1.5804 (0.5); 1.5734 (0.6); 0.8191 (0.8); 0.8159 (1.4); 0.8135 (1.2); 0.8078 (1.2); 0.7983 (0.8); 0.7941 (1.2);
0.7901 (0.8); 0.7874 (1.1); 0.7824 (1.3); 0.7746 (1.2); 0.7686 (1.7); 0.7658 (0.9); 0.7622 (1.2); 0.7568 (1.0); −0.0002 (11.6)
I-85: ¹H NMR(400.6 MHz, CDCl3):
δ = 8.4615 (2.4); 8.4548 (2.4); 8.3409 (2.6); 8.2453 (1.4); 8.2437 (1.5); 8.2420 (1.5); 8.2337 (1.5); 8.2319 (1.6); 8.2303 (1.5);
7.5034 (0.7); 7.4999 (0.8); 7.4826 (1.0); 7.4795 (1.6); 7.4670 (0.9); 7.4624 (1.2); 7.4597 (1.6); 7.4559 (1.7); 7.4449 (0.9);
7.4403 (1.1); 7.4342 (0.8); 7.3267 (0.8); 7.3175 (1.0); 7.3156 (1.1); 7.3060 (1.5); 7.2949 (0.9); 7.2854 (0.7); 7.2644 (4.2);
7.2635 (4.5); 5.9899 (2.2); 5.9870 (5.8); 3.6855 (16.0); 1.6159 (0.6); 1.6089 (0.7); 1.5951 (1.1); 1.5906 (0.6); 1.5817 (0.7);
1.5746 (0.7); 0.8143 (1.9); 0.8085 (1.8); 0.7985 (1.1); 0.7923 (1.9); 0.7862 (2.2); 0.7779 (2.0); 0.7722 (2.2); 0.7661 (2.1);
0.7602 (1.6); 0.0003 (6.3); −0.0002 (6.4)
I-86: ¹H NMR(400.6 MHz, CDCl3):
δ = 8.4179 (1.2); 8.4110 (1.3); 8.3207 (0.7); 8.3169 (1.3); 8.3131 (0.7); 8.1911 (0.6); 8.1892 (0.7); 8.1874 (0.7); 8.1856 (0.6);
8.1794 (0.6); 8.1776 (0.7); 8.1758 (0.7); 8.1739 (0.6); 7.4992 (0.5); 7.4955 (0.5); 7.4786 (0.7); 7.4752 (0.9); 7.4720 (0.6);
7.4551 (0.6); 7.4514 (0.6); 7.4346 (0.6); 7.4303 (0.6); 7.4277 (0.6); 7.4234 (0.5); 7.4120 (0.6); 7.4077 (0.6); 7.4051 (0.6);
7.4008 (0.5); 7.2994 (0.6); 7.2905 (0.7); 7.2878 (0.7); 7.2788 (1.2); 7.2699 (0.6); 7.2671 (0.6); 7.2612 (17.6); 7.2585 (1.1);
6.0152 (5.0); 3.8360 (14.4); 3.6596 (16.0); 1.6081 (0.8); 1.5883 (0.7); 1.5739 (1.2); 0.8234 (0.5); 0.8193 (1.0); 0.8180 (1.0);
0.8164 (0.9); 0.8123 (0.7); 0.7973 (2.2); 0.7929 (1.4); 0.7847 (1.0); 0.7833 (1.1); 0.7811 (0.8); 0.7797 (0.7); 0.7765 (0.7);
0.0079 (0.8); 0.0030 (0.5); 0.0022 (0.9); −0.0002 (24.8); −0.0027 (1.4); −0.0043 (0.6); −0.0085 (0.8)
I-87: ¹H NMR(400.6 MHz, CDCl3):
δ = 8.4182 (1.8); 8.4113 (1.8); 8.3206 (1.1); 8.3171 (1.9); 8.3134 (1.1); 8.1873 (1.0); 8.1774 (1.0); 7.4992 (0.6); 7.4956 (0.6);
7.4785 (0.8); 7.4754 (1.2); 7.4721 (0.7); 7.4551 (0.7); 7.4515 (0.7); 7.4347 (0.6); 7.4303 (0.7); 7.4279 (0.7); 7.4235 (0.6);
7.4121 (0.7); 7.4076 (0.8); 7.4054 (0.7); 7.4009 (0.6); 7.2994 (0.7); 7.2904 (0.8); 7.2878 (0.8); 7.2788 (1.2); 7.2697 (0.8);
7.2669 (0.9); 7.2609 (20.9); 6.0155 (5.3); 3.8362 (15.3); 3.6598 (16.0); 1.6227 (0.6); 1.6084 (1.0); 1.5926 (0.5); 1.5887 (0.8);
1.5742 (0.8); 1.5632 (0.9); 0.8191 (1.5); 0.7975 (3.3); 0.7844 (1.6); 0.7765 (0.9); 0.0080 (1.0); −0.0002 (30.6); −0.0083 (1.1)
I-89: ¹H NMR(400.6 MHz, CDCl3):
δ = 8.2970 (1.4); 8.2954 (1.4); 8.2871 (1.3); 8.2854 (1.4); 8.2837 (1.4); 8.1551 (1.8); 8.1493 (1.9); 7.7802 (0.6); 7.7741 (0.6);
7.7607 (1.0); 7.7547 (1.0); 7.7403 (0.7); 7.7341 (0.6); 7.4986 (0.6); 7.4950 (0.7); 7.4750 (1.4); 7.4546 (0.8); 7.4511 (0.8);
7.3405 (0.8); 7.3311 (1.0); 7.3289 (1.0); 7.3198 (1.3); 7.3083 (0.7); 7.2991 (0.6); 7.2625 (6.7); 6.9433 (1.1); 6.9361 (1.2);
6.9222 (1.1); 6.9149 (1.1); 5.9687 (6.1); 3.7118 (1.0); 3.6904 (16.0); 1.5865 (0.6); 1.5791 (0.6); 1.5654 (1.0); 1.5524 (0.6);
1.5448 (0.6); 0.7882 (2.4); 0.7782 (0.8); 0.7672 (2.5); 0.7569 (1.2); 0.7491 (0.8); 0.7388 (2.3); 0.7271 (2.2); −0.0002 (10.1)
I-90: ¹H NMR(400.6 MHz, CDCl3):
δ = 8.2669 (1.4); 8.2553 (1.5); 8.1579 (1.8); 8.1530 (1.9); 7.7853 (0.6); 7.7793 (0.6); 7.7657 (1.1); 7.7596 (1.1); 7.7453 (0.7);
7.7393 (0.7); 7.5075 (0.6); 7.5048 (0.7); 7.4842 (1.4); 7.4635 (0.8); 7.4607 (0.9); 7.3386 (0.8); 7.3288 (1.1); 7.3179 (1.3);
7.3080 (0.8); 7.2972 (0.6); 7.2611 (15.4); 6.9459 (1.2); 6.9386 (1.3); 6.9248 (1.2); 6.9175 (1.2); 5.9413 (5.6); 5.3004 (1.4);
3.6970 (16.0); 1.5903 (0.6); 1.5826 (0.6); 1.5688 (1.1); 1.5561 (0.7); 1.5484 (0.6); 0.7986 (2.1); 0.7911 (1.0); 0.7818 (1.7);
0.7779 (2.0); 0.7681 (0.8); 0.7550 (0.6); 0.7464 (1.0); 0.7373 (2.2); 0.7337 (2.1); 0.7253 (2.1); 0.0078 (0.9); −0.0002 (23.3)
I-91: ¹H NMR(400.6 MHz, CDCl3):
δ = 8.4575 (1.5); 8.4506 (1.6); 8.3062 (0.8); 8.3025 (1.5); 8.2988 (0.8); 8.2298 (0.6); 8.2280 (0.7); 8.2262 (0.8); 8.2245 (0.7);
8.2182 (0.7); 8.2164 (0.8); 8.2145 (0.8); 8.2129 (0.7); 7.5427 (0.5); 7.5390 (0.6); 7.5312 (0.6); 7.5268 (0.7); 7.5243 (0.7);
7.5220 (0.9); 7.5196 (1.4); 7.5159 (0.7); 7.5090 (0.7); 7.5046 (0.7); 7.5021 (0.7); 7.4986 (1.0); 7.4951 (0.7); 7.3692 (0.7);
7.3602 (0.8); 7.3576 (0.7); 7.3486 (1.2); 7.3395 (0.6); 7.3369 (0.6); 7.3279 (0.5); 7.2613 (13.5); 6.0089 (5.1); 3.8549 (14.6);
3.6899 (16.0); 2.9637 (0.8); 1.5521 (6.4); 0.0080 (0.6); −0.0002 (20.6); −0.0085 (0.6)
I-93: ¹H NMR(400.6 MHz, CDCl3):
δ = 8.1231 (1.2); 8.1170 (1.3); 7.6770 (0.5); 7.6709 (0.5); 7.6579 (0.7); 7.6560 (0.7); 7.6518 (0.7); 7.6499 (0.7); 7.6370 (0.6);
7.6307 (0.6); 7.2621 (6.3); 7.1343 (1.0); 7.1267 (1.0); 7.1178 (0.7); 7.1161 (0.6); 7.1102 (0.7); 7.0413 (0.9); 7.0213 (1.1);
6.9012 (0.8); 6.9000 (0.8); 6.8938 (0.8); 6.8801 (0.8); 6.8788 (0.8); 6.8727 (0.8); 5.9766 (5.2); 3.8364 (15.3); 3.6570 (16.0);
2.1662 (4.3); 2.1610 (4.3); 1.5808 (0.5); 1.5646 (3.6); 1.5534 (0.5); 1.5466 (0.6); 0.7876 (0.9); 0.7845 (1.5); 0.7826 (1.2);
0.7796 (0.9); 0.7769 (1.1); 0.7671 (0.9); 0.7629 (1.4); 0.7608 (1.6); 0.7574 (1.9); 0.7513 (1.5); 0.7440 (1.5); 0.7388 (1.5);
0.7361 (1.1); 0.7332 (1.0); −0.0002 (7.0)
I-94: ¹H NMR(400.6 MHz, CDCl3):
δ = 8.1184 (1.2); 8.1123 (1.3); 7.7743 (0.5); 7.7680 (0.5); 7.7555 (0.7); 7.7531 (0.7); 7.7493 (0.7); 7.7469 (0.7); 7.7343 (0.6);
7.7281 (0.6); 7.2618 (7.4); 7.1883 (0.9); 7.1785 (1.0); 7.1715 (0.7); 7.1617 (0.7); 7.0829 (0.8); 7.0640 (1.1); 6.9478 (0.8);
6.9464 (0.8); 6.9403 (0.8); 6.9390 (0.8); 6.9265 (0.8); 6.9251 (0.8); 6.9191 (0.8); 6.9178 (0.8); 5.9222 (5.4); 4.3392 (0.8);
4.3215 (2.6); 4.3037 (2.7); 4.2860 (1.0); 3.6853 (16.0); 2.1761 (4.2); 2.1709 (4.4); 1.3396 (3.7); 1.3218 (7.8); 1.3039
(3.7); −0.0002 (8.2)
I-95: ¹H NMR(400.6 MHz, CDCl3):
δ = 8.1271 (1.4); 8.1210 (1.4); 7.7780 (0.6); 7.7718 (0.6); 7.7592 (0.7); 7.7569 (0.8); 7.7531 (0.8); 7.7507 (0.7); 7.7381 (0.6);
7.7319 (0.6); 7.2620 (9.7); 7.2151 (0.6); 7.1962 (1.3); 7.1793 (0.9); 7.1758 (0.8); 7.0893 (0.9); 7.0689 (1.2); 7.0493 (0.5);
6.9459 (0.9); 6.9393 (0.9); 6.9247 (0.9); 6.9182 (0.9); 5.9083 (5.6); 4.5288 (2.0); 4.5125 (4.5); 4.4962 (2.2); 4.1309 (0.5);
4.1131 (0.6); 3.6749 (16.0); 3.6623 (15.3); 2.7336 (2.0); 2.7173 (4.2); 2.7010 (2.0); 2.1772 (4.8); 2.1721 (4.8); 2.0454 (2.5);
1.2774 (0.7); 1.2596 (1.3); 1.2417 (0.7); −0.0002 (10.6)
I-96: ¹H NMR(400.6 MHz, CDCl3):
δ = 8.1943 (1.5); 8.1812 (1.6); 7.2619 (7.3); 7.2224 (0.8); 7.2206 (0.8); 7.2067 (0.8); 7.1915 (0.9); 7.1748 (0.6); 7.1099 (0.9);
7.0898 (1.2); 7.0715 (1.2); 7.0682 (1.4); 7.0640 (0.8); 7.0586 (0.8); 7.0548 (1.2); 7.0510 (0.7); 6.8860 (1.8); 5.9138 (5.5);
4.3391 (0.8); 4.3214 (2.7); 4.3036 (2.8); 4.2857 (1.0); 3.6830 (16.0); 2.1935 (4.7); 2.1884 (4.7); 1.3381 (3.7); 1.3203 (7.8);
1.3025 (3.7); −0.0002 (7.9)
I-97: ¹H NMR(400.6 MHz, CDCl3):
δ = 8.1948 (1.6); 8.1818 (1.7); 7.2619 (8.3); 7.2329 (0.6); 7.2221 (1.0); 7.2125 (1.2); 7.1957 (0.8); 7.1143 (0.9); 7.0951 (1.3);
7.0766 (1.2); 7.0733 (1.4); 7.0692 (0.8); 7.0638 (0.8); 7.0599 (1.2); 7.0563 (0.8); 6.8927 (1.9); 5.9011 (5.6); 4.5284 (2.0);
4.5122 (4.4); 4.4959 (2.2); 3.6726 (16.0); 3.6612 (15.4); 2.7313 (2.0); 2.7151 (4.1); 2.6988 (1.9); 2.1942 (4.8); 2.1893
(5.0); −0.0002 (8.7)
I-98: ¹H NMR(400.6 MHz, CDCl3):
δ = 8.4486 (0.8); 8.4419 (0.8); 8.2907 (0.9); 7.4338 (0.5); 7.4294 (0.6); 7.4269 (0.6); 7.4225 (0.5); 7.4115 (0.6); 7.4071 (0.6);
7.4046 (0.6); 7.4002 (0.5); 7.2619 (5.6); 7.2017 (0.6); 7.1975 (0.7); 7.1858 (0.8); 7.1810 (0.6); 7.0932 (0.6); 7.0916 (0.6);
7.0729 (0.9); 5.9636 (5.1); 3.8555 (14.9); 3.6866 (16.0); 2.1799 (3.4); 2.1746 (3.4); −0.0002 (8.7)
I-99: ¹H NMR(400.6 MHz, CDCl3):
δ = 8.1144 (0.6); 8.1127 (0.9); 8.1106 (0.7); 8.1085 (0.7); 8.1064 (1.0); 7.7674 (0.5); 7.7611 (0.5); 7.7487 (0.6); 7.7462 (0.6);
7.7425 (0.6); 7.7399 (0.6); 7.7275 (0.5); 7.7212 (0.5); 7.2626 (3.8); 7.2023 (0.6); 7.2008 (0.6); 7.1980 (0.6); 7.1857 (0.8);
7.1812 (0.6); 7.0939 (0.6); 7.0922 (0.6); 7.0736 (0.9); 6.9528 (0.6); 6.9512 (0.7); 6.9453 (0.7); 6.9437 (0.7); 6.9315 (0.6);
6.9299 (0.7); 6.9240 (0.6); 6.9224 (0.6); 5.9620 (5.0); 3.8550 (14.7); 3.6861 (16.0); 2.1834 (3.3); 2.1780 (3.3); 1.5641
(0.9); −0.0002 (6.2)
I-100: ¹H NMR(400.6 MHz, CDCl3):
δ = 8.4487 (0.8); 8.4419 (0.8); 8.2847 (1.0); 7.4263 (0.6); 7.4219 (0.6); 7.4195 (0.6); 7.4151 (0.6); 7.4040 (0.6); 7.3996 (0.6);
7.3972 (0.6); 7.3928 (0.5); 7.2618 (5.9); 7.2063 (0.6); 7.1964 (0.7); 7.1027 (0.6); 7.1010 (0.6); 7.0824 (0.9); 5.9630 (4.9);
3.8569 (14.8); 3.6877 (16.0); 2.1851 (3.2); 2.1797 (3.3); −0.0002 (9.3)
I-101: ¹H NMR(400.6 MHz, CDCl3):
δ = 8.5863 (1.3); 8.5816 (1.8); 8.5765 (1.4); 8.5667 (1.4); 8.5617 (1.9); 8.5569 (1.4); 8.2174 (1.0); 8.2111 (1.0); 8.1992 (1.0);
8.1930 (1.1); 8.1829 (1.2); 8.1786 (1.3); 8.1712 (1.3); 8.1668 (1.3); 8.1619 (1.3); 8.1576 (1.3); 8.1502 (1.4); 8.1458 (1.3);
7.8112 (0.6); 7.8050 (0.6); 7.8005 (0.6); 7.7927 (0.7); 7.7901 (0.7); 7.7863 (0.7); 7.7838 (0.8); 7.7819 (0.7); 7.7794 (0.7);
7.7756 (0.6); 7.7730 (0.8); 7.7716 (0.7); 7.7652 (0.6); 7.7607 (0.6); 7.7545 (0.6); 7.4473 (1.3); 7.4355 (2.7); 7.4276 (1.3);
7.4238 (1.4); 7.4159 (2.6); 7.4041 (1.4); 7.2622 (24.4); 7.0102 (0.7); 7.0088 (0.7); 7.0028 (0.8); 7.0011 (0.9); 6.9998 (0.9);
6.9983 (0.9); 6.9921 (0.8); 6.9894 (0.9); 6.9877 (0.8); 6.9817 (0.8); 6.9800 (0.8); 6.9787 (0.8); 6.9771 (0.7); 6.9711 (0.7);
6.9696 (0.6); 5.8413 (6.6); 5.3006 (10.0); 3.8970 (14.4); 3.8856 (15.2); 3.6778 (16.0); 3.6573 (15.2); 2.9498 (12.8); 2.8989
(13.6); 2.1343 (0.8); 1.5659 (7.4); 1.2596 (0.6); 1.2552 (0.5); 0.0696 (1.0); 0.0079 (0.8); −0.0002 (29.8); −0.0085 (0.9)
I-102: ¹H NMR(400.6 MHz, CDCl3):
δ = 8.2523 (1.2); 8.2482 (1.3); 8.2405 (1.3); 8.2364 (1.3); 8.1369 (1.0); 8.1349 (0.8); 8.1328 (0.8); 8.1307 (1.0); 8.1288 (0.7);
8.0570 (1.2); 8.0529 (1.2); 8.0371 (1.3); 8.0330 (1.2); 7.8606 (0.6); 7.8543 (0.6); 7.8419 (0.6); 7.8393 (0.7); 7.8356 (0.6);
7.8331 (0.6); 7.8206 (0.6); 7.8143 (0.6); 7.3779 (1.4); 7.3661 (1.3); 7.3580 (1.3); 7.3463 (1.3); 7.2614 (12.5); 6.9485 (0.6);
6.9470 (0.7); 6.9410 (0.7); 6.9395 (0.7); 6.9273 (0.6); 6.9257 (0.7); 6.9198 (0.7); 6.9183 (0.7); 5.9314 (5.2); 5.3003 (2.9);
4.9054 (5.0); 4.9047 (5.1); 3.8522 (14.9); 3.6841 (0.5); 3.6797 (16.0); 1.5539 (0.8); 0.0694 (0.8); −0.0002 (15.5)
I-103: ¹H NMR(400.6 MHz, CDCl3):
δ = 8.6053 (1.0); 8.6009 (1.2); 8.5994 (1.3); 8.5950 (1.3); 8.5856 (1.1); 8.5812 (1.2); 8.5797 (1.4); 8.5753 (1.3); 8.2345 (0.8);
8.2324 (0.6); 8.2303 (0.6); 8.2282 (0.8); 8.2206 (0.9); 8.2185 (0.7); 8.2164 (0.7); 8.2143 (0.9); 8.2047 (1.0); 8.2003 (1.0);
8.1930 (1.1); 8.1884 (1.2); 8.1874 (1.3); 8.1829 (1.2); 8.1755 (1.2); 8.1711 (1.2); 7.8284 (0.5); 7.8221 (0.5); 7.8098 (1.0);
7.8073 (0.6); 7.8035 (0.6); 7.8010 (0.6); 7.7975 (0.5); 7.7949 (0.5); 7.7912 (0.5); 7.7886 (1.0); 7.7824 (0.6); 7.4679 (1.1);
7.4575 (1.4); 7.4563 (1.2); 7.4482 (1.1); 7.4459 (1.3); 7.4379 (1.4); 7.4365 (1.2); 7.4262 (1.2); 7.2630 (16.3); 7.0127 (0.6);
7.0111 (0.7); 7.0036 (1.2); 6.9961 (0.6); 6.9945 (0.6); 6.9915 (0.7); 6.9900 (0.7); 6.9824 (1.1); 6.9749 (0.5); 6.9734 (0.5);
5.8468 (4.1); 5.8443 (4.9); 5.3008 (16.0); 3.8977 (11.6); 3.8884 (13.7); 3.6794 (14.8); 3.6631 (12.4); 2.9632 (10.2); 2.9170
(12.0); 2.1332 (0.6); 1.5755 (2.2); 0.0699 (2.2); 0.0080 (0.5); −0.0002 (20.2); −0.0085 (0.6)
I-104: ¹H NMR(400.6 MHz, CDCl3):
δ = 8.1452 (0.9); 8.1432 (0.7); 8.1411 (0.7); 8.1389 (1.0); 8.1372 (0.7); 8.1203 (1.1); 8.1163 (1.2); 8.1086 (1.2); 8.1046 (1.2);
7.8746 (0.5); 7.8684 (0.5); 7.8558 (0.6); 7.8535 (0.6); 7.8496 (0.6); 7.8472 (0.6); 7.8347 (0.6); 7.8284 (0.6); 7.5995 (0.8);
7.5955 (0.8); 7.5794 (0.9); 7.5755 (0.9); 7.2849 (1.4); 7.2732 (1.3); 7.2650 (1.4); 7.2614 (14.1); 7.2532 (1.2); 6.9241 (0.6);
6.9226 (0.7); 6.9167 (0.7); 6.9152 (0.7); 6.9029 (0.6); 6.9013 (0.7); 6.8955 (0.6); 6.8940 (0.6); 5.9944 (5.1); 5.3003 (3.8);
3.8842 (0.6); 3.8413 (14.8); 3.6781 (16.0); 2.3851 (11.1); 1.5517 (4.6); −0.0002 (17.3); −0.0085 (0.5)
I-105: ¹H NMR(400.6 MHz, CDCl3):
δ = 8.1801 (0.6); 8.1781 (0.7); 8.1754 (0.7); 8.1734 (0.7); 8.1680 (0.6); 8.1660 (0.7); 8.1633 (0.7); 8.1612 (0.7); 7.7539 (0.6);
7.7491 (0.6); 7.7353 (0.7); 7.7334 (0.8); 7.7306 (0.7); 7.7286 (0.8); 7.7149 (0.7); 7.7101 (0.7); 7.4963 (0.8); 7.4940 (1.4);
7.4918 (0.8); 7.4758 (0.7); 7.4735 (1.2); 7.4713 (0.7); 7.3307 (1.3); 7.3252 (0.5); 7.3174 (1.4); 7.3139 (0.6); 7.3121 (0.6);
7.3084 (1.7); 7.3008 (0.6); 7.2952 (1.6); 7.2607 (9.8); 7.1497 (0.7); 7.1472 (0.7); 7.1375 (0.7); 7.1350 (0.7); 7.1312 (0.7);
7.1287 (0.7); 7.1190 (0.7); 7.1165 (0.6); 7.0922 (1.6); 7.0705 (2.6); 7.0653 (0.5); 7.0485 (1.4); 6.0289 (5.0); 3.8670 (15.0);
3.6959 (16.0); 1.5529 (0.9); −0.0002 (11.1)
I-106: ¹H NMR(400.6 MHz, CDCl3):
δ = 8.1190 (0.9); 8.1170 (0.7); 8.1149 (0.8); 8.1128 (1.0); 7.6577 (0.5); 7.6556 (0.6); 7.6515 (0.6); 7.6493 (0.6); 7.3651 (0.7);
7.3607 (0.8); 7.3417 (0.5); 7.2613 (13.1); 7.1655 (0.6); 7.1626 (0.7); 6.9944 (0.7); 6.9905 (0.5); 6.9041 (0.6); 6.9026 (0.7);
6.8966 (0.7); 6.8951 (0.7); 6.8830 (0.6); 6.8814 (0.6); 6.8755 (0.6); 6.8739 (0.6); 5.9353 (5.5); 4.3267 (0.5); 4.3237 (0.6);
4.3089 (1.6); 4.3059 (1.7); 4.2910 (1.7); 4.2882 (1.6); 4.2732 (0.6); 4.2704 (0.5); 3.6887 (0.6); 3.6602 (16.0); 1.5643 (1.0);
1.5544 (5.6); 1.5483 (0.8); 1.5452 (0.6); 1.3335 (3.6); 1.3157 (7.6); 1.2979 (3.5); 0.7856 (1.3); 0.7835 (2.1); 0.7792 (0.6);
0.7745 (0.6); 0.7704 (2.0); 0.7677 (1.5); 0.7638 (2.1); 0.7561 (1.9); −0.0002 (17.8); −0.0085 (0.6)
I-107: ¹H NMR(400.6 MHz, CDCl3):
δ = 8.2002 (1.5); 8.1886 (1.5); 8.1450 (2.0); 8.1406 (2.0); 7.7818 (0.6); 7.7756 (0.6); 7.7625 (1.1); 7.7564 (1.1); 7.7417 (0.6);
7.7356 (0.6); 7.4822 (0.7); 7.4586 (1.4); 7.4382 (0.8); 7.2944 (0.7); 7.2851 (1.2); 7.2739 (1.3); 7.2619 (9.4); 7.2534 (0.6);
6.9265 (1.2); 6.9191 (1.2); 6.9054 (1.1); 6.8980 (1.1); 6.0194 (5.2); 3.8527 (1.0); 3.8342 (15.5); 3.6875 (1.0); 3.6580 (16.0);
1.5948 (0.6); 1.5800 (1.2); 1.5615 (4.5); 0.7929 (2.8); 0.7741 (4.7); 0.7609 (2.3); 0.0079 (0.6); −0.0002 (13.4)
1-108: ¹H NMR(400.6 MHz, CDCl3):
δ = 8.2001 (1.5); 8.1884 (1.5); 8.1449 (1.9); 8.1405 (1.9); 7.7816 (0.6); 7.7755 (0.6); 7.7624 (1.2); 7.7563 (1.2); 7.7417 (0.7);
7.7356 (0.6); 7.4819 (0.7); 7.4584 (1.4); 7.4379 (0.9); 7.2942 (0.8); 7.2849 (1.2); 7.2735 (1.4); 7.2631 (10.3); 7.2614 (12.2);
7.2539 (0.6); 6.9264 (1.2); 6.9189 (1.2); 6.9052 (1.2); 6.8978 (1.2); 6.0193 (5.3); 3.8342 (15.6); 3.6579 (16.0); 1.5946 (0.6);
1.5800 (1.2); 1.5541 (6.2); 0.7930 (2.9); 0.7742 (4.8); 0.7609 (2.3); 0.0078 (0.9); 0.0016 (14.6); −0.0002 (17.7)
I-109: ¹H NMR(400.6 MHz, CDCl3):
δ = 8.4961 (1.7); 8.4894 (1.8); 8.3534 (1.0); 8.3499 (1.8); 8.3466 (1.2); 7.3890 (0.6); 7.3847 (0.7); 7.3822 (0.7); 7.3779 (0.6);
7.3671 (0.7); 7.3628 (0.8); 7.3603 (0.7); 7.3560 (0.7); 7.2627 (5.1); 7.1644 (0.8); 7.1580 (0.5); 7.1511 (0.5); 7.0113 (0.5);
7.0025 (0.6); 6.9928 (0.5); 6.9850 (0.9); 6.9730 (0.6); 6.9622 (0.9); 6.9502 (0.9); 5.9315 (4.5); 3.6850 (16.0); 1.5955 (0.5);
1.5879 (0.6); 1.5746 (0.9); 1.5612 (0.6); 1.5538 (0.6); 0.8819 (0.6); 0.8197 (0.8); 0.8160 (1.3); 0.8096 (0.8); 0.8066 (0.7);
0.7997 (1.2); 0.7940 (1.2); 0.7857 (0.7); 0.7643 (0.8); 0.7590 (0.8); 0.7562 (1.4); 0.7532 (1.1); 0.7505 (1.3); 0.7461 (0.9);
0.7431 (1.6); 0.7402 (1.1); 0.7373 (0.6); −0.0002 (6.4)
I-110: ¹H NMR(400.6 MHz, CDCl3):
δ = 8.4912 (1.8); 8.4843 (1.8); 8.3482 (1.0); 8.3445 (1.8); 8.3409 (1.0); 7.3840 (0.6); 7.3797 (0.7); 7.3771 (0.7); 7.3728 (0.6);
7.3620 (0.7); 7.3576 (0.7); 7.3551 (0.6); 7.3508 (0.6); 7.2620 (8.0); 7.1621 (0.7); 7.0080 (0.6); 6.9982 (0.5); 6.9928 (0.6);
6.9810 (0.6); 6.9702 (0.8); 6.9582 (0.8); 5.9321 (7.1); 3.6938 (16.0); 1.5977 (0.5); 1.5901 (0.5); 1.5768 (0.9); 1.5634 (0.5);
1.5559 (0.6); 1.4322 (0.5); 0.8239 (0.8); 0.8201 (1.2); 0.8131 (0.6); 0.8105 (0.8); 0.8034 (0.8); 0.7997 (1.0); 0.7982 (1.4);
0.7896 (0.5); 0.7616 (0.7); 0.7553 (0.9); 0.7527 (1.4); 0.7495 (0.9); 0.7477 (1.2); 0.7422 (1.0); 0.7396 (1.4); 0.7365 (1.0);
0.7342 (0.6); −0.0002 (10.7); −0.0027 (0.7)
I-111: ¹H NMR(400.6 MHz, CDCl3):
δ = 8.4479 (1.4); 8.4410 (1.4); 8.3101 (0.8); 8.3063 (1.5); 8.3025 (0.8); 7.3489 (0.6); 7.3445 (0.6); 7.3420 (0.6); 7.3376 (0.6);
7.3265 (0.6); 7.3221 (0.6); 7.3196 (0.6); 7.3153 (0.6); 7.2617 (10.9); 7.1483 (0.7); 6.9947 (0.6); 6.9824 (0.6); 6.9701 (0.5);
6.9593 (0.7); 6.9473 (0.7); 5.9490 (5.1); 3.8499 (14.9); 3.6602 (16.0); 1.5943 (0.5); 1.5807 (0.7); 1.5763 (0.6); 1.5675 (3.2);
1.5602 (0.8); 0.8174 (0.6); 0.8145 (1.3); 0.8114 (0.9); 0.8069 (0.9); 0.7967 (0.6); 0.7935 (1.1); 0.7901 (0.8); 0.7864 (0.8);
0.7823 (1.0); 0.7746 (1.0); 0.7702 (0.8); 0.7683 (1.4); 0.7655 (0.8); 0.7630 (0.9); 0.7619 (0.9); 0.7598 (0.8); 0.7571 (0.7);
0.7559 (0.7); −0.0002 (14.1)
I-112: ¹H NMR(400.6 MHz, CDCl3):
δ = 8.4479 (1.3); 8.4410 (1.3); 8.3102 (0.7); 8.3064 (1.4); 8.3026 (0.8); 7.3489 (0.6); 7.3445 (0.6); 7.3420 (0.6); 7.3376 (0.5);
7.3265 (0.6); 7.3222 (0.6); 7.3197 (0.6); 7.3153 (0.5); 7.2621 (7.8); 7.1484 (0.6); 6.9947 (0.5); 6.9826 (0.6); 6.9595 (0.6);
6.9475 (0.6); 5.9491 (5.1); 3.8499 (14.7); 3.6603 (16.0); 1.5809 (0.8); 1.5734 (2.4); 1.5682 (0.6); 1.5603 (0.5); 0.8174 (0.6);
0.8145 (1.2); 0.8114 (0.9); 0.8069 (0.9); 0.7966 (0.5); 0.7935 (1.0); 0.7901 (0.8); 0.7865 (0.8); 0.7823 (1.0); 0.7763 (0.8);
0.7747 (0.9); 0.7735 (0.8); 0.7704 (0.8); 0.7683 (1.3); 0.7655 (0.7); 0.7631 (0.8); 0.7620 (0.8); 0.7599 (0.7); 0.7573 (0.7);
0.7559 (0.7); −0.0002 (11.0)
I-113: ¹H NMR(400.6 MHz, CDCl3):
δ = 8.4165 (1.4); 8.4097 (1.5); 8.3213 (0.8); 8.3174 (1.5); 8.3136 (0.8); 8.1922 (0.6); 8.1903 (0.7); 8.1885 (0.7); 8.1868 (0.7);
8.1806 (0.7); 8.1787 (0.8); 8.1769 (0.7); 8.1752 (0.7); 7.4965 (0.5); 7.4928 (0.6); 7.4759 (0.7); 7.4726 (0.9); 7.4693 (0.6);
7.4524 (0.7); 7.4487 (0.6); 7.4347 (0.6); 7.4303 (0.6); 7.4278 (0.6); 7.4234 (0.6); 7.4121 (0.6); 7.4077 (0.6); 7.4052 (0.6);
7.4008 (0.6); 7.2976 (0.7); 7.2887 (0.7); 7.2860 (0.7); 7.2770 (1.2); 7.2681 (0.6); 7.2653 (0.7); 7.2614 (13.1); 7.2564 (0.7);
5.9921 (5.5); 4.3178 (0.5); 4.3037 (1.6); 4.3000 (1.6); 4.2859 (1.6); 4.2822 (1.6); 4.2680 (0.5); 4.2645 (0.5); 3.6600 (16.0);
1.6083 (0.8); 1.5917 (0.6); 1.5902 (0.5); 1.5741 (0.5); 1.3297 (3.6); 1.3118 (7.6); 1.2940 (3.5); 0.8172 (1.6); 0.8150 (1.7);
0.8104 (1.9); 0.8061 (0.7); 0.7964 (8.0); 0.0080 (0.5); −0.0002 (20.4); −0.0085 (0.6)
I-114: ¹H NMR(400.6 MHz, CDCl3):
δ = 8.1181 (1.1); 8.1161 (0.8); 8.1137 (0.9); 8.1119 (1.1); 7.7721 (0.5); 7.7659 (0.5); 7.7533 (0.6); 7.7509 (0.6); 7.7471 (0.6);
7.7447 (0.6); 7.7322 (0.6); 7.7259 (0.5); 7.2616 (6.5); 7.1917 (0.8); 7.1760 (1.0); 7.1602 (0.5); 7.0854 (0.7); 7.0652 (1.0);
6.9478 (0.7); 6.9464 (0.7); 6.9403 (0.8); 6.9390 (0.7); 6.9265 (0.7); 6.9251 (0.7); 6.9191 (0.7); 6.9177 (0.7); 5.9623 (5.2);
3.8537 (15.2); 3.6848 (16.0); 2.1785 (3.9); 2.1732 (3.8); 1.5548 (2.4); 0.8819 (0.7); −0.0002 (9.6)
I-115: ¹H NMR(400.6 MHz, CDCl3):
δ = 8.1952 (1.4); 8.1822 (1.4); 7.2619 (6.8); 7.2227 (0.7); 7.2085 (0.6); 7.2055 (0.7); 7.1890 (0.7); 7.1724 (0.5); 7.1098 (0.8);
7.0907 (1.1); 7.0701 (1.0); 7.0661 (1.1); 7.0622 (0.7); 7.0570 (0.6); 7.0530 (1.0); 7.0492 (0.6); 6.8848 (1.5); 5.9539 (5.2);
3.8540 (15.3); 3.6823 (16.0); 2.1958 (4.0); 2.1905 (4.0); 1.5599 (2.5); 0.8817 (0.5); −0.0002 (7.8)
I-116: ¹H NMR(400.6 MHz, CDCl3):
δ = 8.1234 (1.4); 8.1174 (1.5); 7.6784 (0.6); 7.6723 (0.6); 7.6577 (0.8); 7.6516 (0.8); 7.6383 (0.6); 7.6321 (0.6); 7.2615 (8.7);
7.1496 (1.0); 7.1314 (2.0); 7.1132 (1.4); 7.0389 (1.0); 7.0195 (1.2); 7.0001 (0.6); 6.8996 (1.0); 6.8922 (1.0); 6.8785 (0.9);
6.8711 (1.0); 5.9413 (5.4); 4.3233 (0.6); 4.3204 (0.7); 4.3055 (2.0); 4.3026 (2.0); 4.2877 (2.1); 4.2849 (2.0); 4.2698 (0.7);
4.2672 (0.7); 3.6572 (16.0); 2.1639 (5.1); 2.1588 (5.2); 1.5804 (0.6); 1.5654 (1.2); 1.5577 (3.2); 1.5492 (0.6); 1.5463 (0.7);
1.3323 (3.8); 1.3145 (7.9); 1.2966 (3.8); 0.7814 (3.0); 0.7765 (1.1); 0.7668 (2.7); 0.7620 (4.3); 0.7530 (2.7); −0.0002 (9.6)
I-117: ¹H NMR(400.6 MHz, CDCl3):
δ = 8.1314 (1.3); 8.1254 (1.4); 7.6847 (0.5); 7.6785 (0.5); 7.6655 (0.7); 7.6636 (0.8); 7.6594 (0.8); 7.6575 (0.7); 7.6446 (0.6);
7.6384 (0.6); 7.2623 (8.3); 7.1583 (0.9); 7.1396 (1.9); 7.1215 (1.4); 7.0448 (0.9); 7.0254 (1.2); 6.9046 (0.8); 6.8974 (0.9);
6.8834 (0.8); 6.8762 (0.8); 5.9270 (5.6); 4.5155 (2.0); 4.4991 (4.5); 4.4827 (2.2); 3.6695 (15.0); 3.6427 (16.0); 2.7274 (1.9);
2.7111 (4.1); 2.6947 (1.9); 2.1662 (4.4); 2.1611 (4.6); 1.5567 (0.8); 0.7854 (0.6); 0.7793 (1.6); 0.7751 (1.2); 0.7708 (0.9);
0.7583 (3.2); 0.7538 (2.3); 0.7493 (1.4); 0.7407 (1.9); 0.7367 (1.1); −0.0002 (9.2)
I-118: ¹H NMR(400.6 MHz, CDCl3):
δ = 8.4843 (1.7); 8.4775 (1.8); 8.2751 (1.0); 8.2715 (1.8); 8.2679 (1.0); 7.4561 (0.6); 7.4516 (0.8); 7.4492 (0.8); 7.4446 (1.1);
7.4402 (0.7); 7.4342 (0.8); 7.4295 (1.0); 7.4276 (1.2); 7.4238 (1.6); 7.4200 (0.8); 7.4076 (0.7); 7.4035 (0.7); 7.3176 (0.5);
7.3134 (0.5); 7.3014 (0.6); 7.2973 (1.2); 7.2932 (0.7); 7.2813 (0.8); 7.2771 (0.7); 7.2618 (8.0); 7.1716 (0.8); 7.1679 (0.8);
7.1514 (1.2); 7.1476 (1.2); 7.1311 (0.5); 7.1273 (0.5); 5.9024 (5.4); 4.3450 (0.8); 4.3272 (2.6); 4.3093 (2.7); 4.2915 (1.0);
3.6869 (16.0); 1.5612 (3.8); 1.3439 (3.8); 1.3260 (7.8); 1.3082 (3.7); −0.0002 (8.9)
I-119: ¹H NMR(400.6 MHz, CDCl3):
δ = 8.4885 (1.5); 8.4817 (1.5); 8.2908 (0.9); 8.2874 (1.5); 8.2840 (1.0); 7.4699 (0.6); 7.4654 (0.7); 7.4630 (0.7); 7.4586 (0.6);
7.4477 (0.9); 7.4432 (1.2); 7.4367 (0.7); 7.4304 (0.6); 7.4264 (1.2); 7.4224 (0.8); 7.4101 (0.6); 7.4059 (0.7); 7.3557 (0.5);
7.3515 (0.5); 7.3395 (0.6); 7.3354 (1.2); 7.3313 (0.7); 7.3193 (0.7); 7.3151 (0.6); 7.2621 (9.6); 7.1823 (0.7); 7.1784 (0.7);
7.1620 (1.2); 7.1581 (1.2); 7.1417 (0.5); 7.1379 (0.5); 5.8870 (5.7); 4.5318 (1.9); 4.5157 (4.3); 4.4996 (2.0); 3.6777 (16.0);
3.6620 (14.6); 2.7321 (1.8); 2.7160 (3.7); 2.6999 (1.7); −0.0002 (10.8)
I-120: ¹H NMR(400.6 MHz, CDCl3):
δ = 8.0869 (0.8); 8.0806 (0.9); 7.3971 (0.6); 7.3936 (0.8); 7.3930 (0.8); 7.3745 (0.8); 7.2615 (8.2); 7.2086 (0.6); 7.2069 (0.7);
7.2052 (0.7); 7.0360 (0.8); 6.9406 (0.6); 6.9390 (0.6); 6.9331 (0.6); 6.9315 (0.6); 6.9194 (0.6); 6.9178 (0.6); 6.9119 (0.6);
6.9103 (0.6); 6.8528 (0.8); 6.7177 (1.5); 6.5827 (0.9); 5.9841 (4.8); 5.2999 (1.5); 3.8538 (14.7); 3.6788 (16.0); 1.5521
(2.4); −0.0002 (9.2)
I-121: ¹H NMR(400.6 MHz, CDCl3):
δ = 8.0989 (1.0); 8.0927 (1.0); 7.7797 (0.5); 7.4320 (0.7); 7.4278 (0.8); 7.4133 (0.5); 7.4089 (0.6); 7.2612 (12.8); 7.2314 (0.5);
7.2297 (0.5); 7.2102 (0.8); 7.0554 (0.5); 7.0523 (0.5); 7.0309 (0.9); 7.0273 (0.5); 6.9390 (0.7); 6.9375 (0.7); 6.9316 (0.7);
6.9301 (0.7); 6.9178 (0.7); 6.9163 (0.7); 6.9103 (0.7); 6.9089 (0.6); 6.8381 (0.8); 6.7030 (1.6); 6.5680 (0.9); 5.9327 (5.5);
4.5268 (1.8); 4.5107 (4.2); 4.4947 (1.9); 3.6696 (16.0); 3.6593 (14.1); 2.7281 (1.7); 2.7121 (3.5); 2.6960 (1.6); 1.5511
(2.5); −0.0002 (18.1); −0.0085 (0.5)
I-122: ¹H NMR(400.6 MHz, CDCl3):
δ = 8.1720 (0.6); 8.1700 (0.6); 8.1673 (0.7); 8.1652 (0.6); 8.1598 (0.6); 8.1578 (0.7); 8.1551 (0.7); 8.1531 (0.6); 7.7473 (0.6);
7.7425 (0.6); 7.7287 (0.7); 7.7268 (0.8); 7.7240 (0.7); 7.7220 (0.8); 7.7082 (0.8); 7.7035 (0.7); 7.4899 (0.8); 7.4876 (1.4);
7.4854 (0.8); 7.4694 (0.7); 7.4672 (1.2); 7.4649 (0.7); 7.3274 (1.3); 7.3219 (0.5); 7.3141 (1.4); 7.3106 (0.6); 7.3088 (0.6);
7.3052 (1.7); 7.2975 (0.6); 7.2919 (1.6); 7.2608 (10.3); 7.1435 (0.7); 7.1409 (0.7); 7.1313 (0.7); 7.1288 (0.7); 7.1249 (0.7);
7.1224 (0.7); 7.1128 (0.7); 7.1103 (0.6); 7.0896 (1.7); 7.0678 (2.7); 7.0626 (0.5); 7.0459 (1.4); 6.0276 (5.0); 3.8665 (15.0);
3.8136 (0.5); 3.6954 (16.0); 1.5531 (3.5); −0.0002 (12.0)

### B. Formulation Examples

a) A dusting product is obtained by mixing 10 parts by weight of a compound of the formula (I) and/or salts thereof and 90 parts by weight of talc as inert substance and comminuting the mixture in an impact mill.
b) A readily water-dispersible, wettable powder is obtained by mixing 25 parts by weight of a compound of the formula (I) and/or salts thereof, 64 parts by weight of kaolin-containing quartz as inert substance, 10 parts by weight of potassium lignosulfonate and 1 part by weight of sodium oleoylmethyltaurate as wetting agent and dispersant and grinding in a pinned-disc mill.
c) A readily water-dispersible dispersion concentrate is obtained by mixing 20 parts by weight of a compound of the formula (I) and/or salts thereof with 6 parts by weight of alkylphenol polyglycol ether (® Triton X 207), 3 parts by weight of isotridecanol polyglycol ether (8 EO) and 71 parts by weight of paraffinic mineral oil (boiling range e.g. about 255 to more than 277° C.) and grinding to a fineness of below 5 microns in an attrition ball mill.
d) An emulsifiable concentrate is obtained from 15 parts by weight of a compound of the formula (I) and/or salts thereof, 75 parts by weight of cyclohexanone as solvent and 10 parts by weight of oxethylated nonylphenol as emulsifier.
e) Water-dispersible granules are obtained by mixing
75 parts by weight of a compound of the formula (I) and/or salts thereof,
10 parts by weight of calcium lignosulfonate,
5 parts by weight of sodium lauryl sulfate,
3 parts by weight of polyvinyl alcohol and
7 parts by weight of kaolin,
grinding the mixture in a pinned-disk mill, and granulating the powder in a fluidized bed by spray application of water as a granulating liquid.
f) Water-dispersible granules are also obtained by homogenizing and precomminuting, in a colloid mill,
25 parts by weight of a compound of the formula (I) and/or salts thereof,
5 parts by weight of sodium 2,2′-dinaphthylmethane-6,6′-disulfonate,
2 parts by weight of sodium oleoylmethyltaurate,
1 part by weight of polyvinyl alcohol,
17 parts by weight of calcium carbonate and
50 parts by weight of water,
then grinding the mixture in a bead mill and atomizing and drying the resulting suspension in a spray tower by means of a one-phase nozzle.

### C. Biological Examples

### 1. Pre-Emergence Herbicidal Effect and Crop Plant Compatibility

Seeds of monocotyledonous and dicotyledonous weed plants and crop plants are placed in plastic or organic planting pots and covered with soil. The compounds of the invention, formulated in the form of wettable powders (WP) or as emulsion concentrates (EC), are then applied to the surface of the covering soil as aqueous suspension or emulsion with addition of 0.5% additive at a water application rate equivalent to 600 l/ha. After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

Tables 1a to 19c below show the effects/crop compatibilities of selected compounds of the general formula (I) on various harmful plants at an application rate corresponding to 20 to 320 g/ha, which were obtained by the trial procedure specified above.

### Plants:

ALOMY: Alopecurus myosuroides SETVI: Setaria viridis
AVEFA: Avena fatua POLCO: Fallopia convolvulus
AMARE: Amaranthus retroflexus ECHCG: Echinochloa crus-galli
LOLRJ: Lolium rigidum STEME: Stellaria media
VERPE: Veronica persica MATIN: Tripleurospermum inodorum
DIGSA: Digitaria sanguinalis ABUTH: Abutylon threophrasti
TRZAS: Triticum aestivum VIOTR: Viola tricolor
BRSNW: Brassica napus ZEAMX: Zea mays
PHBPU: Ipomoea purpurea GLXMA: Glycine max
ORYSA: Oryza sativa HORMU: Hordeum murinum

### 1. Pre-Emergence Efficacy

As shown by the results from Tables 1a to 19c, compounds of the invention have good crop plant compatibility and good herbicidal pre-emergence efficacy against a broad spectrum of weed grasses and broad-leaved weeds.

TABLE 1a
Pre-emergence effect at 20 g/ha against ZEAMX in %

| Example number | Dosage [g/ha] | ZEAMX |
|---|---|---|
| I-49 | 20 | 0 |
| I-76 | 20 | 20 |

TABLE 1b
Pre-emergence effect at 80 g/ha against ZEAMX in %

| Example number | Dosage [g/ha] | ZEAMX |
|---|---|---|
| I-01 | 80 | 0 |
| I-03 | 80 | 20 |
| I-04 | 80 | 0 |
| I-05 | 80 | 20 |
| I-07 | 80 | 10 |
| I-08 | 80 | 0 |
| I-11 | 80 | 0 |
| I-49 | 80 | 0 |

TABLE 1c
Pre-emergence effect at 320 g/ha against ZEAMX in %

| Example number | Dosage [g/ha] | ZEAMX |
|---|---|---|
| I-03 | 320 | 20 |
| I-08 | 320 | 10 |
| I-09 | 320 | 10 |

TABLE 2a
Pre-emergence effect at 80 g/ha against TRZAS in %

| Example number | Dosage [g/ha] | TRZAS |
|---|---|---|
| I-06 | 80 | 10 |
| I-07 | 80 | 20 |
| I-08 | 80 | 0 |
| I-09 | 80 | 0 |
| I-11 | 80 | 0 |

TABLE 2b
Pre-emergence effect at 320 g/ha against TRZAS in %

| Example number | Dosage [g/ha] | TRZAS |
|---|---|---|
| I-03 | 320 | 0 |
| I-08 | 320 | 0 |
| I-09 | 320 | 20 |
| I-11 | 320 | 0 |

TABLE 3a
Pre-emergence effect at 80 g/ha against ORYSA in %

| Example number | Dosage [g/ha] | ORYSA |
|---|---|---|
| I-02 | 80 | 0 |
| I-03 | 80 | 0 |
| I-08 | 80 | 0 |

TABLE 3b
Pre-emergence effect at 320 g/ha against ORYSA in %

| Example number | Dosage [g/ha] | ORYSA |
|---|---|---|
| I-02 | 320 | 0 |
| I-08 | 320 | 0 |

TABLE 4a
Pre-emergence effect at 80 g/ha against GLXMA in %

| Example number | Dosage [g/ha] | GLXMA |
|---|---|---|
| I-01 | 80 | 0 |
| I-02 | 80 | 0 |
| I-03 | 80 | 20 |
| I-05 | 80 | 10 |
| I-06 | 80 | 10 |
| I-07 | 80 | 10 |
| I-08 | 80 | 0 |
| I-09 | 80 | 10 |
| I-11 | 80 | 0 |

TABLE 4b
Pre-emergence effect at 320 g/ha against GLXMA in %

| Example number | Dosage [g/ha] | GLXMA |
|---|---|---|
| I-02 | 320 | 0 |
| I-09 | 320 | 10 |
| I-11 | 320 | 0 |

TABLE 5a
Pre-emergence effect at 80 g/ha against BRSNW in %

| Example number | Dosage [g/ha] | BRSNW |
|---|---|---|
| I-02 | 80 | 0 |
| I-03 | 80 | 20 |
| I-09 | 80 | 0 |

TABLE 5b
Pre-emergence effect at 320 g/ha against BRSNW in %

| Example number | Dosage [g/ha] | BRSNW |
|---|---|---|
| I-02 | 320 | 0 |
| I-09 | 320 | 0 |

TABLE 6a
Pre-emergence effect at 80 g/ha against ABUTH in %

| Example number | Dosage [g/ha] | ABUTH |
|---|---|---|
| I-76 | 80 | 90 |

TABLE 6b
Pre-emergence effect at 320 g/ha against ABUTH in %

| Example number | Dosage [g/ha] | ABUTH |
|---|---|---|
| I-04 | 320 | 80 |
| I-31 | 320 | 80 |
| I-34 | 320 | 90 |
| I-76 | 320 | 90 |

TABLE 7a
Pre-emergence effect at 80 g/ha against ALOMY in %

| Example number | Dosage [g/ha] | ALOMY |
|---|---|---|
| I-04 | 80 | 80 |
| I-34 | 80 | 90 |
| I-76 | 80 | 90 |

TABLE 7b
Pre-emergence effect at 320 g/ha against ALOMY in %

| Example number | Dosage [g/ha] | ALOMY |
|---|---|---|
| I-01 | 320 | 90 |
| I-04 | 320 | 90 |
| I-05 | 320 | 90 |
| I-06 | 320 | 90 |
| I-31 | 320 | 100 |
| I-34 | 320 | 100 |
| I-49 | 320 | 90 |
| I-76 | 320 | 90 |

TABLE 8a
Pre-emergence effect at 20 g/ha against AMARE in %

| Example number | Dosage [g/ha] | AMARE |
|---|---|---|
| I-31 | 20 | 80 |
| I-76 | 20 | 80 |

TABLE 8b
Pre-emergence effect at 80 g/ha against AMARE in %

| Example number | Dosage [g/ha] | AMARE |
|---|---|---|
| I-01 | 80 | 80 |
| I-04 | 80 | 80 |
| I-06 | 80 | 90 |
| I-07 | 80 | 80 |
| I-31 | 80 | 100 |
| I-34 | 80 | 100 |
| I-49 | 80 | 90 |
| I-76 | 80 | 100 |

TABLE 8c
Pre-emergence effect at 320 g/ha against AMARE in %

| Example number | Dosage [g/ha] | AMARE |
|---|---|---|
| I-01 | 320 | 100 |
| I-03 | 320 | 80 |
| I-04 | 320 | 100 |
| I-05 | 320 | 90 |
| I-06 | 320 | 100 |
| I-07 | 320 | 90 |
| I-31 | 320 | 100 |
| I-34 | 320 | 100 |
| I-49 | 320 | 100 |
| I-76 | 320 | 100 |

TABLE 9a
Pre-emergence effect at 20 g/ha against AVEFA in %

| Example number | Dosage [g/ha] | AVEFA |
|---|---|---|
| I-34 | 20 | 80 |

TABLE 9b
Pre-emergence effect at 80 g/ha against AVEFA in %

| Example number | Dosage [g/ha] | AVEFA |
|---|---|---|
| I-31 | 80 | 90 |
| I-34 | 80 | 90 |
| I-76 | 80 | 90 |

TABLE 9c
Pre-emergence effect at 320 g/ha against AVEFA in %

| Example number | Dosage [g/ha] | AVEFA |
|---|---|---|
| I-04 | 320 | 80 |
| I-05 | 320 | 90 |
| I-07 | 320 | 80 |
| I-31 | 320 | 90 |
| I-34 | 320 | 100 |
| I-49 | 320 | 90 |
| I-76 | 320 | 100 |

TABLE 10a
Pre-emergence effect at 80 g/ha against DIGSA in %

| Example number | Dosage [g/ha] | DIGSA |
|---|---|---|
| I-76 | 80 | 80 |

TABLE 10b
Pre-emergence effect at 320 g/ha against DIGSA in %

| Example number | Dosage [g/ha] | DIGSA |
|---|---|---|
| I-05 | 320 | 90 |
| I-07 | 320 | 90 |
| I-31 | 320 | 90 |
| I-34 | 320 | 80 |
| I-49 | 320 | 100 |
| I-76 | 320 | 90 |

TABLE 11a
Pre-emergence effect at 80 g/ha against ECHCG in %

| Example number | Dosage [g/ha] | ECHCG |
|---|---|---|
| I-04 | 80 | 80 |
| I-31 | 80 | 90 |
| I-34 | 80 | 90 |
| I-76 | 80 | 90 |

TABLE 11b
Pre-emergence effect at 320 g/ha against ECHCG in %

| Example number | Dosage [g/ha] | ECHCG |
|---|---|---|
| I-01 | 320 | 100 |
| I-04 | 320 | 100 |
| I-05 | 320 | 90 |
| I-06 | 320 | 100 |
| I-07 | 320 | 90 |
| I-31 | 320 | 100 |
| I-34 | 320 | 100 |
| I-49 | 320 | 90 |
| I-76 | 320 | 100 |

TABLE 12a
Pre-emergence effect at 80 g/ha against LOLRI in %

| Example number | Dosage [g/ha] | LOLRI |
|---|---|---|
| I-01 | 80 | 90 |
| I-34 | 80 | 90 |
| I-76 | 80 | 90 |

TABLE 12b
Pre-emergence effect at 320 g/ha against LOLRI in %

| Example number | Dosage [g/ha] | LOLRI |
|---|---|---|
| I-01 | 320 | 100 |
| I-04 | 320 | 100 |
| I-05 | 320 | 100 |
| I-07 | 320 | 90 |
| I-31 | 320 | 100 |
| I-34 | 320 | 100 |
| I-49 | 320 | 90 |
| I-76 | 320 | 100 |

TABLE 13a
Pre-emergence effect at 80 g/ha against MATIN in %

| Example number | Dosage [g/ha] | MATIN |
|---|---|---|
| I-01 | 80 | 90 |
| I-06 | 80 | 80 |
| I-31 | 80 | 90 |
| I-34 | 80 | 90 |

TABLE 13b
Pre-emergence effect at 320 g/ha against MATIN in %

| Example number | Dosage [g/ha] | MATIN |
|---|---|---|
| I-01 | 320 | 90 |
| I-04 | 320 | 90 |
| I-05 | 320 | 90 |
| I-06 | 320 | 90 |
| I-07 | 320 | 90 |
| I-31 | 320 | 100 |
| I-34 | 320 | 90 |
| I-49 | 320 | 90 |
| I-76 | 320 | 90 |

TABLE 14a
Pre-emergence effect at 80 g/ha against PHBPU in %

| Example number | Dosage [g/ha] | PHBPU |
|---|---|---|
| I-04 | 80 | 80 |
| I-31 | 80 | 90 |
| I-34 | 80 | 90 |

TABLE 14b
Pre-emergence effect at 320 g/ha against PHBPU in %

| Example number | Dosage [g/ha] | PHBPU |
|---|---|---|
| I-04 | 320 | 90 |
| I-05 | 320 | 80 |
| I-06 | 320 | 90 |
| I-07 | 320 | 90 |
| I-31 | 320 | 90 |
| I-34 | 320 | 90 |
| I-76 | 320 | 90 |

TABLE 15a
Pre-emergence effect at 20 g/ha against POLCO in %

| Example number | Dosage [g/ha] | POLCO |
|---|---|---|
| I-31 | 20 | 80 |
| I-34 | 20 | 90 |

TABLE 15b
Pre-emergence effect at 80 g/ha against POLCO in %

| Example number | Dosage [g/ha] | POLCO |
|---|---|---|
| I-01 | 80 | 80 |
| I-04 | 80 | 80 |
| I-31 | 80 | 100 |
| I-34 | 80 | 90 |
| I-76 | 80 | 90 |

TABLE 15c
Pre-emergence effect at 320 g/ha against POLCO in %

| Example number | Dosage [g/ha] | POLCO |
|---|---|---|
| I-01 | 320 | 90 |
| I-04 | 320 | 90 |
| I-05 | 320 | 90 |
| I-06 | 320 | 80 |
| I-07 | 320 | 80 |
| I-31 | 320 | 100 |
| I-34 | 320 | 90 |
| I-49 | 320 | 80 |
| I-76 | 320 | 90 |

TABLE 16a
Pre-emergence effect at 80 g/ha against SETVI in %

| Example number | Dosage [g/ha] | SETVI |
|---|---|---|
| I-04 | 80 | 80 |
| I-76 | 80 | 90 |

TABLE 16b
Pre-emergence effect at 320 g/ha against SETVI in %

| Example number | Dosage [g/ha] | SETVI |
|---|---|---|
| I-01 | 320 | 100 |
| I-02 | 320 | 80 |
| I-04 | 320 | 100 |
| I-05 | 320 | 100 |
| I-07 | 320 | 100 |
| I-31 | 320 | 90 |
| I-34 | 320 | 90 |
| I-49 | 320 | 100 |
| I-76 | 320 | 100 |

TABLE 17
Pre-emergence effect at 320 g/ha against VERPE in %

| Example number | Dosage [g/ha] | VERPE |
|---|---|---|
| I-01 | 320 | 80 |
| I-04 | 320 | 90 |
| I-31 | 320 | 90 |
| I-34 | 320 | 80 |

TABLE 18a
Pre-emergence effect at 20 g/ha against VIOTR in %

| Example number | Dosage [g/ha] | VIOTR |
|---|---|---|
| I-34 | 20 | 80 |

TABLE 18b
Pre-emergence effect at 80 g/ha against VIOTR in %

| Example number | Dosage [g/ha] | VIOTR |
|---|---|---|
| I-04 | 80 | 80 |
| I-31 | 80 | 100 |
| I-34 | 80 | 100 |
| I-76 | 80 | 100 |

TABLE 18c
Pre-emergence effect at 320 g/ha against VIOTR in %

| Example number | Dosage [g/ha] | VIOTR |
|---|---|---|
| I-04 | 320 | 100 |
| I-05 | 320 | 100 |
| I-31 | 320 | 100 |
| I-34 | 320 | 100 |
| I-49 | 320 | 90 |
| I-76 | 320 | 100 |

TABLE 19a
Pre-emergence effect at 20 g/ha against KCHSC in %

| Example number | Dosage [g/ha] | KCHSC |
|---|---|---|
| I-76 | 20 | 90 |

TABLE 19b
Pre-emergence effect at 80 g/ha against KCHSC in %

| Example number | Dosage [g/ha] | KCHSC |
|---|---|---|
| I-76 | 80 | 90 |

TABLE 19c
Pre-emergence effect at 320 g/ha against KCHSC in %

| Example number | Dosage [g/ha] | KCHSC |
|---|---|---|
| I-49 | 320 | 100 |
| I-76 | 320 | 100 |

As the results show, inventive compounds of the general formula (I), in pre-emergence treatment, have good herbicidal efficacy against harmful plants such as Abutilon theophrasti, Alopecurus myosuroides, Amaranthus retroflexus, Avena fatua, Digitaria sanguinalis, Echinochloa crus-galli, Lolium rigidum, Setaria viridis, Stellaria media, Tripleurospermum inodorum, Veronica persica and Fallopia convolvulus at an application rate of between 0.020 and 0.320 kg of active substance per hectare, and good crop plant compatibility with organisms such as Zea mays, Oryza sativa, Brassica napus, Glycine max and Triticum aestivum at an application rate of 0.32 kg or less per hectare.

The compounds of the invention are therefore suitable for control of unwanted plant growth by the pre-emergence method.

### 2. Post-Emergence Herbicidal Effect and Crop Plant Compatibility

Seeds of monocotyledonous and dicotyledonous weeds and crop plants are placed in sandy loam in plastic or organic planting pots, covered with soil and cultivated in a greenhouse under controlled growth conditions. 2 to 3 weeks after sowing, the trial plants are treated at the one-leaf stage. The compounds of the invention, formulated in the form of wettable powders (WP) or as emulsion concentrates (EC), are then sprayed onto the green parts of the plants as aqueous suspension or emulsion with addition of 0.5% additive at a water application rate of 600 l/ha (converted). After the test plants have been kept in the greenhouse under optimum growth conditions for about 3 weeks, the activity of the preparations is rated visually in comparison to untreated controls. For example, 100% activity=the plants have died, 0% activity=like control plants.

Tables 20a to 38c below show the effects/crop compatibilities of selected compounds of the general formula (I) on various harmful plants at an application rate corresponding to 20 to 320 g/ha, which were obtained by the trial procedure specified above.

TABLE 20a
Post-emergence effect at 20 g/ha against ZEAMX in %

| Example number | Dosage [g/ha] | ZEAMX |
|---|---|---|
| I-13 | 20 | 10 |
| I-12 | 20 | 0 |
| I-10 | 20 | 20 |
| I-15 | 20 | 0 |
| I-20 | 20 | 0 |
| I-24 | 20 | 0 |
| I-25 | 20 | 20 |
| I-22 | 20 | 20 |
| I-26 | 20 | 20 |
| I-32 | 20 | 10 |
| I-35 | 20 | 20 |
| I-38 | 20 | 0 |
| I-39 | 20 | 20 |
| I-40 | 20 | 0 |
| I-41 | 20 | 0 |
| I-14 | 20 | 0 |
| I-48 | 20 | 0 |
| I-47 | 20 | 0 |
| I-46 | 20 | 0 |
| I-45 | 20 | 0 |
| I-44 | 20 | 0 |
| I-43 | 20 | 0 |
| I-42 | 20 | 0 |
| I-54 | 20 | 0 |
| I-51 | 20 | 0 |
| I-52 | 20 | 0 |
| I-53 | 20 | 0 |
| I-59 | 20 | 20 |
| I-55 | 20 | 0 |
| I-58 | 20 | 0 |
| I-56 | 20 | 0 |
| I-57 | 20 | 0 |
| I-69 | 20 | 0 |
| I-60 | 20 | 0 |
| I-71 | 20 | 10 |
| I-64 | 20 | 20 |
| I-61 | 20 | 0 |
| I-62 | 20 | 0 |
| I-65 | 20 | 0 |
| I-68 | 20 | 0 |
| I-82 | 20 | 20 |
| I-75 | 20 | 0 |
| I-74 | 20 | 0 |
| I-77 | 20 | 0 |
| I-80 | 20 | 20 |
| I-81 | 20 | 0 |
| I-86 | 20 | 0 |
| I-85 | 20 | 0 |
| I-88 | 20 | 0 |
| I-89 | 20 | 0 |
| I-90 | 20 | 0 |
| I-107 | 20 | 0 |
| I-108 | 20 | 0 |
| I-92 | 20 | 20 |
| I-93 | 20 | 10 |

TABLE 20b
Post-emergence effect at 80 g/ha against ZEAMX in %

| Example number | Dosage [g/ha] | ZEAMX |
|---|---|---|
| I-02 | 80 | 10 |
| I-03 | 80 | 20 |
| I-05 | 80 | 20 |
| I-09 | 80 | 0 |
| I-11 | 80 | 0 |
| I-13 | 80 | 20 |
| I-12 | 80 | 20 |
| I-15 | 80 | 10 |
| I-20 | 80 | 20 |
| I-24 | 80 | 10 |
| I-22 | 80 | 20 |
| I-40 | 80 | 0 |
| I-41 | 80 | 0 |
| I-47 | 80 | 0 |
| I-46 | 80 | 20 |
| I-45 | 80 | 0 |
| I-44 | 80 | 0 |
| I-43 | 80 | 0 |
| I-42 | 80 | 0 |
| I-51 | 80 | 0 |
| I-52 | 80 | 0 |
| I-55 | 80 | 0 |
| I-58 | 80 | 10 |
| I-56 | 80 | 0 |
| I-57 | 80 | 0 |
| I-69 | 80 | 0 |
| I-60 | 80 | 0 |
| I-65 | 80 | 0 |
| I-75 | 80 | 0 |
| I-77 | 80 | 0 |
| I-81 | 80 | 0 |
| I-86 | 80 | 0 |
| I-85 | 80 | 20 |
| I-88 | 80 | 0 |
| I-89 | 80 | 0 |
| I-90 | 80 | 0 |
| I-107 | 80 | 0 |
| I-108 | 80 | 0 |

TABLE 20c
Post-emergence effect at 320 g/ha against ZEAMX in %

| Example number | Dosage [g/ha] | ZEAMX |
|---|---|---|
| I-09 | 320 | 0 |
| I-11 | 320 | 0 |
| I-15 | 320 | 20 |
| I-24 | 320 | 20 |
| I-40 | 320 | 0 |
| I-45 | 320 | 0 |
| I-44 | 320 | 0 |
| I-42 | 320 | 20 |
| I-51 | 320 | 0 |
| I-52 | 320 | 0 |
| I-56 | 320 | 20 |
| I-57 | 320 | 0 |
| I-65 | 320 | 0 |
| I-88 | 320 | 0 |
| I-90 | 320 | 0 |
| I-108 | 320 | 0 |

TABLE 21a
Post-emergence effect at 20 g/ha against TRZAS in %

| Example number | Dosage [g/ha] | TRZAS |
|---|---|---|
| I-12 | 20 | 0 |
| I-15 | 20 | 0 |
| I-16 | 20 | 10 |
| I-17 | 20 | 10 |
| I-19 | 20 | 20 |
| I-24 | 20 | 0 |
| I-25 | 20 | 10 |
| I-22 | 20 | 20 |
| I-28 | 20 | 20 |
| I-33 | 20 | 20 |
| I-32 | 20 | 10 |
| I-36 | 20 | 20 |
| I-35 | 20 | 20 |
| I-38 | 20 | 0 |
| I-39 | 20 | 0 |
| I-47 | 20 | 0 |
| I-46 | 20 | 0 |
| I-45 | 20 | 0 |
| I-44 | 20 | 0 |
| I-43 | 20 | 0 |
| I-42 | 20 | 0 |
| I-54 | 20 | 0 |
| I-51 | 20 | 0 |
| I-52 | 20 | 0 |
| I-53 | 20 | 0 |
| I-59 | 20 | 0 |
| I-55 | 20 | 20 |
| I-56 | 20 | 0 |
| I-57 | 20 | 0 |
| I-69 | 20 | 0 |
| I-60 | 20 | 0 |
| I-71 | 20 | 20 |
| I-64 | 20 | 10 |
| I-65 | 20 | 0 |
| I-78 | 20 | 0 |
| I-81 | 20 | 0 |
| I-85 | 20 | 0 |
| I-88 | 20 | 20 |
| I-89 | 20 | 0 |
| I-107 | 20 | 20 |
| I-114 | 20 | 0 |
| I-115 | 20 | 0 |
| I-92 | 20 | 0 |
| I-93 | 20 | 0 |
| I-94 | 20 | 0 |
| I-95 | 20 | 0 |

TABLE 21b
Post-emergence effect at 80 g/ha against TRZAS in %

| Example number | Dosage [g/ha] | TRZAS |
|---|---|---|
| I-02 | 80 | 10 |
| I-09 | 80 | 0 |
| I-11 | 80 | 0 |
| I-12 | 80 | 10 |
| I-15 | 80 | 10 |
| I-24 | 80 | 0 |
| I-45 | 80 | 0 |
| I-44 | 80 | 0 |
| I-43 | 80 | 0 |
| I-42 | 80 | 0 |
| I-51 | 80 | 0 |
| I-52 | 80 | 0 |
| I-57 | 80 | 20 |
| I-69 | 80 | 10 |
| I-60 | 80 | 0 |
| I-65 | 80 | 0 |
| I-114 | 80 | 20 |
| I-115 | 80 | 10 |
| I-92 | 80 | 10 |
| I-93 | 80 | 10 |
| I-94 | 80 | 10 |
| I-95 | 80 | 0 |

TABLE 21c
Post-emergence effect at 320 g/ha against TRZAS in %

| Example number | Dosage [g/ha] | TRZAS |
|---|---|---|
| I-02 | 320 | 10 |
| I-09 | 320 | 0 |
| I-11 | 320 | 0 |
| I-12 | 320 | 20 |
| I-15 | 320 | 20 |
| I-24 | 320 | 0 |
| I-45 | 320 | 0 |
| I-44 | 320 | 0 |
| I-43 | 320 | 0 |
| I-51 | 320 | 0 |
| I-52 | 320 | 0 |
| I-65 | 320 | 0 |
| I-115 | 320 | 20 |
| I-92 | 320 | 20 |
| I-93 | 320 | 20 |
| I-94 | 320 | 20 |
| I-95 | 320 | 0 |

TABLE 22a
Post-emergence effect at 20 g/ha against ORYSA in %

| Example number | Dosage [g/ha] | ORYSA |
|---|---|---|
| I-13 | 20 | 10 |
| I-12 | 20 | 20 |
| I-10 | 20 | 10 |
| I-15 | 20 | 0 |
| I-16 | 20 | 0 |
| I-17 | 20 | 0 |
| I-19 | 20 | 0 |
| I-20 | 20 | 0 |
| I-24 | 20 | 0 |
| I-25 | 20 | 10 |
| I-22 | 20 | 10 |
| I-23 | 20 | 20 |
| I-26 | 20 | 20 |
| I-28 | 20 | 0 |
| I-29 | 20 | 0 |
| I-30 | 20 | 10 |
| I-33 | 20 | 0 |
| I-32 | 20 | 10 |
| I-36 | 20 | 0 |
| I-35 | 20 | 0 |
| I-37 | 20 | 0 |
| I-38 | 20 | 0 |
| I-39 | 20 | 0 |

TABLE 22b
Post-emergence effect at 80 g/ha against ORYSA in %

| Example number | Dosage [g/ha] | ORYSA |
|---|---|---|
| I-02 | 80 | 0 |
| I-03 | 80 | 0 |
| I-08 | 80 | 10 |
| I-09 | 80 | 10 |
| I-11 | 80 | 0 |
| I-12 | 80 | 20 |
| I-15 | 80 | 10 |
| I-16 | 80 | 10 |
| I-17 | 80 | 20 |
| I-19 | 80 | 0 |
| I-24 | 80 | 0 |
| I-25 | 80 | 10 |
| I-28 | 80 | 10 |
| I-29 | 80 | 0 |
| I-33 | 80 | 20 |
| I-32 | 80 | 20 |
| I-36 | 80 | 10 |
| I-35 | 80 | 0 |
| I-37 | 80 | 0 |
| I-39 | 80 | 20 |

TABLE 22c
Post-emergence effect at 320 g/ha against ORYSA in %

| Example number | Dosage [g/ha] | ORYSA |
|---|---|---|
| I-02 | 320 | 10 |
| I-03 | 320 | 10 |
| I-12 | 320 | 20 |
| I-15 | 320 | 10 |
| I-19 | 320 | 20 |
| I-24 | 320 | 10 |
| I-28 | 320 | 20 |
| I-36 | 320 | 20 |
| I-35 | 320 | 10 |

TABLE 23a
Post-emergence effect at 20 g/ha against GLXMA in %

| Example number | Dosage [g/ha] | GLXMA |
|---|---|---|
| I-12 | 20 | 0 |
| I-15 | 20 | 20 |
| I-24 | 20 | 10 |
| I-36 | 20 | 10 |
| I-35 | 20 | 20 |
| I-45 | 20 | 0 |
| I-44 | 20 | 0 |
| I-43 | 20 | 0 |
| I-51 | 20 | 0 |
| I-52 | 20 | 0 |
| I-60 | 20 | 10 |
| I-65 | 20 | 0 |
| I-92 | 20 | 10 |
| I-93 | 20 | 20 |

TABLE 23b
Post-emergence effect at 80 g/ha against GLXMA in %

| Example number | Dosage [g/ha] | GLXMA |
|---|---|---|
| I-02 | 80 | 20 |
| I-09 | 80 | 10 |
| I-11 | 80 | 20 |
| I-12 | 80 | 20 |
| I-24 | 80 | 10 |
| I-45 | 80 | 0 |
| I-44 | 80 | 0 |
| I-51 | 80 | 0 |
| I-52 | 80 | 0 |
| I-65 | 80 | 0 |

TABLE 23c
Post-emergence effect at 320 g/ha against GLXMA in %

| Example number | Dosage [g/ha] | GLXMA |
|---|---|---|
| I-02 | 320 | 20 |
| I-09 | 320 | 20 |
| I-44 | 320 | 0 |
| I-51 | 320 | 0 |
| I-52 | 320 | 0 |
| I-65 | 320 | 0 |

TABLE 24a
Post-emergence effect at 20 g/ha against BRSNW in %

| Example number | Dosage [g/ha] | BRSNW |
|---|---|---|
| I-12 | 20 | 0 |
| I-24 | 20 | 10 |
| I-25 | 20 | 20 |
| I-45 | 20 | 0 |
| I-44 | 20 | 0 |
| I-43 | 20 | 0 |
| I-51 | 20 | 0 |
| I-52 | 20 | 0 |
| I-65 | 20 | 0 |

TABLE 24b
Post-emergence effect at 80 g/ha against BRSNW in %

| Example number | Dosage [g/ha] | BRSNW |
|---|---|---|
| I-02 | 80 | 20 |
| I-12 | 80 | 20 |
| I-24 | 80 | 10 |
| I-45 | 80 | 0 |
| I-44 | 80 | 0 |
| I-51 | 80 | 0 |
| I-52 | 80 | 0 |
| I-65 | 80 | 0 |

TABLE 24c
Post-emergence effect at 320 g/ha against BRSNW in %

| Example number | Dosage [g/ha] | BRSNW |
|---|---|---|
| I-45 | 320 | 0 |
| I-51 | 320 | 0 |
| I-65 | 320 | 0 |

TABLE 25a
Post-emergence effect at 20 g/ha against ABUTH in %

| Example number | Dosage [g/ha] | ABUTH |
|---|---|---|
| I-10 | 20 | 80 |
| I-17 | 20 | 80 |
| I-37 | 20 | 80 |
| I-73 | 20 | 80 |
| I-82 | 20 | 80 |
| I-75 | 20 | 80 |
| I-74 | 20 | 80 |
| I-78 | 20 | 80 |
| I-79 | 20 | 80 |
| I-87 | 20 | 90 |
| I-83 | 20 | 80 |
| I-109 | 20 | 80 |
| I-110 | 20 | 80 |
| I-112 | 20 | 80 |
| I-113 | 20 | 90 |

TABLE 25b
Post-emergence effect at 80 g/ha against ABUTH in %

| Example number | Dosage [g/ha] | ABUTH |
|---|---|---|
| I-01 | 80 | 80 |
| I-10 | 80 | 90 |
| I-16 | 80 | 90 |
| I-17 | 80 | 90 |
| I-19 | 80 | 90 |
| I-20 | 80 | 80 |
| I-28 | 80 | 90 |
| I-29 | 80 | 80 |
| I-30 | 80 | 80 |
| I-33 | 80 | 90 |
| I-32 | 80 | 80 |
| I-37 | 80 | 90 |
| I-40 | 80 | 90 |
| I-41 | 80 | 90 |
| I-14 | 80 | 90 |
| I-48 | 80 | 80 |
| I-47 | 80 | 80 |
| I-50 | 80 | 80 |
| I-59 | 80 | 90 |
| I-55 | 80 | 80 |
| I-58 | 80 | 80 |
| I-70 | 80 | 80 |
| I-71 | 80 | 90 |
| I-63 | 80 | 90 |
| I-61 | 80 | 80 |
| I-62 | 80 | 80 |
| I-66 | 80 | 80 |
| I-73 | 80 | 80 |
| I-68 | 80 | 80 |
| I-72 | 80 | 80 |
| I-82 | 80 | 90 |
| I-75 | 80 | 80 |
| I-74 | 80 | 80 |
| I-77 | 80 | 80 |
| I-78 | 80 | 80 |
| I-79 | 80 | 80 |
| I-80 | 80 | 80 |
| I-86 | 80 | 80 |
| I-87 | 80 | 90 |
| I-83 | 80 | 90 |
| I-84 | 80 | 80 |
| I-85 | 80 | 80 |
| I-108 | 80 | 80 |
| I-109 | 80 | 80 |
| I-110 | 80 | 90 |
| I-111 | 80 | 80 |
| I-112 | 80 | 80 |
| I-91 | 80 | 80 |
| I-113 | 80 | 90 |
| I-114 | 80 | 80 |
| I-93 | 80 | 80 |
| I-94 | 80 | 80 |
| I-95 | 80 | 80 |

TABLE 25c
Post-emergence effect at 320 g/ha against ABUTH in %

| Example number | Dosage [g/ha] | ABUTH |
|---|---|---|
| I-01 | 320 | 90 |
| I-04 | 320 | 80 |
| I-13 | 320 | 80 |
| I-10 | 320 | 90 |
| I-16 | 320 | 90 |
| I-17 | 320 | 90 |
| I-19 | 320 | 90 |
| I-20 | 320 | 90 |
| I-25 | 320 | 80 |
| I-26 | 320 | 90 |
| I-28 | 320 | 100 |
| I-29 | 320 | 90 |
| I-30 | 320 | 90 |
| I-33 | 320 | 90 |
| I-32 | 320 | 90 |
| I-35 | 320 | 80 |
| I-37 | 320 | 90 |
| I-38 | 320 | 80 |
| I-39 | 320 | 90 |
| I-40 | 320 | 90 |
| I-41 | 320 | 90 |
| I-14 | 320 | 90 |
| I-48 | 320 | 90 |
| I-47 | 320 | 90 |
| I-46 | 320 | 80 |
| I-42 | 320 | 80 |
| I-54 | 320 | 80 |
| I-50 | 320 | 90 |
| I-53 | 320 | 80 |
| I-59 | 320 | 90 |
| I-55 | 320 | 90 |
| I-58 | 320 | 80 |
| I-56 | 320 | 80 |
| I-69 | 320 | 90 |
| I-70 | 320 | 90 |
| I-71 | 320 | 90 |
| I-63 | 320 | 90 |
| I-64 | 320 | 80 |
| I-61 | 320 | 80 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 80 |
| I-78 | 320 | 80 |
| I-79 | 320 | 90 |
| I-80 | 320 | 80 |
| I-81 | 320 | 90 |
| I-86 | 320 | 80 |
| I-87 | 320 | 90 |
| I-83 | 320 | 90 |
| I-84 | 320 | 80 |
| I-85 | 320 | 90 |
| I-88 | 320 | 80 |
| I-90 | 320 | 90 |
| I-107 | 320 | 80 |
| I-108 | 320 | 90 |
| I-109 | 320 | 90 |
| I-110 | 320 | 90 |
| I-111 | 320 | 90 |
| I-112 | 320 | 80 |
| I-91 | 320 | 80 |
| I-113 | 320 | 90 |
| I-114 | 320 | 90 |
| I-115 | 320 | 80 |
| I-93 | 320 | 90 |
| I-94 | 320 | 90 |
| I-95 | 320 | 90 |

TABLE 26a
Post-emergence effect at 20 g/ha against ALOMY in %

| Example number | Dosage [g/ha] | ALOMY |
|---|---|---|
| I-112 | 20 | 80 |

TABLE 26b
Post-emergence effect at 80 g/ha against ALOMY in %

| Example number | Dosage [g/ha] | ALOMY |
|---|---|---|
| I-01 | 80 | 90 |
| I-04 | 80 | 90 |
| I-05 | 80 | 90 |
| I-06 | 80 | 90 |
| I-07 | 80 | 90 |
| I-13 | 80 | 80 |
| I-23 | 80 | 90 |
| I-26 | 80 | 90 |
| I-37 | 80 | 100 |
| I-70 | 80 | 80 |
| I-73 | 80 | 80 |
| I-68 | 80 | 90 |
| I-79 | 80 | 80 |
| I-86 | 80 | 90 |
| I-87 | 80 | 90 |
| I-83 | 80 | 90 |
| I-88 | 80 | 90 |
| I-108 | 80 | 90 |
| I-110 | 80 | 80 |
| I-111 | 80 | 80 |
| I-112 | 80 | 90 |
| I-113 | 80 | 100 |

TABLE 26c
Post-emergence effect at 320 g/ha against ALOMY in %

| Example number | Dosage [g/ha] | ALOMY |
|---|---|---|
| I-01 | 320 | 100 |
| I-04 | 320 | 90 |
| I-05 | 320 | 90 |
| I-06 | 320 | 90 |
| I-07 | 320 | 90 |
| I-08 | 320 | 90 |
| I-13 | 320 | 90 |
| I-10 | 320 | 90 |
| I-16 | 320 | 90 |
| I-17 | 320 | 90 |
| I-19 | 320 | 80 |
| I-20 | 320 | 80 |
| I-22 | 320 | 90 |
| I-23 | 320 | 90 |
| I-26 | 320 | 90 |
| I-28 | 320 | 90 |
| I-29 | 320 | 90 |
| I-30 | 320 | 90 |
| I-33 | 320 | 90 |
| I-35 | 320 | 90 |
| I-37 | 320 | 100 |
| I-38 | 320 | 100 |
| I-39 | 320 | 100 |
| I-40 | 320 | 90 |
| I-41 | 320 | 90 |
| I-14 | 320 | 90 |
| I-48 | 320 | 90 |
| I-46 | 320 | 90 |
| I-42 | 320 | 90 |
| I-54 | 320 | 100 |
| I-50 | 320 | 80 |
| I-53 | 320 | 100 |
| I-59 | 320 | 90 |
| I-55 | 320 | 90 |
| I-58 | 320 | 90 |
| I-70 | 320 | 90 |
| I-71 | 320 | 90 |
| I-63 | 320 | 90 |
| I-64 | 320 | 90 |
| I-61 | 320 | 90 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 80 |
| I-74 | 320 | 90 |
| I-77 | 320 | 90 |
| I-78 | 320 | 90 |
| I-79 | 320 | 90 |
| I-80 | 320 | 90 |
| I-81 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-89 | 320 | 90 |
| I-90 | 320 | 90 |
| I-107 | 320 | 100 |
| I-108 | 320 | 100 |
| I-109 | 320 | 90 |
| I-110 | 320 | 100 |
| I-111 | 320 | 90 |
| I-112 | 320 | 90 |
| I-91 | 320 | 80 |
| I-113 | 320 | 100 |

TABLE 27a
Post-emergence effect at 20 g/ha against AMARE in %

| Example number | Dosage [g/ha] | AMARE |
|---|---|---|
| I-10 | 20 | 80 |
| I-17 | 20 | 80 |
| I-26 | 20 | 80 |
| I-73 | 20 | 80 |
| I-72 | 20 | 80 |
| I-74 | 20 | 80 |
| I-79 | 20 | 80 |
| I-83 | 20 | 90 |
| I-113 | 20 | 80 |
| I-114 | 20 | 80 |

TABLE 27b
Post-emergence effect at 80 g/ha against AMARE in %

| Example number | Dosage [g/ha] | AMARE |
|---|---|---|
| I-01 | 80 | 80 |
| I-10 | 80 | 80 |
| I-16 | 80 | 80 |
| I-17 | 80 | 80 |
| I-19 | 80 | 80 |
| I-20 | 80 | 80 |
| I-25 | 80 | 80 |
| I-23 | 80 | 80 |
| I-26 | 80 | 80 |
| I-29 | 80 | 90 |
| I-30 | 80 | 80 |
| I-37 | 80 | 90 |
| I-38 | 80 | 90 |
| I-46 | 80 | 80 |
| I-70 | 80 | 80 |
| I-66 | 80 | 80 |
| I-73 | 80 | 90 |
| I-72 | 80 | 80 |
| I-82 | 80 | 90 |
| I-74 | 80 | 80 |
| I-78 | 80 | 90 |
| I-79 | 80 | 80 |
| I-86 | 80 | 80 |
| I-87 | 80 | 80 |
| I-83 | 80 | 90 |
| I-84 | 80 | 80 |
| I-111 | 80 | 80 |
| I-113 | 80 | 80 |
| I-114 | 80 | 80 |
| I-94 | 80 | 80 |
| I-95 | 80 | 80 |

TABLE 27c
Post-emergence effect at 320 g/ha against AMARE in %

| Example number | Dosage [g/ha] | AMARE |
|---|---|---|
| I-01 | 320 | 90 |
| I-04 | 320 | 90 |
| I-05 | 320 | 80 |
| I-13 | 320 | 80 |
| I-10 | 320 | 100 |
| I-16 | 320 | 80 |
| I-17 | 320 | 80 |
| I-19 | 320 | 80 |
| I-20 | 320 | 90 |
| I-25 | 320 | 90 |
| I-22 | 320 | 90 |
| I-23 | 320 | 90 |
| I-26 | 320 | 80 |
| I-28 | 320 | 80 |
| I-29 | 320 | 90 |
| I-30 | 320 | 80 |
| I-32 | 320 | 80 |
| I-36 | 320 | 80 |
| I-35 | 320 | 80 |
| I-37 | 320 | 90 |
| I-38 | 320 | 90 |
| I-39 | 320 | 80 |
| I-40 | 320 | 80 |
| I-41 | 320 | 90 |
| I-14 | 320 | 80 |
| I-48 | 320 | 80 |
| I-47 | 320 | 80 |
| I-46 | 320 | 80 |
| I-42 | 320 | 80 |
| I-54 | 320 | 80 |
| I-50 | 320 | 80 |
| I-53 | 320 | 80 |
| I-70 | 320 | 80 |
| I-71 | 320 | 80 |
| I-66 | 320 | 80 |
| I-73 | 320 | 90 |
| I-68 | 320 | 80 |
| I-72 | 320 | 80 |
| I-82 | 320 | 90 |
| I-75 | 320 | 90 |
| I-74 | 320 | 80 |
| I-78 | 320 | 90 |
| I-79 | 320 | 80 |
| I-80 | 320 | 80 |
| I-81 | 320 | 80 |
| I-86 | 320 | 90 |
| I-87 | 320 | 90 |
| I-83 | 320 | 90 |
| I-84 | 320 | 80 |
| I-85 | 320 | 80 |
| I-88 | 320 | 80 |
| I-90 | 320 | 80 |
| I-107 | 320 | 80 |
| I-110 | 320 | 80 |
| I-111 | 320 | 80 |
| I-113 | 320 | 80 |
| I-114 | 320 | 90 |
| I-93 | 320 | 90 |
| I-94 | 320 | 90 |
| I-95 | 320 | 90 |

TABLE 28a
Post-emergence effect at 20 g/ha against AVEFA in %

| Example number | Dosage [g/ha] | AVEFA |
|---|---|---|
| I-58 | 20 | 80 |
| I-86 | 20 | 80 |
| I-112 | 20 | 90 |
| I-113 | 20 | 80 |

TABLE 28b
Post-emergence effect at 80 g/ha against AVEFA in %

| Example number | Dosage [g/ha] | AVEFA |
|---|---|---|
| I-04 | 80 | 80 |
| I-05 | 80 | 80 |
| I-23 | 80 | 80 |
| I-26 | 80 | 80 |
| I-28 | 80 | 80 |
| I-29 | 80 | 90 |
| I-30 | 80 | 90 |
| I-37 | 80 | 80 |
| I-40 | 80 | 80 |
| I-41 | 80 | 80 |
| I-14 | 80 | 90 |
| I-48 | 80 | 90 |
| I-55 | 80 | 90 |
| I-58 | 80 | 80 |
| I-70 | 80 | 90 |
| I-63 | 80 | 80 |
| I-61 | 80 | 80 |
| I-62 | 80 | 90 |
| I-73 | 80 | 90 |
| I-68 | 80 | 90 |
| I-72 | 80 | 90 |
| I-82 | 80 | 90 |
| I-74 | 80 | 90 |
| I-77 | 80 | 90 |
| I-78 | 80 | 90 |
| I-79 | 80 | 90 |
| I-86 | 80 | 90 |
| I-87 | 80 | 90 |
| I-83 | 80 | 90 |
| I-84 | 80 | 90 |
| I-85 | 80 | 90 |
| I-88 | 80 | 90 |
| I-108 | 80 | 90 |
| I-109 | 80 | 80 |
| I-110 | 80 | 90 |
| I-111 | 80 | 90 |
| I-112 | 80 | 90 |
| I-91 | 80 | 90 |
| I-113 | 80 | 100 |

TABLE 28c
Post-emergence effect at 320 g/ha against AVEFA in %

| Example number | Dosage [g/ha] | AVEFA |
|---|---|---|
| I-01 | 320 | 90 |
| I-04 | 320 | 90 |
| I-05 | 320 | 80 |
| I-06 | 320 | 80 |
| I-13 | 320 | 80 |
| I-10 | 320 | 80 |
| I-16 | 320 | 90 |
| I-17 | 320 | 90 |
| I-19 | 320 | 80 |
| I-20 | 320 | 90 |
| I-22 | 320 | 80 |
| I-23 | 320 | 80 |
| I-26 | 320 | 90 |
| I-28 | 320 | 90 |
| I-29 | 320 | 90 |
| I-30 | 320 | 90 |
| I-33 | 320 | 90 |
| I-32 | 320 | 90 |
| I-35 | 320 | 80 |
| I-37 | 320 | 90 |
| I-38 | 320 | 90 |
| I-39 | 320 | 100 |
| I-40 | 320 | 90 |
| I-41 | 320 | 90 |
| I-14 | 320 | 90 |
| I-48 | 320 | 90 |
| I-47 | 320 | 80 |
| I-54 | 320 | 90 |
| I-50 | 320 | 90 |
| I-53 | 320 | 90 |
| I-59 | 320 | 80 |
| I-55 | 320 | 90 |
| I-58 | 320 | 90 |
| I-56 | 320 | 80 |
| I-57 | 320 | 80 |
| I-60 | 320 | 80 |
| I-70 | 320 | 90 |
| I-71 | 320 | 90 |
| I-63 | 320 | 90 |
| I-64 | 320 | 90 |
| I-61 | 320 | 90 |
| I-62 | 320 | 90 |
| I-66 | 320 | 80 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-81 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-89 | 320 | 80 |
| I-90 | 320 | 90 |
| I-107 | 320 | 90 |
| I-108 | 320 | 90 |
| I-109 | 320 | 90 |
| I-110 | 320 | 90 |
| I-111 | 320 | 90 |
| I-112 | 320 | 90 |
| I-91 | 320 | 90 |
| I-113 | 320 | 100 |
| I-114 | 320 | 80 |
| I-115 | 320 | 80 |
| I-94 | 320 | 80 |

TABLE 29a
Post-emergence effect at 80 g/ha against DIGSA in %

| Example number | Dosage [g/ha] | DIGSA |
|---|---|---|
| I-05 | 80 | 90 |
| I-07 | 80 | 80 |
| I-13 | 80 | 90 |
| I-16 | 80 | 80 |
| I-17 | 80 | 80 |
| I-23 | 80 | 90 |
| I-26 | 80 | 80 |
| I-30 | 80 | 90 |
| I-41 | 80 | 80 |
| I-14 | 80 | 80 |
| I-48 | 80 | 80 |
| I-59 | 80 | 80 |
| I-55 | 80 | 90 |
| I-70 | 80 | 80 |
| I-63 | 80 | 90 |
| I-61 | 80 | 80 |
| I-62 | 80 | 90 |
| I-72 | 80 | 90 |
| I-82 | 80 | 90 |
| I-75 | 80 | 80 |
| I-74 | 80 | 80 |
| I-80 | 80 | 80 |
| I-87 | 80 | 80 |
| I-84 | 80 | 80 |

TABLE 29b
Post-emergence effect at 320 g/ha against DIGSA in %

| Example number | Dosage [g/ha] | DIGSA |
|---|---|---|
| I-05 | 320 | 90 |
| I-07 | 320 | 90 |
| I-13 | 320 | 90 |
| I-10 | 320 | 90 |
| I-16 | 320 | 90 |
| I-17 | 320 | 90 |
| I-19 | 320 | 90 |
| I-20 | 320 | 90 |
| I-25 | 320 | 90 |
| I-22 | 320 | 80 |
| I-23 | 320 | 90 |
| I-26 | 320 | 90 |
| I-28 | 320 | 80 |
| I-29 | 320 | 80 |
| I-30 | 320 | 90 |
| I-33 | 320 | 80 |
| I-32 | 320 | 90 |
| I-35 | 320 | 80 |
| I-37 | 320 | 90 |
| I-38 | 320 | 90 |
| I-40 | 320 | 90 |
| I-41 | 320 | 90 |
| I-14 | 320 | 90 |
| I-48 | 320 | 90 |
| I-47 | 320 | 80 |
| I-46 | 320 | 80 |
| I-42 | 320 | 90 |
| I-54 | 320 | 90 |
| I-50 | 320 | 90 |
| I-53 | 320 | 90 |
| I-59 | 320 | 90 |
| I-55 | 320 | 90 |
| I-58 | 320 | 80 |
| I-56 | 320 | 80 |
| I-70 | 320 | 90 |
| I-71 | 320 | 90 |
| I-63 | 320 | 90 |
| I-64 | 320 | 80 |
| I-61 | 320 | 90 |
| I-62 | 320 | 90 |
| I-66 | 320 | 90 |
| I-73 | 320 | 80 |
| I-68 | 320 | 90 |
| I-72 | 320 | 90 |
| I-82 | 320 | 90 |
| I-75 | 320 | 80 |
| I-74 | 320 | 90 |
| I-77 | 320 | 90 |
| I-78 | 320 | 80 |
| I-79 | 320 | 80 |
| I-80 | 320 | 90 |
| I-81 | 320 | 80 |
| I-86 | 320 | 80 |
| I-87 | 320 | 90 |
| I-83 | 320 | 90 |
| I-84 | 320 | 90 |
| I-85 | 320 | 90 |
| I-88 | 320 | 80 |
| I-90 | 320 | 80 |
| I-107 | 320 | 80 |
| I-108 | 320 | 90 |
| I-111 | 320 | 80 |
| I-112 | 320 | 80 |
| I-91 | 320 | 90 |
| I-113 | 320 | 90 |
| I-114 | 320 | 80 |
| I-94 | 320 | 80 |

TABLE 30a
Post-emergence effect at 20 g/ha against ECHCG in %

| Example number | Dosage [g/ha] | ECHCG |
|---|---|---|
| I-13 | 20 | 80 |
| I-10 | 20 | 80 |
| I-23 | 20 | 80 |
| I-30 | 20 | 80 |
| I-54 | 20 | 80 |
| I-50 | 20 | 90 |
| I-71 | 20 | 90 |
| I-63 | 20 | 90 |
| I-73 | 20 | 90 |
| I-68 | 20 | 80 |
| I-72 | 20 | 90 |
| I-82 | 20 | 80 |
| I-74 | 20 | 90 |
| I-78 | 20 | 90 |
| I-79 | 20 | 80 |
| I-80 | 20 | 90 |
| I-86 | 20 | 80 |
| I-87 | 20 | 90 |
| I-83 | 20 | 90 |
| I-84 | 20 | 90 |
| I-85 | 20 | 90 |
| I-88 | 20 | 80 |
| I-109 | 20 | 80 |
| I-110 | 20 | 90 |
| I-111 | 20 | 80 |
| I-112 | 20 | 90 |
| I-91 | 20 | 80 |
| I-113 | 20 | 90 |
| I-114 | 20 | 80 |
| I-94 | 20 | 80 |
| I-95 | 20 | 80 |

TABLE 30b
Post-emergence effect at 80 g/ha against ECHCG in %

| Example number | Dosage [g/ha] | ECHCG |
|---|---|---|
| I-01 | 80 | 90 |
| I-04 | 80 | 80 |
| I-06 | 80 | 80 |
| I-07 | 80 | 80 |
| I-13 | 80 | 90 |
| I-10 | 80 | 90 |
| I-16 | 80 | 90 |
| I-17 | 80 | 80 |
| I-19 | 80 | 80 |
| I-20 | 80 | 80 |
| I-23 | 80 | 80 |
| I-26 | 80 | 80 |
| I-28 | 80 | 90 |
| I-29 | 80 | 80 |
| I-30 | 80 | 90 |
| I-33 | 80 | 80 |
| I-32 | 80 | 90 |
| I-35 | 80 | 80 |
| I-37 | 80 | 100 |
| I-40 | 80 | 90 |
| I-14 | 80 | 90 |
| I-48 | 80 | 90 |
| I-47 | 80 | 90 |
| I-46 | 80 | 80 |
| I-54 | 80 | 90 |
| I-50 | 80 | 90 |
| I-53 | 80 | 90 |
| I-59 | 80 | 90 |
| I-55 | 80 | 90 |
| I-58 | 80 | 90 |
| I-56 | 80 | 80 |
| I-70 | 80 | 90 |
| I-71 | 80 | 90 |
| I-63 | 80 | 90 |
| I-64 | 80 | 80 |
| I-61 | 80 | 90 |
| I-62 | 80 | 90 |
| I-66 | 80 | 90 |
| I-73 | 80 | 90 |
| I-68 | 80 | 90 |
| I-72 | 80 | 90 |
| I-82 | 80 | 90 |
| I-75 | 80 | 90 |
| I-74 | 80 | 90 |
| I-77 | 80 | 90 |
| I-78 | 80 | 90 |
| I-79 | 80 | 90 |
| I-80 | 80 | 90 |
| I-86 | 80 | 90 |
| I-87 | 80 | 90 |
| I-83 | 80 | 90 |
| I-84 | 80 | 90 |
| I-85 | 80 | 90 |
| I-88 | 80 | 90 |
| I-90 | 80 | 90 |
| I-108 | 80 | 90 |
| I-109 | 80 | 90 |
| I-110 | 80 | 90 |
| I-111 | 80 | 90 |
| I-112 | 80 | 90 |
| I-91 | 80 | 80 |
| I-113 | 80 | 90 |
| I-114 | 80 | 90 |
| I-92 | 80 | 80 |
| I-94 | 80 | 100 |
| I-95 | 80 | 90 |

TABLE 30c
Post-emergence effect at 320 g/ha against ECHCG in %

| Example number | Dosage [g/ha] | ECHCG |
|---|---|---|
| I-01 | 320 | 90 |
| I-03 | 320 | 80 |
| I-04 | 320 | 80 |
| I-05 | 320 | 80 |
| I-06 | 320 | 80 |
| I-07 | 320 | 80 |
| I-08 | 320 | 80 |
| I-13 | 320 | 90 |
| I-10 | 320 | 100 |
| I-16 | 320 | 90 |
| I-17 | 320 | 90 |
| I-19 | 320 | 90 |
| I-20 | 320 | 90 |
| I-25 | 320 | 80 |
| I-22 | 320 | 90 |
| I-23 | 320 | 90 |
| I-26 | 320 | 90 |
| I-28 | 320 | 90 |
| I-29 | 320 | 90 |
| I-30 | 320 | 90 |
| I-33 | 320 | 90 |
| I-32 | 320 | 90 |
| I-36 | 320 | 90 |
| I-35 | 320 | 90 |
| I-37 | 320 | 100 |
| I-38 | 320 | 100 |
| I-39 | 320 | 90 |
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 90 |
| I-54 | 320 | 90 |
| I-50 | 320 | 90 |
| I-53 | 320 | 90 |
| I-59 | 320 | 90 |
| I-55 | 320 | 90 |
| I-58 | 320 | 90 |
| I-56 | 320 | 90 |
| I-57 | 320 | 90 |
| I-69 | 320 | 90 |
| I-70 | 320 | 90 |
| I-71 | 320 | 90 |
| I-63 | 320 | 90 |
| I-64 | 320 | 90 |
| I-61 | 320 | 90 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-81 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-90 | 320 | 90 |
| I-107 | 320 | 90 |
| I-108 | 320 | 90 |
| I-109 | 320 | 90 |
| I-110 | 320 | 90 |
| I-111 | 320 | 90 |
| I-112 | 320 | 90 |
| I-91 | 320 | 90 |
| I-113 | 320 | 90 |
| I-114 | 320 | 100 |
| I-115 | 320 | 90 |
| I-92 | 320 | 90 |
| I-94 | 320 | 100 |
| I-95 | 320 | 100 |

TABLE 31a
Post-emergence effect at 20 g/ha against LOLRI in %

| Example number | Dosage [g/ha] | LOLRI |
|---|---|---|
| I-86 | 20 | 80 |

TABLE 31b
Post-emergence effect at 80 g/ha against LOLRI in %

| Example number | Dosage [g/ha] | LOLRI |
|---|---|---|
| I-04 | 80 | 80 |
| I-05 | 80 | 80 |
| I-07 | 80 | 80 |
| I-26 | 80 | 90 |
| I-37 | 80 | 90 |
| I-40 | 80 | 90 |
| I-14 | 80 | 90 |
| I-48 | 80 | 90 |
| I-58 | 80 | 90 |
| I-73 | 80 | 90 |
| I-75 | 80 | 80 |
| I-77 | 80 | 90 |
| I-78 | 80 | 90 |
| I-79 | 80 | 90 |
| I-86 | 80 | 90 |
| I-87 | 80 | 90 |
| I-84 | 80 | 90 |
| I-85 | 80 | 80 |
| I-88 | 80 | 90 |
| I-109 | 80 | 90 |
| I-111 | 80 | 90 |
| I-112 | 80 | 90 |
| I-91 | 80 | 90 |
| I-113 | 80 | 90 |

TABLE 31c
Post-emergence effect at 320 g/ha against LOLRI in %

| Example number | Dosage [g/ha] | LOLRI |
|---|---|---|
| I-01 | 320 | 90 |
| I-04 | 320 | 80 |
| I-05 | 320 | 90 |
| I-07 | 320 | 80 |
| I-13 | 320 | 80 |
| I-10 | 320 | 90 |
| I-17 | 320 | 80 |
| I-25 | 320 | 90 |
| I-23 | 320 | 90 |
| I-26 | 320 | 90 |
| I-28 | 320 | 90 |
| I-29 | 320 | 90 |
| I-30 | 320 | 90 |
| I-33 | 320 | 90 |
| I-32 | 320 | 90 |
| I-36 | 320 | 80 |
| I-35 | 320 | 80 |
| I-37 | 320 | 100 |
| I-38 | 320 | 100 |
| I-39 | 320 | 100 |
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 90 |
| I-50 | 320 | 90 |
| I-53 | 320 | 90 |
| I-55 | 320 | 90 |
| I-58 | 320 | 90 |
| I-70 | 320 | 90 |
| I-71 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-89 | 320 | 90 |
| I-90 | 320 | 90 |
| I-107 | 320 | 90 |
| I-108 | 320 | 90 |
| I-109 | 320 | 90 |
| I-110 | 320 | 90 |
| I-111 | 320 | 90 |
| I-112 | 320 | 90 |
| I-91 | 320 | 90 |
| I-113 | 320 | 100 |
| I-114 | 320 | 80 |

TABLE 32a
Post-emergence effect at 20 g/ha against MATIN in %

| Example number | Dosage [g/ha] | MATIN |
|---|---|---|
| I-10 | 20 | 80 |
| I-17 | 20 | 80 |
| I-23 | 20 | 80 |
| I-26 | 20 | 80 |
| I-28 | 20 | 80 |
| I-29 | 20 | 80 |
| I-37 | 20 | 80 |
| I-54 | 20 | 80 |
| I-78 | 20 | 80 |
| I-83 | 20 | 80 |
| I-111 | 20 | 80 |
| I-112 | 20 | 80 |

TABLE 32b
Post-emergence effect at 80 g/ha against MATIN in %

| Example number | Dosage [g/ha] | MATIN |
|---|---|---|
| I-01 | 80 | 80 |
| I-04 | 80 | 80 |
| I-06 | 80 | 80 |
| I-13 | 80 | 80 |
| I-10 | 80 | 80 |
| I-16 | 80 | 80 |
| I-17 | 80 | 80 |
| I-20 | 80 | 80 |
| I-23 | 80 | 80 |
| I-26 | 80 | 80 |
| I-28 | 80 | 90 |
| I-29 | 80 | 90 |
| I-30 | 80 | 80 |
| I-33 | 80 | 80 |
| I-32 | 80 | 90 |
| I-37 | 80 | 90 |
| I-48 | 80 | 80 |
| I-54 | 80 | 90 |
| I-70 | 80 | 80 |
| I-71 | 80 | 80 |
| I-63 | 80 | 80 |
| I-61 | 80 | 80 |
| I-62 | 80 | 80 |
| I-66 | 80 | 80 |
| I-73 | 80 | 90 |
| I-68 | 80 | 80 |
| I-82 | 80 | 80 |
| I-75 | 80 | 80 |
| I-74 | 80 | 80 |
| I-77 | 80 | 80 |
| I-78 | 80 | 90 |
| I-79 | 80 | 80 |
| I-87 | 80 | 80 |
| I-83 | 80 | 90 |
| I-84 | 80 | 80 |
| I-110 | 80 | 80 |
| I-111 | 80 | 80 |
| I-112 | 80 | 80 |
| I-91 | 80 | 80 |
| I-113 | 80 | 80 |
| I-114 | 80 | 80 |
| I-94 | 80 | 80 |
| I-95 | 80 | 80 |

TABLE 32c
Post-emergence effect at 320 g/ha against MATIN in %

| Example number | Dosage [g/ha] | MATIN |
|---|---|---|
| I-01 | 320 | 90 |
| I-04 | 320 | 80 |
| I-05 | 320 | 80 |
| I-06 | 320 | 80 |
| I-07 | 320 | 80 |
| I-08 | 320 | 80 |
| I-13 | 320 | 80 |
| I-10 | 320 | 90 |
| I-16 | 320 | 90 |
| I-17 | 320 | 80 |
| I-19 | 320 | 80 |
| I-20 | 320 | 90 |
| I-25 | 320 | 80 |
| I-22 | 320 | 90 |
| I-23 | 320 | 90 |
| I-26 | 320 | 80 |
| I-28 | 320 | 90 |
| I-29 | 320 | 90 |
| I-30 | 320 | 90 |
| I-33 | 320 | 90 |
| I-32 | 320 | 90 |
| I-37 | 320 | 90 |
| I-40 | 320 | 80 |
| I-41 | 320 | 80 |
| I-14 | 320 | 90 |
| I-48 | 320 | 90 |
| I-54 | 320 | 90 |
| I-50 | 320 | 80 |
| I-53 | 320 | 80 |
| I-59 | 320 | 80 |
| I-69 | 320 | 80 |
| I-70 | 320 | 80 |
| I-71 | 320 | 80 |
| I-63 | 320 | 80 |
| I-64 | 320 | 80 |
| I-61 | 320 | 80 |
| I-62 | 320 | 80 |
| I-66 | 320 | 90 |
| I-73 | 320 | 90 |
| I-68 | 320 | 90 |
| I-82 | 320 | 90 |
| I-75 | 320 | 80 |
| I-74 | 320 | 80 |
| I-77 | 320 | 80 |
| I-78 | 320 | 90 |
| I-79 | 320 | 80 |
| I-86 | 320 | 90 |
| I-87 | 320 | 80 |
| I-83 | 320 | 90 |
| I-84 | 320 | 80 |
| I-109 | 320 | 80 |
| I-110 | 320 | 80 |
| I-111 | 320 | 80 |
| I-112 | 320 | 80 |
| I-91 | 320 | 80 |
| I-113 | 320 | 80 |
| I-114 | 320 | 90 |
| I-94 | 320 | 90 |
| I-95 | 320 | 80 |

TABLE 33a
Post-emergence effect at 20 g/ha against PHBPU in %

| Example number | Dosage [g/ha] | PHBPU |
|---|---|---|
| I-16 | 20 | 80 |
| I-17 | 20 | 90 |
| I-19 | 20 | 80 |
| I-26 | 20 | 80 |
| I-28 | 20 | 80 |
| I-29 | 20 | 80 |
| I-33 | 20 | 80 |
| I-37 | 20 | 90 |
| I-55 | 20 | 80 |
| I-71 | 20 | 80 |
| I-63 | 20 | 80 |
| I-62 | 20 | 90 |
| I-66 | 20 | 80 |
| I-73 | 20 | 80 |
| I-72 | 20 | 80 |
| I-74 | 20 | 80 |
| I-78 | 20 | 80 |
| I-79 | 20 | 90 |
| I-86 | 20 | 80 |
| I-87 | 20 | 90 |
| I-83 | 20 | 90 |
| I-84 | 20 | 80 |
| I-85 | 20 | 80 |
| I-110 | 20 | 90 |
| I-111 | 20 | 80 |
| I-112 | 20 | 90 |
| I-91 | 20 | 80 |
| I-113 | 20 | 90 |
| I-94 | 20 | 80 |

TABLE 33b
Post-emergence effect at 80 g/ha against PHBPU in %

| Example number | Dosage [g/ha] | PHBPU |
|---|---|---|
| I-01 | 80 | 90 |
| I-04 | 80 | 80 |
| I-06 | 80 | 80 |
| I-10 | 80 | 80 |
| I-16 | 80 | 90 |
| I-17 | 80 | 90 |
| I-19 | 80 | 90 |
| I-20 | 80 | 80 |
| I-23 | 80 | 80 |
| I-26 | 80 | 80 |
| I-28 | 80 | 90 |
| I-29 | 80 | 80 |
| I-30 | 80 | 80 |
| I-33 | 80 | 90 |
| I-37 | 80 | 90 |
| I-38 | 80 | 90 |
| I-39 | 80 | 90 |
| I-40 | 80 | 90 |
| I-41 | 80 | 90 |
| I-14 | 80 | 80 |
| I-48 | 80 | 90 |
| I-47 | 80 | 80 |
| I-54 | 80 | 90 |
| I-50 | 80 | 90 |
| I-53 | 80 | 90 |
| I-59 | 80 | 90 |
| I-55 | 80 | 80 |
| I-58 | 80 | 80 |
| I-70 | 80 | 90 |
| I-71 | 80 | 90 |
| I-63 | 80 | 90 |
| I-64 | 80 | 80 |
| I-61 | 80 | 90 |
| I-62 | 80 | 90 |
| I-66 | 80 | 80 |
| I-73 | 80 | 90 |
| I-68 | 80 | 90 |
| I-72 | 80 | 90 |
| I-82 | 80 | 80 |
| I-75 | 80 | 80 |
| I-74 | 80 | 90 |
| I-77 | 80 | 90 |
| I-78 | 80 | 90 |
| I-79 | 80 | 90 |
| I-80 | 80 | 90 |
| I-86 | 80 | 90 |
| I-87 | 80 | 90 |
| I-83 | 80 | 90 |
| I-84 | 80 | 90 |
| I-85 | 80 | 90 |
| I-107 | 80 | 80 |
| I-108 | 80 | 80 |
| I-109 | 80 | 90 |
| I-110 | 80 | 90 |
| I-111 | 80 | 90 |
| I-112 | 80 | 90 |
| I-91 | 80 | 80 |
| I-113 | 80 | 90 |
| I-114 | 80 | 80 |
| I-94 | 80 | 90 |
| I-95 | 80 | 90 |

TABLE 33c
Post-emergence effect at 320 g/ha against PHBPU in %

| Example number | Dosage [g/ha] | PHBPU |
|---|---|---|
| I-01 | 320 | 90 |
| I-04 | 320 | 80 |
| I-05 | 320 | 90 |
| I-06 | 320 | 80 |
| I-07 | 320 | 80 |
| I-13 | 320 | 90 |
| I-10 | 320 | 90 |
| I-15 | 320 | 80 |
| I-16 | 320 | 90 |
| I-17 | 320 | 90 |
| I-19 | 320 | 90 |
| I-20 | 320 | 90 |
| I-25 | 320 | 90 |
| I-22 | 320 | 90 |
| I-23 | 320 | 90 |
| I-26 | 320 | 90 |
| I-28 | 320 | 90 |
| I-29 | 320 | 90 |
| I-30 | 320 | 90 |
| I-33 | 320 | 90 |
| I-32 | 320 | 90 |
| I-36 | 320 | 80 |
| I-37 | 320 | 90 |
| I-38 | 320 | 90 |
| I-39 | 320 | 90 |
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 80 |
| I-54 | 320 | 90 |
| I-50 | 320 | 90 |
| I-53 | 320 | 90 |
| I-59 | 320 | 90 |
| I-55 | 320 | 90 |
| I-58 | 320 | 90 |
| I-56 | 320 | 80 |
| I-57 | 320 | 80 |
| I-69 | 320 | 80 |
| I-60 | 320 | 90 |
| I-70 | 320 | 90 |
| I-71 | 320 | 90 |
| I-63 | 320 | 90 |
| I-64 | 320 | 90 |
| I-61 | 320 | 90 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-81 | 320 | 80 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-90 | 320 | 90 |
| I-107 | 320 | 80 |
| I-108 | 320 | 90 |
| I-109 | 320 | 90 |
| I-110 | 320 | 90 |
| I-111 | 320 | 90 |
| I-112 | 320 | 90 |
| I-91 | 320 | 80 |
| I-113 | 320 | 100 |
| I-114 | 320 | 90 |
| I-115 | 320 | 80 |
| I-92 | 320 | 80 |
| I-93 | 320 | 80 |
| I-94 | 320 | 90 |
| I-95 | 320 | 90 |

TABLE 34a
Post-emergence effect at 20 g/ha against POLCO in %

| Example number | Dosage [g/ha] | POLCO |
|---|---|---|
| I-10 | 20 | 80 |
| I-48 | 20 | 80 |
| I-70 | 20 | 80 |
| I-73 | 20 | 80 |
| I-72 | 20 | 80 |
| I-82 | 20 | 80 |
| I-86 | 20 | 80 |
| I-87 | 20 | 80 |
| I-85 | 20 | 80 |
| I-88 | 20 | 80 |
| I-110 | 20 | 80 |

TABLE 34b
Post-emergence effect at 80 g/ha against POLCO in %

| Example number | Dosage [g/ha] | POLCO |
|---|---|---|
| I-01 | 80 | 80 |
| I-07 | 80 | 80 |
| I-13 | 80 | 80 |
| I-10 | 80 | 80 |
| I-17 | 80 | 80 |
| I-19 | 80 | 80 |
| I-30 | 80 | 80 |
| I-37 | 80 | 90 |
| I-38 | 80 | 90 |
| I-39 | 80 | 90 |
| I-40 | 80 | 90 |
| I-41 | 80 | 90 |
| I-14 | 80 | 80 |
| I-48 | 80 | 80 |
| I-47 | 80 | 80 |
| I-54 | 80 | 80 |
| I-53 | 80 | 80 |
| I-55 | 80 | 80 |
| I-58 | 80 | 80 |
| I-69 | 80 | 80 |
| I-70 | 80 | 80 |
| I-71 | 80 | 80 |
| I-63 | 80 | 80 |
| I-61 | 80 | 80 |
| I-62 | 80 | 80 |
| I-73 | 80 | 80 |
| I-68 | 80 | 80 |
| I-72 | 80 | 80 |
| I-82 | 80 | 80 |
| I-77 | 80 | 90 |
| I-78 | 80 | 90 |
| I-79 | 80 | 90 |
| I-86 | 80 | 80 |
| I-87 | 80 | 90 |
| I-83 | 80 | 80 |
| I-84 | 80 | 90 |
| I-85 | 80 | 90 |
| I-88 | 80 | 90 |
| I-107 | 80 | 90 |
| I-109 | 80 | 80 |
| I-110 | 80 | 80 |
| I-111 | 80 | 80 |
| I-112 | 80 | 80 |
| I-113 | 80 | 80 |

TABLE 34c
Post-emergence effect at 320 g/ha against POLCO in %

| Example number | Dosage [g/ha] | POLCO |
|---|---|---|
| I-01 | 320 | 80 |
| I-05 | 320 | 80 |
| I-07 | 320 | 90 |
| I-13 | 320 | 80 |
| I-10 | 320 | 80 |
| I-16 | 320 | 80 |
| I-17 | 320 | 80 |
| I-19 | 320 | 80 |
| I-20 | 320 | 80 |
| I-23 | 320 | 80 |
| I-28 | 320 | 80 |
| I-29 | 320 | 80 |
| I-30 | 320 | 80 |
| I-33 | 320 | 80 |
| I-32 | 320 | 80 |
| I-36 | 320 | 80 |
| I-37 | 320 | 90 |
| I-38 | 320 | 90 |
| I-39 | 320 | 90 |
| I-40 | 320 | 90 |
| I-41 | 320 | 90 |
| I-14 | 320 | 80 |
| I-48 | 320 | 80 |
| I-47 | 320 | 80 |
| I-46 | 320 | 80 |
| I-42 | 320 | 80 |
| I-54 | 320 | 80 |
| I-50 | 320 | 80 |
| I-53 | 320 | 80 |
| I-55 | 320 | 90 |
| I-58 | 320 | 80 |
| I-56 | 320 | 80 |
| I-57 | 320 | 80 |
| I-69 | 320 | 80 |
| I-60 | 320 | 90 |
| I-70 | 320 | 80 |
| I-71 | 320 | 80 |
| I-63 | 320 | 90 |
| I-61 | 320 | 80 |
| I-62 | 320 | 80 |
| I-66 | 320 | 80 |
| I-73 | 320 | 90 |
| I-68 | 320 | 80 |
| I-72 | 320 | 90 |
| I-82 | 320 | 90 |
| I-75 | 320 | 80 |
| I-74 | 320 | 90 |
| I-77 | 320 | 90 |
| I-78 | 320 | 90 |
| I-79 | 320 | 90 |
| I-80 | 320 | 90 |
| I-81 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-89 | 320 | 90 |
| I-90 | 320 | 80 |
| I-107 | 320 | 90 |
| I-108 | 320 | 80 |
| I-109 | 320 | 90 |
| I-110 | 320 | 90 |
| I-111 | 320 | 80 |
| I-112 | 320 | 80 |
| I-91 | 320 | 80 |
| I-113 | 320 | 80 |

TABLE 35a
Post-emergence effect at 20 g/ha against SETVI in %

| Example number | Dosage [g/ha] | SETVI |
|---|---|---|
| I-30 | 20 | 80 |
| I-73 | 20 | 80 |
| I-72 | 20 | 90 |
| I-82 | 20 | 90 |
| I-79 | 20 | 90 |
| I-87 | 20 | 90 |
| I-83 | 20 | 90 |
| I-84 | 20 | 80 |
| I-111 | 20 | 80 |
| I-91 | 20 | 80 |
| I-113 | 20 | 90 |

TABLE 35b
Post-emergence effect at 80 g/ha against SETVI in %

| Example number | Dosage [g/ha] | SETVI |
|---|---|---|
| I-01 | 80 | 90 |
| I-04 | 80 | 80 |
| I-05 | 80 | 80 |
| I-06 | 80 | 80 |
| I-13 | 80 | 80 |
| I-10 | 80 | 80 |
| I-16 | 80 | 80 |
| I-17 | 80 | 80 |
| I-20 | 80 | 80 |
| I-23 | 80 | 80 |
| I-28 | 80 | 80 |
| I-29 | 80 | 90 |
| I-30 | 80 | 90 |
| I-33 | 80 | 90 |
| I-37 | 80 | 90 |
| I-48 | 80 | 90 |
| I-54 | 80 | 80 |
| I-50 | 80 | 90 |
| I-59 | 80 | 80 |
| I-70 | 80 | 80 |
| I-71 | 80 | 80 |
| I-63 | 80 | 90 |
| I-61 | 80 | 90 |
| I-62 | 80 | 90 |
| I-73 | 80 | 90 |
| I-68 | 80 | 90 |
| I-72 | 80 | 90 |
| I-82 | 80 | 90 |
| I-75 | 80 | 80 |
| I-74 | 80 | 90 |
| I-78 | 80 | 90 |
| I-79 | 80 | 90 |
| I-80 | 80 | 90 |
| I-86 | 80 | 90 |
| I-87 | 80 | 90 |
| I-83 | 80 | 90 |
| I-84 | 80 | 90 |
| I-85 | 80 | 90 |
| I-110 | 80 | 80 |
| I-111 | 80 | 80 |
| I-112 | 80 | 80 |
| I-91 | 80 | 80 |
| I-113 | 80 | 90 |

TABLE 35c
Post-emergence effect at 320 g/ha against SETVI in %

| Example number | Dosage [g/ha] | SETVI |
|---|---|---|
| I-01 | 320 | 90 |
| I-03 | 320 | 80 |
| I-04 | 320 | 80 |
| I-05 | 320 | 80 |
| I-06 | 320 | 80 |
| I-07 | 320 | 80 |
| I-08 | 320 | 80 |
| I-13 | 320 | 90 |
| I-10 | 320 | 90 |
| I-16 | 320 | 80 |
| I-17 | 320 | 90 |
| I-19 | 320 | 80 |
| I-20 | 320 | 80 |
| I-25 | 320 | 90 |
| I-22 | 320 | 90 |
| I-23 | 320 | 90 |
| I-26 | 320 | 90 |
| I-28 | 320 | 90 |
| I-29 | 320 | 90 |
| I-30 | 320 | 90 |
| I-33 | 320 | 90 |
| I-32 | 320 | 90 |
| I-36 | 320 | 80 |
| I-35 | 320 | 80 |
| I-37 | 320 | 90 |
| I-38 | 320 | 90 |
| I-39 | 320 | 90 |
| I-40 | 320 | 90 |
| I-41 | 320 | 90 |
| I-14 | 320 | 90 |
| I-48 | 320 | 90 |
| I-46 | 320 | 90 |
| I-42 | 320 | 80 |
| I-54 | 320 | 90 |
| I-50 | 320 | 90 |
| I-53 | 320 | 90 |
| I-59 | 320 | 90 |
| I-55 | 320 | 80 |
| I-58 | 320 | 90 |
| I-56 | 320 | 80 |
| I-70 | 320 | 80 |
| I-71 | 320 | 90 |
| I-63 | 320 | 90 |
| I-64 | 320 | 90 |
| I-61 | 320 | 90 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-81 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-90 | 320 | 90 |
| I-107 | 320 | 90 |
| I-108 | 320 | 90 |
| I-109 | 320 | 80 |
| I-110 | 320 | 80 |
| I-111 | 320 | 80 |
| I-112 | 320 | 80 |
| I-91 | 320 | 80 |
| I-113 | 320 | 90 |
| I-114 | 320 | 90 |
| I-115 | 320 | 90 |
| I-92 | 320 | 80 |
| I-94 | 320 | 90 |
| I-95 | 320 | 90 |

TABLE 36a
Post-emergence effect at 20 g/ha against VERPE in %

| Example number | Dosage [g/ha] | VERPE |
|---|---|---|
| I-17 | 20 | 80 |
| I-23 | 20 | 80 |
| I-30 | 20 | 80 |
| I-37 | 20 | 90 |
| I-38 | 20 | 80 |
| I-54 | 20 | 80 |
| I-78 | 20 | 80 |
| I-79 | 20 | 90 |
| I-87 | 20 | 80 |
| I-83 | 20 | 90 |
| I-109 | 20 | 80 |
| I-110 | 20 | 80 |
| I-111 | 20 | 80 |
| I-112 | 20 | 80 |
| I-113 | 20 | 80 |
| I-114 | 20 | 80 |

TABLE 36b
Post-emergence effect at 80 g/ha against VERPE in %

| Example number | Dosage [g/ha] | VERPE |
|---|---|---|
| I-01 | 80 | 80 |
| I-04 | 80 | 90 |
| I-07 | 80 | 80 |
| I-13 | 80 | 80 |
| I-10 | 80 | 90 |
| I-17 | 80 | 80 |
| I-19 | 80 | 80 |
| I-20 | 80 | 80 |
| I-25 | 80 | 80 |
| I-22 | 80 | 90 |
| I-23 | 80 | 90 |
| I-26 | 80 | 90 |
| I-29 | 80 | 90 |
| I-30 | 80 | 80 |
| I-33 | 80 | 80 |
| I-32 | 80 | 90 |
| I-37 | 80 | 100 |
| I-38 | 80 | 80 |
| I-48 | 80 | 80 |
| I-47 | 80 | 80 |
| I-54 | 80 | 80 |
| I-50 | 80 | 90 |
| I-53 | 80 | 80 |
| I-59 | 80 | 80 |
| I-70 | 80 | 90 |
| I-71 | 80 | 90 |
| I-63 | 80 | 80 |
| I-64 | 80 | 80 |
| I-61 | 80 | 80 |
| I-62 | 80 | 80 |
| I-66 | 80 | 80 |
| I-73 | 80 | 90 |
| I-68 | 80 | 90 |
| I-82 | 80 | 90 |
| I-75 | 80 | 80 |
| I-74 | 80 | 90 |
| I-78 | 80 | 90 |
| I-79 | 80 | 90 |
| I-80 | 80 | 80 |
| I-86 | 80 | 80 |
| I-87 | 80 | 90 |
| I-83 | 80 | 90 |
| I-84 | 80 | 90 |
| I-108 | 80 | 80 |
| I-109 | 80 | 80 |
| I-110 | 80 | 80 |
| I-111 | 80 | 80 |
| I-112 | 80 | 80 |
| I-91 | 80 | 80 |
| I-113 | 80 | 90 |
| I-114 | 80 | 80 |
| I-94 | 80 | 80 |
| I-95 | 80 | 80 |

TABLE 36c
Post-emergence effect at 320 g/ha against VERPE in %

| Example number | Dosage [g/ha] | VERPE |
|---|---|---|
| I-01 | 320 | 90 |
| I-03 | 320 | 90 |
| I-04 | 320 | 90 |
| I-05 | 320 | 80 |
| I-07 | 320 | 80 |
| I-08 | 320 | 80 |
| I-13 | 320 | 80 |
| I-10 | 320 | 90 |
| I-16 | 320 | 80 |
| I-17 | 320 | 90 |
| I-19 | 320 | 80 |
| I-20 | 320 | 90 |
| I-25 | 320 | 90 |
| I-22 | 320 | 90 |
| I-23 | 320 | 90 |
| I-26 | 320 | 90 |
| I-28 | 320 | 80 |
| I-29 | 320 | 90 |
| I-30 | 320 | 90 |
| I-33 | 320 | 90 |
| I-32 | 320 | 90 |
| I-37 | 320 | 100 |
| I-38 | 320 | 100 |
| I-39 | 320 | 90 |
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 80 |
| I-54 | 320 | 90 |
| I-50 | 320 | 90 |
| I-53 | 320 | 90 |
| I-59 | 320 | 80 |
| I-55 | 320 | 90 |
| I-58 | 320 | 80 |
| I-56 | 320 | 80 |
| I-69 | 320 | 80 |
| I-60 | 320 | 80 |
| I-70 | 320 | 90 |
| I-71 | 320 | 90 |
| I-63 | 320 | 80 |
| I-64 | 320 | 80 |
| I-61 | 320 | 80 |
| I-62 | 320 | 80 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-90 | 320 | 80 |
| I-107 | 320 | 80 |
| I-108 | 320 | 80 |
| I-109 | 320 | 80 |
| I-110 | 320 | 90 |
| I-111 | 320 | 80 |
| I-112 | 320 | 80 |
| I-91 | 320 | 80 |
| I-113 | 320 | 90 |
| I-114 | 320 | 90 |
| I-115 | 320 | 80 |
| I-94 | 320 | 90 |
| I-95 | 320 | 90 |

TABLE 37a
Post-emergence effect at 20 g/ha against VIOTR in %

| Example number | Dosage [g/ha] | VIOTR |
|---|---|---|
| I-16 | 20 | 80 |
| I-17 | 20 | 90 |
| I-23 | 20 | 80 |
| I-28 | 20 | 80 |
| I-29 | 20 | 80 |
| I-30 | 20 | 80 |
| I-33 | 20 | 80 |
| I-37 | 20 | 80 |
| I-48 | 20 | 80 |
| I-71 | 20 | 90 |
| I-61 | 20 | 80 |
| I-73 | 20 | 90 |
| I-72 | 20 | 90 |
| I-82 | 20 | 80 |
| I-74 | 20 | 80 |
| I-78 | 20 | 80 |
| I-79 | 20 | 90 |
| I-87 | 20 | 90 |
| I-83 | 20 | 90 |
| I-110 | 20 | 80 |
| I-112 | 20 | 80 |
| I-91 | 20 | 80 |
| I-113 | 20 | 90 |

TABLE 37b
Post-emergence effect at 80 g/ha against VIOTR in %

| Example number | Dosage [g/ha] | VIOTR |
|---|---|---|
| I-01 | 80 | 80 |
| I-04 | 80 | 80 |
| I-10 | 80 | 80 |
| I-16 | 80 | 80 |
| I-17 | 80 | 90 |
| I-19 | 80 | 80 |
| I-20 | 80 | 80 |
| I-23 | 80 | 80 |
| I-26 | 80 | 80 |
| I-28 | 80 | 90 |
| I-29 | 80 | 90 |
| I-30 | 80 | 90 |
| I-33 | 80 | 80 |
| I-32 | 80 | 90 |
| I-37 | 80 | 90 |
| I-38 | 80 | 80 |
| I-39 | 80 | 80 |
| I-40 | 80 | 90 |
| I-41 | 80 | 80 |
| I-14 | 80 | 90 |
| I-48 | 80 | 90 |
| I-47 | 80 | 80 |
| I-46 | 80 | 90 |
| I-54 | 80 | 80 |
| I-50 | 80 | 80 |
| I-53 | 80 | 80 |
| I-59 | 80 | 90 |
| I-55 | 80 | 80 |
| I-58 | 80 | 80 |
| I-70 | 80 | 90 |
| I-71 | 80 | 90 |
| I-63 | 80 | 80 |
| I-61 | 80 | 90 |
| I-62 | 80 | 90 |
| I-73 | 80 | 90 |
| I-68 | 80 | 90 |
| I-72 | 80 | 90 |
| I-82 | 80 | 90 |
| I-75 | 80 | 80 |
| I-74 | 80 | 90 |
| I-77 | 80 | 90 |
| I-78 | 80 | 90 |
| I-79 | 80 | 90 |
| I-80 | 80 | 80 |
| I-86 | 80 | 90 |
| I-87 | 80 | 90 |
| I-83 | 80 | 90 |
| I-84 | 80 | 90 |
| I-85 | 80 | 80 |
| I-88 | 80 | 80 |
| I-108 | 80 | 90 |
| I-109 | 80 | 90 |
| I-110 | 80 | 80 |
| I-111 | 80 | 80 |
| I-112 | 80 | 80 |
| I-91 | 80 | 80 |
| I-113 | 80 | 90 |
| I-94 | 80 | 80 |
| I-95 | 80 | 80 |

TABLE 37c
Post-emergence effect at 320 g/ha against VIOTR in %

| Example number | Dosage [g/ha] | VIOTR |
|---|---|---|
| I-01 | 320 | 90 |
| I-04 | 320 | 80 |
| I-05 | 320 | 80 |
| I-06 | 320 | 80 |
| I-13 | 320 | 80 |
| I-10 | 320 | 90 |
| I-15 | 320 | 80 |
| I-16 | 320 | 90 |
| I-17 | 320 | 90 |
| I-19 | 320 | 80 |
| I-20 | 320 | 90 |
| I-25 | 320 | 80 |
| I-22 | 320 | 80 |
| I-23 | 320 | 80 |
| I-26 | 320 | 80 |
| I-28 | 320 | 90 |
| I-29 | 320 | 90 |
| I-30 | 320 | 90 |
| I-33 | 320 | 90 |
| I-32 | 320 | 90 |
| I-37 | 320 | 90 |
| I-38 | 320 | 90 |
| I-39 | 320 | 90 |
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 90 |
| I-54 | 320 | 80 |
| I-50 | 320 | 90 |
| I-51 | 320 | 80 |
| I-53 | 320 | 80 |
| I-59 | 320 | 90 |
| I-55 | 320 | 90 |
| I-58 | 320 | 80 |
| I-56 | 320 | 90 |
| I-57 | 320 | 80 |
| I-69 | 320 | 90 |
| I-70 | 320 | 90 |
| I-71 | 320 | 90 |
| I-63 | 320 | 90 |
| I-61 | 320 | 90 |
| I-62 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-78 | 320 | 90 |
| I-79 | 320 | 90 |
| I-80 | 320 | 90 |
| I-86 | 320 | 90 |
| I-87 | 320 | 90 |
| I-83 | 320 | 90 |
| I-84 | 320 | 90 |
| I-85 | 320 | 90 |
| I-88 | 320 | 90 |
| I-90 | 320 | 90 |
| I-107 | 320 | 90 |
| I-108 | 320 | 90 |
| I-109 | 320 | 90 |
| I-110 | 320 | 80 |
| I-111 | 320 | 80 |
| I-112 | 320 | 80 |
| I-91 | 320 | 80 |
| I-113 | 320 | 90 |
| I-114 | 320 | 90 |
| I-94 | 320 | 90 |
| I-95 | 320 | 90 |

TABLE 38a
Post-emergence effect at 20 g/ha against KCHSC in %

| Example number | Dosage [g/ha] | KCHSC |
|---|---|---|
| I-70 | 20 | 80 |
| I-62 | 20 | 80 |
| I-73 | 20 | 80 |
| I-68 | 20 | 90 |
| I-72 | 20 | 90 |
| I-82 | 20 | 90 |
| I-77 | 20 | 80 |
| I-80 | 20 | 90 |
| I-87 | 20 | 90 |
| I-84 | 20 | 90 |
| I-90 | 20 | 80 |
| I-91 | 20 | 80 |
| I-113 | 20 | 90 |

TABLE 38b
Post-emergence effect at 80 g/ha against KCHSC in %

| Example number | Dosage [g/ha] | KCHSC |
|---|---|---|
| I-40 | 80 | 90 |
| I-41 | 80 | 80 |
| I-14 | 80 | 90 |
| I-48 | 80 | 80 |
| I-47 | 80 | 90 |
| I-46 | 80 | 80 |
| I-55 | 80 | 90 |
| I-58 | 80 | 90 |
| I-70 | 80 | 90 |
| I-71 | 80 | 80 |
| I-61 | 80 | 80 |
| I-62 | 80 | 90 |
| I-66 | 80 | 80 |
| I-73 | 80 | 90 |
| I-68 | 80 | 90 |
| I-72 | 80 | 90 |
| I-82 | 80 | 90 |
| I-77 | 80 | 90 |
| I-80 | 80 | 90 |
| I-86 | 80 | 90 |
| I-87 | 80 | 90 |
| I-84 | 80 | 90 |
| I-85 | 80 | 90 |
| I-88 | 80 | 80 |
| I-90 | 80 | 90 |
| I-108 | 80 | 90 |
| I-91 | 80 | 80 |
| I-113 | 80 | 90 |

TABLE 38c
Post-emergence effect at 320 g/ha against KCHSC in %

| Example number | Dosage [g/ha] | KCHSC |
|---|---|---|
| I-40 | 320 | 90 |
| I-41 | 320 | 90 |
| I-14 | 320 | 90 |
| I-48 | 320 | 90 |
| I-47 | 320 | 90 |
| I-46 | 320 | 90 |
| I-42 | 320 | 90 |
| I-54 | 320 | 80 |
| I-50 | 320 | 80 |
| I-53 | 320 | 80 |
| I-59 | 320 | 80 |
| I-55 | 320 | 90 |
| I-58 | 320 | 90 |
| I-56 | 320 | 90 |
| I-57 | 320 | 90 |
| I-69 | 320 | 80 |
| I-70 | 320 | 90 |
| I-71 | 320 | 90 |
| I-63 | 320 | 90 |
| I-64 | 320 | 90 |
| I-61 | 320 | 90 |
| I-62 | 320 | 90 |
| I-66 | 320 | 90 |
| I-73 | 320 | 90 |
| I-68 | 320 | 90 |
| I-72 | 320 | 90 |
| I-82 | 320 | 90 |
| I-75 | 320 | 90 |
| I-74 | 320 | 90 |
| I-77 | 320 | 90 |
| I-79 | 320 | 80 |
| I-80 | 320 | 90 |
| I-81 | 320 | 90 |
| I-86 | 320 | 90 |
| I-87 | 320 | 90 |
| I-83 | 320 | 80 |
| I-84 | 320 | 90 |
| I-85 | 320 | 90 |
| I-88 | 320 | 80 |
| I-89 | 320 | 90 |
| I-90 | 320 | 90 |
| I-107 | 320 | 90 |
| I-108 | 320 | 90 |
| I-109 | 320 | 80 |
| I-110 | 320 | 80 |
| I-112 | 320 | 80 |
| I-91 | 320 | 90 |
| I-113 | 320 | 90 |

As the results show, inventive compounds of the general formula (I), in post-emergence treatment, have good herbicidal efficacy against harmful plants such as Abutilon theophrasti, Alopecurus myosuroides, Amaranthus retroflexus, Avena fatua, Digitaria sanguinalis, Echinochloa crus-galli, Lolium rigidum, Setaria viridis, Stellaria media, Tripleurospermum inodorum, Veronica persica and Fallopia convolvulus at an application rate of between 0.020 and 0.320 kg of active substance per hectare, and good crop plant compatibility with organisms such as Zea mays, Oryza sativa, Brassica napus, Glycine max and Triticum aestivum at an application rate of 0.32 kg or less per hectare.

The compounds of the invention are therefore suitable for control of unwanted plant growth by the post-emergence method.

3. Comparative Herbicidal Effect of an Inventive Compound (I-01) with Structurally Similar Compounds from WO2020/245044.

TABLE 39
Substances tested in the comparative tests

| | Structural formula |
|---|---|
| substance | |
| I-01 (inventive) | 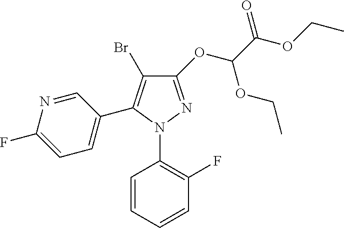 [SMILES: CCOC(=O)C(OCC)Oc1nn(-c2ccccc2F)c(-c2ccc(F)nc2)c1Br] |
| Comparative | |
| substance | |
| A | 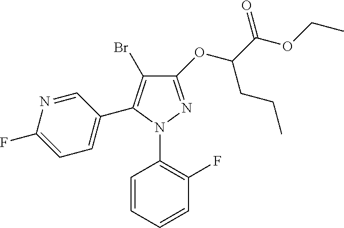 [SMILES: CCCC(Oc1nn(-c2ccccc2F)c(-c2ccc(F)nc2)c1Br)C(=O)OCC] |
| B | 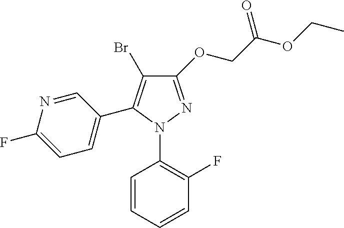 [SMILES: CCOC(=O)COc1nn(-c2ccccc2F)c(-c2ccc(F)nc2)c1Br] |
| C | 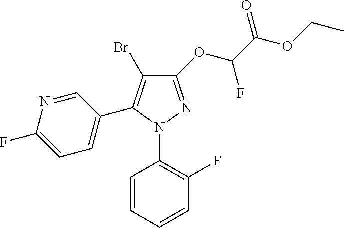 [SMILES: CCOC(=O)C(F)Oc1nn(-c2ccccc2F)c(-c2ccc(F)nc2)c1Br] |

Tables 40a and 40b below show the effects of the inventive compound (I-01) with structurally similar compounds (from WO2020/245044) on various harmful plants at an application rate corresponding to 320 g/ha or less, which have been obtained by the trial procedure specified hereinafter. The inventive compound (I-01) differs here by variance of a significant structural feature from the structurally similar compound with regard to the R² radical.

### Pre-Emergence Herbicidal Effect and Crop Plant Compatibility (PE)

Seeds of monocotyledonous and dicotyledonous weed plants and crop plants are placed in plastic or organic planting pots and covered with soil. The compounds of the invention, formulated in the form of wettable powders (WP) or as emulsion concentrates (EC), are then applied to the surface of the covering soil as aqueous suspension or emulsion with addition of 0.5% additive at a water application rate equivalent to 600 l/ha. After the treatment, the pots are placed in a greenhouse and kept under good growth conditions for the trial plants. After about 3 weeks, the effect of the preparations is scored visually in comparison with untreated controls as percentages. For example, 100% activity=the plants have died, 0% activity=like control plants.

Effects/crop compatibilities of selected compounds (Table 39) on various harmful plants at an application rate corresponding to 80 to 320 g/ha that have been obtained by the trial procedure specified hereinabove are shown in Tables 40a and 40b below.

TABLE 40a
Pre-emergence effect at 320 g/ha against various unwanted plants

| (Comparative) | | Test object | | | | | |
|---|---|---|---|---|---|---|---|
| substance | Concentration | Measurement | ALOMY | LOLRI | MATIN | SETVI | ECHCG |
| I-01 (inventive) | 320 | Effect % | 90 | 100 | 90 | 100 | 100 |
| A | 320 | Effect % | 60 | 30 | 30 | 70 | 80 |
| C | 320 | Effect % | 80 | 90 | 80 | 70 | 70 |

TABLE 40b
Pre-emergence effect at 80 g/ha against various unwanted plants

| (Comparative) | | Test object | | | | |
|---|---|---|---|---|---|---|
| substance | Concentration | Measurement | LOLRI | MATIN | ECHCG | POLCO |
| I-01 (inventive) | 80 | Effect % | 90 | 90 | 70 | 80 |
| A | 80 | Effect % | 20 | 0 | 0 | 20 |
| C | 80 | Effect % | 10 | 70 | 0 | 50 |

As shown by the results displayed in Tables 40a and 40b, inventive compound I-01, by comparison to the structurally similar compounds, has distinctly improved herbicidal efficacy against different harmful plants at an application rate of 320 g or less per hectare. Tables 41a and 41b below show the effects of the inventive compound (I-01) with structurally similar compounds (WO2020/245044) on various harmful plants at an application rate corresponding to 320 g/ha or less, which have been obtained by the trial procedure specified hereinafter. The inventive compound (I-01) differs here by variance of a significant structural feature from the structurally similar compound with regard to the R² radical.

### Post-Emergence Herbicidal Effect and Crop Plant Compatibility (PO)

Seeds of monocotyledonous and dicotyledonous weeds and crop plants are placed in sandy loam in plastic or organic planting pots, covered with soil and cultivated in a greenhouse under controlled growth conditions. 2 to 3 weeks after sowing, the trial plants are treated at the one-leaf stage. The compounds of the invention, formulated in the form of wettable powders (WP) or as emulsion concentrates (EC), are then sprayed onto the green parts of the plants as aqueous suspension or emulsion with addition of 0.5% additive at a water application rate of 600 l/ha (converted). After the test plants have been kept in the greenhouse under optimum growth conditions for about 3 weeks, the activity of the preparations is rated visually in comparison to untreated controls. For example, 100% activity=the plants have died, 0% activity=like control plants.

Effects/crop compatibilities of selected compounds (see Table 39) on various harmful plants at an application rate corresponding to 80 to 320 g/ha that have been obtained by the trial procedure specified hereinafter are shown in Tables 41a and 41b below.

TABLE 41a
Post-emergence effect at 320 g/ha against various unwanted plants

| (Comparative) | | Test object | | | | | |
|---|---|---|---|---|---|---|---|
| substance | Concentration | Measurement | ALOMY | LOLRI | AVEFA | SETVI | ECHCG |
| I-01 (inventive) | 320 | Effect % | 100 | 90 | 90 | 90 | 90 |
| A | 320 | Effect % | 50 | 20 | 40 | 70 | 80 |

TABLE 41b
Post-emergence effect at 80 g/ha against various unwanted plants

| (Comparative) | | Test object | | | | | |
|---|---|---|---|---|---|---|---|
| substance | Concentration | Measurement | ALOMY | LOLRI | AVEFA | SETVI | ECHCG |
| I-01 (inventive) | 80 | Effect % | 90 | 70 | 90 | 90 | 90 |
| A | 80 | Effect % | 0 | 0 | 30 | 20 | 50 |
| B | 80 | Effect % | 70 | 30 | 80 | 70 | 50 |
| C | 80 | Effect % | 50 | 10 | 60 | 70 | 80 |

As shown by the results displayed in Tables 41a and 41b, inventive compound I-01, by comparison to the structurally similar compounds, has distinctly improved herbicidal efficacy against different harmful plants at an application rate of 320 g or less per hectare.

1. A (1,4,5-Trisubstituted 1H-pyrazol-3-yl)oxy-2-alkoxyalkyl acid, and derivatives thereof, of the general formula (I) 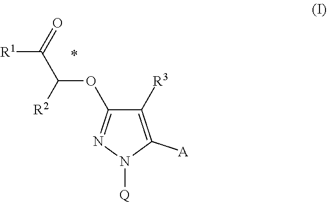
and their agrochemically acceptable salts, N-oxides, hydrates, and hydrates of the salts and N-oxides, where
A is selected from the group consisting of A1, A2 and A3 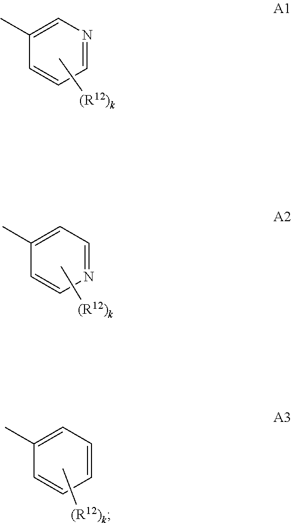
Q is selected from the group consisting of Q1-Q16: 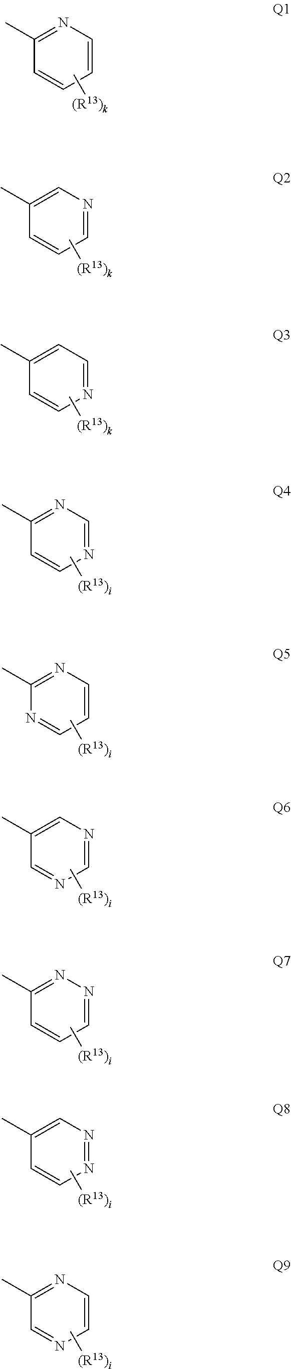 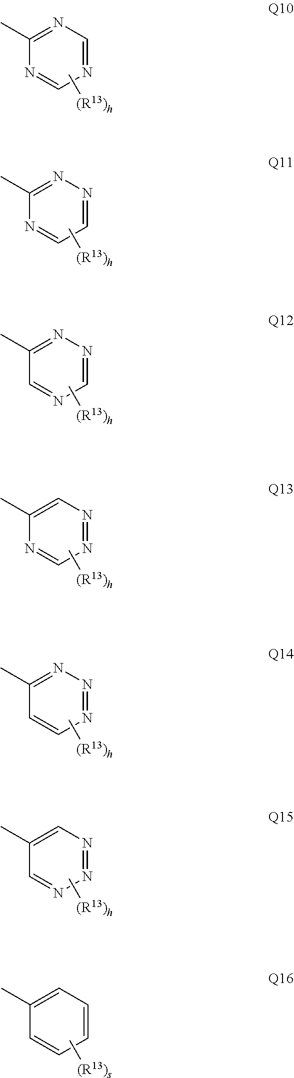
R¹ is OR^{1a}, NR⁹R¹⁰;
R^{1a} is hydrogen or
is (C₁-C₆)-alkyl, (C₃-C₆)-cycloalkyl, which is unsubstituted or in each case independently substituted by “m” radicals selected from the group consisting of COOR⁸, halogen, (C₁-C₆)-alkyl, (C₁-C₆)-haloalkyl, (C₃-C₆)-cycloalkyl, (C₁-C₆)-alkoxy, cyano and nitro or
is (C₂-C₄)-alkenyl, (C₂-C₄)-alkynyl or
is (C₁-C₆)-alkyl-SO—(C₁-C₆)-alkyl-, (C₁-C₆)-alkyl-SO₂—(C₁-C₆)-alkyl- or
is heterocyclyl, heteroaryl, aryl or
is heterocyclyl-(C₁-C₄)-alkyl-, heteroaryl-(C₁-C₄)-alkyl-, aryl-(C₁-C₄)-alkyl-, which is unsubstituted or in each case independently substituted by “m” radicals selected from the group consisting of halogen, (C₁-C₆)-alkyl, (C₁-C₆)-haloalkyl;
R⁹ is hydrogen, (C₁-C₁₂)-alkyl;
R¹⁰ is hydrogen, aryl, heteroaryl, heterocyclyl, (C₁-C₁₂)-alkyl, (C₃-C₈)-cycloalkyl, (C₃-C₈)-cycloalkyl-(C₁-C₇)-alkyl-, (C₂-C₁₂)-alkenyl, (C₈-C₇)-cycloalkenyl, (C₂-C₁₂)-alkynyl, S(O)ₙR⁵, cyano, OR⁵, SO₂NR⁶R⁷, CO₂R⁸, COR⁸, where the abovementioned alkyl, cycloalkyl, alkenyl, cycloalkenyl and alkynyl radicals are unsubstituted or each independently substituted by “m” radicals selected from the group consisting of optionally mono- or polysubstituted aryl, halogen, cyano, nitro, OR⁵, S(O)ₙR⁵, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, NR⁶SO₂R⁸, NR⁶SO₂NR⁶R⁸, C(R⁶)═NOR⁸;
or
R⁹ and R¹⁰ together with the nitrogen atom to which they are bonded form a saturated or partly or fully unsaturated five-, six- or seven-membered ring which is optionally substituted by “m” radicals from the group consisting of halogen, (C₁-C₆)-alkyl, (C₁-C₆)-haloalkyl, OR⁵, S(O)ₙR⁸, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl, aryl;
R⁶ is hydrogen, (C₁-C₆)-alkyl, (C₃-C₆)-cycloalkyl, (C₁-C₆)-haloalkyl, aryl;
R⁷ is hydrogen, (C₁-C₆)-alkyl, (C₃-C₆)-cycloalkyl, (C₃-C₄)-alkenyl, (C₃-C₄)-alkynyl;
R⁸ is hydrogen, (C₁-C₆)-alkyl, (C₃-C₆)-cycloalkyl, (C₃-C₄)-alkenyl, (C₁-C₆)-alkyl-COO(C₁-C₂)-alkyl or (C₃-C₄)-alkynyl;
R² is methoxy, ethoxy;
R³ is halogen, cyano, isocyano, NO₂, (C₁-C₆)-alkyl, (C₃-C₆)-cycloalkyl, (C₁-C₆)-haloalkyl, (C₃-C₆)-halocycloalkyl, (C₁-C₆)-alkylcarbonyl-, (C₁-C₆)-haloalkylcarbonyl-, (C₁-C₆)-alkyloxycarbonyl-, (C₂-C₃)-alkenyl, (C₂-C₃)-haloalkenyl, (C₂-C₃)-alkynyl, (C₂-C₃)-haloalkynyl, (C₁-C₆)-alkyl-S(O)ₙ and (C₁-C₆)-haloalkyl-S(O)ₙ, CHO and NH₂;
R¹² is halogen, cyano, NO₂, (C₁-C₆)-alkyl, (C₁-C₆)-haloalkyl;
R¹³ is halogen, cyano, NO₂, (C₁-C₆)-alkyl, (C₁-C₆)-haloalkyl, (C₁-C₆)-alkylcarbonyl, (C₁-C₆)-haloalkylcarbonyl, (C₁-C₆)-alkoxycarbonyl, (C₁-C₆)-alkoxy, (C₁-C₆)-haloalkoxy, (C₁-C₆)-alkylS(O)ₙ, (C₂-C₃)-alkenyl, (C₂-C₃)-haloalkenyl, (C₂-C₃)-alkynyl, (C₂-C₃)-haloalkynyl;
h is 0, 1 or 2;
i is 0, 1, 2 or 3;
k is 0, 1, 2, 3 or 4;
m is 0, 1 or 2;
n is 0, 1 or 2;
o is 0, 1 or 2;
p is 0 or 1;
q is 0 or 1;
r is 3, 4, 5 or 6;
s is 0, 1, 2, 3, 4 or 5.

2. A compound of the formula (I) according to claim 1 or an agrochemically acceptable salt, N-oxide, hydrate, or hydrate of the salt or N-oxide thereof, where
A is A1-1, A1-2, A1-3, A1-4, A2-1, A3-1, A3-2, A3-3, A3-4 and A3-5 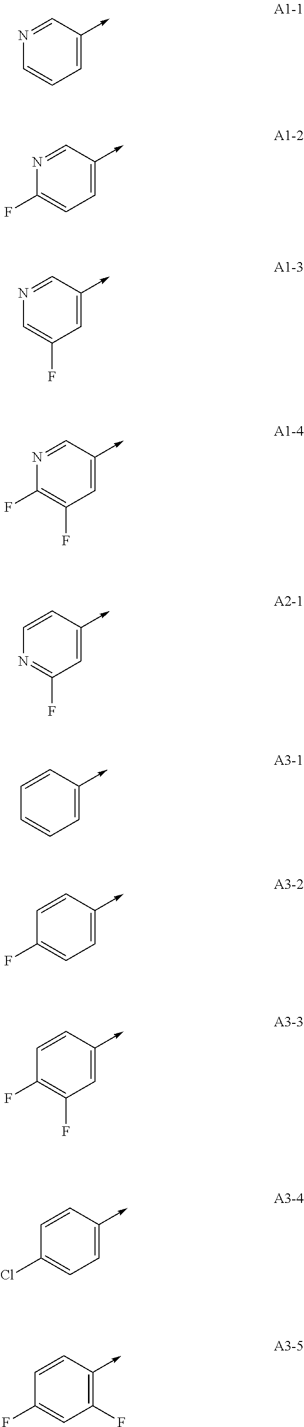
Q is selected from the group consisting of Q1, Q2, Q9 and Q16 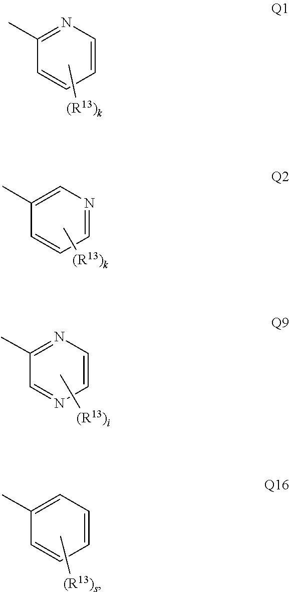
R¹ is OR^{1a}, NR⁹R¹⁰,
R^{1a} is hydrogen or
is (C₁-C₆)-alkyl, (C₃-C₆)-cycloalkyl, which is unsubstituted or in each case independently substituted by “m” radicals selected from the group consisting of COOR⁸, halogen, (C₁-C₆)-alkyl, (C₁-C₆)-haloalkyl, (C₃-C₆)-cycloalkyl, (C₁-C₆)-alkoxy, cyano and nitro or
is (C₂-C₄)-alkenyl, (C₂-C₄)-alkynyl or
is (C₁-C₆)-alkyl-SO—(C₁-C₆)-alkyl-, (C₁-C₆)-alkyl-SO₂—(C₁-C₆)-alkyl-, aryl-(C₁-C₄)-alkyl-, which is unsubstituted or in each case independently substituted by “m” radicals selected from the group consisting of halogen, (C₁-C₆)-alkyl, (C₁-C₆)-haloalkyl;
R⁹ is hydrogen, (C₁-C₆)-alkyl;
R¹⁰ is hydrogen, phenyl, (C₁-C₆)-alkyl, (C₃-C₆)-cycloalkyl, (C₃-C₆)-cycloalkyl-(C₁-C₄)-alkyl-, (C₂-C₄)-alkenyl, (C₅-C₇)-cycloalkenyl, (C₂-C₄)-alkynyl, S(O)ₙR⁸, cyano, OR⁵, SO₂NR⁶R⁷, CO₂R⁸, COR⁸, where the abovementioned alkyl, cycloalkyl, alkenyl, cycloalkenyl and alkynyl radicals are unsubstituted or each independently substituted by “m” radicals selected from the group consisting of optionally mono- or polysubstituted phenyl, halogen, cyano, nitro, OR⁵, S(O)ₙR⁸, SO₂NR⁶R⁷, CO₂R⁸, CONR⁶R⁸, COR⁶, NR⁶R⁸, NR⁶COR⁸, NR⁶CONR⁸R⁸, NR⁶CO₂R⁸, or
R⁹ and R¹⁰ together with the nitrogen atom to which they are bonded form a saturated or partly or fully unsaturated five-, six- or seven-membered ring which is optionally substituted by “m” radicals from the group consisting of halogen, (C₁-C₆)-alkyl, (C₁-C₆)-haloalkyl, OR⁵, S(O)ₙR⁸, CO₂R⁸, CONR⁶R⁸, COR⁶ and C(R⁶)═NOR⁸ and which, in addition to this nitrogen atom, contains “r” carbon atoms, “o” oxygen atoms, “p” sulfur atoms and “q” elements from the group consisting of NR⁷, CO and NCOR⁷ as ring atoms;
R⁵ is (C₁-C₆)-alkyl, (C₃-C₆)-cycloalkyl, (C₁-C₆)-haloalkyl or phenyl;
R⁶ is hydrogen, (C₁-C₆)-alkyl, (C₃-C₆)-cycloalkyl, (C₁-C₆)-haloalkyl or phenyl;
R⁷ is hydrogen, (C₁-C₆)-alkyl, (C₃-C₆)-cycloalkyl, (C₃-C₄)-alkenyl or (C₃-C₄)-alkynyl;
R⁸ is hydrogen, (C₁-C₆)-alkyl, (C₃-C₆)-cycloalkyl, (C₃-C₄)-alkenyl or (C₃-C₄)-alkynyl;
R² is methoxy, ethoxy;
R³ is halogen, cyano, isocyano, NO₂, (C₁-C₄)-alkyl, (C₃-C₆)-cycloalkyl, (C₁-C₆)-haloalkyl, (C₃-C₆)-halocycloalkyl, (C₂-C₃)-alkenyl, (C₂-C₃)-haloalkenyl, (C₂-C₃)-alkynyl, (C₂-C₃)-haloalkynyl;
R¹³ is halogen, cyano, nitro, (C₁-C₆)-alkyl, (C₁-C₆)-haloalkyl, (C₁-C₆)-alkoxy, (C₁-C₆)-haloalkoxy, (C₁-C₆)-alkylS(O)ₙ, (C₂-C₃)-alkenyl, (C₂-C₃)-haloalkenyl, (C₂-C₃)-alkynyl, (C₂-C₃)-haloalkynyl;
i is 0, 1 or 2;
k is 0, 1, 2, 3 or 4;
m is 0, 1, 2;
n is 0, 1, 2;
o is 0, 1, 2;
p is 0 or 1;
q is 0 or 1;
r is 3, 4, 5 or 6;
s is 0, 1, 2, 4, 5.

3. A compound of the formula (I) according to claim 1 or an agrochemically acceptable salt, N-oxide, hydrate, or hydrate of the salt or N-oxide thereof, where
A is A1-1, A1-2, A1-3, A1-4, A2-1, A3-1, A3-2, A3-3, A3-4 and A3-5 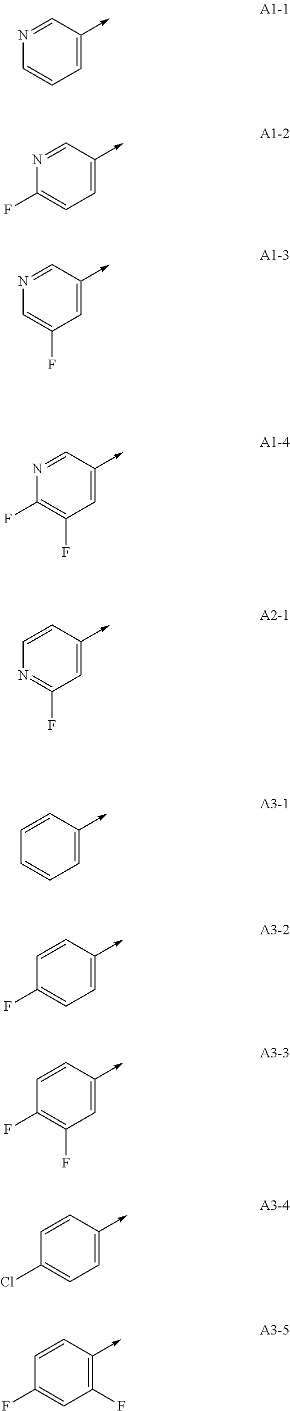
Q is selected from the group consisting of Q1, Q2, Q9 and Q16 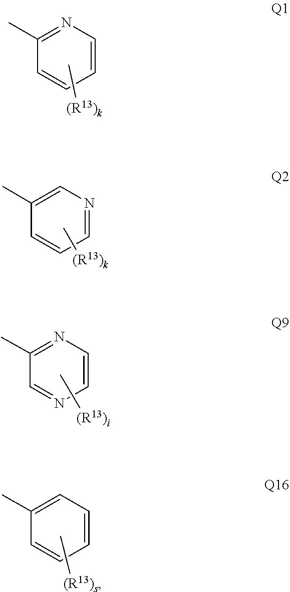
R¹ is OR^{1a}, NR⁹R¹⁰;
R^{1a} is hydrogen or
is (C₁-C₆)-alkyl, (C₃-C₆)-cycloalkyl, which is unsubstituted or in each case independently substituted by “m” radicals selected from the group consisting of COOR⁵, halogen, (C₁-C₄)-alkyl, (C₁-C₄)-haloalkyl or
is aryl-(C₁-C₄)-alkyl- which is unsubstituted or in each case independently substituted by “m” radicals selected from the group consisting of halogen, (C₁-C₄)-alkyl, (C₁-C₄)-haloalkyl;
R⁹ is hydrogen;
R¹⁰ is (C₁-C₄)-alkyl, S(O)ₙR⁵, SO₂NR⁶R⁷, CO₂R⁸, where the abovementioned radicals are unsubstituted or each independently substituted by “m” radicals selected from the group consisting of phenyl, S(O)ₙR⁵, SO₂NR⁶R⁷, CO₂R⁸, NR⁶CO₂R⁸;
R⁵ is ethyl, methyl, CF₃ or CH₂CF₃;
R⁶ is hydrogen;
R⁷ is hydrogen, methyl or ethyl;
R⁸ is methyl or ethyl;
R² is methoxy, ethoxy;
R³ is halogen, cyano, nitro, (C₁-C₄)-alkyl, (C₃-C₆)-cycloalkyl, (C₁-C₄)-haloalkyl, (C₃-C₆)-halocycloalkyl;
R¹³ is fluorine, chlorine, bromine, cyano, methyl, ethyl, methoxy, ethoxy, CF₃, OCF₃;
i is 0, 1 or 2;
k is 0, 1 or 2;
m is 0, 1 or 2;
n is 0, 1 or 2;
s is 0, 1 or 2.

4. A compound of the formula (I) according to claim 1 or an agrochemically acceptable salt, N-oxide, hydrate, or hydrate of the salt or N-oxide thereof, where
A is selected from the group consisting of
A is A1-1, A1-2, A1-3, A1-4, A2-1, A3-1, A3-2, A3-3, A3-4 and A3-5 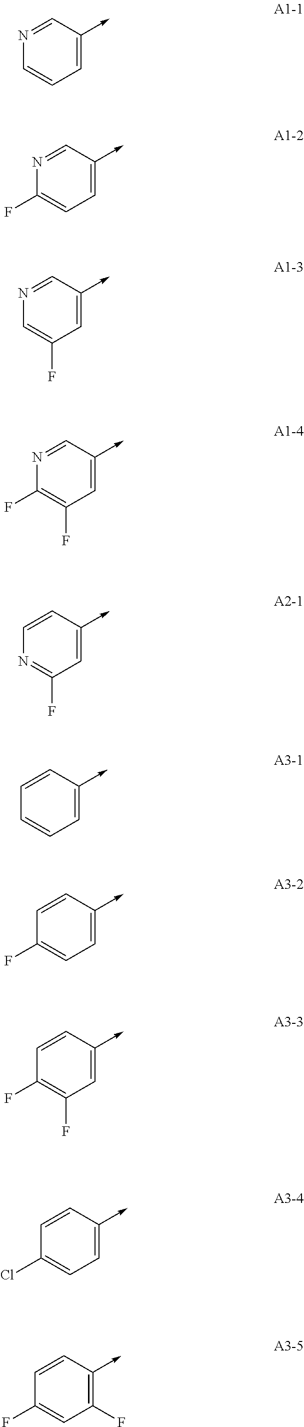
Q is selected from the group consisting of Q1, Q9 and Q16 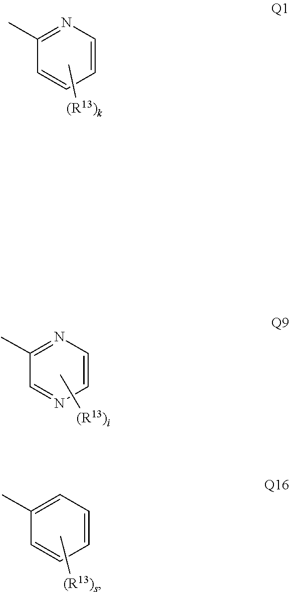
R¹ is OR^{1a},
R^{1a} is hydrogen, ethyl, methyl, —CH₂CH(CH₃)COOmethyl, —CH₂CH₂COOmethyl;
R² is ethoxy, methoxy;
R³ is chlorine, bromine, iodine, cyano, cyclopropyl, CF₂CF₃, CHF₂ or CF₃;
R¹³ is fluorine, chlorine, methyl, MeS(O), MeS or CF₃;
i is 0, 1 or 2;
k is 0, 1 or 2;
s is 0, 1 or 2.

5. A process for preparing the compounds of the formula (Ic) or an agrochemically acceptable salt thereof according to claim 1 by converting compounds of the general formulae (III) and (IV) 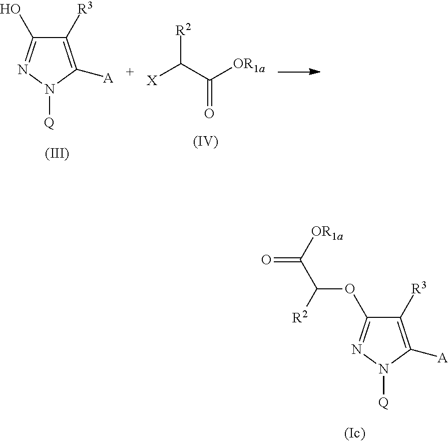
in which R², R^{1a}, R³, A, and Q have the definition given above and X is chlorine, bromine or iodine, in the presence of a sulfurizing reagent, for example phosphorus pentasulfide or Lawesson's reagent.

6. A process for preparing the compounds of the formula (Ia) or an agrochemically acceptable salt thereof according to claim 1 by converting a compound of the general formula (Ic) 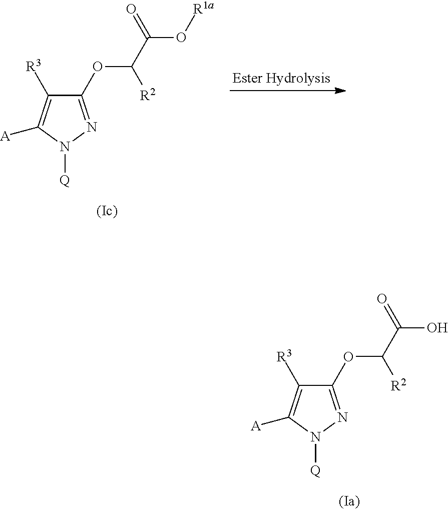
in which R², R^{1a}, R³, A and Q have the definitions given above, in the presence of a base or a Lewis acid.

7. A process for preparing the compounds of the formula (Ib) or an agrochemically acceptable salt thereof according to claim 1 by converting compounds of the general formulae (Ia) and (II) 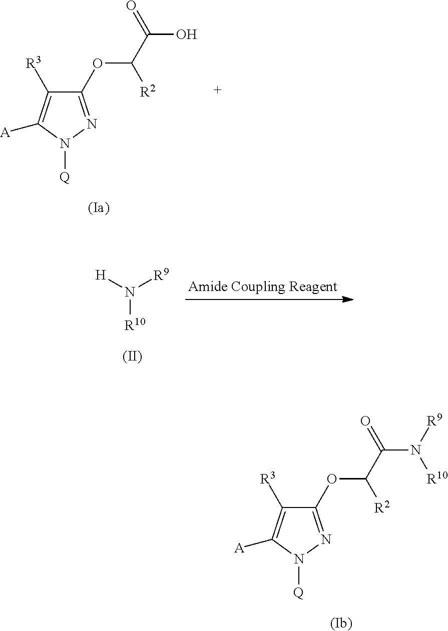
in which R⁹, R¹⁰, R², R^{1a}, R³, A and Q have the definitions given above, in the presence of an amide coupling reagent.

8. An agrochemical composition comprising a) at least one compound of the formula (I) or an agrochemically acceptable salt thereof as defined in claim 1 and b) auxiliaries and additives customary in crop protection.

9. An agrochemical composition comprising
a) at least one compound of the formula (I) or an agrochemically acceptable salt thereof as defined in claim 1,
b) one or more active agrochemical ingredients other than component a), and optionally
c) auxiliaries and additives customary in crop protection.

10. A method of controlling unwanted plants or for regulating the growth of plants, wherein an effective amount of at least one compound of the formula (I) or an agrochemically acceptable salt thereof, as defined in claim 1, is applied to the plants, the seed or the area in which the plants grow.

11. The use of compounds of the formula (I) or an agrochemically acceptable salt thereof, as defined in claim 1, as herbicides or plant growth regulators.

12. The use according to claim 11, wherein the compounds of the formula (I) or an agrochemically acceptable salt thereof are used for controlling harmful plants or for regulating growth in plant crops.

13. The use according to claim 12, wherein the crop plants are transgenic or nontransgenic crop plants.
